(19) United States
(12) Patent Application Publication
(10) Pub. No.: US 2010/0298392 A1
(43) Pub. Date: Nov. 25, 2010

(54) Isoindoline Derivatives For The Treatment Of Arrhythmias

(75) Inventors: Annika Bjore, Molndal (SE); Jonas Bostrom, Molndal (SE); Ojvind Davidsson, Molndal (SE); Hans Emtenas, Molndal (SE); Ulrik Gran, Molndal (SE); Tommy Iliefski, Molndal (SE); Johan Kajanus, Molndal (SE); Roine Olsson, Molndal (SE); Lars Sandberg, Sodertalje (SE); Gert Strandlund, Lindome (SE); Johan Sundell, Molndal (SE); Zhong-Qing Yuan, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/373,065

(22) PCT Filed: Jul. 12, 2007
(86) PCT No.: PCT/SE2007/000683

§ 371 Date: Feb. 9, 2009

Related U.S. Application Data
(60) Provisional application No. 60/830,243, filed on Jul. 12, 2006.
(60) Provisional application No. 60/913,367, filed on Apr. 23, 2007.

(51) Int. Cl. A61K 31/4035; C07D 413/12; C07D 209/46; C07D 405/12; A61K 31/422; A61P 9/06

(52) U.S. Cl. 514/380; 548/246; 548/472; 548/454; 514/416; 514/414

(57) ABSTRACT

There is provided compounds of formula I, wherein R¹ to R⁷ have meanings given in the description, which are useful in the prophylaxis and in the treatment of arrhythmias, in particular atrial and ventricular arrhythmias.

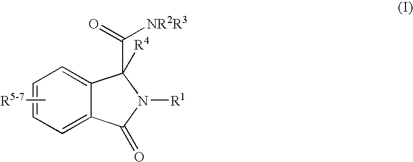

### FIELD OF THE INVENTION

This invention relates to novel pharmaceutically useful 3-oxoisoindoline-1-carboxamide compounds, in particular compounds that are useful in the treatment of cardiac arrhythmias.

### BACKGROUND

Cardiac arrhythmias may be defined as abnormalities in the rate, regularity, or site of origin of the cardiac impulse or as disturbances in conduction which causes an abnormal sequence of activation. Arrhythmias may be classified clinically by means of the presumed site of origin (i.e. as supraventricular, including atrial and atrioventricular, arrhythmias and ventricular arrhythmias) and/or by means of rate (i.e. bradyarrhythmias (slow) and tachyarrhythmias (fast)).

In the treatment of cardiac arrhythmias, the negative outcome in clinical trials (see, for example, the outcome of the Cardiac Arrhythmia Suppression Trial (CAST) reported in New England Journal of Medicine, 321, 406 (1989)) with “traditional” antiarrhythmic drugs, which act primarily by slowing the conduction velocity (class I antiarrhythmic drugs), has prompted drug development towards compounds which selectively delay cardiac repolarization, thus prolonging the QT interval. Class III antiarrhythmic drugs may be defined as drugs which prolong the trans-membrane action potential duration (which can be caused by a block of outward K⁺ currents or from an increase of inward ion currents) and refractoriness, without affecting cardiac conduction. The rapidly and slow activating delayed rectifier potassium currents I_{Kr} and I_{Ks}, respectively, are the main currents involved in the overall repolarisation process during the action potential plateau and most class III agents predominantly block I_{Kr} One of the key disadvantages of hitherto known drugs which act by delaying repolarization by a block of I_{Kr} (class III or otherwise) is that almost all are known to exhibit a unique form of ventricular proarrhythmia known as torsades de pointes (turning of points), which may, on occasion be fatal. From the point of view of safety, the minimisation of this phenomenon (which has also been shown to be exhibited as a result of administration of non-cardiac drugs such as phenothiazines, tricyclic antidepressants, antihistamines and antibiotics) is a key problem to be solved in the provision of effective antiarrhythmic drugs. In human atrial myocytes, an ultra-rapidly activating delayed rectifier potassium current, I_{Kur} also known as Iₛₒ or Iₛᵤₛ, has been identified. The gene most likely coding the I_{Kur} channel protein has been identified, and is termed Kv1.5 (Wang et al (1993) Circ Res73:1061-0176, Feng et al (1997) Circ Res 80:572-579). Due to the slow inactivation of the current, I_{Kur} persists during the plateau phase and contributes significantly to action potential repolarisation in atrial myocytes. Most interestingly, voltage clamp studies investigating repolarising currents have failed to demonstrate the presence of I_{Kur} in human ventricular myocytes (Amos et al J Physiol (1996) 491(1):31-50). Thus, a selective blocker of I_{Kur}, that is a compound which block Kv1.5, is of great interest for the therapy of atrial arrhythmia, since such an agent should delay repolarisation in human atrial myocardium only, circumventing ventricular proarrhythmias (i.e. torsades de pointes,) associated with delayed ventricular repolarisation.

Kv1.5 blockers exhibiting these properties have been described (Peukert et al J Med Chem (2003) 46:486-498; Knobloch et al Naunyn-Schmiedeberg's Arch Pharmacol (2002) 366:482-487).

Some 3-oxoisoindoline-1-carboxamide derivatives are known. 3-oxoisoindoline-1-carboxamide derivatives are an ideal target for multicomponent reactions (MCRs). Tetrahedron Letters (1998), 39(18), 2725-2728 discloses some 3-oxoisoindoline-1-carboxamide derivatives prepared by so-called Ugi reactions (N-tert-butyl-3-oxo-2-propylisoindoline-1-carboxamide; N-tert-butyl-1-methyl-3-oxo-2-propylisoindoline-1-carboxamide; N,1-dimethyl-3-oxo-2-propylisoindoline-1-carboxamide; N-cyclohexyl-3-oxo-2-propylisoindoline-1-carboxamide; 2-benzyl-N-tert-butyl-3-oxoisoindoline-1-carboxamide; 2-benzyl-N,1-dimethyl-3-oxoisoindoline-1-carboxamide; 2-benzyl-N-tert-butyl-1-methyl-3-oxoisoindoline-1-carboxamide; 2-benzyl-N,1-dimethyl-3-oxoisoindoline-1-carboxamide.

Also Journal of Organic Chemistry (1999), 64(3), 1074-1076 discloses such compounds (tert-butyl{4-[1-(tert-butylcarbamoyl)-3-oxo-1,3-dihydro-2H-isoindol-2-yl]butyl}carbamate; 2-benzyl-3-oxo-N-(2-phenylethyl)isoindoline-1-carboxamide; 2-benzyl-N-butyl-3-oxoisoindoline-1-carboxamide; 2-benzyl-N-(2-methoxyethyl)-3-oxoisoindoline-1-carboxamide; 2-(2-hydroxyethyl)-3-oxo-N-(2-phenylethyl)isoindoline-1-carboxamide; N-butyl-2-(2-hydroxyethyl)-3-oxoisoindoline-1-carboxamide; 2-(2-hydroxyethyl)-N-(2-methoxyethyl)-3-oxoisoindoline-1-carboxamide; 2-[3-(1H-imidazol-1-yl)propyl]-3-oxo-N-(2-phenylethyl)isoindoline-1-carboxamide; N-butyl-2-[3-(1H-imidazol-1-yl)propyl]-3-oxoisoindoline-1-carboxamide; 2-[3-(1H-imidazol-1-yl)propyl]-N-(2-methoxyethyl)-3-oxoisoindoline-1-carboxamide; 2-cyclohexyl-3-oxo-N-(2-phenylethyl)isoindoline-1-carboxamide; N-butyl-2-cyclohexyl-3-oxoisoindoline-1-carboxamide; 2-cyclohexyl-N-(2-methoxyethyl)-3-oxoisoindoline-1-carboxamide) and in Bioorganic & Medicinal Chemistry Letters (2002), 12(14), 1813-1816 (2-cyclohexyl-N-hexyl-3-oxoisoindoline-1-carboxamide; N,2-dihexyl-3-oxoisoindoline-1-carboxamide; N-hexyl-2-(2-hydroxyethyl)-3-oxoisoindoline-1-carboxamide; N-hexyl-2-(4-hydroxybutyl)-3-oxoisoindoline-1-carboxamide; N,2-dicyclohexyl-3-oxoisoindoline-1-carboxamide; N-cyclohexyl-2-hexyl-3-oxoisoindoline-1-carboxamide; N-cyclohexyl-2-(2-hydroxyethyl)-3-oxoisoindoline-1-carboxamide; N-cyclohexyl-2-(4-hydroxybutyl)-3-oxoisoindoline-1-carboxamide; tert-butyl{4-[1-(Cyclohexylcarbamoyl)-3-oxo-1,3-dihydro-2H-isoindol-2-yl]butyl}carbamate; N-adamantan-1-yl-2-cyclohexyl-3-oxoisoindoline-1-carboxamide; N-adamantan-1-yl-2-hexyl-3-oxoisoindoline-1-carboxamide; N-adamantan-1-yl-2-(2-hydroxyethyl)-3-oxoisoindoline-1-carboxamide; N-adamantan-1-yl-2-(2-morpholin-4-ylethyl)-3-oxoisoindoline-1-carboxamide; N-(2,6-dimethylphenyl)-2-hexyl-3-oxoisoindoline-1-carboxamide). Also Tetrahedron, vol. 53, No. 19, 6653-6679 discloses 3-oxoisoindoline-1-carboxamide derivatives prepared by so-called Ugi reactions (6-{[(2-allyl-1-methyl-3-oxo-2,3-dihydro-1H-isoindol-1-yl)carbonyl]amino}hexanoic acid). No pharmaceutical use of the prepared compounds is contemplated in those references. Tetrahedron Letters (2002), 43(6), 943-946 discloses some 3-oxoisoindoline-1-carboxamide derivatives prepared by intramolecular Diels-Alder type reactions (N,2-dibenzyl-5-hydroxy-4-methyl-3-oxoisoindoline-1-carboxamide; N-benzyl-2-tert-butyl-5-hydroxy-3-oxo-4-phenylisoindoline-1-carboxamide; N-benzyl-2-tert-butyl-5-hydroxy-4-methyl-3-oxoisoindoline-1-carboxamide; N,2-dibenzyl-5-hydroxy-3-oxo-4-phenylisoindoline-1-carboxamide; N-benzyl-2-tert-butyl-5-hydroxy-3-oxoisoindoline-1-carboxamide). Also Journal of Organic Chemistry (2004), 69(4), 1207-1214 discloses the compound (N,2-dibenzyl-5-[(2-nitrophenyl)sulfonyl]amino}-3-oxoisoindoline-1-carboxamide). No pharmaceutical use of the prepared compounds is contemplated.

Journal of Heterocyclic Chemistry (1997), 34(4), 1371-1374 discloses some symmetrically substituted 3-oxoisoindoline-1-carboxamide derivatives prepared by carbonylative cyclization of 2-bromobenzaldehyde with primary amines (N,2-dibenzyl-3-oxoisoindoline-1-carboxamide ; N,2-diethyl-3-oxoisoindoline-1-carboxamide; N,2-dibutyl-3- oxoisoindoline-1-carboxamide; N,2-didodecyl-3-oxoisoindoline-1-carboxamide; N,2-bis(4-methoxybenzyl)-3-oxoisoindoline-1-carboxamide; 3-oxo-N,2-dipropylisoindoline-1-carboxamide; N,2-diheptyl-3-oxoisoindoline-1-carboxamide; 3-oxo-N,2-diphenylisoindoline-1-carboxamide). No pharmaceutical use of the prepared compounds is contemplated. Some additional 3-oxoisoindoline-1-carboxamide derivatives are disclosed in Zhurnal Obshchei Khimii (1965), 1(7), 1292-7; Yakugaku Zasshi (1969), 89(3), 418-21; Journal of the Chemical Society, Perkin Transactions 1: Organic and Bio-Organic Chemistry (1972-1999) (1980), (4), 846-8 (2-(4-nitrophenyl)-3-(pyrrolidin-1-ylcarbonyl)isoindolin-1-one); EP1566378 A1 (2-(3-fluorophenyl)-5,6-dimethyl-3-[(4-methylpiperazin-1-yl)-carbonyl]isoindolin-1-one); EP 1661898; CHEMCATS (Chemical Catalogs Online provided by STN) (N-[2-(3,4-dimethoxylphenyl)ethyl]-3-oxo-2-(1-phenylethyl)isoindoline-1 -carboxamide; N-cyclopentyl-2-(3-methoxybenzyl)-3-oxoisoindoline-1-carboxamide; 2-(1,3-benzodioxol-5-ylmethyl)-N-{[(4-methylphenyl)sulfonyl]methyl}-3-oxoisoindoline-1-carboxamide; N-cyclohexyl-3-oxo-2-(2-thienylmethyl)isoindoline-1-carboxamide; 2-benzyl-N-cyclohexyl-3-oxoisoindoline-1-carboxamide; N-{[(4-methylphenyl)sulfonyl]methyl }-3-oxo-2-(2-thienylmethyl)isoindoline-1-carboxamide ; 2-(4-chlorobenzyl)-N-{[(4-methylphenyl)sulfonyl]methyl}-3-oxoisoindoline-1-carboxamide; N-cyclohexyl-2-(2-furylmethyl)-3-oxoisoindoline-1-carboxamide; 2-(4-chlorobenzyl)-N-cyclohexyl-3-oxoisoindoline-1-carboxamide; WO03/040096 (tert-butyl{1-benzyl-2-hydroxy-3-[(2-hydroxy-3-{[(3-oxo-2,3-dihydro-1H-isoindol-1-yl)carbonyl]amino}-4-phenylbutyl)amino]propyl}carbamate); U.S. Pat. No. 5,559,256; Chemical & Pharmaceutical Bulletin (1988), 36(1), 190-201; Journal of the Chemical Society (1972-1999), (1972), (6), 835-840; Justus Liebigs Annalen Der Chemie (1978), vol 2, 283-288 (1-hydroxy-2-methyl-3-oxo-N-(pyridin-2-ylmethyl)isoindoline-1-carboxamide; N-[3-(dimethylamino)propyl]1-hydroxy-2-(2-hydroxyethyl)-3-oxoisoindoline-1-carboxamide; N-(3-azepan-1-ylpropyl)-1-hydroxy-3-oxo-2-phenylisoindoline-1-carboxamide, 1-hydroxy-2-methyl-3-oxoisoinoline-1-carbohydrazide; 1-hydroxy-3-oxo-phenylisoindoline-1-carbohydrazide); Zeitschrift for Naturforschung. B, 1993, vol 48:8, 1094-1104 (2-benzoyl-1-hydroxy-3-oxo-N-phenylisoindoline-1-carboxamide); J. Prakt. Chem. 2, 159, 1941, 241, 244, 254; Heterocycles Vol 38; No 8; 1994, 1828-1838; J. Org. Chem. 17, 1952, 4, 8, 1-13; Tetrahedron, EN, 53, 19, 1997, 6653-6680; Tetrahedron Letters, vol 38, No 3, 1997, 359-362 (6-{[(2-allyl-1-methyl-3-oxo-2,3-dihydro-1H-isoindol-1-yl)carbonyl]amino}hexanoic acid and 6-{[(1-methyl-2-octyl-3-oxo-2,3-dihydro-1H-isoindol-1-yl)carbonyl]amino}hexanoic acid). EP 1566378 A1 discloses isoindoline derivatives having anestetic effect, EP 1661898 A1 isoindoline derivatives to be used in treatment of cancer, and EP 1749817 A1 isoindoline derivatives controlling neturophatic pain. US 2007/0099930 discloses substituted dihydroisoindolones having an effect as glucokinase modulators. Further isoindoline derivatives are described in SYNTHESIS 2006, No 23, pp 4046-4052 (methyl[1-(tert-butylcarbamoyl)-3-oxo-1,3-dihydro-2H-isoindol-2-yl]acetate); and in J. Org. Chem 2006, 71, 9544-9547 (N1-cyclopentyl-N4-(2,6-difluorophenyl)-2-(2,4-dimethylphenyl)-5-methyl-3-oxoisoindoline-1,4-dicarboxamide).

The compounds disclosed in the documents listed above are disclaimed from the compound claims of the present application by proviso b). Compounds of proviso c) did not demonstrate activity at the concentrations at which they were tested.

Copending case no U.S. 60/830,186 describes proviso a).

Further, the following compound is known in Chemical Abstracts but no reference is given: 3-oxo-N,2-diphenylisoindoline-1-carboxamide.

We have surprisingly found that a novel group of 3-oxoisoindoline-1-carboxamide compounds exhibit electrophysiological activity, preferably Kv1.5 blocking activity, and are therefore expected to be useful in the treatment of cardiac arrhythmias.

### DISCLOSURE OF THE INVENTION

According to the invention there is provided a compound of formula I,

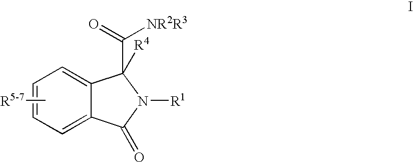

or a pharmaceutically acceptable salt thereof.

wherein

R¹ represents C₁-C₁₂ alkyl (which alkyl group is optionally substituted by one or more groups selected from halogen, C₂-C₆ alkenyl, C₃-C₈ cycloalkyl, cyano, oxo, —OR⁸, —COR⁹, —SR¹⁰, —COXR¹¹, —N(R^{12a}),(R^{12b}), —N(R^{13a})C(O)OR^{13b}, —OC(O)N(R^{14a})(R^{14b}), —SO₂R¹⁵, aryl or Het¹); further R¹ represents aryl or Het²;

R⁸ to R¹¹, R^{13a}, R^{13b}, R¹⁵ independently represent, at each occurrence, hydrogen, C₁-C₆ alkyl, aryl or Het⁹ (which C₁-C₆ alkyl, aryl and Het⁹ groups are optionally substituted with one or more substituents selected from —OH, halogen, cyano, nitro, C₁-C₆ alkyl, aryl and Het¹⁰);

R^{12a} and R¹² independently represent, at each occurrence, hyhdrogen, C₁-C₆ alkyl, aryl or Het¹¹ (which C₁-C₆ alkyl, aryl and Het¹¹ groups are optionally substituted with one or more substituents selected from —OH, halogen, cyano, nitro, C₁-C₆ alkyl, aryl and Het¹²), or together represent C₃-C₆ alkylene, optionally interrupted by an O atom;

R^{14a} and R^{14b} independently represent, at each occurrence, hydrogen, C₁-C₆ alkyl, aryl or Het¹³ (which C₁-C₆ alkyl, aryl and Het¹³ groups are optionally substituted with one or more substituents selected from —OH, halogen, cyano, nitro, C₁-C₆ alkyl, aryl and Het¹⁴), or together represent C₃-C₆ alkylene, optionally interrupted by an O atom;

R² represents C₁-C₁₂ alkyl (which alkyl group is optionally substituted by one or more groups selected from halogen, —OR¹⁶, —COR¹⁷, C₂-C₆ alkenyl, C₃-C₈ cycloalkyl, cyano, trialkylsilyl, —COXR¹⁸, aryl or Het³);

further R² represents —(CH₂)ₖN(R^{19a})(R^{19b}), —(CH₂)ₖNR^{20a}C(O)N(R^{20b})(R^{20c}), —(CH₂)ₙNR^{2la}SO₂R^{21b}, —(CH₂)ₙSO₂R²², —(CH₂)ₖN(R^{23a})C(O)OR^{23b}, —OC(O)N(R^{24a})(R^{24b}), C₃-C₈ cycloalkyl, aryl or Het⁴;

R¹⁶ to R¹⁸, R²¹,R²², R^{23a}, R^{23b} independently represent, at each occurrence, hydrogen, C₁-C₆ alkyl, aryl or Het¹⁵ (which C₁-C₆ alkyl, aryl and Het¹⁵ groups are optionally substituted with one or more substituents selected from —OH, halogen, cyano, nitro, C₁-C₆ alkyl, aryl and Het¹⁶);

R^{19a} and R^{19b} independently represent, at each occurrence, hydrogen, C₁-C₆ alkyl, aryl or Het¹⁹ (which C₁-C₆ alkyl, aryl and Het¹⁹ groups are optionally substituted with one or more substituents selected from —OH, halogen, cyano, nitro, C₁-C₆ alkyl, aryl and Het²⁰) or together represent C₃-C₆ alkylene, optionally interrupted by an O atom;

R^{20a}, R^{20b} and R^{20c} independently represent, at each occurrence, hydrogen, C₁-C₆ alkyl, aryl or Het²¹ (which C₁-C₆ alkyl, aryl and Het²¹ groups are optionally substituted with one or more substituents selected from —OH, halogen, cyano, nitro, C₁-C₆ alkyl, aryl and Het²²);

R^{20b} and R^{20c} may together represent C₃-C₆ alkylene, optionally interrupted by an O atom;

R³ represents hydrogen, C₁-C₁₂ alkyl (which alkyl group is optionally substituted by one or more groups selected from halogen, —OR²⁵, —COR²⁶, C₂-C₆ alkenyl, C₃-C₈ cycloalkyl, trialkylsilyl, —COXR²⁷, aryl or Het⁵);

further R³ represents —(CH₂)ₖN(R^{28a})(R^{28b}), —(CH₂)ₖN(R^{29a})C(O)N(R^{29b})(R^{29c}), —(CH₂)ₙNR^{30a}SO₂R^{30b}, —(CH₂)ₙSO₂R³¹, —(CH₂)ₖN(R^{32a})C(O)OR^{32b}, —OC(O)N(R^{33a})(R^{33b}), C₃-C₈ cycloalkyl, aryl or Het⁶;

R²⁵ to R²⁷, R³⁰, R³¹, R^{32a}, R^{32b} independently represent, at each occurrence, hydrogen, C₁-C₆ alkyl, aryl or Het²³ (which C₁-C₆ alkyl, aryl and Het²³ groups are optionally substituted with one or more substituents selected from —OH, halogen, cyano, nitro, C₁-C₆ alkyl, aryl and Het²⁴);

R^{28a} and R^{28b} independently represent, at each occurrence, hydrogen, C₁-C₆ alkyl, aryl or Het²⁵ (which C₁-C₆ alkyl, aryl and Het²⁵ groups are optionally substituted with one or more substituents selected from —OH, halogen, cyano, nitro, C₁-C₆ alkyl, aryl and Het²⁶), or together represent C₃-C₆ alkylene, optionally interrupted by an O atom;

R^{33a} and R^{33b} independently represent, at each occurrence, hydrogen, C₁-C₆ alkyl, aryl or Het²⁷ (which C₁-C₆ alkyl, aryl and Het²⁷ groups are optionally substituted with one or more substituents selected from —OH, halogen, cyano, nitro, C₁-C₆ alkyl, aryl and Het²⁸) or together represent C₃-C₆ alkylene, optionally interrupted by an O atom;

R^{29a}, R^{29b} , and R^{29c} independently represent, at each occurrence, hydrogen, C₁-C₆ alkyl, aryl or Het²⁹ (which C₁-C₆ alkyl, aryl and Het²⁹ groups are optionally substituted with one or more substituents selected from —OH, halogen, cyano, nitro, C₁-C₆ alkyl, aryl and Het³⁰); R^{29b} and R^{29c} may together represent C₃-C₆ alkylene, optionally interrupted by an O atom;

R⁴ represents hydrogen, —OH, aryl, C₁-C₆ alkyl (which alkyl group is optionally substituted by one or more groups selected from halogen, hydroxy, C₂-C₄ alkenyl, trialkylsilyl), —OR³⁴, —(CH₂)ₘR³⁵;

R³⁴ independently represent, at each occurrence, hydrogen, C₁-C₆ alkyl, aryl or Het³¹ (which C₁-C₆ alkyl, aryl and Het³¹ groups are optionally substituted with one or more substituents selected from —OH, halogen, cyano, nitro, C₁-C₆ alkyl, aryl and Het³²);

R³⁵ independently represent aryl or Het³³(which aryl and Het³³ groups are optionally substituted with one or more substituents selected from —OH, halogen, cyano, nitro, C₁-C₆ alkyl, aryl and Het³⁴);

R⁵ to R⁷ independently represent, at each occurence, hydrogen, —OH, halogen, cyano, nitro, C₁₋₆ alkyl, —OR³⁶, —N(R^{37a})(R^{37b}), —C(O)R³⁸, —C(O)OR³⁹, —C(O)N(R^{4O})(R^{40b}), —NC(O)OR⁴¹, —OC(O)N(R^{42a})(R^{42b}), —N(R^{43a})C(O)R^{43b}, —N(R^{44a})S(O)²R^{44b}, —S(O)₂R⁴⁵, —OS(O)₂R⁴⁶, (CH₂)ₙN(R^{47a})(R^{47b}), —(CH₂)ₙNR^{48a}C(O)N(R^{48b})(R^{48c}), CH₂)ₙNR^{49a}SO₂R^{49b}, trialkylsilyl, aryl or Het⁷;

R³⁶, R³⁸, R³⁹, R⁴¹, R⁴³, R^{44a}, R^{44b}, R⁴⁵, R⁴⁶, R^{49a} and R^{49b} independently represent, at each occurrence, hydrogen, C₁-C₆ alkyl, aryl or Het³⁵ (which C₁-C₆ alkyl, aryl and Het³⁵ groups are optionally substituted with one or more substituents selected from —OH, halogen, cyano, nitro, C₁-C₆ alkyl, aryl and Het³⁶);

R^{37a} and R^{37b} independently represent, at each occurrence, hydrogen, C₁-C₆ alkyl, aryl or Het³⁷ (which C₁-C₆ alkyl, aryl and Het³⁷ groups are optionally substituted with one or more substituents selected from —OH, halogen, cyano, nitro, C₁-C₆ alkyl, aryl and Het³⁸), or together represent C₃-C₆ alkylene, optionally interrupted by an O atom;

R^{40a} and R^{40b} independently represent, at each occurrence, hydrogen, C₁-C₆ alkyl, aryl or Het³⁹ (which C₁-C₆ alkyl, aryl and Het³⁹ groups are optionally substituted with one or more substituents selected from —OH, halogen, cyano, nitro, C₁-C₆ alkyl, aryl and Het⁴⁰), or together represent C₃-C₆ alkylene, optionally interrupted by an O atom;

R^{42a} and R^{42b} independently represent, at each occurrence, hydrogen, C₁-C₆ alkyl, aryl or Het⁴¹ (which C₁-C₆ alkyl, aryl and Het⁴¹ groups are optionally substituted with one or more substituents selected from —OH, halogen, cyano, nitro, C₁-C₆ alkyl, aryl and Het⁴²), or together represent C₃-C₆ alkylene, optionally interrupted by an O atom;

R^{47a} and R^{47b} independently represent, at each occurrence, hydrogen, C₁-C₆ alkyl, aryl or Het⁴³ (which C₁-C₆ alkyl, aryl and Het⁴³ groups are optionally substituted with one or more substituents selected from —OH, halogen, cyano, nitro, C₁-C₆ alkyl, aryl and Het⁴⁴), or together represent C₃-C₆ alkylene, optionally interrupted by an O atom;

R^{48a}, R^{48b} and R^{48c} independently represent, at each occurrence, hydrogen, C₁-C₆ alkyl, aryl or Het⁴⁵ (which C₁-C₆ alkyl, aryl and Het⁴⁵ groups are optionally substituted with one or more substituents selected from —OH, halogen, cyano, nitro, C₁-C₆ alkyl, aryl and Het⁴⁶);

R^{48b} and R^{48c} may together represent C₃-C₆ alkylene, optionally interrupted by an O atom;

aryl is, at each occurrence, optionally substituted by —OH, halogen, cyano, nitro, C₁-C₆ alkyl, C₃-C₈ cycloalkyl, C₂-C₆ alkenyl, aryl, Het⁸, —OR⁵⁰, —(CH₂)ₘR⁵¹, —SR⁵², —C(O)R⁵³, —COXR⁵⁴, —N(R^{55a})(R^{55b}), SO₂R⁵⁶, —OS(O)₂R⁵⁷, —(CH₂)ₘN(R^{58a})(R^{58b}), —CH₂)ₘNR^{59a}C(O)N(R^{59b})(R^{59c}), —C(O)OR⁶⁰, —C(O)N(R^{61a})(R^{61b}, —N(R^{62a}C(O)R^{62b}, —N(R^{63a})C(O)OR^{63b}, —OC(O)N(R^{64a})(R^{64b}), —N(R^{65a})S(O)₂R^{65b} and OC(O)R⁶⁶;

R⁵⁰ to R⁵⁴, R⁵⁶, R⁵⁷, R⁶⁰, R^{62a}, R^{62b}, R^{63a}, R^{63b}, R^{65a}, R^{65b} and R⁶⁶ independently represent, at each occurrence, hydrogen, C₁-C₆ alkyl, aryl or Het⁴⁷ (which C₁-C₆ alkyl, aryl and Het⁴⁷ groups are optionally substituted with one or more substituents selected from —OH, halogen, cyano, nitro, C₁-C₆ alkyl, aryl and Het“);

R⁵¹ independently represent aryl or Het⁴⁹ (which aryl and Het⁴⁹ groups are optionally substituted with one or more substituents selected from —OH, halogen, cyano, nitro, C₁-C₆ alkyl, aryl and Het⁵⁰);

R^{55a} and R^{55b} independently represent, at each occurrence, hydrogen, C₁-C₆ alkyl, aryl or Het⁵¹ (which C₁-C₆ alkyl, aryl and Het⁵¹ groups are optionally substituted with one or more substituents selected from —OH, halogen, cyano, nitro, C₁-C₆ alkyl, aryl and Het⁵²), or together represent C₃-C₆ alkylene, optionally interrupted by an O atom;

R^{58a} and R^{58b} independently represent, at each occurrence, hydrogen, C₁-C₆ alkyl, aryl or Het⁵³ (which C₁-C₆ alkyl, aryl and Het⁵³ groups are optionally substituted with one or more substituents selected from —OH, halogen, cyano, nitro, C₁-C₆ alkyl, aryl and Het⁵⁴), or together represent C₃-C₆ alkylene, optionally interrupted by an O atom;

R^{59a}, independently represent, at each occurrence, hydrogen, C₁-C₆ alkyl, aryl or Het⁵⁵ (which C₁-C₆ alkyl, aryl and Het⁵⁵ groups are optionally substituted with one or more substituents selected from —OH, halogen, cyano, nitro, C₁-C₆ alkyl, aryl and Het⁵⁶);

R^{59b} and R^{59c} may together represent C₃-C₆ alkylene, optionally interrupted by an O atom;

R^{61a} and R^{61b} independently represent, at each occurrence, hydrogen, C₁-C₆ alkyl, aryl or Het⁵⁷ (which C₁-C₆ alkyl, aryl and Het⁵⁷ groups are optionally substituted with one or more substituents selected from —OH, halogen, cyano, nitro, C₁-C₆ alkyl, aryl and Het⁵⁸);

or together represent C₃-C₆ alkylene, optionally interrupted by an O atom;

R^{64a} and R^{64b} independently represent, at each occurrence, hydrogen, C₁-C₆ alkyl, aryl or Het⁵⁹ (which C₁-C₆ alkyl, aryl and Het⁵⁹ groups are optionally substituted with one or more substituents selected from —OH, halogen, cyano, nitro, C₁-C₆ alkyl, aryl and Het60);

Het¹ to Het⁶⁰ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₂₋₆ alkenyl, aryl, a further Het, —OR⁶⁷, —(CH₂)ₘR⁶⁸, —SR⁶⁹, —COXR⁷⁰, —N(R^{7la})(R^{71b})SO₂R⁷², —(CH₂)ₘN(R^{73a})(R^{73b}), —(CH₂)ₘNR^{74a}C(O)N(R^{74b})(R^{74c}), —C(O)R⁷⁵, —C(O)OR⁷⁶, —C(O)N(R^{77a})(R^{77b}), —N(R^{78a})C(O)R^{78b}, —N(R^{79a})S(O)₂R^{79b}, OC(O)R⁸⁰, —NC(O)OR⁸¹, —OC(O)N(R^{82a}(R^{82b});

R⁶⁷, R⁶⁹, R⁷⁰, R⁷², R⁷⁵, R⁷⁶, R^{78a}, R^{78b}, R^{79a}, R^{79b}, R⁸⁰ or r⁸¹ independently represent, at each occurrence, hydrogen, C₁-C₆ alkyl, aryl or Het⁶¹ (which C₁-C₆ alkyl, aryl and Het⁶¹ groups are optionally substituted with one or more substituents selected from —OH, halogen, cyano, nitro, C₁-C₆ alkyl, aryl and Het⁶²);

R⁶⁸ represents aryl or Het⁶³ (which aryl and Het⁶³ groups are optionally substituted with one or more substituents selected from —OH, halogen, cyano, nitro, C₁-C₆ alkyl, aryl and Het⁶⁴);

R^{71a} and R^{71b} independently represent, at each occurrence, hydrogen, C₁-C₆ alkyl, aryl or Het⁶⁵ (which C₁-C₆ alkyl, aryl and Het⁶⁵ groups are optionally substituted with one or more substituents selected from —OH, halogen, cyano, nitro, C₁-C₆ alkyl, aryl and Het⁶⁶), or together represent C₃-C₆ alkylene, optionally interrupted by an O atom;

R^{73a} and R^{73b} independently represent, at each occurrence, hydrogen, C₁-C₆ alkyl, aryl or Het⁶⁷ (which C₁-C₆ alkyl, aryl and Het⁶⁷ groups are optionally substituted with one or more substituents selected from —OH, halogen, cyano, nitro, C₁-C₆ alkyl, aryl and Het⁶⁸);

or together represent C₃-C₆ alkylene, optionally interrupted by an O atom;

R^{74a}, R^{74b} and R^{74c} independently represent, at each occurrence, hydrogen, C₁-C₆ alkyl, aryl or Het⁶⁹ (which C₁-C₆ alkyl, aryl and Het⁶⁹ groups are optionally substituted with one or more substituents selected from —OH, halogen, cyano, nitro, C₁-C₆ alkyl, aryl and Het⁷⁰);

R^{74b} and R^{74c} may together represent C₃-C₆ alkylene, optionally interrupted by an O atom;

R^{77a}, and R^{77b} independently represent, at each occurrence, hydrogen, C₁-C₆ alkyl, aryl or Het⁷¹ (which C₁-C₆ alkyl, aryl and Het⁷¹ groups are optionally substituted with one or more substituents selected from —OH, halogen, cyano, nitro, C₁-C₆ alkyl, aryl and Het⁷²);

or together represent C₃-C₆ alkylene, optionally interrupted by an O atom;

R^{82a}, and R^{82b} independently represent, at each occurrence, hydrogen, C₁-C₆ alkyl, aryl or Het⁷³ (which C₁-C₆ alkyl, aryl and Het⁷³ groups are optionally substituted with one or more substituents selected from —OH, halogen, cyano, nitro, C₁-C₆ alkyl, aryl and Het⁷⁴) or together represent C₃-C₆ alkylene, optionally interrupted by an O atom;

Het⁶¹ to Het⁷⁴ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl;

X represents a nitrogen or oxygen atom;

m is an integer of 0 to 10;

n is an integer of 0 to 4;

k is an integer of 1 to 5;

provided that

a) R² or R³ does not represent a fragment of formula

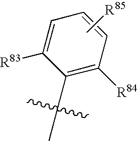

wherein

R⁸³ and R⁸⁴ represent independently, at each occurrence, halogen, C₁-C₁₂ alkyl, C₁-C₁₂ alkoxy, C₁-C₁₂ haloalkyl, C₁-C₁₂ haloalkoxy, cyano, —SR⁸⁶, —N(R^{87a})R^{87b}, C₂-C₆ alkynyl, aryl or Het⁷⁵;

R⁸⁵ represents hydrogen, C₁-C₁₂ alkyl group or C₁-C₁₂ alkoxy group (which C₁-C₁₂ alkyl and C₁-C₁₂ alkoxy groups are optionally substituted by one or more groups selected from halogen, C₂₋C₆ alkenyl, C₂-C₆ alkynyl, cyano, oxo, aryl, Het⁷⁶, —0R⁸⁸, —SR⁸⁹, —COXR⁹⁰, —N(R^{91a})R^{91b}, —SO₂R⁹²);

Het⁷⁵ to Het⁷⁶ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₁₋₆ alkoxy, aryl, aryloxy, —N(R^{93a})R^{93b}, —C(O)R^{93c}, —C(O)OR^{93d}, —C(O)N(R^{93e})R^{93f}, —N(R^{93g})C(O)R^{93h} and —N(R⁹³ⁱ)S(O)₂R^{93j}, OC(O)R^{93k} and a further Het;

R⁸⁶ to R⁹³ represent independently, at each occurrence, hydrogen or C₁₋₆ alkyl;

X represent O or N;

b) the compound is not:

2-(4-nitrophenyl)-3-(pyrrolidin-1-ylcarbonyl)isoindolin-1-one;

N,2-dibenzyl-3-oxoisoindoline-1-carboxamide;

N,2-diethyl-3-oxoisoindoline-1-carboxamide;

N,2-dibutyl-3-oxoisoindoline-1-carboxamide;

N,2-didodecyl-3-oxoisoindoline-1-carboxamide;

N,2-bis(4-methoxybenzyl)-3-oxoisoindoline-1-carboxamide;

3-oxo-N,2-dipropylisoindoline-1-carboxamide;

N,2-diheptyl-3-oxoisoindoline-1-carboxamide;

3-oxo-N,2-diphenylisoindoline-1-carboxamide;

N-(tert-butyl)-3-oxo-2-propylisoindoline-1-carboxamide;

N-(tert-butyl)-1-methyl-3-oxo-2-propyl-isoindoline-1-carboxamide;

N,1-dimethyl-3-oxo-2-propylisoindoline-1-carboxamide;

N-cyclohexyl-3-oxo-2-propylisoindoline-1-carboxamide;

N-(phenyl)-3-oxo-2-propylisoindoline-1-carboxamide;

2-benzyl-N-tert-butyl-3-oxoisoindoline-1-carboxamide:

2-benzyl-N,1-dimethyl-3-oxoisoindoline-1-carboxamide; 2-benzyl-N-tert-butyl-1-methyl-3-oxoisoindoline-1-carboxamide;

2-benzyl-N,1-dimethyl-3-oxoisoindoline-1-carboxamide;

tert-butyl (4-{1-[(tert-butylamino)carbonyl]-3-oxo-1,3-dihydro-2H-isoindol-2-yl}butyl)carbamate;

2-benzyl-3-oxo-N-(2-phenylethyl)isoindoline-1-carboxamide;

2-benzyl-N-butyl-3-oxoisoindoline-1-carboxamide;

2-benzyl-N-(2-methoxyethyl)-3-oxoisoindoline-1-carboxamide;

2-(2-hydroxyethyl)-3-oxo-N-(2-phenylethyl)isoindoline-1-carboxamide;

N-butyl-2-(2-hydroxyethyl)-3-oxoisoindoline-1-carboxamide;

2-(2-hydroxyethyl)-N-(2-methoxyethyl)-3-oxoisoindoline-1-carboxamide;

2-(3-(1H-imidazol-1-yl)propyl)-3-oxo-N-(2-phenylethyl)isoindoline-1-carboxamide;

N-butyl-2-[3-(1H-imidazol-1-yl)propyl]-3-oxoisoindoline-1-carboxamide;

2-[3-(1H-imidazol-1-yl)propyl]-N-(2-methoxyethyl)-3-oxoisoindoline-1-carboxamide

2-(Cyclohexyl)-3-oxo-N-(2-phenylethyl)isoindoline-1-carboxamide;

N-butyl-2-cyclohexyl-3-oxoisoindoline-1-carboxamide;

2-cyclohexyl-N-(2-methoxyethyl)-3-oxoisoindoline-1-carboxamide;

N,2-dibenzyl-5-hydroxy-4-methyl-3-oxoisoindoline-1-carboxamide;

N-benzyl-2-tert-butyl-5-hydroxy-3-oxo-4-phenylisoindoline-1-carboxamide;

N-benzyl-2-tert-butyl-5-hydroxy-4-methyl-3-oxoisoindoline-1-carboxamide;

N,2-dibenzyl-5-hydroxy-3-oxo-4-phenylisoindoline-1-carboxamide;

N-benzyl-2-tert-butyl-5-hydroxy-3-oxoisoindoline-1-carboxamide;

2-cyclohexyl-N-hexyl-3-oxoisoindoline-1-carboxamide;

N,2-dihexyl-3-oxoisoindoline-1-carboxamide;

N-hexyl-(2-hydroxyethyl)-3-oxoisoindoline-1-carboxamide;

N-hexyl-2-(4-hydroxybutyl)-3-oxoisoindoline-1-carboxamide;

N,2-dicyclohexyl-3-oxoisoindoline-1-carboxamide;

N-cyclohexyl-2-hexyl-3-oxoisoindoline-1-carboxamide;

N-cyclohexyl-2-(2-hydroxyethyl)-3-oxoisoindoline-1-carboxamide;

N-cyclohexyl-2-(4-hydroxybutyl)-3-oxoisoindoline-1-carboxamide;

tert-butyl (4-{1-[(cyclohexylamino)carbonyl]-3-oxo-1,3-dihydro-2H-isoindol-2-yl}butyl)carbamate;

N-adamantan-1-yl-2-cyclohexyl-3-oxoisoindoline-1-carboxamide;

N-adamantan-1-yl-2-hexyl-3-oxoisoindoline-1-carboxamide;

N-adamantan-1-yl-2-(2-hydroxyethyl)-3-oxoisoindoline-1-carboxamide ;

N-adamantan-1-yl-2-(2-morpholin-4-ylethyl)-3-oxoisoindoline-1-carboxamide;

N,2-dibenzyl-5-{[(2-nitrophenyl)sulfonyl]amino}-3-oxoisoindoline-1-carboxamide;

ethyl[1-(tert-butylcarbamoyl)-3-oxo-1,3-dihydro-2H-isoindol-2-yl]acetate;

N-[2-(3,4-dimethoxyphenyl)ethyl]-3-oxo-2-(1-phenylethyl)isoindoline-1-carboxamide

N-cyclopentyl-2-(3-methoxybenzyl)-3-oxoisoindoline-1-carboxamide;

2-(1,3-benzodioxol-5-ylmethyl)-N-{[(4-methylphenyl)sulfonyl]methyl}-3-oxoisoindoline-1-carboxamide;

N-cyclohexyl-3-oxo-2-(2-thienylmethyl)isoindoline-1-carboxamide;

2-benzyl-N-cyclohexyl-3-oxoisoindoline-1-carboxamide;

N-{[(4-methylphenyl)sulfonyl]methyl}-3-oxo-2-(2-thienylmethyl)isoindoline-1-carboxamide;

2-(4-chlorobenzyl)-N-{[[(4-methylphenyl)sulfonyl]methyl}-3-oxoisoindoline-1-carboxamide;

N-cyclohexyl-2-(2-furylmethyl)-3-oxoisoindoline-1-carboxamide;

2-(4-chlorobenzyl)-N-cyclohexyl-3-oxoisoindoline-1-carboxamide;

tert-butyl{1-benzyl-2-hydroxy-3-[(2-hydroxy-3-{[[(3-oxo-2,3-dihydro-1H-isoindol-1- yl)carbonyl]amino}-4-phenylbutyl)amino]propyl}carbamate;

1-hydroxy-2-methyl-3-oxo-N-(pyridin-2-ylmethyl)isoindoline-1-carboxamide;

N-[3-(dimethylamino)propyl]-1-hydroxy-2-(2-hydroxyethyl)-3-oxoisoindoline-1-carboxamide;

N-(3-azepan-1-ylpropyl)-1-hydroxy-3-oxo-2-phenylisoindoline-1-carboxamide;

2-benzoyl-1-hydroxy-3-oxo-N-phenylisoindoline-1-carboxamide;

3-oxo-N,2-diphenylisoindoline-1-carboxamide;

6-{[[(1-methyl-2-octyl-3-oxo-2,3-dihydro-1H-isoindol-1-yl)carbonyl]amino}hexanoic acid

N-(methyl)-2-benzoyl-1-hydroxy-3-oxoindoline-1-carboxamide;

N-(phenyl)-2-benzoyl-1-hydroxy-3-oxoisoindoline-1-carboxamide;

6-[(2-allyl-1-methyl-3-oxoisoindoline-1-carbonyl)-amino]-hexanoic acid;

N-{(1S,2R)-1-(3,5-difluorobenzyl)-3-[(3-ethylbenzyl)amino]-2-hydroxypropyl}-2-ethyl-3-oxoisoindoline-1-carboxamide;

N1-cyclopentyl-N4-(2,6-difluorophenyl)-2-(2,4-dimethylphenyl)-5-methyl-3-oxoisoindoline-1,4-dicarboxamide;

methyl[1-(tert-butylcarbamoyl)-3-oxo-1,3-dihydro-2H-isoindol-2-yl]acetate;

1-hydroxy-2-methyl-3-oxoisoinoline-1-carbohydrazide;

1-hydroxy-3-oxo-phenylisoindoline-1-carbohydrazide;

or a pharmaceutically acceptable derivative thereof;

c) the compound is not:

2-(2-ethoxyethyl)-N-isopropyl-3-oxoisoindoline-1-carboxamide;

N-(tert-butyl)-3-oxo-2-(3-pyrrolidin-1-ylpropyl)isoindoline-1-carboxamide;

N-(tert-butyl)-3-oxo-2-(tetrahydrofuran-2-ylmethyl)isoindoline-1-carboxamide;

2-[1-(hydroxymethyl)butyl]-N-isopropyl-3-oxoisoindoline-1-carboxamide;

N-isopropyl-2-(3-methylbutyl)-3-oxoisoindoline-1-carboxamide;

N-(tert-butyl)-2-cyclohexyl-3-oxoisoindoline-1-carboxamide;

N-(tert-butyl)-2-(3-methylbutyl)-3-oxoisoindoline-1-carboxamide;

methyl N-{[2-(3-methylbutyl)-3-oxo-2,3-dihydro-1H-isoindol-1-yl]carbonyl}glycinate;

tert-butyl N-(2-[1-(hydroxymethyl)butyl]-3-oxo-2,3-dihydro-1H-isoindol-1-yl}carbonyl)glycinate;

tert-butyl N-{[2-(3-methylbutyl)-3-oxo-2,3-dihydro-1H-isoindol-1-yl]carbonyl}glycinate;

N-(tert-butyl)-2-[1-(methoxymethyl)propyl]3-oxoisoindoline-1-carboxamide;

N-(tert-butyl)-2-[2-(diethylamino)ethyl]-3-oxoisoindoline-1-carboxamide;

N-(tert-butyl)-2-[1-(hydroxymethyl)butyl]-3-oxoisoindoline-1-carboxamide;

tert-butyl N-{[3-oxo-2-(2-thienylmethyl)-2,3-dihydro-1H-isoindol-1-yl]carbonyl}glycinate;

tert-butyl N-((2-[2-(methylthio)ethyl]-3-oxo-2,3-dihydro-1H-isoindol-1-yl}carbonyl)glycinate;

methyl N-{[2-(Cyclopropylmethyl)-3-oxo-2,3-dihydro-1H-isoindol-1-yl]carbonyl glycinate; or

2-(2,2-dimethylpropyl)-3-oxo-N-(4,4,4-trifluorobutyl)isoindoline-1-carboxamide.

or a pharmaceutically acceptable derivative thereof.

The compounds of formula I as defined above are referred to hereinafter as “the compounds of the invention”.

In one embodiment R¹ represents C₁-C₇ alkyl (which alkyl group is optionally substituted by one or more groups selected from halogen, C₂-C₆ alkenyl, C₃-C₈ cycloalkyl, cyano, oxo, —OR⁸, —COXR¹¹, aryl or Het¹); further R¹ represents Het².

In one embodiment R¹ represents C₁-C₇ alkyl (which alkyl group is optionally substituted by one or more groups selected from halogen, C₂-C₆ alkenyl, C₃-C₈ cycloalkyl, cyano, oxo, —OR⁸, —COXR¹¹, phenyl, naphthalenyl or Het¹).

In one embodiment R¹ represents (1-benzylpyrrolidin-3-yl); (1-fluoro-3-phenyl-propan-2-yl); (1-methyl-5-phenyl-pyrazol-3-yl)methyl; (1-methylpyrrol-2-yl)methyl; (2,3-difluorophenyl)methyl; (2,4-difluorophenyl)methyl; (2,5-dimethoxyphenyl)methyl; (2,5-dimethylphenyl)methyl; (2-bromophenyl)methyl; (2-chloro-4-fluoro-phenyl)methyl; (2-chloro-6-phenoxy-phenyl)methyl; (2-chlorophenyl)methyl; (2-dimethylamino-2-phenyl-ethyl); (2-ethoxyphenyl)methyl; (2-fluorophenyl)methyl; (2-methoxyphenyl)methyl; (2-methyl-2-phenyl-propyl); (2-methylphenyl)methyl; (2-phenoxyphenyl)methyl; (2-phenylphenyl)methyl; (2-pyridin-3-ylphenyl)methyl; (3,4-dichlorophenyl)methyl; (3,4-difluorophenyl)methyl; (3,5-dimethoxyphenyl)methyl; (3-chlorophenyl)methyl; (3-cyano-4-fluoro-phenyl)methyl; (3-cyanophenyl)methyl; (3-fluorophenyl)methyl; (3-hydroxy-2,2-dimethyl-propyl); (3-methoxyphenyl)methyl; (3-phenyl1,2-oxazol-5-yl)methyl; (3-phenylphenyl)methyl; (3-pyrrol-1-ylphenyl)methyl; (4-chlorophenyl)methyl; (4-dimethylaminophenyl)methyl; (4-fluorophenyl)methyl; (4-hydroxyphenyl)methyl; (4-methoxycarbonylphenyl)methyl; (4-phenoxyphenyl)methyl; (4-phenylphenyl)methyl; (5-methyl-2-phenyl-1,3-oxazol-4-yl)methyl; (5-methyl-3-phenyl-1,2-oxazol-4-yl)methyl; (phenyl-pyridin-2-yl-methyl); [(1R)-1-(4-methoxyphenyl)ethyl]; [(1S)-1-phenylethyl]; [(1R)-1-phenylethyl]; [(1R)-2-(4-chlorophenyl)-1-(4,4,4-trifluorobutylcarbamoyl)ethyl]; [(1R)-2-(4-chlorophenyl)-1-methoxycarbonyl-ethyl]; [(1S)-1-naphthalen-1-ylethyl]; [(2R)-2-(4-chlorophenyl)propyl]; [(2S)-2-(4-chlorophenyl)propyl]; [(4-chlorophenyl)-pyridin-4-yl-methyl]; [(4-fluorophenyl)-pyridin-3-yl-methyl]; [(4-fluorophenyl)-pyridin-3-yl-methyl]; [(4-fluorophenyl)-pyridin-3-yl-methyl]; [2-(2,4-dichlorophenyl)phenyl]methyl; [2-(2,4-difluorophenyl)phenyl]methyl; [2-(2,5-difluorophenyl)phenyl]methyl; [2-(2-chlorophenyl)phenyl]methyl; [2-(3,4-dichlorophenyl)phenyl]methyl; [2-(3,4-difluorophenyl)phenyl]methyl; [2-(3-chloro-4-fluoro-phenyl)phenyl]methyl; [2-(3-fluorophenyl)phenyl]methyl; [2-(4-chloro-2-methyl-phenyl)-2,2-difluoro-ethyl]; [2-(4-chlorophenyl)phenyl]methyl; [2-(4-fluoro-2-methyl-phenyl)phenyl]methyl; [2-(4-fluorophenoxy)phenyl]methyl; [2-(4-fluorophenyl)phenyl]methyl; [2-(4-methoxyphenyl)-2-oxo-ethyl]; [2-(4-methoxyphenyl)phenyl]methyl; [2-(4-methylphenyl)phenyl]methyl; [2-(trifluoromethyl)phenyl]methyl; [2-[4-(trifluoromethyl)phenoxy]phenyl]methyl; [3-(difluoromethoxy)phenyl]methyl; [3,5-bis(trifluoromethyl)phenyl]methyl; [4-(difluoromethoxy)phenyl]methyl; [4-(trifluoromethyl)phenyl]methyl; 1-(1H-indol-3-yl)propan-2-yl; 1-(4-fluorophenyl)ethyl; 1-naphthalen-1-ylethyl; 1-naphthalen-2-ylethyl; 1-phenylethyl; 1-phenylpropyl; 2-(1-cyclohexenyl)ethyl; 2-(2-ethoxyphenyl)ethyl; 2-(2-methoxyphenyl)ethyl; 2-(2-phenoxyphenyl)ethyl; 2-(3,4-dichlorophenyl)ethyl; 2-(3,5-dimethoxyphenyl)ethyl; 2-(3-bromo-4-methoxy-phenyl)ethyl; 2-(3-fluorophenyl)ethyl; 2-(4-bromophenyl)ethyl; 2-(4-chlorophenyl)ethyl; 2-(4-chlorophenyl)propyl; 2-(4-fluorophenoxy)propyl; 2-(4-fluorophenyl)ethyl; 2-(4-fluorophenyl)propyl; 2-(4-phenoxyphenyl)ethyl; 2-(4-methoxyphenyl)ethyl; 2-(4-methoxyphenyl)ethyl; 2-(4-phenylphenyl)ethyl; 2-(5-bromo-2-methoxy-phenyl)ethyl; 2-(6-chloro-1H-indol-3-yl)ethyl; 2,2-dimethylpropyl; 2,2-diphenylethyl; 2-[2-(trifluoromethoxy)phenyl]ethyl; 2-(3-(trifluoromethyl)phenyl]ethyl; 2-[4-(diethylcarbamoyl)phenyl]ethyl; 2-[4-(trifluoromethyl)phenyl]ethyl; 2-benzo[1,3]dioxol-5-ylethyl; 2-methylbutyl; 2-methylpropyl; 2-naphthalen-1-ylpropyl; 2-phenoxypropyl; 2-phenylpropyl; 2-thiophen-2-ylethyl; 3,3-dimethylbutyl; 3-phenylpropyl; 3-pyrrolidin-1-ylpropyl; 4-phenylbutan-2-yl;

4-phenylbutyl; 9H-fluoren-9-yl; benzhydryl; benzyl; cycloheptyl; cyclohexyl; cyclohexylmethyl; naphthalen-1-ylmethyl; pentan-3-yl; phenethyl; thiophen-2-ylmethyl; 2-phenylpropan-2-yl; 1-phenylpropyl; [2-(4-chlorophenyl)-2-methyl-propyl]; [4-fluoro-2-(4-fluorophenyl)phenyl]methyl; (4-fluoro-2-phenyl-phenyl)methyl; [5-fluoro-2-(4-fluorophenyl)phenyl]methyl; (5-fluoro-2-phenyl-phenyl)methyl; 1-(4-fluorophenyl)ethyl; 2-(4-chlorophenyl)propan-2-yl; 2-(4-fluorophenyl)propan-2-yl or 1-(4-chlorophenyl)ethyl.

(The naming of the radicals was made using a program from Lexichem package from Openeye, version 4.)

In one embodiment R² represents C₁-C₆ alkyl (which alkyl group is optionally substituted by one or more groups selected from halogen, C₂-C₆ alkenyl, C₃-C₈ cycloalkyl, —COR¹⁷, trimethylsilyl, —COXR¹⁸, aryl or Het³);

further R² represent aryl or Het⁴.

In one embodiment R² represents (1-benzylpyrrolidin-3-yl); (1-methylpyrrol-2-yl)methyl; (2,2-difluorobenzo[1,3]dioxol-5-yl)methyl; (2,3-dimethylcyclohexyl); (2,4-difluorophenyl)methyl; (2-chloro-4-methylsulfonyl-phenyl)methyl; (2-chlorophenyl)methyl; (2-fluoro-4-methylsulfonyl-phenyl)methyl; (2-hydroxyphenyl)methyl; (2-methylpropan-2-yl)oxycarbonylmethyl; (3,4-dichlorophenyl)methyl; (3,4-difluorophenyl)methyl; (3,4-imethoxyphenyl)methyl; (3-carbamoyl-4-fluoro-phenyl)methyl; (3-chlorophenyl)methyl; (3-cyano-4-fluoro-phenyl)methyl; (3-cyanophenyl)methyl; (3-methoxyphenyl); (3-methyl-5-phenyl-1,2-oxazol-4-yl)methyl; (4-amino-2-methyl-pyrimidin-5-yl)methyl; (4-carbamoylphenyl); (4-carbamoylphenyl)methyl; (4-cyano-2,6-difluoro-phenyl)methyl; (4-cyanophenyl); (4-cyanophenyl)methyl; (4-dimethylaminophenyl)methyl; (4-fluorophenyl)methyl; (4-hydroxyphenyl)methyl; (4-methylcyclohexyl); (4-methylsulfonylphenyl)methyl; (5-methyl-1,2-oxazol-3-yl)methyl; (5-methyl-2-furyl)methyl; (5-methyl-2-phenyl-1,3-oxazol-4-yl)methyl; (5-methylpyrazin-2-yl)methyl; [2-(trifluoromethyl)phenyl]methyl; [3-(aminomethyl)-4-fluoro-phenyl]methyl; [3-(difluoromethoxy)phenyl]methyl; [3-(dimethylcarbamoyl)-4-fluoro-phenyl)methyl; [3-(trifluoromethyl)phenyl]methyl; [3,5-bis(trifluoromethyl)phenyl]methyl; [3-[[(2,2-difluoroacetyl)amino]methyl]-4-fluoro-phenyl]methyl; [4-(acetamidomethyl)phenyl]methyl; [4-(aminomethyl)phenyl]; [4-(difluoromethoxy)phenyl]methyl; [4-(trifluoromethyl)phenyl]methyl; [4[[(2,2-difluoroacetyl)amino]methyl]phenyl]; [4-[[(2-fluoroacetyl)amino]methyl]phenyl)methyl; [5-(2-furyl)1,2-oxazol-3-yl]methyl; [6-(trifluoromethyl)pyridin-3-yl]methyl; 1H-indol-3-ylmethyl; 1-pyridin-4-ylethyl; 2-(1H-indol-3-yl)ethyl; 2-(2,4-dichlorophenyl)ethyl; 2-(2,6-dichlorophenyl)ethyl; 2-(2-chlorophenyl)ethyl; 2-(3,4-dichlorophenyl)ethyl; 2-(3,4-dimethoxyphenyl)ethyl; 2-(3-chlorophenyl)ethyl; 2-(3-fluorophenyl)ethyl; 2-(4-benzoylpiperazin-1-yl)ethyl; 2-(4-chlorophenyl)ethyl; 2-(4-fluorophenyl)ethyl;

2-(4-methoxyphenyl)ethyl; 2[3-(trifluoromethyl)phenyl]ethyl; 2-benzo[1,3]dioxol-5-ylethyl; 2-ethoxycarbonylethyl; 2-furylmethyl; 2-methoxyethyl; 2-pyridin-2-ylethyl; 2-pyridin-4-ylethyl; 2-thiophen-2-ylethyl; 3-imidazol-1-ylpropyl; 3-methoxypropyl; 4,4,4-trifluorobutyl; 4,4-difluorobutyl; benzo[1,3]dioxol-5-ylmethyl; benzotriazol-1-ylmethyl; benzyl; butyl; cyclohexyl; ethyl; methoxycarbonylmethyl; phenethyl; propan-2-yl; propyl; pyridin-3-ylmethyl; pyridin-4-ylmethyl; tert-butyl; trimethylsilylmethyl; (5-oxo-1-propan-2-yl-pyrrolidin-3-yl)methyl; propan-2-ylcarbamoylmethyl, (2-fluorophenyl)methyl; (3-fluorophenyl)methyl; 1-phenylethyl; 2-phenylpropan-2-yl; or 5-cyanopentyl.

(The naming of the radicals was made using a program from Lexichem package from Openeye, version 4.)

In one embodiment R¹ represents C₁-C₇ alkyl (which alkyl group is optionally substituted by one or more groups selected from halogen, C₂-C₆ alkenyl, C₃-C₈ cycloalkyl, cyano, oxo, —OR⁸, —COXR¹¹, aryl or Het¹); further R¹ represents Het²; and

R² represents C₁-C₆ alkyl (which alkyl group is optionally substituted by one or more groups selected from halogen, C₂-C₆ alkenyl, C₃-C₇ cycloalkyl, —COR¹⁷ , trimethylsilyl, —COXR¹⁸, aryl or Het³); further R² represents aryl or Het⁴.

In one embodiment R¹ represents C₃-C₈ cycloalkyl (which cycloalkyl group is optionally substituted by one or more groups selected from halogen, C₂-C₆ alkenyl, C₃-C₈ cycloalkyl, cyano, oxo, —OR⁸, —COXR¹¹, aryl or Het'); and

R² represents C₁-C₆ alkyl (which alkyl group is optionally substituted by one or more groups selected from halogen, C₂-C₆ alkenyl, C₃-C₇ cycloalkyl, —COR¹⁷, trimethylsilyl, —COXR¹⁸, aryl or Het³); further R² represents aryl or Het⁴.

In one embodiment R³ represents hydrogen, C₁-C₄ alkyl (which alkyl group is optionally substituted by one or more groups selected from fluoro, C₂-C₆ alkenyl, trialkylsilyl, —COXR²⁷, aryl or Het⁵).

In one embodiment R³ represents hydrogen.

In one embodiment R⁴ represents hydrogen.

In one embodiment R⁵ to R⁷ independently represent, at each occurence, hydrogen, —OH, halogen, cyano, C₁₋₆ alkyl, —OR³⁶, —C(O)N(R^{40a})(R^{40b}), —N(R^{44a})S(O)₂R⁴⁴b.

In one embodiment aryl is, independently, at each occurrence, optionally substituted by —OH, halogen, cyano, nitro, C₁-C₆ alkyl, —OR⁵⁰, C₂-C₆ alkenyl, phenyl, Het⁸; wherein R⁵⁰ represents C₁-C₆ alkyl or aryl.

In one embodiment aryl is, at each occurrence, phenyl.

In an embodiment of this invention the compound of formula I is

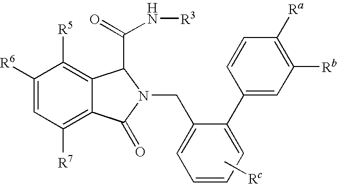

in which R^{a} is hydrogen or fluoro;

R^{b} is hydrogen or fluoro;

R^{c} is hydrogen or fluoro;

R³ is C₁₋₄ alkyl optionally terminally substituted by 1, 2 or 3 fluoro;

R⁵ is hydrogen or C₁₋₄ alkyl,

R⁶ is hydrogen, OH, halo or C₁₋₄alkoxy;

R⁷ is hydrogen or halo.

In an embodiment of this invention the compound of formula I is

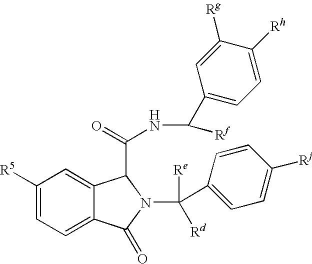

in which

R^{d} is hydrogen or C₁₋₄ alkyl;

R^{e} is hydrogen or C₁₋₄ alkyl;

R^{f} is hydrogen or C₁₋₄ alkyl;

R^{g} is hydrogen or halo;

R^{h} is hydrogen or halo;

Rⁱ is hydrogen or halo;

R⁵ is hydrogen or halo.

In an embodiment of this invention the compound of formula I is

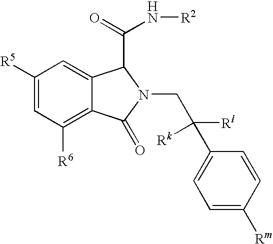

in which

R^{k} is hydrogen or C₁₋₄ alkyl;

R^{l} is hydrogen or C₁₋₄ alkyl;

R^{m} is hydrogen or halo;

R² is C₃₋₆ alkyl;

R⁵ is hydrogen or halo;

R⁶ is hydrogen or halo.

In an embodiment of this invention the compound of formula I is

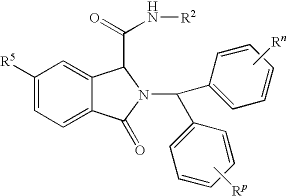

in which

Rⁿ is hydrogen or halo;

R^{p} is hydrogen or halo;

R² is C₃₋₆ alkyl or benzyl, optionally substituted by halo in the phenyl ring;

R⁵ is hydrogen or halo.

In an embodiment of this invention R¹ is (2-phenylphenyl)methyl (biphenylmethyl), optionally substituted by one to three fluoro;

R² is selected from ethyl, propyl, n-butyl, tert-butyl, 4,4,4-trifluorobutyl, 4,4-difluorobutyl, 4-fluorobutyl, benzo[1,3]dioxol-5-yl-methyl, (2,2-difluorobenzo[1,3]dioxol-5-yl)methyl, benzyl, (2-chlorophenyl)methyl, (4-fluorophenyl)methyl, (2-trifluoromethylphenyl)methyl, (3-cyanophenyl)methyl, (4-cyanophenyl)methyl, (3-cyano-4-fluorophenyl)methyl, (4-carbamoylphenyl)methyl, (5-methylpyrazin-2-yl)methyl, pyridin-3-ylmethyl, (4-amino-2-methyl-pyrimidin-5-yl)methyl, [6-(trifluoromethyl)pyridin-3-yl]methyl, pyridin-3-ylmethyl, [6-(trifluoromethyl)pyridin-3-yl]methyl, pyridin-4-ylmethyl, [4-[[(2,2-difluoroacetyl)amino]methyl]phenyl]methyl, [4-(acetamidomethyl)phenyl]methyl, [4-[[(2-fluoroacetyl)amino]methyl]phenyl]methyl, 2-phenylethyl or 2-(4-fluorophenyl)ethyl;

R⁵ to R⁷ are independently selected from —OH, methyl, methoxy, chloro, fluoro, cyano, methylsulfonylamino, fluoromethoxy, difluoromethoxy, trifluoromethanesulfonate;

R³ is hydrogen;

R⁴ is hydrogen;

R⁵ to R⁷ are independently selected from hydrogen, —OH, methyl, methoxy, fluoro or chloro;

or an enantiomer thereof.

In an embodiment of this invention R¹ is benzhydryl (diphenylmethyl), optionally substituted by one or more substitutents selected from fluoro or chloro;

R² is selected from ethyl, propyl, butyl, tert-butyl, 4,4-difluorobutyl, 4,4,4-trifluorobutyl, benzyl, (2-chloro-4-methylsulfonyl-phenyl)methyl, (4-methylsulfonylphenyl)methyl, (2-fluoro-4-methylsulfonyl-phenyl)methyl, (4-methylsulfonylphenyl)methyl, (2-hydroxyphenyl)methyl, [2-(trifluoromethyl)phenyl]methyl, (2,4-difluorophenyl)methyl, (2-chlorophenyl)methyl, 2-(4-fluorophenyl)ethyl

R³ is hydrogen;

R⁴ is hydrogen;

R⁵ to R⁷ are independently selected from hydrogen, —OH, methyl, methoxy, fluoro or chloro;

or an enantiomer thereof.

In an embodiment of this invention R¹ is 4-phenylbutan-2-yl, optionally substituted by one or more substitutents selected from fluoro or chloro;

R² is selected from (2-chlorophenyl)methyl, [2-(trifluoromethyl)phenyl]methyl, benzyl, 2-phenylethyl;

R³ is hydrogen;

R⁴ is hydrogen;

R⁵ to R⁷ are independently selected from hydrogen, —OH, methyl, methoxy, fluoro or chloro;

or an enantiomer thereof.

In an embodiment of this invention R¹ is 3,3-dimethylbutyl;

R² is selected from [3-(difluoromethoxy)phenyl]methyl, [3-(trifluoromethoxy)phenyl]methyl, 2-(1H-indol-3-yl)ethyl, 1H-indol-3-ylmethyl, (3-chlorophenyl)methyl, (3,4-dichlorophenyl)methyl, [4-(difluoromethoxy)phenyl]methyl, 2-(3-fluorophenyl)ethyl, 2-benzo[1,3]dioxol-5-ylethyl, 2-[3-(trifluoromethyl)phenyl]ethyl, 2-(3,4-dichlorophenyl)ethyl, 2-(2,4-dichlorophenyl)ethyl, 2-(2,6-dichlorophenyl)ethyl, 2-(4-chlorophenyl)ethyl, 2-(3-chlorophenyl)ethyl or 2-(2-chlorophenyl)ethyl;

R³ is hydrogen;

R⁴ is hydrogen;

R⁵ to R⁷ are independently selected from hydrogen, —OH, methyl, methoxy, fluoro or chloro;

or an enantiomer thereof.

In an embodiment of this invention R¹ is benzyl (phenylmethyl), optionally substituted by one or more substiutents selected from fluoro, chloro, cyano;

R² is selected from ethyl, n-propyl, isopropyl, n-butyl, sec-butyl, tert-butyl, benzo[1,3]dioxol-5-yl-methyl, benzyl, 1-phenylethyl, 2-phenylethyl, cyclopentyl, which groups are optionally substituted by one or more substiutents selected from fluoro, chloro, cyano, trifluoromethyl;

further R² represents pyridin-3-ylmethyl, pyridin-4-ylmethyl, [3-[[(2,2-difluoroacetyl)amino]methyl]-4-fluoro-phenyl)methyl, [4-(difluoromethoxy)phenyl]methyl, (4-dimethylaminophenyl)methyl, [5-(2-furyl)1,2-oxazol-3-yl]methyl, [5-(2-furyl)1,2-oxazol-3-yl]methyl, 2-(3,4-dimethoxyphenyl)ethyl, butan-2-yl, cyclopentyl, (2,3-dimethylcyclohexyl), (4-hydroxyphenyl)methyl, [2-(trifluoromethyl)phenyl]methyl;

R³ is hydrogen;

R⁴ is hydrogen; R⁵ to Ware independently selected from hydrogen, —OH, methyl, methoxy, bromo, chloro, fluoro, trimethylsilyl; or an enantiomer thereof.

In an embodiment of this invention R¹ is (2-cyclopentylphenyl)methyl; R² is selected from 4,4-difluorobutyl, methyl; R³ is hydrogen;

R⁴ is hydrogen; R⁵ to R⁷ are independently selected from bromo, fluoro, chloro or cyano; or an enantiomer thereof.

In an embodiment of this invention R¹ is 1-phenylethyl, optionally substituted by one or more substiutents selected from fluoro, chloro, cyano, methoxy; R² is selected from ethyl, propyl, tert-butyl, 4,4-difluorobutyl, 4,4,4-trifluorobutyl, 4- methylsulfonyl, benzyl, which benzyl group is optionally substituted by one or more substituents selected from fluoro, chloro, cyano; further R² represents pyridinmethyl, ((2,2-difluoroacetyl)amino)methyl, difluoromethoxy, dimethylamino, 5-(2-furyl)1,2-oxazol-3-yl-methyl, cyclopentyl R³ is hydrogen; R⁴ is hydrogen; R⁵ to R⁷ are independently selected from bromo, fluoro, chloro or cyano; or an enantiomer thereof.

In an embodiment of this invention R¹ is 3-hydroxy-2,2-dimethylpropyl;

R² is selected from [3-(difluoromethoxy)phenyl]methyl, (3,4-dichlorophenyl)methyl, (3- chlorophenyl)methyl, [3-(trifluoromethyl)phenyl]methyl; R³ is hydrogen; R⁴ is hydrogen; R⁵ to R⁷ are independently selected from hydrogen, fluoro, chloro; or an enantiomer thereof.

In an embodiment of this invention R¹ is 2-(4-chlorophenyl)propyl;

R² is selected from methyl, ethyl, n-propyl, propan-2-yl, butyl, (4-amino-2-methyl-pyrimidin-5-yl)methyl, (5-methylpyrazin-2-yl)methyl, pyridin-3-ylmethyl, [6-(trifluoromethyl)pyridin-3-yl]methyl, (4-amino-2-methyl-pyrimidin-5-yl)methyl, [6-(trifluoromethyl)pyridin-3-yl]methyl, (5-methyl-2-phenyl-1,3-oxazol-4-yl)methyl, 4,4,4-trifluorobutyl, (4-methylsulfonylphenyl)methyl, benzyl (2,2-difluorobenzo[1,3]dioxol-5-yl)methyl, (4-methylsulfonylphenyl)methyl, butyl, 2-(1H-indol-3-yl)ethyl; (4-carbamoylphenyl)methyl, (4-cyanophenyl)methyl, [3-(dimethylcarbamoyl)-4-fluoro-phenyl]methyl, [3-(dimethylcarbamoyl)-4-fluoro-phenyl]methyl, [4-(aminomethyl)phenyl], [4-[[(2,2-difluoroacetyl)amino]methyl]phenyl], (4-carbamoylphenyl), pyridin-4-ylmethyl, 3-methoxypropyl, (3-cyano-4-fluoro-phenyl)methyl, [3-[[(2,2-difluoroacetyl)amino]methyl]-4-fluoro-phenyl]methyl, [3-(aminomethyl)-4-fluoro-phenyl]methyl, (3-carbamoyl-4-fluoro-phenyl)methyl, 2-pyridin-4-ylethyl, (1-methylpyrrol-2-yl)methyl, [4-(difluoromethoxy)phenyl]methyl, (1-benzylpyrrolidin-3-yl), 3-imidazol-1-ylpropyl, (4-dimethylaminophenyl)methyl, (4-methylsulfonylphenyl)methyl, 3-imethylaminopropyl, 1-pyridin-3-ylethyl, (3-methoxyphenyl), 1-pyridin-4-ylethyl, (4-cyanophenyl), 3-methoxypropyl, benzo[1,3]dioxol-5-ylmethyl, (3,4-dimethoxyphenyl)methyl, (3-methyl-5-phenyl-1,2-oxazol-4-yl)methyl, (5-methyl 1,2-oxazol-3-yl)methyl, [2-(trifluoromethyl)phenyl]methyl, (2-chlorophenyl)methyl, 2-(3,4-dimethoxyphenyl)ethyl, 2-thiophen-2-ylethyl, 2-(4-methoxyphenyl)ethyl, 2-phenylethyl, 2-methoxyethyl, (4-fluorophenyl)methyl, methoxycarbonylmethyl or benzotriazol-1-ylmethyl;

R³ is hydrogen;

R⁴ is hydrogen;

R⁵ to R⁷ are independently selected from hydrogen, bromo, fluoro, chloro;

or an enantiomer thereof.

In an embodiment of this invention R¹ is 2-(4-chlorophenyl)propyl;

R² represents tert-butyl;

R³ is hydrogen;

R⁴ is hydrogen;

R⁵ to R⁷ are independently selected from hydrogen, —OH, bromo, fluoro, chloro, methyl, —OCH₃, —OCH₂F, trimethylsilyl;

or an enantiomer thereof.

In an embodiment of this invention R¹ is 2-(4-fluorophenyl)propyl;

R² represents 4,4,4-trifluorobutyl, benzyl, tert-butyl, butyl,

R³ is hydrogen;

R⁴ is hydrogen; R⁵ to R⁷ are independently selected from hydrogen, —OH, bromo, fluoro, chloro, methoxy; or an enantiomer thereof.

In an embodiment of this invention R¹ is 2,2-dimethylpropyl; R² represents [3-(trifluoromethoxy)phenyl]methyl [3-(difluoromethoxy)phenyl]methyl, (3,4-dichlorophenyl)methyl, 2-[3- (trifluoromethyl)phenyl]ethyl, 2-(1H-indol-3-yl)ethyl, (3-chlorophenyl)methyl, [4- (difluoromethoxy)phenyl]methyl, [3-(trifluoromethyl)phenyl]methyl, 2-(3- fluorophenyl)ethyl, 2-(2-chlorophenyl)ethyl, 2-(3-chlorophenyl)ethyl, 2-(2,4-dichlorophenyl)ethyl, 2-(4- chlorophenyl)ethyl, 2-(2,6-dichlorophenyl)ethyl, benzo[1,3]dioxol-5-ylmethyl, or benzyl; R³ is hydrogen; R⁴ is hydrogen; R⁵ to R⁷ are independently selected from hydrogen, bromo, fluoro, chloro, or an enantiomer thereof.

In an embodiment of this invention R¹ is 2-phenylpropan-2-yl;

R² represents benzyl, 1-phenylethyl, (4-fluorophenyl)methyl,4,4,4-trifluorobutyl; R³ is hydrogen; R⁴ is hydrogen; R⁵ to R⁷ are independently selected from hydrogen, bromo, fluoro, chloro, or an enantiomer thereof.

In an embodiment of this invention R¹ is 1-phenylpropyl; R² is benzyl, (2-chlorophenyl)methyl, [2-(trifluoromethyl)phenyl]methyl or(4- dimethylaminophenyl)methyl; R³ is hydrogen;

R⁴ is hydrogen;

R⁵ to R⁷ are independently selected from hydrogen, bromo, fluoro, chloro;

or an enantiomer thereof.

In an embodiment of this invention R¹ is [2-(4-chlorophenyl)-2-methyl-propyl];

R² represents n-butyl;

R³ is hydrogen;

R⁴ is hydrogen;

R⁵ to R⁷ are independently selected from hydrogen, bromo, fluoro or chloro;

or an enantiomer thereof.

In an embodiment of this invention R² is (2-chlorophenyl)methyl;

R¹ represents benzhydryl, (2-pyridin-3-ylphenyl)methyl, (3,4-difluorophenyl)methyl 1-(1H-indol-3-yl)propan-2-yl, 2-(4-chlorophenyl)propyl, (2,5-dimethylphenyl)methyl, [(1R)-1-(4-methoxyphenyl)ethyl], 2-(1H-indol-3-yl)propyl, [(1R)-1-(3-methoxyphenyl)ethyl], [(1S)-1-naphthalen-1-ylethyl], 1-phenylpropyl, 2-phenylpropyl, 3-phenylpropyl, 2-phenethyl,

4-phenylbutan-2-yl, (2-phenylphenyl)methyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is (3,4-dichlorophenyl)methyl;

R¹ is(3-hydroxy-2,2-dimethyl-propyl), 2,2-dimethylpropyl, 2-methylpropyl or 3,3-imethylbutyl;

R³ is hydrogen;

R⁴is hydrogen;

R⁵ to R⁷ are independently selected from hydrogen, bromo, fluoro and chloro;

or an enantiomer thereof.

In an embodiment of this invention R² is (3-chlorophenyl)methyl;

R¹ represents (3-hydroxy-2,2-dimethyl-propyl), 2-methylpropyl, 2,2-dimethylpropyl, 3,3-dimethylbutyl, (4-hydroxyphenyl)methyl or (3-cyanophenyl)methyl;

R³ is hydrogen;

R⁴is hydrogen;

R⁵ to R⁷ are independently selected from hydrogen, bromo, fluoro and chloro;

or an enantiomer thereof.

In an embodiment of this invention R² is (3-cyano-4-fluoro-phenyl)methyl;

R¹ is (2-chloro-4-fluoro-phenyl)methyl, [3,5-bis(trifluoromethyl)phenyl]methyl, (3-cyano-4-fluoro-phenyl)methyl, 2-phenylethyl, benzyl, (3,4-difluorophenyl)methyl, (2-phenylphenyl)methyl or 2-(4-chlorophenyl)propyl;

R³ is hydrogen;

R⁴ is hydrogen;

R⁵ to R⁷ are independently selected from hydrogen, bromo, fluoro and chloro; or an enantiomer thereof.

In an embodiment of this invention R² is (3-cyanophenyl)methyl;

R¹ is (2-phenylphenyl)methyl, (3-chlorophenyl)methyl, (3,4-difluorophenyl)methyl, [3,5-bis(trifluoromethyl)phenyl]methyl or [4-(trifluoromethyl)phenyl]methyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is (4-fluorophenyl)methyl;

R¹ is [(4-chlorophenyl)-pyridin-4-yl-methyl], [(4-fluorophenyl)-pyridin-3-yl-methyl], (phenyl-pyridin-2-yl-methyl), 2-(4-methoxyphenyl)ethyl, (4-chlorophenyl)methyl, (2-phenylphenyl)methyl, benzhydryl, (2-pyridin-3-ylphenyl)methyl, (3,4-difluorophenyl)methyl, (1-fluoro-3-phenyl-propan-2-yl), (1-methylpyrrol-2-yl)methyl, (2-phenylphenyl)methyl, 2-(4-chlorophenyl)propyl, 1-(4-chlorophenyl)ethyl, 2-(4-chlorophenyl)propan-2-yl, 2-(4-fluorophenyl)propan-2-yl, 2-phenylpropan-2-yl;

R³ is hydrogen;

R⁴ is hydrogen;

R⁵ to R⁷ are independently selected from hydrogen, bromo, fluoro and chloro;

or an enantiomer thereof.

In an embodiment of this invention R² is (4-hydroxyphenyl)methyl;

R¹ is (3,4-difluorophenyl)methyl, (3-chlorophenyl)methyl, [3,5-bis(trifluoromethyl)phenyl]methyl or [4-(trifluoromethyl)phenyl)methyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is [2-trifluoromethyl)phenyl]methyl;

R¹ is (2-methoxyphenyl)methyl, (2-fluorophenyl)methyl, benzhydryl, 2-(4-chlorophenyl)ethyl, [4-(piperidine-1-carbonyl)phenyl]methyl, 2-(4-chlorophenyl)propyl, (2-phenylphenyl)methyl, 1-phenylpropyl, 2-phenylpropyl, 4-phenylbutan-2-yl, 2-phenylethyl,

3-phenylpropyl, 2-methylbutyl, cyclohexylmethyl, (3-fluorophenyl)methyl, (2-ethoxyphenyl)methyl, [4-(trifluoromethoxy)phenyl]methyl or (3,4-difluorophenyl)methyl

R³ is hydrogen;

R⁴ is hydrogen;

R⁵ to R⁷ are independently selected from hydrogen, bromo, fluoro, chloro, methoxy or methyl;

or an enantiomer thereof.

In an embodiment of this invention R² is [3-difluoromethoxy)phenyl]methyl;

R¹ is 1-phenylethyl, (3-hydroxy-2,2-dimethyl-propyl), 3,3-dimethylbutyl or 2,2-dimethylpropyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is [3-trifluoromethoxy)phenyl]methyl;

R¹ represents 3,3-dimethylbutyl or 2,2-dimethylpropyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is [3-trifluoromethyl)phenyl]methyl;

R^{l} is (3-hydroxy-2,2-dimethyl-propyl), 2-methylpropyl, 3,3-dimethylbutyl, 2,2-dimethylpropyl or (1-methylpyrrol-2-yl)methyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is [4-difluoromethoxy)phenyl]methyl;

R¹ is 2-methylpropyl, 3,3-dimethylbutyl, 2,2-dimethylpropyl, (2-chloro-4-fluoro-phenyl)methyl, 2-(4-chlorophenyl)propyl or [3,5-bis(trifluoromethyl)phenyl]methyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is [6-(trifluoromethyl)pyridin-3-yl]methyl;

R¹ is 2-(4-chlorophenyl)propyl or (2-phenylphenyl)methyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is 2-(1H-indol-3-yl)ethyl;

R¹ is 2-(4-chlorophenyl)propyl, 2-(2-phenoxyphenyl)ethyl, 2-[4-(diethylcarbamoyl)phenyl]ethyl, 2-(3-fluorophenyl)ethyl, 2-[2-(trifluoromethoxy)phenyl]ethyl, 2-(4-fluorophenyl)ethyl, 2-(3,5-dimethoxyphenyl)ethyl, 2-(4-phenylphenyl)ethyl, 2-(4-phenoxyphenyl)ethyl, 2-(2-ethoxyphenyl)ethyl or 2-benzo[1,3]dioxol-5-ylethyl, 2,2-dimethylpropyl, 3,3-dimethylbutyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is 2-(2,4-dichlorophenyl)ethyl;

R¹ is 2-methylpropyl, 3,3-dimethylbutyl or 2,2-dimethylpropyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is 2-(2,6-dichlorophenyl)ethyl;

R¹ is 2-methylpropyl, 3,3-dimethylbutyl or 2,2-dimethylpropyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is 4,4,4-trifluorobutyl;

R¹ is [2-(trifluoromethyl)phenyl]methyl, [(1R)-1-phenylethyl], benzhydryl, 2-(4-chlorophenyl)propyl, (2-phenylphenyl)methyl, (2-phenoxyphenyl)methyl, (2-phenylphenyl)methyl, 2-(4-chlorophenyl)ethyl, 2-(4-fluorophenyl)ethyl, 2-(4-fluorophenyl)propyl, 2-(4-chlorophenyl)propan-2-yl, 2-(4-fluorophenyl)propan-2-yl or 2-phenylpropan-2-yl;

R³ is hydrogen;

R⁴ is hydrogen;

R⁵ to R⁷ are independently selected from hydrogen, —OH, methyl, bromo, fluoro and chloro;

or an enantiomer thereof.

In an embodiment of this invention R² is 4,4-difluorobutyl;

R¹ is (2-cyclopentylphenyl)methyl, [2-(trifluoromethyl)phenyl]methyl, [(1R)-1-phenylethyl], (2-phenylphenyl)methyl, benzhydryl, 2-(4-chlorophenyl)propyl or (2-phenylphenyl)methyl;

R³ is hydrogen;

R⁴ is hydrogen;

R⁵ to R⁷ are independently selected from hydrogen, bromo, fluoro and chloro;

or an enantiomer thereof.

In an embodiment of this invention R² is benzyl;

R¹ represents benzyl, benzhydryl, [2-(trifluoromethyl)phenyl]methyl, (2-pyridin-3-ylphenyl)methyl, (4-phenoxyphenyl)methyl, (2,4-difluorophenyl)methyl, [4-(difluoromethoxy)phenyl]methyl, [3-(difluoromethoxy)phenyl]methyl, (3-pyrrol-1-ylphenyl)methyl, (3-fluorophenyl)methyl, (4-cyanophenyl)methyl, (3,5-dimethoxyphenyl)methyl, (2-methoxyphenyl)methyl, (2-ethoxyphenyl)methyl, [4-(trifluoromethyl)phenyl]methyl, (3,4-difluorophenyl)methyl, (2,5-dimethylphenyl)methyl, [3,5-bis(trifluoromethyl)phenyl]methyl, (2-methylphenyl)methyl, (2,3-difluorophenyl)methyl, (2-bromophenyl)methyl, [(4-fluorophenyl)-pyridin-3-yl-methyl], [(4-chlorophenyl)-pyridin-4-yl-methyl], (phenyl-pyridin-2-yl-methyl), (1-methyl-5-phenyl-pyrazol-3-yl)methyl, (5-methyl-2-phenyl-1,3-oxazol-4-yl)methyl, (5-methyl-3-phenyl-1,2-oxazol-4-yl)methyl, (3-phenyl1,2-oxazol-5-yl)methyl, 2-(4-chlorophenyl)ethyl, 2-(4-fluorophenyl)ethyl, 2[4-(trifluoromethyl)phenyl]ethyl, 2-(5-bromo-2-methoxy-phenyl)ethyl, 2-(3-bromo-4-methoxy-phenyl)ethyl, 2-(4-fluorophenyl)propyl, 2-(4-chlorophenyl)propyl, 4-phenylbutan-2-yl, [2-(4-chloro-2-methyl-phenyl)-2,2-difluoro-ethyl], 2-naphthalen-1-ylpropyl, (2-methyl-2-phenyl-propyl), 2-phenoxypropyl, 2-(4-fluorophenoxy)propyl, 2-phenylpropan-2-yl, cycloheptyl, 2-(2-methoxyphenyl)ethyl, 1-naphthalen-1-ylethyl, 2-[3-(trifluoromethyl)phenyl]ethyl, 2-(6-chloro-1H-indol-3-yl)ethyl, 2-(4-chlorophenyl)propyl, [(1R)-1-(4-methoxyphenyl)ethyl], [(1R)-1-(3-methoxyphenyl)ethyl], 4-phenylbutan-2-yl, 1-phenylethyl, 2-phenylethyl, 1-naphthalen-2-ylethyl, 2-(1-cyclohexenyl)ethyl, 1-(4-fluorophenyl)ethyl, 2-(4-fluorophenyl)propan-2-yl, 2-phenylpropan-2-yl, 1-phenylpropyl or (2-phenylphenyl)methyl;

R³ is hydrogen;

R⁴ is hydrogen;

R⁵—R⁷ are independently selected from hydrogen, bromo, fluoro and chloro, —OH, methyl, or methoxy;

or an enantiomer thereof.

In an embodiment of this invention R² is n-butyl;

R¹ is (2-phenylphenyl)methyl, (2-phenoxyphenyl)methyl, [2-(4-fluorophenoxy)phenyl]methyl, 2-(3-fluorophenyl)ethyl, 2-(4-fluorophenyl)ethyl, 2-(4-chlorophenyl)ethyl, 2-[4-(trifluoromethyl)phenyl]ethyl, 2-(4-chlorophenyl)propyl, 2-(4-fluorophenyl)propyl, (2-phenylphenyl)methyl, 2-(4-phenylphenyl)ethyl, 2-naphthalen-1-ylpropyl, 2-(2-ethoxyphenyl)ethyl, 2-(2-phenoxyphenyl)ethyl, 2-(4-phenoxyphenyl)ethyl, 2-[2-(trifluoromethoxy)phenyl]ethyl, 2-(3,5-dimethoxyphenyl)ethyl, 2-benzo[1,3]dioxol-5-ylethyl, (1-fluoro-3-phenyl-propan-2-yl), 2-(4-chlorophenyl)propyl, naphthalen-1-ylmethyl, 1-naphthalen-2-ylethyl, (2-phenylphenyl)methyl, [2-(4-chlorophenyl)-2-methyl-propyl];

R³ is hydrogen;

R⁴ is hydrogen;

R⁵ to R⁷ are independently selected from hydrogen, bromo, fluoro, chloro, —OH, methyl, methoxy;

or an enantiomer thereof.

In an embodiment of this invention R² is ethyl;

R¹ is benzhydryl, (2-phenylphenyl)methyl or 2-(4-chlorophenyl)propyl

R³ is hydrogen;

R⁴ is hydrogen;

R⁵ to R⁷ are independently selected from hydrogen, bromo, fluoro, chloro, —OH, methyl, methoxy;

or an enantiomer thereof.

In an embodiment of this invention R² is 2-phenylethyl;

R¹ is (2-phenylphenyl)methyl, 2-(4-methoxyphenyl)ethyl, (4-chlorophenyl)methyl, 2-(4-chlorophenyl)ethyl, 2-(3,4-dichlorophenyl)ethyl, (3,4-difluorophenyl)methyl, 2-(4-chlorophenyl)propyl, (2-chloro-4-fluoro-phenyl)methyl or 4-phenylbutan-2-yl;

R³ is hydrogen;

R⁴ is hydrogen;

R⁵ to R⁷ are independently selected from hydrogen, bromo, fluoro, chloro, —OH, methyl, methoxy;

or an enantiomer thereof.

In an embodiment of this invention R² is propyl;

R¹ is (2-phenylphenyl)methyl, benzhydryl or 2-(4-chlorophenyl)propyl;

R³ is hydrogen;

R⁴ is hydrogen;

R⁵ to R⁷ are independently selected from hydrogen, bromo, fluoro, chloro, —OH, methyl, methoxy;

or an enantiomer thereof.

In an embodiment of this invention R² is pyridin-3-ylmethyl or pyridin-4-ylmethyl;

R¹ is (2-phenylphenyl)methyl, 2-(4-chlorophenyl)propyl, (3,4-difluorophenyl)methyl, (2-chloro-4-fluoro-phenyl)methyl or 1-(4-fluorophenyl)ethyl;

R³ is hydrogen;

R⁴ is hydrogen;

R⁵ to R⁷ are independently selected from hydrogen, bromo, fluoro, chloro, —OH;

or an enantiomer thereof.

In an embodiment of this invention R² is tert-butyl;

R¹ is (2-phenylphenyl)methyl, [2-(trifluoromethyl)phenyl]methyl, [4-(difluoromethoxy)phenyl]methyl, (2-chlorophenyl)methyl, (2-methoxyphenyl)methyl, (3,4-difluorophenyl)methyl, (3,4-difluorophenyl)methyl, (4-phenoxyphenyl)methyl, [3,5-bis(trifluoromethyl)phenyl]methyl, (4-fluoro-2-phenyl-phenyl)methyl, (5-fluoro-2-phenyl-phenyl)methyl, 1-phenylethyl, 2-(4-chlorophenyl)ethyl, 2-(2-phenoxyphenyl)ethyl, 2-[2-(trifluoromethoxy)phenyl]ethyl, 2,2-diphenylethyl, 2-(4-fluorophenyl)propyl, 2-(4-chlorophenyl)propyl, (2-phenylphenyl)methyl, 2-(4-phenylphenyl)ethyl, [2-(3-fluorophenyl)phenyl]methyl, [2-(4-fluorophenyl)phenyl]methyl, [2-[3,4-difluorophenyl)phenyl]methyl, [2-(2,4-difluorophenyl)phenyl]methyl, [2-[2,5-difluorophenyl)phenyl]methyl, [2-(2,4-dichlorophenyl)phenyl]methyl, [2-(3,4-dichlorophenyl)phenyl]methyl, [2-(2-chlorophenyl)phenyl]methyl, [2-(4-chlorophenyl)phenyl]methyl, [2-(4-methylphenyl)phenyl]methyl, [2-(4-fluoro-2-methyl-phenyl)phenyl]methyl, [2-(4-methoxyphenyl)phenyl]methyl, [4-fluoro-2-(4-fluorophenyl)phenyl]methyl, [2-(3-chloro-4-fluoro-phenyl)phenyl]methyl, [2-(4-fluoro-2-methyl-phenyl)phenyl]methyl, [5-fluoro-2-(4-fluorophenyl)phenyl]methyl, benzhydryl, [(1R)-2-(4-chlorophenyl)-1-(4,4,4-trifluorobutylcarbamoyl)ethyl], [3,5-bis(trifluoromethyl)phenyl]methyl, 9H-fluoren-9-yl, [2-[4-(trifluoromethyl)phenoxy]phenyl]methyl, 2-naphthalen-1-ylpropyl, [(1R)-2-(4-chlorophenyl)-1-methoxycarbonyl-ethyl], (1-methyl-5-phenyl-pyrazol-3-yl)methyl or [2-(4-chloro-2-methyl-phenyl)-2,2-difluoro-ethyl], (3-phenylphenyl)methyl, (4-fluorophenyl)methyl, (4-phenylphenyl)methyl, [(4-chlorophenyl)-pyridin-4-yl-methyl], 2-(4-fluorophenyl)propyl, 2-(4-phenoxyphenyl)ethyl;

R³ is hydrogen;

R⁴ is hydrogen;

R⁵ to R⁷ are independently —OH, bromo, chloro, fluoro, methyl, methoxy methylsulfonylamino, trimethylsilyl, cyano, —OCHF₂, —OCH₂F, —OSO₂CF₃,

or an enantiomer thereof.

In an embodiment of this invention R² is trimethylsilylmethyl;

R¹ is [3-(difluoromethoxy)phenyl]methyl, [4-(difluoromethoxy)phenyl]methyl, naphthalen-1-ylmethyl, 1-naphthalen-1-ylethyl, 2-(4-bromophenyl)ethyl, (2-chloro-6-phenoxy-phenyl)methyl or (3,4-dichlorophenyl)methyl;

R³ is hydrogen;

R⁴ is hydrogen;

R⁵ is R⁷ are independently selected from hydrogen, bromo, fluoro, chloro;

or an enantiomer thereof.

In one embodiment of the invention the compound of the invention is according to formula I as defined above but also including proviso d), in addition to provisos a), b) and c), that when R² represents —(CH₂)ₖN(R^{19a})(R^{19b}),wherein k represents 2; and R^{19a} and R^{19b}) represent methyl;

or R² represents Het⁴ selected from thiazolyl or pyridyl;

or R² represents phenyl substituted by dimethylamino;

and R⁵—R⁷ are selected from C₁-C₃ alkyl, —OR³⁶, wherein R³⁶ is selected from C₁-C₃ alkyl; then R¹ does not represent phenyl, benzyl, pyridyl, pyridylmethyl, pyrimidinyl, cyclohexyl, methylpiperazinyl, indanyl or naphthyl, optionally substituted by one to three substituents selected from halogen such as fluoro, chloro, bromo, iodo, hydroxy, C₁-C₄ alkyl such as methyl, ethyl, propyl, isopropyl, butyl, C₁-C₄ alkoxy such as methoxy, ethoxy, propoxy, isopropoxy and butoxy, trifluoromethyl, C₁-C₃ alkyl substituted by at least one fluorine atoms, such as trifluoromethoxy, trifluoroethoxy and trifluoropropoxy, amide, carboxy, cyano, C₁-C₄ alkylthio such as methylthio, ethylthio, propylthio and butylthio, nitro, amino, methylamino, dimethylamino, dimethylaminomethyl, dipropylaminomethyl, methylenedioxy, phenoxy, benzyloxy, C₂-C₅ alkanoyloxy such as acetoxy, propionyloxy and butyryloxy, C₁-C₃ ω-hydroxyallkyl such as hydroxymethyl, hydroxyethyl, C₂-C₅ alkanoyloxy-C₁-C₃ alkyl such acetyloxymethyl, acetylocyethyl and propionyloxymethyl, C₂-C₅ alkanoylamino such as acetylamino and propionylamino; alkoxycarbonyl such as methoxycarbonyl, ethoxycarbonyl, propoxycarbonyl, isopropoxycarbonyl and butoxycarbonyl, phenoxycarbonyl and benzyloxycarbonyl.

Unless otherwise specified, alkyl groups and alkoxy groups as defined herein may be straight-chain or, when there is a sufficient number (i.e. a minimum of three) of carbon atoms be branched-chain, or cyclic. Further, when there is a sufficient number (i.e. a minimum of four) of carbon atoms, such alkyl and alkoxy groups may also be part cyclic/acyclic. Unless otherwise specified, alkyl and alkoxy groups may also be substituted by one or more halogen atoms, and especially fluoro atoms. Unless otherwise specified the cyclic alkyl, for example C₃-C₈ cycloalkyl may optionally be substituted by one or more substituents selected from —OH, oxo, halo, cyano, nitro, amino, alkylamino, C₁₋₆ alkyl, C₁₋₆ alkoxy, aryl, aryloxy or a Het group.

Alkylene groups as defined herein are divalent and may be straight-chain or, when there is a sufficient number (i.e. a minimum of three) of carbon atoms, be branched-chain. Unless otherwise specified, alkylene groups may also be substituted by one or more halogen atoms, and especially fluoro atoms.

The term “aryl”, when used herein, includes C₆₋₁₀ aryl groups such as phenyl, naphthyl and the like. The term “aryloxy”, when used herein includes C₆₋₁₀ aryloxy groups such as phenoxy, naphthoxy and the like. For the avoidance of doubt, aryloxy groups referred to herein are attached to the rest of the molecule via the O-atom of the oxy-group. Unless otherwise specified, aryl and aryloxy groups may be substituted by one or more substituents including —OH, halo, cyano, nitro, C₁₋₆ alkyl, C₁₋₆ alkoxy, sulfamoyl, methylsulfonyl, aryl, anilino and methylsulfinyl. When substituted, aryl and aryloxy groups are preferably substituted by between one and three substitutents.

The terms “halo” and “halogen”, when used herein, include fluoro, chloro, bromo and iodo.

Het (Het^{l}-Het⁷⁶) groups that may be mentioned include those containing 1 to 4 heteroatoms (selected from the group oxygen, nitrogen and/or sulfur) and in which the total number of atoms in the ring system are between five and twelve. Het groups may be fully saturated, wholly aromatic, partly aromatic and/or bicyclic in character. Heterocyclic groups that may be mentioned include benzodioxanyl, benzodioxepanyl, benzodioxolyl, benzofuranyl, benzimidazolyl, benzomorpholinyl, benzotriazol, benzoxazinonyl, benzothiophenyl, chromanyl, cinnolinyl, dioxanyl, dioxothiolanyl, furanyl, imidazolyl, imidazo[1,2-c]pyridinyl, indolyl, isoquinolinyl, isoxazolyl, morpholinyl, oxopyrrolidinyl, oxopiperidinyl, oxazolyl, phthalazinyl, piperazinyl, piperidinyl, purinyl, pyranyl, pyrazinyl, pyrazolyl, pyridinyl, pyrimindinyl, pyrrolidinonyl, pyrrolidinyl, pyrrolinyl, pyrrolyl, quinazolinyl, quinolinyl, tetrahydropyranyl, tetrahydrofuranyl, tetrazole, thiazolyl, thienyl, thiochromanyl, triazolyl and the like. Substituents on Het groups may, where appropriate, be located on any atom in the ring system including a heteroatom. The point of attachment of Het groups may be via any atom in the ring system including (where appropriate) a heteroatom, or an atom on any fused carbocyclic ring that may be present as part of the ring system. Het groups may also be in the N- or S-oxidised form.

Unless otherwise specified, the Het group may optionally be substituted by one or more substituents selected from —OH, oxo, halo, cyano, nitro, C₁₋₆ alkyl, C₁₋₆ alkoxy, aryl, aryloxy or a further Het group.

Further, the term “hydrocarbon” refers to any structure comprising only carbon and hydrogen atoms.

The term “hydrocarbon radical” or “hydrocarbyl” refers to any structure as a result of removing one or more hydrogens from a hydrocarbon.

The term “alkenyl” refers to a monovalent straight or branched chain alkyl group having at least one carbon-carbon double bond. The double bond of an alkenyl can be unconjugated or conjugated to another unsaturated group. Unless otherwise specified, alkenyl groups as defined herein may be straight-chain or, when there is a sufficient number (i.e. a minimum of three) of carbon atoms be branched-chain, or cyclic. Further, when there are a sufficient number (i.e. a minimum of four) of carbon atoms, such alkenyl group may also be part cyclic/acyclic. Unless otherwise specified, alkenyl groups may also be substituted by one or more halogen atoms, and especially fluoro atoms.

The term “heteroalkyl” refers to a radical formed as a result of replacing one or more carbon atom of an alkyl with one or more heteroatoms selected from N, O and S.

The compounds of the invention may exhibit tautomerism. All tautomeric forms and mixtures thereof are included within the scope of the invention.

The compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention. All enantiomers, and mixtures thereof, are included within the scope of the invention.

Abbreviations are listed at the end of this specification.

Illustrative examples of any substient, R group or any part of such groups include, but are not limited to:

C₁-C₆ alkyl: methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 2-ethyl-1-butyl, butyl, isobutyl, t-butyl, pentyl, isopentyl, neopentyl, and hexyl;

C₂-C₆ alkenyl: vinyl, allyl, butenyl, pentenyl, hexenyl, cyclohexenyl, butadienyl, pentadienyl, and hexadienyl;

C₃-C₈ cycloalkyl: cyclopropyl, cyclobutyl, cyclopentyl, cyclohexyl and cyclooctyl;

Illustrative examples of substituent Het are benzodioxanyl, benzotriazol, furanyl, imidazolyl, indolyl, oxazolyl, piperazinyl, pyrazinyl, pyrazolyl, pyridinyl, pyrimindinyl, pyrrolidinyl, pyrrolyl and thienyl.

Compounds of the invention that may be mentioned are those wherein R¹ represents (2-phenylphenyl)methyl, (4-phenoxyphenyl)methyl, 2-phenoxyphenyl methyl, 2-(4-chlorophenyl)propyl, 2-(trifluoromethyl)phenylmethyl, 2,2-dimethylpropyl, benzhydryl, 1-phenylethyl or 2,2-dimethylpropyl, [2-(3,4)-difluorophenyl)phenyl]-methyl, [2-(4-chlorophenyl)-2-methyl-propyl], [4-fluoro-2-(4-fluorophenyl)phenyl]methyl, (4-fluoro-2-phenyl-phenyl)methyl, [5-fluoro-2-(4-fluorophenyl)phenyl]methyl, (5-fluoro-2-phenyl-phenyl)methyl, 1-(4-fluorophenyl)ethyl, 2-(4-chlorophenyl)propan-2-yl, 2-(4-fluorophenyl)propan-2-yl or 1-(4-chlorophenyl)ethyl.

Compounds of the invention that may be mentioned are those wherein R² represents ethyl, propyl, butyl, tert-butyl, 4,4-difluorobutyl, 4,4,4-trifluorobutyl, methoxycarbonylmethyl, benzyl, 3,4-dichlorophenylmethyl, (4-fluorophenyl)methyl, [3-(difluoromethoxy)phenyl]methyl, (5-oxo-1-propan-2-yl-pyrrolidin-3-yl)methyl, propan-2-ylcarbamoylmethyl, (2-fluorophenyl)methyl, (3-fluorophenyl)methyl, 1-phenylethyl, 2-phenylpropan-2-yl or 5-cyanopentyl.

Compounds of the invention that may be mentioned include those in which aryl is phenyl, optionally substituted by one or more of the following fluoro, chloro, hydroxy, methoxy, cyano, carbamoyl, dialkylamino, methylsulfonyl, trifluoromethyl, aminoalkyl, difluoromethoxy.

Compounds of the invention that may be mentioned are those where at least one of the substituents in R¹ and R² is an aryl.

Compounds of the invention that may be mentioned are those having R¹ selected from bulky and branched sidechains, for example biphenyls, benzhydryls (diphenylmethyl), branched phenethyls and tertiary butyl groups; and R² is selected from benzyl and lipophilic groups. Lipophilic groups are selected from, for example, tertiary butyl, 4,4-difluorobutyl, 4,4,4-trifluorobutyl and n-butyl.

In one embodiment the compound of the invention is according to formula I wherein

R¹ represents C₁-C₁₂ alkyl (which alkyl group is optionally substituted by one or more groups selected from halogen, C₂-C₆ alkenyl, cyano, oxo, —OR⁸, —SR¹⁰, —COXR¹¹, —N(R^{12a})(R¹²), —N(R^{13a})C(O)OR^{13b}, —OC(O)N(R^{14a})(R^{14b}), —SO₂R¹⁵, aryl or Het¹); further R¹ represents aryl or Het² ;

R⁸, R¹⁰, R¹¹, R^{13a}, R^{13b}, R¹⁵ independently represent, at each occurrence, hydrogen, C₁-C₆ alkyl, aryl or Het⁹ (which C₁-C₆ alkyl, aryl and Het⁹ groups are optionally substituted with one or more substituents selected from —OH, halogen, cyano, nitro, C₁-C₆ alkyl, aryl and Het¹⁰);

R^{12a} and R^{12b} independently represent, at each occurrence, hydrogen, C₁-C₆ alkyl, aryl or Het¹¹ (which C₁-C₆ alkyl, aryl and Het¹¹ groups are optionally substituted with one or more substituents selected from —OH, halogen, cyano, nitro, C₁-C₆ alkyl, aryl and Het¹²);

R^{14a} and R^{14b} independently represent, at each occurrence, hydrogen, C₁-C₆ alkyl, aryl or Het¹³ (which C₁-C₆ alkyl, aryl and Het¹³ groups are optionally substituted with one or more substituents selected from —OH, halogen, cyano, nitro, C₁-C₆ alkyl, aryl and Het14);

R² represents C₁-C₁₂ alkyl (which alkyl group is optionally substituted by one or more groups selected from halogen, C₂-C₆ alkenyl, trialkylsilyl, —COXR¹⁸, aryl or Het³);

further R² represents —(CH₂)ₖN(R^{19a})(R^{19b}), —(CH₂)ₖNR^{20a}C(O)N(R^{20b})(R^{20c}), —(CH₂)ₙNR^{21a}SO₂R^{21b}, CH₂)ₙSO₂R²², —OC(O)N(R^{24a})(R^{24b}), aryl or Het⁴;

R¹⁸, R²¹, R²² independently represent, at each occurrence, hyhdrogen, C₁-C_{6 alkyl, aryl or Het}¹⁵ (which C₁-C₆ alkyl, aryl and Het¹⁵ groups are optionally substituted with one or more substituents selected from —OH, halogen, cyano, nitro, C₁-C₆ alkyl, aryl and Het¹⁶);

R^{19a} and R^{19b} independently represent, at each occurrence, hydrogen, C₁-C₆ alkyl, aryl or Het¹⁹ (which C₁-C₆ alkyl, aryl and Het¹⁹ groups are optionally substituted with one or more substituents selected from —OH, halogen, cyano, nitro, C₁-C₆ alkyl, aryl and Het²⁰)

R^{20a}, R^{20b} and R^{20c} independently represent, at each occurrence, hydrogen, C₁-C₆ alkyl, aryl or Het²¹ (which C₁-C₆ alkyl, aryl and Het²¹ groups are optionally substituted with one or more substituents selected from —OH, halogen, cyano, nitro, C₁-C₆ alkyl, aryl and Het²²);

R³ represents hydrogen, C₁-C₁₂ alkyl (which alkyl group is optionally substituted by one or more groups selected from halogen, C₂-C₆ alkenyl, trialkylsilyl, —COXR²⁷, aryl or Het⁵); further R³ represents —(CH₂)ₖN(R^{28a})(R^{28b}), —(CH₂)ₖN(R^{29a})C(O)N(R^{29b})(R^{29c}), —CH₂)ₙNR^{30a}SO₂R^{30b}, —(CH₂)ₙSO₂R³¹, —OC(O)N(R^{33a})(R^{33b}), aryl or Het⁶;

R²⁷, R^{30a}, R^{30b}, R³¹ independently represent, at each occurrence, hydrogen, C₁-C₆ alkyl, aryl or Het²³ (which C₁-C₆ alkyl, aryl and Het²³ groups are optionally substituted with one or more substituents selected from —OH, halogen, cyano, nitro, C₁-C₆ alkyl, aryl and Het²⁴);

R^{28a} and R^{28b} independently represent, at each occurrence, hydrogen, C₁-C₆ alkyl, aryl or Het²⁵ (which C₁-C₆ alkyl, aryl and Het²⁵ groups are optionally substituted with one or more substituents selected from —OH, halogen, cyano, nitro, C₁-C₆ alkyl, aryl and Het²⁶);

R^{33a} and R^{33b} independently represent, at each occurrence, hydrogen, C₁-C₆ alkyl, aryl or Het²⁷ (which C₁-C₆ alkyl, aryl and Het²⁷ groups are optionally substituted with one or more substituents selected from —OH, halogen, cyano, nitro, C₁-C₆ alkyl, aryl and Het²⁸) ;

R^{29a}, R^{29b}, and R^{29c} independently represent, at each occurrence, hydrogen, C₁-C₆ alkyl, aryl or Het²⁹ (which C₁-C₆ alkyl, aryl and Het²⁹ groups are optionally substituted with one or more substituents selected from —OH, halogen, cyano, nitro, C₁-C₆ alkyl, aryl and Het³⁰);

R⁴ represents hydrogen, —OH, aryl, C₁-C₆ alkyl (which alkyl group is optionally substituted by one or more groups selected from halogen, hydroxy, C₂-C₄ alkenyl, trialkylsilyl), —OR³⁴, —(CH₂)ₘR³⁵;

R³⁴ independently represent, at each occurrence, hydrogen, C₁-C₆ alkyl, aryl or Het³¹ (which C₁-C₆ alkyl, aryl and Het³¹ groups are optionally substituted with one or more substituents selected from —OH, halogen, cyano, nitro, C₁-C₆ alkyl, aryl and Het³²);

R³⁵ independently represent aryl or Het³³ (which aryl and Het³³ groups are optionally substituted with one or more substituents selected from —OH, halogen, cyano, nitro, C₁-C₆ alkyl, aryl and Het³⁴);

R⁵ to R⁷ independently represent, at each occurence, hydrogen, —OH, halogen, cyano, nitro, C₁₋₆ alkyl, —OR³⁶, —N(R^{37a})(R^{37b}), —C(O)R³⁸, —C(O)OR³⁹, —C(O)N(R^{40a})(R^{40b}), —NC(O)OR⁴¹, —OC(O)N(R^{42a})(R^{42b}), —N(R^{43a})C(O)R^{43b}, —N(R^{44a})S(O)₂R^{44b}, —S(O)₂R⁴⁵, —OS(O)₂R⁴⁶, —(CH₂)ₙN(R^{47a})(R^{47b}), —(CH₂)ₙNR^{48a}C(O)N(R^{48b})(R^{48c}), —(CH₂)ₙNR^{49a}SO₂R^{49b}, trialkylsilyl, aryl or Het⁷;

R³⁶, R³⁸, R³⁹, R⁴¹, R⁴³, R^{44a}, R^{44b}, R⁴⁵, R⁴⁶, R^{49a} and R^{49b} independently represent, at each occurrence, hydrogen, C₁-C₆ alkyl, aryl or Het³⁵ (which C₁-C₆ alkyl, aryl and Het³⁵ groups are optionally substituted with one or more substituents selected from —OH, halogen, cyano, nitro, C₁-C₆ alkyl, aryl and Het³⁶);

R^{37a} and R^{37b} independently represent, at each occurrence, hydrogen, C₁-C₆ alkyl, aryl or Het³⁷ (which C₁-C₆ alkyl, aryl and Het³⁷ groups are optionally substituted with one or more substituents selected from —OH, halogen, cyano, nitro, C₁-C₆ alkyl, aryl and Het³⁸);

R^{40a} and R^{40b} independently represent, at each occurrence, hydrogen, C₁-C₆ alkyl, aryl or Het³⁹ (which C₁-C₆ alkyl, aryl and Het³⁹ groups are optionally substituted with one or more substituents selected from —OH, halogen, cyano, nitro, C₁-C₆ alkyl, aryl and Het⁴⁰);

R^{42a} and R^{42b} independently represent, at each occurrence, hydrogen, C₁-C₆ alkyl, aryl or Het⁴¹ (which C₁-C₆ alkyl, aryl and Het⁴¹ groups are optionally substituted with one or more substituents selected from —OH, halogen, cyano, nitro, C₁-C₆ alkyl, aryl and Het⁴²);

R^{47a} and R^{47b} independently represent, at each occurrence, hydrogen, C₁-C₆ alkyl, aryl or Het³ (which C₁-C₆ alkyl, aryl and Het⁴³ groups are optionally substituted with one or more substituents selected from —OH, halogen, cyano, nitro, C₁-C₆ alkyl, aryl and Het⁴⁴);

R^{48a}, R^{48b} and R^{48c} independently represent, at each occurrence, hydrogen, C₁-C₆ alkyl, aryl or Het⁴⁵ (which C₁-C₆ alkyl, aryl and Het⁴⁵ groups are optionally substituted with one or more substituents selected from —OH, halogen, cyano, nitro, C₁-C₆ alkyl, aryl and Het46);

aryl is, at each occurrence, optionally substituted by —OH, halogen, cyano, nitro, C₁-C₆ alkyl, C₃-C₈ cycloalkyl, C₂-C₆ alkenyl, aryl, Het⁸, —OR⁵⁰ , —(CH₂)ₘR⁵¹, —SR⁵², —C(O)R⁵³, —COXR⁵⁴, —N(R^{55a})(R^{55b}), —SO₂R⁵⁶, —OS(O)₂R⁵⁷, —(CH₂)ₘN(R^{58a})(R^{58b}), —CH₂)ₘNR^{59a}C(O)N(R^{59b})(R^{59c}), —C(O)OR⁶⁰, —C(O)N(R^{61a})(R^{61b}), —N(R^{62a}C(O)R^{62b}), —N(R^{63a})C(O)OR^{63b}, —OC(O)N(R^{64a}), —N(R^{65a})S(O)₂R^{65b} and OC(O)R⁶⁶;

R⁵⁰ to R⁵⁴, R⁵⁶, R⁵⁷, R⁶⁰, R^{62a}, R^{62b}), R^{63a}, R^{63b}, R^{65a}, R^{65b} and R⁶⁶ independently represent, at each occurrence, hydrogen, C₁-C₆ alkyl, aryl or Het⁴⁷ (which C₁-C₆ alkyl, aryl and Het⁴⁷ groups are optionally substituted with one or more substituents selected from —OH, halogen, cyano, nitro, C₁-C₆ alkyl, aryl and Het^{“});

R⁵¹ independently represent aryl or Het⁴⁹ (which aryl and Het⁴⁹ groups are optionally substituted with one or more substituents selected from —OH, halogen, cyano, nitro, C₁-C₆ alkyl, aryl and Het⁵⁰);

R^{55a} and R^{55b} independently represent, at each occurrence, hydrogen, C₁-C₆ alkyl, aryl or Het⁵¹ (which C₁-C₆ alkyl, aryl and Het⁵¹ groups are optionally substituted with one or more substituents selected from —OH, halogen, cyano, nitro, C₁-C₆ alkyl, aryl and Het⁵²),

R^{58a} and R^{58b} independently represent, at each occurrence, hydrogen, C₁-C₆ alkyl, aryl or Het⁵³ (which C₁-C₆ alkyl, aryl and Het⁵³ groups are optionally substituted with one or more substituents selected from —OH, halogen, cyano, nitro, C₁-C₆ alkyl, aryl and Het⁵⁴),

R^{59a}, independently represent, at each occurrence, hydrogen, C₁-C₆ alkyl, aryl or Het⁵⁵ (which C₁-C₆ alkyl, aryl and Het⁵⁵ groups are optionally substituted with one or more substituents selected from —OH, halogen, cyano, nitro, C₁-C₆ alkyl, aryl and Het56);

R^{6la} and R^{61b} independently represent, at each occurrence, hydrogen, C₁-C₆ alkyl, aryl or Het⁵⁷ (which C₁-C₆ alkyl, aryl and Het⁵⁷ groups are optionally substituted with one or more substituents selected from —OH, halogen, cyano, nitro, C₁-C₆ alkyl, aryl and Het⁵⁸);

R^{64a} and R^{64b} independently represent, at each occurrence, hydrogen, C₁-C₆ alkyl, aryl or Het⁵⁹ (which C₁-C₆ alkyl, aryl and Het⁵⁹ groups are optionally substituted with one or more substituents selected from —OH, halogen, cyano, nitro, C₁-C₆ alkyl, aryl and Het⁶⁰);

Het¹ to Het⁶⁰ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₂₋₆ alkenyl, aryl, a further Het, —OR⁶⁷, —(CH₂)ₘR⁶⁸, —SR⁶⁹, —COXR⁷⁰, —N(R^{71a})(R^{71b}, —SO₂R⁷², —(CH₂)ₘN(R^{73a})(R^{73b}), —(CH₂)ₘNR^{74a}C(O)N(R^{74b})(R^{74c}), —C(O)R⁷⁵, —C(O)OR⁷⁶, —C(O)N(R^{77a})(R^{77b}), —N(R^{78a})C(O)R^{78b}, —N(R^{79a})S(O)₂R^{79b}, OC(O)R⁸⁰, —NC(O)OR⁸¹, —OC(O)N(R^{82a})(R^{82b});

R⁶⁷, R⁶⁹, R⁷⁰, R⁷², R⁷⁵, R⁷⁶, R^{78a}, R^{78b}, R^{79a}, R^{79b}, R⁸⁰ or R⁸¹ independently represent, at each occurrence, hydrogen, C₁-C₆ alkyl, aryl or Het⁶¹ (which C₁-C₆ alkyl, aryl and Het⁶¹ groups are optionally substituted with one or more substituents selected from —OH, halogen, cyano, nitro, C₁-C₆ alkyl, aryl and Het⁶²);

R⁶⁸ represents aryl or Het⁶³ (which aryl and Het⁶³ groups are optionally substituted with one or more substituents selected from —OH, halogen, cyano, nitro, C₁-C₆ alkyl, aryl and Het⁶⁴);

R^{7la} and R^{71b} independently represent, at each occurrence, hydrogen, C₁-C₆ alkyl, aryl or Het⁶⁵ (which C₁-C₆ alkyl, aryl and Het⁶⁵ groups are optionally substituted with one or more substituents selected from —OH, halogen, cyano, nitro, C₁-C₆ alkyl, aryl and Het⁶⁶),

R^{73a} and R^{73b} independently represent, at each occurrence, hydrogen, C₁-C₆ alkyl, aryl or Het⁶⁷ (which C₁-C₆ alkyl, aryl and Het⁶⁷ groups are optionally substituted with one or more substituents selected from —OH, halogen, cyano, nitro, C₁-C₆ alkyl, aryl and Het⁶⁸);

R^{74a}, R^{74b} and R^{74c} independently represent, at each occurrence, hydrogen, C₁-C₆ alkyl, aryl or Het⁶⁹ (which C₁-C₆ alkyl, aryl and Het⁶⁹ groups are optionally substituted with one or more substituents selected from —OH, halogen, cyano, nitro, C₁-C₆ alkyl, aryl and Het⁷⁰);

R^{77a}, and R^{77b} independently represent, at each occurrence, hydrogen, C₁-C₆ alkyl, aryl or Het⁷¹ (which C₁-C₆ alkyl, aryl and Het⁷¹ groups are optionally substituted with one or more substituents selected from —OH, halogen, cyano, nitro, C₁-C₆ alkyl, aryl and Het⁷²);

R^{82a}, and R^{82b} independently represent, at each occurrence, hydrogen, C₁-C₆ alkyl, aryl or Het⁷³ (which C₁-C₆ alkyl, aryl and Het⁷³ groups are optionally substituted with one or more substituents selected from —OH, halogen, cyano, nitro, C₁-C₆ alkyl, aryl and Het⁷⁴)

Het⁶¹ to Het⁷⁴ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl;

X represents a nitrogen or oxygen atom;

m is an integer of 0 to 10;

n is an integer of 0 to 4;

k is an integer of 1 to 5;

and with the provisos a), b) and c) as defined above.

In one embodiment the compound of the invention is according to formula I

wherein

R⁴ represents —OH, aryl, C₁-C₆ alkyl (which alkyl group is optionally substituted by one or more groups selected from halogen, hydroxy, C₂-C₄ alkenyl, trialkylsilyl), —OR³⁴, ≧(CH₂)ₘR³⁵.

Preparation

According to the invention there is also provided a process for the preparation of compounds of formula I which comprises:

reaction of a compound of formula II,

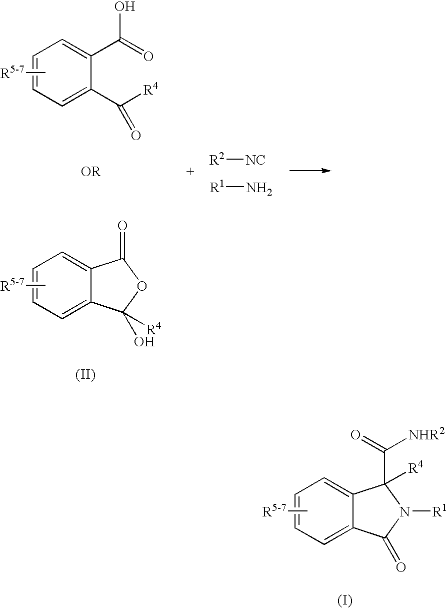

wherein R³ to R⁵ are as hereinbefore defined, with an amine R¹—NH₂ and an isonitrile R²—NC under standard Ugi reaction conditions to give compounds of Formula I wherein R¹ to R⁵ are as hereinbefore defined

According to the invention there is also provided a process for the preparation of compounds of formula I which comprises reaction of a compound of formula III with an amine under standard amidecoupling reaction conditions.

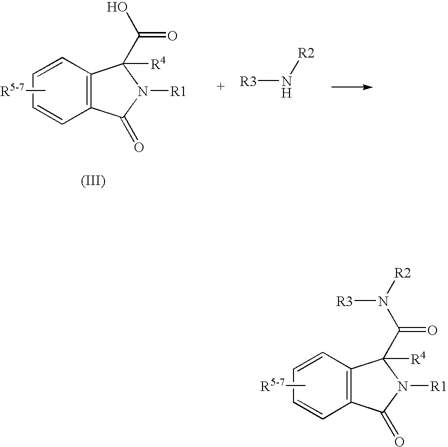

wherein R¹ to R⁷ are as hereinbefore defined.

According to the invention there is also provided a process for the preparation of a compound of formula I which comprises a four-component UGI reaction with an amine, acid, aldehyde and isonitrile to the intermediate compound (IV)

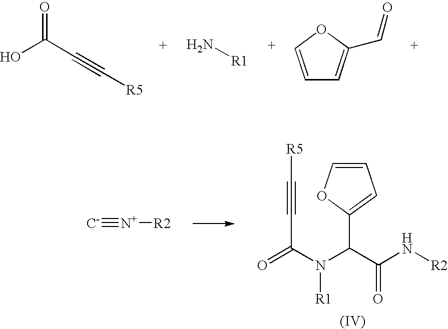

Compound (IV) is then undergoing intramolecular Diels-Alder reaction according to literature methods to give compounds of formula I.

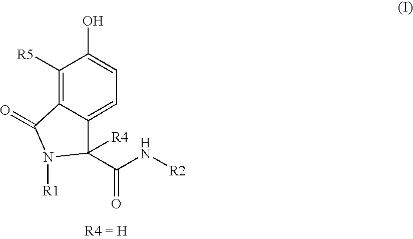

The synthetic sequence originated in a published procedure: D. L. Wright, C. V. Robotham and K. Aboud, Tetrahedron Lett. 2002, 43, 943-946.

According to the invention there is also provided a process for the preparation of a compound of formula 1 which comprises a four-component UGI reaction with an amine, acid, aldehyde and isonitrile to the intermediate compound (V)

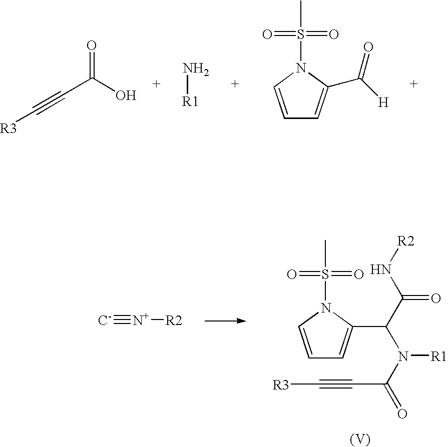

Compound (V) is then undergoing intramolecular Diels-Alder reaction according to literature methods to give compounds of formula I

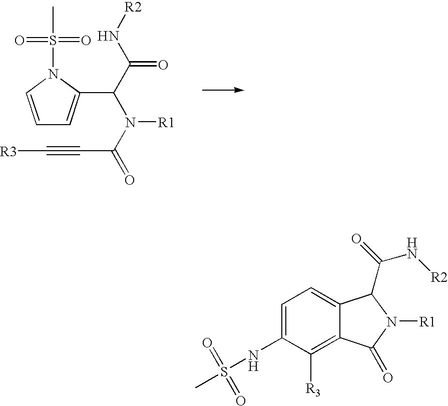

A similar reaction is described in J. Org. Chem. 2004, 69, 1207-1214.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

The prefixes n-, s-, t-, tert- have their usual meanings: normal, secondary, iso and tertiary.

The skilled person will also appreciate that various standard substituent or functional group interconversions and transformations within certain compounds of formula I will provide other compounds of formula I. For example, carbonyl may be reduced to hydroxy or alkylene, and hydroxy may be converted to halogen, and iodo, bromo and chloro may be converted to cyano.

The compounds of the invention may be isolated from their reaction mixtures using conventional techniques.

It will be appreciated by those skilled in the art that, in the process described above, the functional groups of intermediate compounds may be, or may need to be, protected by protecting groups.

Functional groups which it is desirable to protect include hydroxy, amino and carboxylic acid.

Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include benzyl, sulfonamido (e.g. benzenesulfonamido), tert-butyloxycarbonyl, 9-fluorenyl-methoxycarbonyl or benzyloxycarbonyl. Suitable protecting groups for amidino and guanidino include benzyloxycarbonyl. Suitable protecting groups for carboxylic acid include C₁₋₆ alkyl or benzyl esters.

The protection and deprotection of functional groups may take place before or after any of the reaction steps described hereinbefore.

Protecting groups may be removed in accordance with techniques which are well known to those skilled in the art and as described hereinafter.

The use of protecting groups is fully described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

Persons skilled in the art will appreciate that, in order to obtain compounds of the invention in an alternative, and, on some occasions, more convenient, manner, the individual process steps mentioned herein may be performed in a different order, and/or the individual reactions may be performed at a different stage in the overall route (i.e. substituents may be added to and/or chemical transformations performed upon, different intermediates to those associated hereinbefore with a particular reaction). This will depend inter alia on factors such as the nature of other functional groups present in a particular substrate, the availability of key intermediates and the protecting group strategy (if any) to be adopted. Clearly, the type of chemistry involved will influence the choice of reagent that is used in the said synthetic steps, the need, and type, of protecting groups that are employed, and the sequence for accomplishing the synthesis.

It will also be appreciated by those skilled in the art that, although certain protected derivatives of compounds of formula I, which may be made prior to a final deprotection stage, may not possess pharmacological activity as such, they may be administered parenterally or orally and thereafter metabolised in the body to form compounds of the invention which are pharmacologically active. Such derivatives may therefore be described as “prodrugs”. Moreover, certain compounds of formula I may act as prodrugs of other compounds of formula I.

All prodrugs of compounds of formula I are included within the scope of the invention.

Medical and Pharmaceutical Use

Compounds of the invention are useful because they possess pharmacological activity. They are therefore indicated as pharmaceuticals.

Thus, according to a further aspect of the invention there is provided the compounds of the invention for use as pharmaceuticals.

In particular, the compounds of the invention exhibit potassium channel inhibiting activity, especially Kv1.5 blocking activity, for example as demonstrated in the test described below.

The compounds of the invention are thus expected to be useful in both the prophylaxis and the treatment a condition which is effected or facilitated by Kv1.5 inhibition, in particular cardiac arrhythmias, eg.atrial fibrillation, atrial flutter, atrial arrhythmia, atrial tachycardia.

The compounds of the invention are thus indicated in the treatment or prophylaxis of cardiac diseases, or in indications related to cardiac diseases, in which arrhythmias, eg atrial fibrillation, atrial flutter, atrial arrhythmia and atrial tachycardia, are believed to play a major role, including ischaemic heart disease, sudden heart attack, myocardial infarction, heart failure, cardiac surgery and thromboembolic events.

According to a further aspect of the invention, there is provided a method of treatment of an arrhythmia which method comprises administration of a therapeutically effective amount of a compound of the invention to a person suffering from, or susceptible to, such a condition.

Pharmaceutical Preparations

The compounds of the invention will normally be administered orally, subcutaneously, intravenously, intraarterially, transdermally, intranasally, by inhalation, or by any other parenteral route, in the form of pharmaceutical preparations comprising the active ingredient. In a pharmaceutically acceptable dosage form. Depending upon the disorder and patient to be treated, as well as the route of administration, the compositions may be administered at varying doses.

The compounds of the invention may also be combined with any other drugs useful in the treatment of arrhythmias and/or other cardiovascular disorders.

According to a further aspect of the invention there is thus provided a pharmaceutical formulation including a compound of the invention in admixture with a pharmaceutically acceptable adjuvant, diluent or carrier.

Suitable daily doses of the compounds of the invention in therapeutic treatment of humans are about 0.005 to 25.0 mg/kg body weight at oral administration and about 0.005 to 10.0 mg/kg body weight at parenteral administration. Examples of daily doses of the compounds of the invention in therapeutic treatment of humans are about 0.005 to 10.0 mg/kg body weight at oral administration and about 0.005 to 5.0 mg/kg body weight at parenteral administration.

The compounds of the invention have the advantage that they are effective against cardiac arrhythmias.

The compounds of the invention may also be combined with any other drugs useful in the treatment of arrhythmias and/or other cardiovascular disorders.

The compounds of the present invention may be employed alone or in combination with each other and/or other suitable therapeutic agents useful in the treatment of the aforementioned disorders or other disorders, including: other antiarrhythmic agents such as Class I agents (e.g. propafenone), Class II agents (e.g., carvadiol and propranolol), Class III agents (e.g. sotalol, dofetilide, amiodarone, azimilide and ibutilide), Class IV agents (e.g. diltiazem and verapamil), 5 HT antagonists (e.g. sulamserol, serraline and trosetron), dronedarone, atrial selective compounds such as RSD1235, cardiac glycosides including digitalis and ouabain, calcium channel blockers (both L-type and T-type) such as diltiazem, verapamil, nifedipine, amlopdipine and mybefradil.

In another aspect of the invention, the compound of formula (I), or a pharmaceutically acceptable salt or solvate thereof, or a solvate of such a salt, may be administered in association with an antithrombotic agents for example anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab and zolimomab aritox or pharmaceutically acceptable derivative thereof.

In another aspect of the invention, the compound of formula (I), or a pharmaceutically acceptable salt or solvate thereof, or a solvate of such a salt, may be administered in association with other agents that act as or deliver a Factor IIa agonist for example 3DP-4815, AZD-0837, melagatran, ximelagatran, ART-123, lepirudin, AVE-5026, bivaluridin, dabigatran etexilate, E-4444, odiparcil, ardeparin sodium, pegmusirudin, LB-30870, dermatan sulfate, argatroban, MCC-977, desirudin, deligoparin sodium, PGX-100, idraparinux sodium, SR-123781, SSR-182289A, SCH-530348, TRIB50, TGN-167, TGN-255, and compounds described in WO94/29336, WO97/23499 and WO02/44145, which are incorporated hereby by reference.

In another aspect of the invention, the compound of formula (I), or a pharmaceutically acceptable salt or solvate thereof, or a solvate of such a salt, may be administered in association with a fibrinogen receptor antagonists for example roxifiban acetate, fradafiban, orbofiban, lotrafiban hydrochloride, tirofiban, xemilofiban, monoclonal antibody 7E3 and sibrafiban, or pharmaceutically acceptable derivative thereof.

In another aspect of the invention, the compound of formula (I), or a pharmaceutically acceptable salt or solvate thereof, or a solvate of such a salt, may be administered in association with a platelet inhibitors for example cilostezol, clopidogrel bisulfate, epoprostenol, epoprostenol sodium, ticlopidine hydrochloride, aspirin, ibuprofen, naproxen, sulindae, indomethacin, mefenamate, droxicam, diclofenac, sulfinpyrazone and piroxicam, dipyridamole, or pharmaceutically acceptable derivative thereof.

In another aspect of the invention, the compound of formula (I), or a pharmaceutically acceptable salt or solvate thereof, or a solvate of such a salt, may be administered in association with a platelet aggregation inhibitors for example acadesine, beraprost, beraprost sodium, ciprostene calcium, itezigrel, lifarizine, lotrafiban hydrochloride, orbofiban acetate, oxagrelate, fradafiban, orbofiban, tirofiban and xemilofiban or pharmaceutically acceptable derivative thereof.

In another aspect of the invention, the compound of formula (I), or a pharmaceutically acceptable derivative thereof, may be administered in association with a hemorrheologic agents for example pentoxifylline or pharmaceutically acceptable derivative thereof.

In another aspect of the invention, the compound of formula (I), or a pharmaceutically acceptable derivative thereof, may be administered in association with lipoprotein associated coagulation inhibitors; or pharmaceutically acceptable derivative thereof.

In another aspect of the invention, the compound of formula (I), or a pharmaceutically acceptable salt or solvate thereof, or a solvate of such a salt, may be administered in association with a Factor Vlla inhibitor or pharmaceutically acceptable derivative thereof.

Cyclooxygenase inhibitors (i.e. COX-1 and/or COX-2 inhibitors) such as aspirin, indomethacin, ibuprofen, piroxicam, Naproxen ®, Celebrex® and NSAIDs; diuretics such as chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, and spironolactone; anti-hypertensive agents such as alpha adrenergic blockers, beta adrenergic blockers, calcium channel blockers, diuretics, renin inhibitors, ACE inhibitors, (e.g. captopril, zofenopril, fosinopril, enalapril, ceranopril, cilazopril, delapril, pentopril, quinapril, ramipril, lisinopril), A II antagonists (e.g. losartan, irbesartan, valsartan), ET antagonists (e.g. sitaxsentan, atrsentan and compounds disclosed in U.S. Pat. Nos. 5,612,359 and 6,043,265), Dual ET/AII antagonist (e.g. compounds disclosed in WO 00/01389), neutral endopeptidase (NEP) inhibitors, vasopepsidase inhibitors (dual NEP-ACE inhibitors) (e.g. omapatrilat and gemopatrilat), nitrates and combinations of such antihypertensive agents; HMG-CoA reductase inhibitors such as pravastatin, lovastatin, atorvastatin, simvastatin, NK-104 (a.k.a.itavastatin, or nisvastatin or nisbastatin) and ZD-4522 (a.k.a. rosuvastatin, or atavastatin or visastatin); other cholesterol/lipid lowering agents such as LDL lowering agents such as torcetrapid (Pfizer), exetimibe, a combination of atorvastatin and torcetrapid, a combination of simvastatin and ezetimibe, squalene synthetase inhibitors, fibrates, and bile acid sequestrants (e.g. questran).

In another aspect of the invention, the compound of formula (I), or a pharmaceutically acceptable salt or solvate thereof, or a solvate of such a salt, may be administered in association with an anti-obesity compound, or a pharmaceutically acceptable derivative thereof, for example a pancreatic lipase inhibitor e.g. orlistat (EP 129,748), ATL-962, GT-389255 or an appetite (satiety) controlling substance for example sibutramine (Meridia®, Reductil®, GB 2,184,122 and U.S. Pat. No. 4,929,629), PYY 3-36 (amylin), APD-356, 1426, Axokine, T-71, a cannabinoid 1 (CB1) antagonist or inverse agonist, or pharmaceutically acceptable salts, solvates, solvates of such salts or prodrugs thereof, for example rimonabant (EP 656354), AVE-1625, CP945598, SR-147778, SLV-319, and as described in WO01/70700, or a Fatty Acid Synthesis (FAS) inhibitor, or pharmaceutically acceptable salts, solvates, solvates of such salts or prodrugs thereof or a melanin concentrating hormone (MCH) antagonist, or pharmaceutically acceptable salts, solvates, solvates of such salts or prodrugs thereof, for example 856464 and as described in WO 04/004726, anti-diabetic agents such as biguanides (e.g. metformin), glucosidase inhibitors (e.g. acarbose), insulines, meglitinides (e.g. repaglinide), sulfonylureas (e.g. glimepridie, glyburide and glipizide), biguanide/glyburide combinations (i.e. glucovance), thiozolidinediones (e.g. troglitazone, rosiglitazone and pioflitazone), PPAR-gamma agonists, aP2 inhibitors, and DP4 inhibitors; thyroid mimetics (including thyroid receptor antagonists) (e.g. thyrotropin, polythyroid, KB-130015, and dronedarone).

Compounds in the invention can also be administered as the sole active ingredient or in combination with a pacemaker or defribillator device.

In another aspect of the invention, the compound of formula (I), or a pharmaceutically acceptable salt or solvate thereof, or a solvate of such a salt, may be administered in association with an anti-coagulants selected from argatroban, bivalirudin, dalteparin sodium, desirudin, dicumarol, lyapolate sodium, nafamostat mesylate, phenprocoumon, tinzaparin sodium and warfarin sodium or pharmaceutically acceptable derivative thereof.

According to a further aspect of the invention, there is provided a combination product comprising:
(A) a compound of the invention, as hereinbefore defined, or a pharmaceutically acceptable derivative thereof; and
(B) an anticoagulant,

wherein each of components (A) and (B) is formulated in admixture with a pharmaceutically-acceptable adjuvant, diluent or carrier. Component B may also be any of the previously mentioned therapeutic agents.

Such combination products provide for the administration of compounds of the invention in conjunction with the other therapeutic agent, and may thus be presented either as separate formulations, wherein at least one of those formulations comprises a compound of the invention and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, or a pharmaceutically acceptable derivative thereof, an anticoagulant, and a pharmaceutically-acceptable adjuvant, diluent or carrier; and

(2) a kit of parts comprising components:
(a) a pharmaceutical formulation including a compound of the invention, as hereinbefore defined, or a pharmaceutically acceptable derivative thereof, in admixture with a pharmaceutically-acceptable adjuvant, diluent or carrier; and
(b) a pharmaceutical formulation including an anticoagulant with a pharmaceutically- acceptable adjuvant, diluent or carrier,
which components (a) and (b) are each provided in a form that is suitable for administration in conjunction with the other.

When used herein, the term “an anticoagulant” includes references to one a substance selected from the group consisting of aspirin, warfarin, enoxaparin, heparin, low molecular weight heparin, cilostazol, clopidogrel, ticlopidine, tirofiban, abciximab, dipyridamole, plasma protein fraction, human albumin, low molecular weight dextran, hetastarch, reteplase, alteplase, streptokinase, urokinase, dalteparin, filgrastin, immunoglogulin, ginkolide B, hirudins, foropafant, rocepafant, bivalirudin, dermatan sulfate mediolanum, eptilibatide, tirofiban, thrombomodulin, abcxmab, low molecular weight dermatan sulfate-opocrin, eptacog alfa, argatroban, fondaparinux sodium, tifacogin, lepirudin, desirudin, OP2000, roxifiban, parnaparin sodium, human hemoglobin (Hemosol), bovine hemoglobin (Biopure), human hemoglobin (Northfield), antithrombin III, RSR 13, heparin-oral (Emisphere) transgenic antithrombin III, H37695, enoxaparin sodium, mesoglycan, CTC 111, bivalirudin, and any derivatives and/or combinations thereof.

Particular anticoagulants that may be mentioned include aspirin and warfarin.

The term “an anticoagulant” also includes references to thrombin inhibitors. Thrombin inhibitors that may be mentioned include low molecular weight thrombin inhibitors. The term “low molecular weight thrombin inhibitors” will be understood by those skilled in the art, and includes references to any composition of matter (e.g. chemical compound) that inhibits thrombin to an experimentally determinable degree (as determined by in vivo and/or in vitro tests), and which possesses a molecular weight of below about 2,000, preferably below about 1,000.

Preferred low molecular weight thrombin inhibitors include low molecular weight peptide-based, amino acid-based, and/or peptide analogue-based, thrombin inhibitors, as well as derivatives thereof.

The term “low molecular weight peptide-based, amino acid-based, and/or peptide analogue-based, thrombin inhibitors” will be well understood by one skilled in the art to include references to low molecular weight thrombin inhibitors with one to four peptide linkages, and includes those described in the review paper by Claesson in Blood Coagul. Fibrin. 5, 411 (1994), as well as those disclosed in U.S. Pat. No 4,346,078, International Patent Applications WO 93/11152, WO 93/18060, WO 93/05069, WO 94/20467, WO 94/29336, WO 95/35309, WO 95/23609, WO 96/03374, WO 96/06832, WO 96/06849, WO 96/25426, WO 96/32110, WO 97/01338, WO 97/02284, WO 97/15190, WO 97/30708, WO 97/40024, WO 97/46577, WO 98/06740, WO 97/49404, WO 97/11693, WO 97/24135, WO 97/47299, WO 98/01422, WO 98/57932, WO 99/29664, WO 98/06741, WO 99/37668, WO 99/37611, WO 98/37075, WO 99/00371, WO 99/28297, WO 99/29670, WO 99/40072, WO 99/54313, WO 96/31504, WO 00/01704 and WO 00/08014; and European Patent Applications 648 780, 468 231, 559 046, 641 779, 185 390, 526 877, 542 525, 195 212, 362 002, 364 344, 530 167, 293 881, 686 642, 669 317, 601 459 and 623 596, the disclosures in all of which documents are hereby incorporated by reference.

In the present application, derivatives of thrombin inhibitors include chemical modifications, such as esters, prodrugs and metabolites, whether active or inactive, and pharmaceutically acceptable salts and solvates, such as hydrates, of any of these, and solvates of any such salt.

Preferred low molecular weight peptide-based thrombin inhibitors include those known collectively as the “gatrans”. Particular gatrans which may be mentioned include HOOC—CH₂—(R)Cha-Pic-Nag-H (known as inogatran) and HOOC—CH₂—R)Cgl-Aze-Pab-H (known as melagatran) (see International Patent Application WO 93/11152 and WO 94/29336, respectively, and the lists of abbreviations contained therein).

International Patent Application WO 97/23499 discloses a number of compounds which have been found to be useful as prodrugs of thrombin inhibitors. Said prodrugs have the general formula

R^{a}OOC—CH₂—(R)Cgl-Aze-Pab-R^{b}

wherein R^{a} represents H, benzyl or C₁₋₁₀ alkyl, R^{b} (which replaces one of the hydrogen atoms in the amidino unit of Pab-H) represents OH, OC(O)R^{c} or C(O)OR^{d}, R^{c} represents C₁₋₁₇ alkyl, phenyl or 2-naphthyl and R^{d} represents C₁₋₁₂ alkyl, phenyl, C₁₋₃alkylphenyl, or 2-naphthyl. Preferred compounds include R^{a}OOC—CH₂—(R)Cgl-Aze-Pab—OH, wherein R^{a} represents benzyl or C₁₋₁₀ alkyl, e.g. ethyl or isopropyl, especially EtOOC—CH₂—(R)Cgl-Aze-Pab-OH. The active thrombin inhibitors themselves are disclosed in WO 94/29336.

Further low molecular weight thrombin inhibitors that may be mentioned include those disclosed in WO 02/44145, such as compounds of the following general formula,

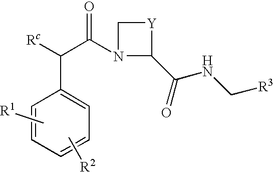

wherein

R^{c} represents —OH or —CH₂OH;

R¹ represents at least one optional halo substituent;

R² represents one or two C₁₋₃ alkoxy substituents, the alkyl parts of which substituents are themselves substituted with one or more fluoro substituents (i.e. R² represents one or two fluoroalkoxy(C₁₋₃) groups);

Y represents —CH₂— or —(CH₂)₂—; and

R³ represents a structural fragment of formula I(i) or I(ii):

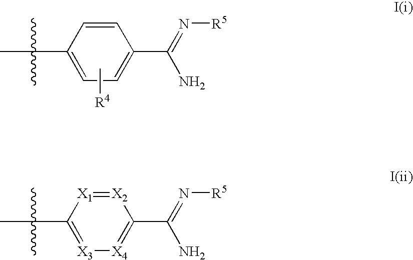

wherein

R⁴ represents H or one or more fluoro substituents;

R⁵ represents H, OR⁶ or C(O)OR⁷;

R⁶ represents H, C₁₋₁₀ alkyl, C₁₋₃ alkylaryl or C₁₋₃ alkyloxyaryl (the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents);

R⁷ represents C₁₋₁₀ alkyl (which latter group is optionally interrupted by one or more oxygen atoms), or C₁₋₃ alkylaryl or C₁₋₃ alkyloxyaryl (the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents); and

one or two of X₁, X₂, X₃ and X₄ represent —N— and the others represent —CH—;

or a pharmaceutically-acceptable derivative thereof.

Compounds of the above general formula in which R⁵ is other than H have been found to be useful as prodrugs of thrombin inhibitors (which thrombin inhibitors include the corresponding compounds of the above general formula in which R⁵ is H).

Particular compounds disclosed in WO 02/44145 that may be mentioned include those of the following general formula:

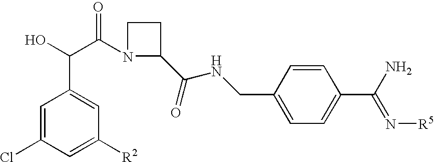

wherein

R² represents —OCHF₂, —OCF₃, —OCH₂CH₂F or —OCH₂CHF₂;

R⁵ represents H or OR⁶; and

R⁶ represents methyl, ethyl, n-propyl, i-propyl or cyclobutyl.

In this respect, more particular compounds disclosed in WO 02/44145 that may be mentioned include the thrombin inhibitor

Ph(3-Cl)(5-OCHF₂)—(R)CH(OH)C(O)-Aze-Pab

and its methoxyamidino prodrug

Ph(3-Cl)(5-OCHF₂)—(R)CH(OH)C(O)-Aze-Pab(OMe).

The compounds of the invention have the advantage that they are effective against cardiac arrhythmias.

Compounds of the invention have advantageous properties compared to compounds of the prior art, in particular enhanced potency, enhanced selectivity, and/or reduction of total clearance. These advantages may provide for corresponding useful properties in practice. For example, when used as pharmaceutical agents, compounds of the present invention may have a lower daily clinical dose, longer duration of action, and/or an improved side effect profile.

Compounds of the invention may also have the advantage that they may be more efficacious than, be less toxic than, have a broader range of activity than, be more potent than, be longer acting than, produce fewer side effects (including a lower incidence of proarrhythmias such as torsades de pointes) than, be more easily absorbed than, or that they may have other useful pharmacological properties over, compounds known in the prior art.

Biological Tests

Test A

Rb⁺ Efflux Assay

This assay identifies compounds that block the human Kv1.5 channel potassium channel heterologously expressed in Chinese Hamster Ovary (CHO) cells by means of Rb+ion efflux using Flame Atomic Absorption Spectroscopy. For experimental studies, CHO cells stably transfected with cDNA for human Kv1.5 were grown as confluent layers in Falcon, 384-well tissue culture-treated black-walled clear-bottomed plates and the plates were incubated overnight at 37° C. in a cell culture incubator.

After incubating overnight the cell plates were washed and a buffer containing Rb+ ions were added to the cell plates. The plates were then incubated for another 3-4 hours in a CO2-incubator (37° C.). Following this incubation period plates were washed, compounds were added and subsequently a buffer containing elevated K+ concentrations were added in order to activate the Kv1.5 channel. Following a short incubation time, aliquots of the supernatants were transferred to supernatant plates for subsequent determination of the Rb+ content using Atomic Absorption Spectrometry (ICR8000 instrument, Aurora Biomed Inc.). The basal Rb+ efflux (conc. mg/L in wells receiving only wash buffer) was defined as 100% inhibition and the stimulated Rb+ efflux (conc. mg/L in wells receiving only buffer containing elevated concencentrations of K⁺ions) was defined as 0% inhibition.

Test B

Electrophysiological recordings of potassium currents in cells stably expressing the the human Kv1.5 potassium channel confirms activity and provides a functional measure of the potency of compounds that specifically affect Kv1.5 channels. Electrophysiological studies were performed using the high throughput planar patch clamp assay (Schroeder et al, J Biomol. Screen (2003)8(1);50-64; Willumsen, Am Biotech Lab (2006)24(4);20-21) or the standard whole cell configuration of the patch clamp technique (Hamill et al, Pflugers Archiv (1981) 391:85). CHO cells, stably transfected with cDNA for human Kv1.5, were then exposed to the drugs and Kv1.5 channels were activated 4 1.7 N-benzyl-2-(1-methyl-1-phenylethyl)-3-oxoisoindoline-1-carboxamide by a test protocol adapted from Pelsson et al (Cardiavasc Pharmacol (2005)46:7-17). Data analysis was performed off-line, paired comparisions between pre-drug and post-drug were used to determine the inhibitory effect of each compound.

The title compounds of the above Examples were tested in Test A and Test B. Most of the compounds of the invention have an activity when tested in Test A, and most of them were found to exhibit an IC₅₀ of <30 μM, preferably an IC₅₀ of <10 μM, or an inhibition of >20% a a concentration of 30 μM, or

Activity of IC50<10 μMin test A:

| | IC50 | |
|---|---|---|
| The compound of the invention | Test A | example |
| N-benzyl-2-(1-methyl-1-phenylethyl)-3-oxoisoindoline- | 1.7 | |
| 1-carboxamide | | |
| N-benzyl-3-oxo-2-(1-phenylpropyl)isoindoline-1- | 0.4 | |
| carboxamide | | |
| N-[3-(difluoromethoxy)benzyl]-3-oxo-2-(1- | 2.4 | |
| phenylethyl)isoindoline-1-carboxamide | | |
| N,2-dibenzyl-6-bromo-3-oxoisoindoline-1-carboxamide | 0.84 | |
| (1R or 1S)-N-benzyl-3-oxo-2-[(1S or 1R)-1- | 1.5 | 25 |
| phenylethyl]isoindoline-1-carboxamide (E4) | | |
| 6-bromo-2-(2-cyclopentylbenzyl)-N-methyl-3- | 2.7 | |
| oxoisoindoline-1-carboxamide | | |
| 6-chloro-2-(2-cyclopentylbenzyl)-N-(4,4-difluorobutyl)- | 1.3 | |
| 3-oxoisoindoline-1-carboxamide | | |
| N-benzyl-6-chloro-3-oxo-2-[(1S)-1- | 2.2 | |
| phenylethyl]isoindoline-1-carboxamide | | |
| N-benzyl-5-bromo-3-oxo-2-[(1R)-1- | 2.1 | |
| phenylethyl]isoindoline-1-carboxamide | | |
| N-benzyl-6-bromo-3-oxo-2-[(1R)-1- | 0.21 | |
| phenylethyl]isoindoline-1-carboxamide | | |
| N-[3-(difluoromethoxy)benzyl]-6-fluoro-2-(3-hydroxy- | 1.4 | |
| 2,2-dimethylpropyl)-3-oxoisoindoline-1-carboxamide | | |
| N-(3-chlorobenzyl)-6-fluoro-2-(3-hydroxy-2,2- | 0.34 | |
| dimethylpropyl)-3-oxoisoindoline-1-carboxamide | | |
| 6-fluoro-2-(3-hydroxy-2,2-dimethylpropyl)-3-oxo-N-[3- | 1.7 | |
| (trifluoromethyl)benzyl]isoindoline-1-carboxamide | | |
| 6-chloro-N-[3-(difluoromethoxy)benzyl]-2-(3-hydroxy- | 0.41 | |
| 2,2-dimethylpropyl)-3-oxoisoindoline-1-carboxamide | | |
| 6-chloro-N-(3-chlorobenzyl)-2-(3-hydroxy-2,2- | 2.1 | |
| dimethylpropyl)-3-oxoisoindoline-1-carboxamide | | |
| N-(tert-butyl)-3-oxo-2-[2-(trifluoromethyl)benzyl] | 1.9 | |
| isoindoline-1-carboxamide | | |
| N-(tert-butyl)-6-chloro-3-oxo-2-[2- | 1.1 | |
| (trifluoromethyl)benzyl]isoindoline-1-carboxamide | | |
| 6-chloro-3-oxo-N-(4,4,4-trifluorobutyl)-2-[2- | 2.5 | |
| (trifluoromethyl)benzyl]isoindoline-1-carboxamide | | |
| 6-chloro-3-oxo-2-[(1R)-1-phenylethyl]-N-(4,4,4- | 2 | |
| trifluorobutyl)isoindoline-1-carboxamide | | |
| 2-(biphenyl-2-ylmethyl)-N-(tert-butyl)-6-cyano-3- | 2.4 | 36 |
| oxoisoindoline-1-carboxamide | | |
| 2-[(4-chlorophenyl)(pyridin-4-yl)methyl]-N-(4- | 2.1 | |
| fluorobenzyl)-3-oxoisoindoline-1-carboxamide | | |
| 2-(biphenyl-2-ylmethyl)-6-chloro-N-ethyl-3- | 1 | |
| oxoisoindoline-1-carboxamide | | |
| 2-(biphenyl-2-ylmethyl)-6-chloro-3-oxo-N- | 1.1 | |
| propylisoindoline-1-carboxamide | | |
| 2-(biphenyl-2-ylmethyl)-6-fluoro-3-oxo-N- | 2.8 | |
| propylisoindoline-1-carboxamide | | |
| 6-chloro-2-[2-(4-chlorophenyl)propyl]-3-oxo-N- | 0.68 | |
| (pyridin-3-ylmethyl)isoindoline-1-carboxamide | | |
| 6-chloro-2-[2-(4-chlorophenyl)propyl]-3-oxo-N-{[6- | 0.42 | |
| (trifluoromethyl)pyridin-3-yl]methyl}isoindoline-1- | | |
| carboxamide | | |
| 2-(biphenyl-2-ylmethyl)-6-chloro-3-oxo-N-{[6- | 1.5 | |
| (trifluoromethyl)pyridin-3-yl]methyl}isoindoline-1- | | |
| carboxamide | | |
| 2-[2-(4-chlorophenyl)propyl]-3-oxo-N-{[6- | 0.78 | |
| (trifluoromethyl)pyridin-3-yl]methyl}isoindoline-1- | | |
| carboxamide | | |
| 2-(biphenyl-2-ylmethyl)-3-oxo-N-{[6- | 0.94 | |
| (trifluoromethyl)pyridin-3-yl]methyl}isoindoline-1- | | |
| carboxamide | | |
| N-benzyl-6-chloro-3-oxo-2-[(1R)-1- | 0.55 | 9 |
| phenylethyl]isoindoline-1-carboxamide | | |
| N-benzyl-6-fluoro-3-oxo-2-[(1R)-1- | 1.3 | |
| phenylethyl]isoindoline-1-carboxamide | | |
| N-benzyl-6-fluoro-3-oxo-2-[2- | 1.4 | |
| (trifluoromethyl)benzyl]isoindoline-1-carboxamide | | |
| 2-[2-(4-chlorophenyl)propyl]-6-fluoro-3-oxo-N- | 2.3 | |
| propylisoindoline-1-carboxamide | | |
| 2-[2-(4-chlorophenyl)propyl]-3-oxo-N- | 1.3 | |
| propylisoindoline-1-carboxamide | | |
| 2-[2-(4-chlorophenyl)propyl]-3-oxo-N-(4,4,4- | 1.8 | |
| trifluorobutyl)isoindoline-1-carboxamide | | |
| 2-(biphenyl-2-ylmethyl)-3-oxo-N-(4,4,4- | 1.5 | |
| trifluorobutyl)isoindoline-1-carboxamide | | |
| N-(tert-butyl)-2-[(1-methyl-5-phenyl-1H-pyrazol-3- | 2.5 | 28 |
| yl)methyl]-3-oxoisoindoline-1-carboxamide | | |
| 2-(biphenyl-2-ylmethyl)-6-bromo-N-(tert-butyl)-3- | 2.4 | 29 |
| oxoisoindoline-1-carboxamide | | |
| N-(tert-butyl)-2-[(3′,4′-difluorobiphenyl-2-yl)methyl]-5- | 2 | |
| hydroxy-4-methyl-3-oxoisoindoline-1-carboxamide | | |
| N-(tert-butyl)-2-[(3′,4′-dichlorobiphenyl-2-yl)methyl]-5- | 0.72 | |
| hydroxy-4-methyl-3-oxoisoindoline-1-carboxamide | | |
| N-[3-(difluoromethoxy)benzyl]-2-(3,3-dimethylbutyl)- | 1.8 | |
| 3-oxoisoindoline-1-carboxamide | | |
| N-[3-(difluoromethoxy)benzyl]-2-(2,2-dimethylpropyl)- | 1.3 | |
| 3-oxoisoindoline-1-carboxamide | | |
| N-(tert-butyl)-6-chloro-2-(diphenylmethyl)-3- | 2.8 | |
| oxoisoindoline-1-carboxamide | | |
| 2-(biphenyl-2-ylmethyl)-6-chloro-3-oxo-N-(4,4,4- | 0.61 | |
| trifluorobutyl)isoindoline-1-carboxamide | | |
| N-benzyl-2-(biphenyl-2-ylmethyl)-6-chloro-3- | 0.42 | |
| oxoisoindoline-1-carboxamide | | |
| 2-(biphenyl-2-ylmethyl)-N-(tert-butyl)-6-chloro-3- | 1.6 | |
| oxoisoindoline-1-carboxamide | | |
| 6-chloro-2-[2-(4-chlorophenyl)propyl]-3-oxo-N-(4,4,4- | 0.46 | |
| trifluorobutyl)isoindoline-1-carboxamide | | |
| 6-chloro-2-[2-(4-chlorophenyl)propyl]-N-[4- | 2.8 | |
| (methylsulfonyl)benzyl]-3-oxoisoindoline-1- | | |
| carboxamide | | |
| N-(tert-butyl)-6-chloro-2-[2-(4-chlorophenyl)propyl]-3- | 0.91 | |
| oxoisoindoline-1-carboxamide | | |
| N-benzyl-6-chloro-2-[2-(4-chlorophenyl)propyl]-3- | 0.15 | |
| oxoisoindoline-1-carboxamide | | |
| N-(4,4-difluorobutyl)-2-(diphenylmethyl)-3- | 8 | 22 |
| oxoisoindoline-1-carboxamide | | |
| 2-[2-(4-chlorophenyl)propyl]-N-[(2,2-difluoro-1,3- | 0.58 | |
| benzodioxol-5-yl)methyl]-3-oxoisoindoline-1- | | |
| carboxamide | | |
| N-(3,4-dichlorobenzyl)-2-(2,2-dimethylpropyl)-3- | 2.5 | |
| oxoisoindoline-1-carboxamide | | |
| (R or S)2-(biphenyl-2-ylmethyl)-N-(tert-butyl)-5- | 1.1 | 3 |
| hydroxy-4-methyl-3-oxoisoindoline-1-carboxamide(E1) | | |
| (S o R) 2-(biphenyl-2-ylmethyl)-N-(tert-butyl)-5- | 17 | 3 |
| hydroxy-4-methyl-3-oxoisoindoline-1-carboxamide(E2) | | |
| 2-(biphenyl-2-ylmethyl)-6-fluoro-3-oxo-N-(4,4,4- | 2.7 | 19 |
| trifluorobutyl)isoindoline-1-carboxamide | | |
| 2-(biphenyl-2-ylmethyl)-N-(tert-butyl)-6-fluoro-3- | 6.7 | |
| oxoisoindoline-1-carboxamide | | |
| N-benzyl-2-(biphenyl-2-ylmethyl)-6-fluoro-3- | 1 | |
| oxoisoindoline-1-carboxamide | | |
| 2-(biphenyl-2-ylmethyl)-N-(tert-butyl)-5- | 1.7 | 14 |
| (fluoromethoxy)-4-methyl-3-oxoisoindoline-1- | | |
| carboxamide | | |
| 2-(biphenyl-2-ylmethyl)-N-(tert-butyl)-5-methoxy-4- | 2.2 | |
| methyl-3-oxoisoindoline-1-carboxamide | | |
| N-(3-chlorobenzyl)-2-(2,2-dimethylpropyl)-3- | 2.9 | |
| oxoisoindoline-1-carboxamide | | |
| N-benzyl-2-[2-(4-chlorophenyl)ethyl]-N-ethyl-3- | 0.93 | |
| oxoisoindoline-1-carboxamide | | |
| N-benzyl-2-[2-(4-chlorophenyl)ethyl]-N-methyl-3- | 0.89 | |
| oxoisoindoline-1-carboxamide | | |
| N-(tert-butyl)-5-hydroxy-4-methyl-3-oxo-2-{2-[4- | 2 | |
| (trifluoromethyl)phenoxy]benzyl}isoindoline-1- | | |
| carboxamide | | |
| N-benzyl-2-[2-(4-chlorophenyl)ethyl]-3-oxoisoindoline- | 0.56 | |
| 1-carboxamide | | |
| 2-(diphenylmethyl)-N-(4-fluorobenzyl)-3- | 2.5 | |
| oxoisoindoline-1-carboxamid | | |
| N-benzyl-2-[2-(4-chlorophenyl)propyl]-5-methoxy-3- | 0.71 | |
| oxoisoindoline-1-carboxamide | | |
| N-benzyl-2-[2-(4-chlorophenyl)propyl]-5,6-dimethoxy- | 0.74 | |
| 3-oxoisoindoline-1-carboxamide | | |
| N-benzyl-N-butyl-2-[2-(4-chlorophenyl)ethyl]-3- | 0.34 | |
| oxoisoindoline-1-carboxamide | | |
| N-(tert-butyl)-2-[(3′,4′-difluorobiphenyl-2-yl)methyl]-3- | 2.2 | 46 |
| oxoisoindoline-1-carboxamide | | |
| N-(tert-butyl)-2-[(4′-fluorobiphenyl-2-yl)methyl]-3- | 7.3 | |
| oxoisoindoline-1-carboxamide | | |
| 2-[2-(4-chlorophenyl)propyl]-N-[2-(1H-indol-3- | 0.62 | |
| yl)ethyl]-3-oxoisoindoline-1-carboxamide | | |
| 2-(biphenyl-2-ylmethyl)-N-(tert-butyl)-7-hydroxy-3- | 3 | 42 |
| oxoisoindoline-1-carboxamide | | |
| 2-(biphenyl-2-ylmethyl)-N-(4-{[(difluoroacetyl)- | 2.4 | 17 |
| amino]methyl}benzyl)-3-oxoisoindoline-1- | | |
| carboxamide | | |
| 2-(3-chlorobenzyl)-N-(3-cyanobenzyl)-3- | 2 | |
| oxoisoindoline-1-carboxamide | | |
| 2-[2-(4-chlorophenyl)propyl]-N-(4-cyanobenzyl)-3- | 1.2 | |
| oxoisoindoline-1-carboxamide | | |
| 2-(biphenyl-2-ylmethyl)-N-(4-cyanobenzyl)-3- | 2.8 | 7 |
| oxoisoindoline-1-carboxamide | | |
| N-(tert-butyl)-2-[2-(4-chlorophenyl)propyl]-5- | 3 | |
| (fluoromethoxy)-4-methyl-3-oxoisoindoline-1- | | |
| carboxamide | | |
| 2-[3,5-bis(trifluoromethyl)benzyl]-N-[4- | 1.8 | |
| (dimethylamino)benzyl]-3-oxoisoindoline-1- | | |
| carboxamide | | |
| 2-[2-(4-chlorophenyl)propyl]-N-[4- | 1.8 | |
| (dimethylamino)benzyl]-3-oxoisoindoline-1- | | |
| carboxamide | | |
| N-(1,3-benzodioxol-5-ylmethyl)-2-[2-(4- | 0.62 | |
| chlorophenyl)propyl]-3-oxoisoindoline-1-carboxamide | | |
| (1S or 1R)-N-butyl-2-[(2R or 2S)-2-(4- | 0.73 | 13 |
| chlorophenyl)propyl]-6-fluoro-3-oxoisoindoline-1- | | |
| carboxamide (E2) | | |
| (1R or 1S)-N-butyl-2-[(2S or 2R)-2-(4- | 1.7 | 13 |
| chlorophenyl)propyl]-6-fluoro-3-oxoisoindoline-1- | | |
| carboxamide (E4) | | |
| (1R or 1S)-N-butyl-2-[(2R or 2S)-2-(4- | 0.5 | 13 |
| chlorophenyl)propyl]-6-fluoro-3-oxoisoindoline-1- | | |
| carboxamide (E3) | | |
| (1S or 1R)-N-butyl-2-[(2S or 2R)-2-(4- | 15 | 13 |
| chlorophenyl)propyl]-6-fluoro-3-oxoisoindoline-1- | | |
| carboxamide (E1) | | |
| N-benzyl-3-oxo-2-[(3-phenylisoxazol-5- | 1 | |
| yl)methyl]isoindoline-1-carboxamide | | |
| N-butyl-2-[2-(4-chlorophenyl)propyl]-6-fluoro-3- | 1.3 | 12 |
| oxoisoindoline-1-carboxamide | | |
| N-(tert-butyl)-5,6-dichloro-2-[2-(4- | 1.8 | |
| chlorophenyl)propyl]-3-oxoisoindoline-1-carboxamide | | |
| 2-[2-(4-chlorophenyl)propyl]-3-oxo-N-[2- | 0.7 | |
| (trifluoromethyl)benzyl]isoindoline-1-carboxamide | | |
| N-(2-chlorobenzyl)-2-[2-(4-chlorophenyl)propyl]-3- | 0.75 | |
| oxoisoindoline-1-carboxamide | | |
| (R or S)2-(biphenyl-2-ylmethyl)-N-(tert-butyl)-3- | 3.4 | 34 |
| oxoisoindoline-1-carboxamide | | |
| 2-[2-(4-chlorophenyl)propyl]-N-(4-fluorobenzyl)-3- | 1.9 | |
| oxoisoindoline-1-carboxamide | | |
| N-benzyl-2-[2-(4-chlorophenyl)propyl]-4,5-dimethoxy- | 0.61 | |
| 3-oxoisoindoline-1-carboxamide | | |
| N-benzyl-2-[2-(4-chlorophenyl)propyl]-3- | 0.51 | |
| oxoisoindoline-1-carboxamide | | |
| N-benzyl-3-oxo-2-(1-phenylethyl)isoindoline-1- | 6 | 24 |
| carboxamide | | |
| N-benzyl-2-(2-bromobenzyl)-3-oxoisoindoline-1- | 2.5 | |
| carboxamide | | |
| 2-(biphenyl-2-ylmethyl)-N-(tert-butyl)-3- | 3.3 | 33 |
| oxoisoindoline-1-carboxamide | | |
| N-benzyl-2-(2-cyclohex-1-en-1-ylethyl)-3- | 1.8 | |
| oxoisoindoline-1-carboxamide | | |
| N-benzyl-2-(biphenyl-2-ylmethyl)-3-oxoisoindoline-1- | 1 | |
| carboxamide | | |
| N-butyl-2-[2-(4-chlorophenyl)propyl]-3-oxoisoindoline- | 0.73 | |
| 1-carboxamide | | |
| N-butyl-2-[2-(4-chlorophenyl)-2-methylpropyl]-3- | 0.53 | 49 |
| oxoisoindoline-1-carboxamide | | |
| N-(tert-butyl)-2-[(4′,5-difluorobiphenyl-2-yl)methyl]-3- | 9.3 | |
| oxoisoindoline-1-carboxamide | | |
| N-(tert-butyl)-2-[(5-fluorobiphenyl-2-yl)methyl]-3- | 13 | |
| oxoisoindoline-1-carboxamide | | |
| N-(tert-butyl)-2-[(4,4′-difluorobiphenyl-2-yl)methyl]-3- | 5.6 | 48 |
| oxoisoindoline-1-carboxamide | | |
| N-(tert-butyl)-2-[(4-fluorobiphenyl-2-yl)methyl]-3- | 5.2 | |
| oxoisoindoline-1-carboxamide | | |
| (1R or 1S)-N-(tert-butyl)-2-[(3′,4′-difluorobiphenyl-2- | 13 | 47 |
| yl)methyl]-3-oxoisoindoline-1-carboxamide | | |
| (1S or 1R)-N-(tert-butyl)-2-[(3′,4′-difluorobiphenyl-2- | 5.5 | 47 |
| yl)methyl]-3-oxoisoindoline-1-carboxamide | | |

The invention is illustrated by way of the following examples.

### EXAMPLES

The naming of compounds in this patent application was made using a program from ACD Labs (version 9.0, Name Batch or labs).

### Examples

Abbreviations

AIBN 2,2′-Azobis(2-methylpropionitril)

C Celsius

BOC-anhydride di-tert-Butyl dicarbonate

Dess-Martin Reagent 1,1,1-Triacetoxy-1,1-dihydro-1,2-benziodoxol-3(1H)-one

DCM dichloromethane

DMF N,N′-dimethylformamide

DME dimethoxyethane

DMAP Dimetylaminopyridine

DMSO dimethylsulfoxide

ES electrospray

ESI electrospray ionisation

EtOAc ethyl acetate

EtOH ethanol

DME Dimetoxyethane

HPLC high performance liquid chromatography

HRMS high resolution mass spectrometry

LAH Lithium aluminiumhydride

MeCN acetonitrile

MeOH methanol

MTBE Methyl tert-butyl ether

K2CO3 Potassium carbonate

MS mass spectroscopy

NMR nuclear magnetic resonance

TEA triethylamine

TFA trifluoroacetic acid

THF tetrahydrofuran

UV ultra violet

atm atmosphere

rt room temperature

h hour(s)

mins minutes

br broad

br s broad singlet

s singlet

d doublet

t triplet

q quartet

m multiplet

sep septett

dd double doublet

dm double multiplet

td triple doublet

General Experimental Procedures

Flash column chromatography was performed using normal phase silica gel 60 (0.040-0.063 mm, Merck) or SP1™ Purification System from Biotage™ using silica FLASH+™ Cartridges unless otherwise stated.

¹H NMR and ¹³C NMR measurements were performed on a BRUKER ACP 300 or on a. Varian Unity Plus 400, 500 or 600 spectrometer, operating at ¹H frequencies of 300, 400, 500, 600 MHz, respectively, and ¹³C frequencies of 75, 100, 125 and 150 MHz, Alternatively, ¹³C NMR measurements were performed on a BRUKER ACE 200 spectrometer at a frequency of 50.3 MHz.

Rotamers may or may not be denoted in spectra depending upon ease of interpretation of spectra.

Chemical shifts are given in 5 values (ppm) with the solvents used as internal standard, unless otherwise stated.

*The solutions are taken from a concentrated sample solved in (CH₃)₂SO and are diluted with (CD₃)₂SO. Since an substantial amount of (CH₃)₂SO is present in the sample, first a pre-scan is run and analysed to automatically suppress the (CH₃)₂SO (2.54 ppm) and H2O (3.3 ppm) peaks. This means that in this so-called wet1D experiment the intensity of peaks that reside in these areas around 3.3 ppm and 2.54 ppm is reduced. Furthermore impurities are seen in the spectrum which gives rise to a triplet at 1.12 ppm, a singlet at 2.96 and two multiplets between 2.76-2.70 ppm and 2.61-2.55 ppm. Most probably these impurities are dimethylsulfone and diethylsulfoxide.

Microwave heating was performed using single node heating in a Smith Creator or Emrys Optimizer from Personal Chemistry, Uppsala, Sweden.

Mass spectral (MS) data were obtained using ZQ, a quadrupole instrument from Waters and, where appropriate, either positive ion data or negative ion data were collected.

Accurate mass (HRMS) spectral data were obtained using TOF-MS on a LCT, Q-TOF micro or LCTP system, all from Waters.

Syringe filter from Advantec MFS Inc. with a pore size of 0.5 μm was used.

Cation exchange columns from Isolute® was used.

Synthesis of Intermediates

Preparation A

### 2-(4-Fluoro-phenyl)-propylamine

### (i) 1-Fluoro-4-isopropenyl-benzene

Potassium tert-butoxide (52.9 g, 0.47 mol) was added to the suspension of methyl triphenylphosphonium iodide (190 g, 0.47 mol) in THF (400 ml) at ice-cooled condition. After 1 h stirring at ice-cooled condition, a solution of 4-Fluoro acetophenone (30 g, 0.199 mol) in THF (100 ml) was added dropwise. Then the reaction mixture was stirred at room temperature for 2 h and quenched with sat. ammonium chloride solution. THF was removed under reduced pressure and the reaction mixture was extracted with petether, washed with water, brine and dried over anh. sodium sulphate ntrated. The sub-title compound (30 g, 100%) was obtained as pale yellow liquid by concentration of petroleum ether layer

### (ii) 2-(4-Fluoro-phenyl)-propylamine

Borane in THF (96 ml, 0.096 mol, 1M solution) was added dropwise to the solution of 1-Fluoro-4-isopropenyl-benzene from step (i) above (30 g, 0.24 mol) in THF (350 ml) at 0° C. and the reaction mixture was stirred at room temperature for 3 h. The reaction mixture was cooled to 0° C. and hydroxylamine-O-sulphonic acid (27.72 g, 0.24 mol) was added portionwise. The reaction mixture was refluxed for overnight. Then the reaction mixture was quenched with water and concentrated, acidified with 1.5 N HCl. The reaction mixture was extracted with ethylacetate, aqueous layer was neutralized with 10% sodium hydroxide solution and extracted with dichloromethane. The dichloromethane layer was washed with, water, brine and evaporated to give the title compound (8.5 g, 23%)

Preparation B

### [2-(4-chlorophenyl)propyl]amine

### (i) 1-chloro-4-isopropenylbenzene

Potassium tert-butoxide (72.4 g, 0.646 mol) was added to the suspension of methyl triphenylphosphonium iodide (261 g, 0.646 mol) in THF (500 ml) at ice-cooled condition. After 1 h stirring at ice-cooled condition, a solution of 4-chloroacetophenone (50 g, 0.323 mol) in THF (100 ml) was added dropwise. Then the reaction mixture was stirred at room temperature for 1 h. The reaction mixture was diluted with pet ether and filtered, filtrate was concentrated. The sub-title compound was obtained as pale yellow liquid by column chromatographic purification of the crude product using 6% ethyl acetate in pet ether as eluent. Yield (42 g, 86%)

### (ii) [2-(4-chlorophenyl)propyl]amine

Borane in THF (124 ml, 1M solution) was added dropwise to the solution of 1-chloro-4-isopropenylbenzene from step (i) above (41.5 g, 0.272 mol) in THF (500 ml) at 0° C. and the reaction mixture was stirred at room temperature for 3 h. The reaction mixture was cooled to 0° C. and hydroxylamine-O-sulphonic acid (30.76 g, 0.272 mol) was added portionwise. The reaction mixture was refluxed overnight. The reaction mixture was quenched with water and extracted with ethyl acetate. The ethyl acetate layer was washed with water, brine, dried over anh. sodium sulphate and concentrated. The concentrated mass was dissolved in dry diethyl ether (20 ml) and stirred with saturated HCl in diethyl ether for ½ h. The solid salt was isolated by filtration, neutralized with sodium bicarbonate solution and free amine was extracted with diethyl ether. The diethyl ether layer was washed with brine, dried over anh. sodium sulphate and concentrated to give the title compound (18 g, 39%)

Preparation C

### 1-(4’-fluorobiphenyl-2-yl)methanamine

### (i) tert-butyl N-[[2-(4-fluorophenyl)phenyl]methyl]carbamate

N-(Tert-butoxycarbonyl)-2-Bromobensylathine (1.74 mmol, 0.5 g) and, Tetrakis(Triphenylphosfin)palladium (0.087 mmol, 0.101 g) and 4-fluorobenzeneboronic acid (2.096 mmol, 0.293 g) were dissolved in (DME, 10 ml) in a vial suitable for use in a microwave oven. Cesiumcarbonate (3.49 mmol, 1.14 g) was dissolved in 2 ml water and then added to the mixture. Ar (g) was bubbled through the mixture for 5 minutes. The reaction was performed in a micro oven (10 min, 130 deg). The crude (0.5 g, 1.65 mmol) of the sub-title compound was taken to the next step without further purification

### (ii) 2-(4-fluorophenyl)phenyl]methaneamine

Tert-butyl N-[[2-(4-fluorophenyl)phenyl]methyl]carbamate (1.6591 mmol, 0.5 g) from step (i) above was dissolved in HCl saturated EtOAc and stirred at rt for 2 h. The solvent was removed by evaporation and the HCl-salt was purified by flashcromatography (started with isocratic heptane/DCM 50/50 and then the DCM concentration was increased to 100% then the product was eluated with 10% MeOH (saturated with NH3), (silica gel 60 0.004-0.063 mm) The product containing fractions was pooled and the solvent was removed by evaporation, to give 320 mg (1.59 mmol) of the title compound.

Preparation D

The following amines was made according to Preparation C above:

1-(4′-methylbiphenyl-2-yl)methanamine

1-(4′-methoxybiphenyl-2-yl)methanamine

1-(4′-fluoro-2′-methylbiphenyl-2-yl)methanamine

Preparation E

### (2-cyclopentylbenzyl)amine hydrochloric acid salt

### (i) 2-cyclopentylphenyl trifluoroacetate

To a solution of 2-cyclopentyl phenol (10 g, 0.09 mol) in dry DCM (150 ml) was added pyridine (8 ml, 0.14 mol) and cooled to 0° C. followed by the addition of trifluoroacteic acid anhydride (15.6 ml, 0.14 mol) drop wise. The reaction mixture was stirred at room temperature overnight. The reaction mixture was quenched with water and extracted with DCM (200 ml). The organic layer was washed with water (2×50 ml) and brine solution (1×50 ml) and concentrated to afford step 1 product (18 g, 99.4%) as brown liquid. The crude product was taken as such taken for next step.

### (ii) 2-cyclopentylbenzonitrile

To a solution of 2-cyclopentylphenyl trifluoroacetate (18 g, 0.06 mol) in dry DMF (150 ml) was added Zn(CN)₂ (7.2 g;0.06 mol) followed by Pd(PPh₃₎₄ (5.6 g;0.005 mol) and refluxed at 130° C. overnight under nitrogen atmosphere. Reaction mixture was then cooled to room temperature, quenched with water (200 ml) and diluted with EtOAc (200 ml) and filtered. The filtrate was washed well with water (3×50 ml) and brine solution (1×50 ml). The organic layer was concentrated and the crude purified through silica gel column chromatography using 5% EtOAc in pet. ether to afford step 2 product (10.4 g, 99.3%) as pale yellow solid.

### (iii) (2-cyclopentylbenzyl)amine

To suspension of lithium aluminium hydride (5.7 g, 0.15 mol) in dry THF (50 ml) at 0° C. was added 2-cyclopentylbenzonitrile (10.4 g, 0.06 mol) dissolved in dry THF (100 ml) drop wise. Reaction was stirred at room temperature overnight. Reaction mass cooled to 0° C. and quenched with 6 M KOH and diluted with THF. It was then filtered through celite and filtrate concentrated. To a solution of crude product in diethyl ether (100 ml) was added saturtated. HCl in diethyl ether (50 ml) and stirred for 10 minutes. It was then filtered and precipitate was washed with petroleum ether to afford the title compound (10.3 g, 98.1%) as white solid.

Preparation F

### (2-cyclohexylbenzyl)amine hydro chloric acid salt

### (i) 2-cyclohexylbenzonitrile

To a solution of 2-cyclohexyl bromobenzene (12 g, 0.05 mol) in Dry DMF (100 ml) was added Zn(CN)₂ (5.9 g, 0.05 mol) followed by Pd(PPh₃)₄ (5.8 g, 0.005 mol) and refluxed at 130° C. for overnight. Reaction mass was then cooled to room temperature, quenched with water (200 ml) and diluted with EtOAc (200 ml) and filtered. The filtrate was washed well with water (3×50 ml) and brine solution (1×50 ml). Organic layer was concentrated to afford step 1 product (10 g) as yellow gummy liquid. The crude product was used in the next step.

### (ii) (2-cyclohexylbenzyl)amine

To a suspension of lithium aluminium hydride (5.1 g, 0.13 mol) in dry THF (60 ml) at 0° C. was added 2-cyclohexylbenzonitrile from step (i) above (10 g, 0.054 mol) dissolved in Dry THF (140 ml) drop wise. After stirring the reaction mixture for overnight at RT, it was cooled to 0° C. and quenched with 6 M KOH. It was further diluted with THF and filtered through celite. The filtrate was concentrated under vacuum. To a solution of crude product in Diethyl ether (100 ml) was added satd. HCl in diethyl ether (20 ml) and stirred for 10 minutes. It was then filtered and precipitate was washed with petroleum ether to afford the title compound (8 g, 78.4%) as white solid.

Preparation G

### 4-fluorobutyl)amine hydrochloride

### (i) (4-{[tert-butyl(dimethyl)silyl]oxy}butyl)amine

To the solution of 4-aminobutanol (20 g, 0.224 mol) in dichloromethane (200 ml) was added triethylamine at 0° C. tert-butyldimethylchlorosilane (33.8 g, 0.224 mol) was added at the same temperature and stirred at room temperature for 4 h. The reaction mixture was diluted with water. The organic layer was washed with water, brine, dried over anh. sodium sulfate and concentrated. The crude product (42 g, 92%) was used for next step without purification.

### (ii) tert-butyl(4-{[tert-butyl(dimethyl)silyl]oxy}butyl)carbamate

To a solution of (4-{[tert-butyl(dimethyl)silyl]oxy}butyl)amine (42 g, 0.20 mol) in dichloromethane (400 ml) and triethylamine (58 ml, 0.413 mol) was added BOC-anhydride (54.12 g, 0.248 mol) at 0° C. under nitrogen atmosphere. The reaction mixture was stirred at room temperature for 1 h. The reaction mixture was diluted with ice-cold water. The organic layer was washed with water and brine. The organic layer was then dried over anh. sodium sulfate and concentrated. The residue was purified by silica gel chromatography using (EtOAc/pet ether) to give (60 g, 95%) of the sub-title compound as a colorless oil.

### (iii) di-tert-butyl (4-{[tert-butyl(dimethyl)silyl]oxy}butyl)imidodicarbonate

To a solution of tert-butyl (4-{[tert-butyl(dimethyl)silyl]oxy}butyl)carbamate (from step (ii) above (60 g, 95%) in dry acetonitrile (600 ml) was added DMAP (36.23 g, 0.29 mol). BOC-anhydride (64.7 g, 0.29 mol) was added and the reaction mixture was stirred at room temperature for 24 h. Then DMAP (36.23 g, 0. 29 mol) and BOC-anhydride (64.7 g, 0.29 mol) were added once more. The reaction mixture was stirred at room temperature for another 4 days. The reaction mixture was concentrated and the residue was purified by silica gel chromatography using (EtOAc/pet ether) to give (62 g, 77%) of the sub-title compound as a colorless oil.

### (iv) di-tert-butyl (3-hydroxypropyl)imidodicarbonate

To a solution of di-tert-butyl (4-{[tert-butyl(dimethyl)silyl]oxy}butyl)imidodicarbonate (60 g, 0.148) from step (iii) above in THF (600 ml) was added tetrabutylammonium fluoride (150 ml, 1M solution THF) at 0° C. under nitrogen atmosphere. The reaction mixture was stirred at room temperature for overnight. The reaction mixture was concentrated and concentrated mass was dissolved in ethyl acetate. The ethyl acetate layer was washed successively with water, 10% citric acid, 10% sodium bicarbonate and brine solution. The reaction mixture was concentrated and the residue was purified by silica gel chromatography using (EtOAc/pet ether) to give (37 g, 86%) of the sub-title compound as a pale yellow liquid.

### (v) di-tert-butyl(3-fluoropropyl)imidodicarbonate

To a solution of di-tert-butyl (3-hydroxypropyl)imidodicarbonate (35 g, 0.121 mol) from step (iv) above in dry THF (400 ml) was added triethylamine (84.9 mol, 0.60 ml) and (diethylamino) sulfur trifluoride (102 g, 0.60 mol) at −40° C. under nitrogen atmosphere. The reaction mixture was slowly allowed to warm up to room temperature and stirred at room temperature for 3 days. The reaction was quenched by addition of methanol at 0° C. The reaction mixture was concentrated and residue was purified by silica gel chromatography using (EtOAc/pet ether) to give (11.8 g, 33%) of the sub-title compound as a pale yellow liquid.

### (vi) 4-fluorobutyl)amine hydrochloride

To a solution of di-tert-butyl (3-fluoropropyl)imidodicarbonate (11.8 g, 0.040 mol) from step (v) above in dry diethylether (30 ml) was added saturated. HCl in ether (200 ml) at 0° C. The reaction mixture was stirred at room temperature for 24 h. The reaction mixture was concentrated and the title compound (4.1 g, 80%) was obtained as pale yellow solid by recrystallization of with acetone/diethylether.

Preparation H

### (4,4-difluorobutyl)amine hydrochloride

### (i) di-tert-butyl(3-oxopropyl)imidodicarbonate

To a solution of Dess-Martin reagent (66 g, 0.155 mol) in dichloromethane (300 ml) was added di-tert-butyl(3-hydroxypropyl)imidodicarbonate (30 g, 0.103 mol) in dichloromethane at −78° C. under nitrogen atmosphere. Then the reaction mixture was allowed to warm up to room temperature and stirred at the same temperature for overnight. The reaction mixture was filtered through celite and the filtrate was concentrated. The concentrated mass was stirred with diethyl ether and ether layer was separated and concentrated. The concentrated crude was purified by column chromatography using (EtOAc/pet. ether) to give (23 g, 77%) of the desired intermediate as pale yellow liquid

### (ii) di-tert-butyl(4,4-difluorobutyl)imidodicarbonate

To a solution of step (i) intermediate (23 g, 0.080 mol) in dry dichloromethane (200 ml) was added (diethylamino) sulfur trifluoride (38.7 g, 0.24 mol) at −40° C. under nitrogen atmosphere. The reaction mixture was slowly allowed to warm up to room temperature and stirred at room temperature for 2 h. The reaction mixture was slowly poured into cold water. The organic layer separated was washed with water, brine and concentrated. The residue was purified by silica gel chromatography using (EtOAc/pet ether) to give (11.12 g, 45%) of the sub-title compound as a pale yellow liquid.

### (iii) (4,4-difluorobutyl)amine hydrochloride

To a solution of step (ii) intermediate (11.2 g, 0.035 mol) in dry diethylether (30 ml) was added satd. HCl in ether (150 ml) at 0° C. The reaction mixture was stirred at room temperature for 24 h. The reaction mixture was concentrated and the title compound (4.5 g, 86%) was obtained as pale yellow solid by recrystallization of the crude mass with acetone/diethylether.

Preparation I

### (4,4-difluorobutyl)formamide

(4,4-difluorobutyl)amine (Prep. H above) (6 g, 0.041 mol) and triethyl amine (14.46 ml, 0.103 mol) was refluxed in ethylformate (150 ml) overnight. The reaction mixture was cooled to room temperature and filtered. The filtrate was concentrated and the crude product was purified by column chromatography using (EtOAc/pet ether) to give the title compound (4.2 g, 74%) as pale yellow liquid.

Preparation J

### (4,4,4-trifluorobutyl)formamide

### (i) (4,4,4-trifluorobutyl)amine hydrochloride

A solution of diethyldiazodicarboxylate (31.1 ml, 0.195 mol) in toluene (150 ml) was added slowly to the solution of 4,4.4-trifluorobutanol (25 g, 0.195 ml), triphenylphosphine (51 g, 0.195 mol) and di-tert-butyliminodicarboxylate (38 g, 0.175 mol) in toluene (300 ml) at room temperature. The reaction mixture was stirred at the same temperature for 24 h. Trifluoroacetic acid (30 ml) was added to the reaction mixture at ice-cold condition and then stirred at room temperature for another 24 h. The reaction mixture was diluted with water (500 ml). The aqueous layer separated was washed with diethyl ether and then made alkaline with 5N sodium hydroxide solution. The alkaline solution was extracted with diethyl ether. The diethyl ether layer was dried over anhydride magnesium sulphate. Then saturated hydrochloric acid in ether was added the above ether solution and stirred for overnight. The ether was removed under reduced pressure and the resulting mass was azeotroped with toluene to give the title compound (11.8 g, 37%).

### (ii) (4,4,4-trifluorobutyl)formamide

A mixture of (4,4,4-trifluorobutyl)amine (8.7 g) from step (i) above and triethyl amine (14.6 ml, 0.14 mol) was refluxed in ethylformate (250 ml) for 24 h. The reaction mixture was cooled to room temperature and filtered. The filtrate was concentrated and the crude product was purified by column chromatography using 50% ethyl acetate in pet ether as eluent to give the title compound (5.5 g, 67%, 3.84 g as a colorless liquid.

Preparation K

### (4-fluorobutyl)formamide

4-fluorobutyl)amine hydrochloride (Prep.G above) (4.12 g, 0.032 mol) and triethyl amine (14.6 ml, 0.14 mol) was refluxed in ethylformate (13.6 ml, 0.097 mol) for 24 h. Then the reaction mixture was cooled to room temperature and filtered. The filtrate was concentrated and the crude product was purified by column chromatography using 50% ethyl acetate in pet ether as eluent to give the title (3.12 g, 80%) as pale yellow liquid.

Preparation L

### 3-Isocyanomethyl-5-methyl-isoxazole

### (i) N-(5-methyl-isoxazol-3-ylmethyl)-formamide

A vial suitable for use in a microwave oven was charged with a solution of (5-methyl-isoxazol-3-yl)-methylamine (1 g, 8.9 mmol) and ethyl formate (17 ml, 212 mmol). The mixture was heated under a nitrogen atmosphere at 150° C. for 15 min. After irradiation the mixture was cooled to room temperature and evaporated. 1.378 g of the crude product, N-(5-methyl-isoxazol-3-ylmethyl)-formamide (99% yield) was obtained and used without further purification.

[M+1] (ES) 141.0

¹H NMR (500 MHz, CDCl₃) δ 8.57 (br s, 1H); 8.12 (s, 1H); 6.11 (s, 1H); 4.29 (d, 2H); 2.37 (s, 3H)

### (ii) 3-Isocyanomethyl-5-methyl-isoxazole

(methoxycarbonylsulfamoyl)triethylammonium hydroxide, inner salt, (4.2 g, 17.7 mmol) was added to a solution of N-(5-methyl-isoxazol-3-ylmethyl)-formamide (1.378 g; 8.8 mmol from step (i) above) in acetonitrile (15 ml). The mixture was stirred at 50° C. for 3 h under a nitrogen atmosphere and then cooled to room temperature.

The solution of 3-Isocyanomethyl-5-methyl-isoxazole was used to make compounds in the examples without further purification.

Preparation M (i) (isocyanomethyl)benzene

### (i) N-benzylformamide

Benzylamine (2.07 mL, 19 mmol) was dissolved in Ethyl formate (24 mL) and the reaction was left stirring at 45° C. for 20 h. The solvent was evaporated and the product was concentrated under reduced pressure to give the title compound (2.44 g, 95.2%). ¹H-NMR (500 MHz, CDCl₃) δ 8.36-8.25 (s, 1 H), 7.5-7.2 (m, 5 H), 6.02-5.57 (s, 1H), 4.56-4.46 (d, 2H).

### (ii) (isocyanomethyl)benzene

N-benzylformamide From step (i) above (2.44 g, 18.1 mmol) and (Methoxycarbonylsulfamoyl)triethylammonium hydroxide inner salt (Burgess reagent) (4.31 g, 18.1 mmol) was added to a flask and dry MeCN (25 mL) was added. The reaction mixture was left stirring for 3 h at 50° C. and the crude title compound was used without futher purification in the next step

Preparation N

### 1-chloro-3-(isocyanomethyl)benzene

### (i) N-(3-chlorobenzyl)formamide

1-(3-chlorophenyl)methanamine (0.283 g, 2.0 mmol) was dissolved in Ethyl formate (8 mL) and the reaction was left stirring at 45° C. for 16 h. The solvent was evaporated and the product was concentrated under reduced pressure to give the title compound (0.350 g, 103%). ¹H-NMR (500 MHz, CDCl₃) δ 8.23-8.09 (m, 1H), 7.38-7.18 (m, 3H), 4.8-4.34 (s, 2H).

### (ii)1-chloro-3-(isocyanomethyl)benzene

N-(3-chlorobenzyl)formamide (0.128 g, 0.755 mmol) and (Methoxycarbonylsulfamoyl)triethylammonium hydroxide inner salt (Burgess reagent) (0.182 g, 0.764 mmol) was added to a flask and MeCN (6 mL) was added. The reaction mixture was left stirring for 3 h at 50° C. and the crude was used without futher purification in the next step

### Preparation O

### 4-(isocyanomethyl)-5-methyl-2-phenyl-1,3-oxazole

### (i) N-[(5-methyl-2-phenyl-1,3-oxazol-4-yl)methyl]formamide

1-(5-methyl-2-phenyl-1,3-oxazol-4-yl)methanamine (0.512 g, 2.72 mmol) was dissolved in Ethyl formate (8 mL) and the reaction was left stirring at 45° C. for 22 h. The solvent was evaporated and the product was concentrated under reduced pressure to give the sub-title compound (0.559 g, 95%). ¹H-NMR (500 MHz, CDCl₃) δ 8.3-8.18 (m, 1H), 8.07-7.99 (m, 2H), 7.62-7.34 (m, 3H), 6.51-5.91 (m, 1H),4.46-4.32 (d, 2H), 2.62-2.38 (m, 2H); MS (ESI) m/z 217 ([M+H]⁺).

### (ii) 4-(isocyanomethyl)-5-methyl-2-phenyl-1,3-oxazole

N-[(5-methyl-2-phenyl-1,3-oxazol-4-yl)methyl]formamide from step (i) above (0.259 g, 1.20 mmol) and (Methoxycarbonylsulfamoyl)triethylammonium hydroxide inner salt (Burgess reagent) (0.288 g, 1.21 mmol) was added to a flask and MeCN (6 mL) was added. The reaction mixture was left stirring for 3 h at 50° C. and the crude was used without futher purification in the next step

Preparation P

### 1,1-difluoro-4-isocyanobutane

N-(4,4-difluorobutyl)formamide (from prep. I above) (0.076 g, 0.554 mmol) and (Methoxycarbonylsulfamoyl)triethylammonium hydroxide inner salt (Burgess reagent) (0.132 g, 0.554 mmol) was added to a flask and MeCN (2 mL) was added. The reaction mixture was left stirring for 3 h at 50° C. and the crude was used without futher purification for making compounds in examples below.

Preparation Q

### [(2,2-difluoro-1,3-benzodioxol-5-yl)methyl]amine hydrochloride

### (i) 2,2-difluoro-1,3-benzodioxole-5-carbaldehyde oxime

A solution of 2,2-difluoro-1,3-benzodioxole-5-carbaldehyde (8 g, 0.0429 mol) in methanol (50 ml) was added dropwise to the well-stirred solution of hydroxylamine hydrochloride (4.18 g, 0.060 mol) and sodium acetate (4.9 g, 0.060 mol) in methanol (100 ml) at room temperature. The reaction mixture was stirred at room temperature for 6 h. Then the reaction mixture was concentrated under reduced pressure and concentrated mass was diluted with water, extracted with dichloromethane. The dichloromethane layer was washed with water, brine, dried over anh. sodium sulfate and concentrated to give crude intermediate. The title compound (6.2 g, 72%) was obtained on crystallization of crude intermediate from dichloromethane/hexane solvent.

### (ii) 1-(2,2-difluoro-1,3-benzodioxol-5-yl)methanamine hydrochloride

A solution of 2,2-difluoro-1,3-benzodioxole-5-carbaldehyde oxime (6.2 g, 0.030 mol) from step (i) above in THF (25 ml) was added slowly to the suspension of LAH (2.9 g, 0.077 mol) in THF (100 ml) at 0° C. under nitrogen atmosphere. Then the reaction mixture was allowed to stir at room temperature for 8 h. The reaction mixture was cooled to 0° C. and quenched with 6 (M) KOH solution (3 ml). The reaction mixture was filtered through celite and solid residue was washed several times with ethylacetate. The combine filtrate was concentrated to give the crude product. The crude product was dissolved in ether and stirred with satd. HCl in ether (20 ml) for 2 h. Then the solution was filtered and residue was dried to give the title compound (4.8 g, 83.3%) as white powder.

Preparation R

### (2,2-difluoro-1,3-benzodioxol-5-yl)methyl isocyanide

### (i) 1-(2,2-difluoro-1,3-benzodioxol-5-yl)methanamine

1-(2,2-difluoro-1,3-benzodioxol-5-yl)methanamine hydrochloride (2.0 g, 8.94 mmol) was added to a flask, a 2 M solution of K2CO3 (30 mL) and DCM (30 mL) was added. The reaction mixture was left stirring in r.t. 2 h. The two phases were separated and the water phase was extracted once more with DCM (20 mL). The organic phases were collected and dried with MgSO4 and the salt was filtered off. The organic phase was evaporated and the product was concentrated under reduced pressure to give the title compound (1.53 g, 91.4%).

¹H-NMR (500 MHz, CDCl₃) δ 7.15-6.95 (m, 3H), 4.07-3.78 (s, 2H); MS (ESI) m/z 188 ([M+1.1]⁺).

### (ii) N[(2,2-difluoro-1,3-benzodioxol-5-yl)methyl]formamide

1-(2,2-difluoro-1,3-benzodioxol-5-yl)methanamine (from step (i) above (1.53 g, 8.18 mmol) was dissolved in Ethyl formate (40 mL) and the reaction was left stirring at 45° C. for 20 h. The solvent was evaporated and the product was concentrated under reduced pressure to give the title compound (1.72 g, 97.6%).

¹H-NMR (500 MHz, CDCl₃) δ 8.35-8.25, 7.09-6.95 (m, 3H), 6.01-5.61 (m 1H),4.53-4.45 (d, 2H); MS (ESI) m/z 214 ([M+H]⁺).

### (iii) (2,2-difluoro-1,3-benzodioxol-5-yl)methyl isocyanide

N-[(2,2-difluoro-1,3-benzodioxol-5-yl)methyl]formamide from step (ii) above (0.320 g, 1.49 mmol) and (Methoxycarbonylsulfamoyl)triethylammonium hydroxide inner salt (Burgess reagent) (0.359 g, 1.51 mmol) was added to a flask and dry MeCN (8 mL) was added. The reaction mixture was left stirring for 3 h at 50° C. and the crude was used without futher purification in the next step (AZ12609901).

Preparation S

### 1,2-dichloro-4-(isocyanomethyl)benzene

### (i) N-(3,4-dichlorobenzyl)formamide

To 1-(3,4-dichlorophenyl)methanamine (352 mg, 2.00 mmol) was added ethyl formate (8.0 mL, 99.4 mmol). The mixture was stirred at 45° C. over night (16 h) and then concentrated in vacuo to give 433 mg of the crude product. The crude product was used in the next step without further purification.

¹H-NMR (500 MHz, CD₃OD) δ 8.18 (s, 1H), 7.51-7.47 (m, 2H), 7.27-7.23 (m, 1H), 4.40 (s, 2H).

### (ii) 1,2-dichloro-4-(isocyanomethyl)benzene

To N-(3,4-dichlorobenzyl)formamide (from step (i) above) (245 mg, 1.20 mmol) dissolved in MeCN (6 mL) was added (methoxycarbonylsulfamoyl)triethylammonium hydroxide, inner salt (Burgess reagent) (286 mg, 1.20 mmol). The mixture was stirred at 50° C. for 3 h. After cooling to rt, the mixture was transferred directly to the next reaction step without any work-up.

Preparation T

Not commercially available isonitriles were made from the corresponding amines via formamides in analogy with preparation L, M,N,O,P,Q,R above, and used to make compounds in examples below

Preparation U

### 4-fluoro-3-hydroxy-2-benzofuran-1(3H)-one

### (i) 3-fluoro-N-(2-hydroxy-1,1-dimethylethyl)benzamide

To a solution of 3-fluorobenzoic acid (20 g, 0.142 mol) in dichloromethane (200 ml) was added oxalyl chlorode (14.6 ml, 0.171 mol) followed by a drop of DMF at 0° C. under nitrogen atmosphere. The reaction mixture was stirred for 2 h at room temperature. Then the reaction mixture was concentrated and added dropwise to the solution of 2-amino-2-methyl propanol (28 g, 0.314 mol) in dichloromethane (100 ml) at 0° C. under nitrogen atmosphere. The resulting solution was stirred at room temperature for another 2 h. The reaction mixture was filtered and the filtrate was concentrated. The concentrated white solid (30 g) was used for the next step without purification.

### (ii) 2-(3-fluorophenyl)-4,4-dimethyl-4,5-dihydro-1,3-oxazole

Thionyl chloride (60 g) was added dropwise to the 3-fluoro-N-(2-hydroxy-1,1-dimethylethyl)benzamide (from step (i) above) (30 g) under stirring at room temperature and stirred for another 15 minutes. The yellow solution was poured into dry diethyl ether (200 ml) and the reaction mixture was neutralized with 20% cold sodium hydroxide solution. The diethyl ether layer was washed with water, brine, dried over anh. sodium sulfate and concentrated. The residue was purified by silica gel chromatography using (EtOAc/pet ether) to give the sub-title compound (18.5 g, 67.3%) as brown liquid

### (iii)) 4-fluoro-3-hydroxy-2-benzofuran-1(3H)-one

To a solution of 2-(3-fluorophenyl)-4,4-dimethyl-4,5-dihydro-1,3-oxazole (18.5 g, 0.958 mol) (from step (ii) above)in diethyl ether (200 ml) was added dropwise sec-BuLi (103 ml, 1.4 M in cyclohexane) at −78° C. under nitrogen atmosphere and stirred for another 30 minute at the same temperature. Then dry DMF (20 ml) was added and the reaction mixture was allowed to stir at room temperature for another 2 h. The reaction was quenched with 6(N) HCl and concentrated. The concentrated mass was refluxed with 6(N)HCl (400 ml) for overnight. Then the reaction mixture was cooled to room temperature and stirred with diethyl ether (200 ml). The organic layer was separated and washed with water, brine and dried over anh. sodium sulfate and concentrated to give the crude cyclic product. The crude mass was purified by recrystallization (diethyl ether/pet ether) to give the title compound (8 g, 49.6%) as white solid

Preparation V

### 5-chloro-3-hydroxy-2-benzofuran-1(3H)-one

### (i) 4-chloro-N-(2-hydroxy-1,1-dimethylethyl)benzamide

To a solution of 4-chloroorobenzoic acid (50 g, 0.319 mol) in dichloromethane (400 ml) was added oxalyl chloride (34 ml, 0.383 mol) followed by a drop of DMF at 0° C. under nitrogen atmosphere. The reaction mixture was stirred for 2 h at room temperature. Then the reaction mixture was concentrated and added dropwise to the solution of 2-amino-2-methyl propanol (62.8 g, 0.702 mol) in dichloromethane (200 ml) at 0° C. under nitrogen atmosphere. The resulting solution was stirred at room temperature for another 2 h. The reaction mixture was filtered and the filtrate was concentrated. The residue was purified by silica gel chromatography using (MeOH/CHCl₃) to give the desired intermediate (68 g, 93.5%) as brown liquid

### (ii) 2-(4-chlorophenyl)-4,4-dimethyl-4,5-dihydro-1,3-oxazole

Thionyl chloride (120.8 g, 1.05 mol) was added dropwise to 4-chloro-N-(2-hydroxy-1,1-dimethylethyl)benzamide (68 g, 0.30 mol) (from step (i) above under stirring at room temperature and stirred for another 15 minutes. Then the yellow solution was poured into dry diethyl ether (500 ml) and the reaction mixture was neutralized with 20% cold sodium hydroxide solution. The diethyl ether layer was washed with water, brine, dried over anh. sodium sulfate and concentrated. The residue was purified by silica gel chromatography using (EtOAc/pet ether) to give the sub-title compound (53 g, 84.6%) as brown liquid

### (iii) 5-chloro-2-(4,4-dimethyl-4,5-dihydro-1,3-oxazol-2-yl)benzaldehyde

To a solution of 2-(4-chlorophenyl)-4,4-dimethyl-4,5-dihydro-1,3-oxazole (19 g, 0.906 mol) (from step (ii) above) in diethyl ether (400 ml) was added dropwise sec-BuLi (110 ml, 1.4 M in cyclohexane) at −78° C. under nitrogen atmosphere and the reaction mixture was allowed to warm up to 0° C. and stirred at 0° C. for another 1 h. The reaction mixture was again cooled down to −78° C. and dry DMF (10 ml) was added and warmed up to room temperature overnight. The reaction was quenched and diluted with water. The separated organic layer was washed water and brine. Then the organic layer was dried over anhydrous sodium sulfate and concentrated in vacuum to give the crude sub-title compound (20 g) as yellow liquid.

### (iv) 5-chloro-3-hydroxy-2-benzofuran-1(3H)-one

5-chloro-2-(4,4-dimethyl-4,5-dihydro-1,3-oxazol-2₇yl)benzaldehydefrom step (iii) above (20 g) was heated at 80° C. for overnight with HCl in water (300 ml water, 150 ml Conc. HCl). Then the reaction mixture was cooled to room temperature and stirred with diethyl ether (500 ml). The diethyl ether layer was washed with water and brine. The organic layer was dried over anh. sodium sulfates and concentrated under vacuum. The crude mass was recrystallized from ethylacetate/pet ether solvent system to give the title compound (4.9 g, 31.6%) as a light brown solid

Preparation W

### 5-bromo-3-hydroxy-2-benzofuran-1(3H)-one

### (i) 4-bromo-2-(hydroxymethyl)benzoic acid

5-Bromo phthalide (9 g, 0.042 mol) was refluxed with 2 (N) aqueous sodium hydroxide (100 ml) in methanol (150 ml) for overnight. The reaction mixture was concentrated under reduced pressure. Cocnentrated mass was diluted with water and acidified with dilute HCl. The solid precipitates was filtered and residue was washed with cold water and cold ethyl acetate and dried to give the sub-title compound (9.7 g, 100%) as white solid.

### (i) 5-bromo-3-hydroxy-2-benzofuran-1(3H)-one

To a solution of 4-bromo-2-(hydroxymethyl)benzoic acid from step (i) above (9.7 g, 0.042 mol) in acetonitrile (300 ml) was added Dess-Martin periodinane (26.8 g, 0.0633 mol) at 0° C. under nitrogen atmosphere. The reaction mixture was stirred at room temperature for 14 h. The reaction mixture was filtered and the precipitates were washed with dichloromethane. The filtrate was concentrated. The concentrated mass was heated with 200 ml of Conc. HCl for 6 h at 70° C. The reaction mixture was diluted with large volume of water and extracted with ethyl acetate. The organic layer was washed with brine, dried over anh. sodium sulfate and concentrated. The concentrated mass was purified by recrystallization from ethyl acetate/pet ether to give the title compound (4.9 g, 51%) as white solid.

Preparation X

### 5-fluoro-3-hydroxy-2-benzofuran-1(3H)-one

### (i) 4-fluoro-N-(2-hydroxy-1,1-dimethylethyl)benzamide

To a solution of 4-fluorobenzoic acid (50 g, 0.357 mol) in dichloromethane (400 ml) was added oxalyl chlorode (34 ml, 0.393 mol) followed by a drop of DMF at 0° C. under nitrogen atmosphere. The reaction mixture was stirred for 2 h at room temperature. Then the reaction mixture was concentrated and added dropwise to the solution of 2-amino-2-methyl propanol (70 g, 0.785 mol) in dichloromethane (200 ml) at 0° C. under nitrogen atmosphere. The resulting solution was stirred at room temperature for another 2 h. The reaction mixture was filtered and the filtrate was concentrated. The concentrated liquid mass (75 g) was used for the next step without purification.

### (ii) 2-(4-fluorophenyl)-4,4-dimethyl-4,5-dihydro-1,3-oxazole

Thionyl chloride (144 g) was added dropwise to 4-fluoro-N-(2-hydroxy-1,1-dimethylethyl)benzamide (75 g) (from step (i) above under stirring at room temperature and stirred for another 15 minutes. Then the yellow solution was poured into dry diethyl ether (500 ml) and the reaction mixture was neutralized with 20% cold sodium hydroxide solution. The diethyl ether layer was washed with water, brine and dried over anh. sodium sulfate and concentrated. The residue was purified by silica gel chromatography using (EtOAc/pet ether) to give the sub-title compound (65 g, 95%) as brown liquid.

### (iii) 2-(4,4-dimethyl-4,5-dihydro-1,3-oxazol-2-yl)-5-fluorobenzaldehyde

To 2-(4-fluorophenyl)-4,4-dimethyl-4,5-dihydro-1,3-oxazole (40 g, 0.206 mol) (from step (ii) above in diethyl ether (800 ml) was added dropwise sec-BuLi (192 ml, 1.4 M in cyclohexane) at −78° C. under nitrogen atmosphere and stirred for another 3 h at the same temperature. Then dry DMF (40 ml) was added and warmed up to room temperature overnight. The reaction was quenched and diluted with water. The separated organic layer was washed water and brine. Then the organic layer was dried over anhydrous sodium sulfate and concentrated in vacuum to give crude intermediate (36 g) as yellow liquid.

### (iv) 5-fluoro-3-hydroxy-2-benzofuran-1(3H)-one

2-(4,4-dimethyl-4,5-dihydro-1,3-oxazol-2-yl)-5-fluorobenzaldehyde (36 g) from step (iii) above was heated at 80° C. for overnight with HCl in water (400 ml water, 200 ml Conc. HCl). The reaction mixture was cooled to room temperature and stirred with diethyl ether (500 ml). The diethyl ether layer was washed with water and brine. The organic layer was dried over anh. sodium sulfates and concentrated under vacuum. The crude mass was recrystallized from ethylacetate/pet ether solvent system to give the title compound (7 g, 26%) an off white solid.

Preparation Y

### 2-[2-(4-chlorophenyl)propyl]-3-oxoisoindoline-1-carboxylic acid

### (i) Ethyl 2-(2-ethoxy-2-oxoethyl)benzoate

2-Carboxymethyl benzoic acid (25 g) was dissolved in absolute ethanol (250 ml) and con. Sulfuric acid (1 ml) was added. The resulting solution was refluxed in a Dean & Stark apparatus for 2 days and the solvent was replaced by absolute ethanol for 3 times. Ethanol was removed under reduced pressure and the concentrated mass was dissolved in ethyl acetate. Ethyl acetate layer was washed with saturated sodium bicarbonate solution, water, and brine, dried over anh. Sodium sulfate and concentrated to afford the sub-title compound (30 g) as yellow oil.

### (ii) ethyl 2-(1-bromo-2-ethoxy-2-oxoethyl)benzoate

ethyl 2-(2-ethoxy-2-oxoethyl)benzoate (30 g, 0.127 mol) from step (i) above was mixed with N-bromosuccinamide (22.5 g, 0.127 mol) in carbon tetrachloride (300 ml). AIBN was added and refluxed for 2 days. Then the reaction mixture was washed with water, dried over anh. sodium sulfate and concentrated. The crude intermediate was purified by column purification by using 2% ethyl acetate in pet ether to give the sub-title compound (26 g, 65%) as brown liquid

### (iii) ethyl 2-[2-(4-chlorophenyl)propyl]-3-oxoisoindoline-1-carboxylate

[2-(4-chlorophenyl)propyl]amine (20 g, 0.118 mol) was added dropwise to the solution of step 2 intermediate (18.6 g, 0.059 mol) in acetonitrile (150 ml) at 0° C. under nitrogen atmosphere. Then the reaction mixture was stirred at room temperature for overnight. The reaction mixture was filtered, residue was washed with dichloromethane and combined filtrate was concentrated. The crude product was purified using 10% ethyl acetate in pet ether as eluent to give the sub-title compound (23 g, 100%) as white solid.

### (iv) 2-[2-(4-chlorophenyl)propyl]-3-oxoisoindoline-1-carboxylic acid

An aqueous solution of sodium hydroxide (3 g, in 50 ml water, 0.075 mol) was added to the well stirred solution of ethyl 2-[2-(4-chlorophenyl)propyl]-3-oxoisoindoline-1-carboxylate (13 g, 0.036 mol) in ethanol (100 ml) at 0° C. The reaction mixture was allowed to stirred at room temperature for 2 h. The reaction mixture was concentrated under reduced pressure and diluted with water and extracted with ethyl acetate. The aqueous layer was acidified with 2M HCl and extracted with ethyl acetate. The ethyl acetate layer was washed with brine, dried over anh. sodium sulfate and concentrated under reduced pressure to give the title compound (11.5 g, 95.8%)

Preparation Z

### ethyl 2-[2-(4-chlorophenyl)ethyl]-3-oxoisoindoline-1-carboxylate

2-(4-chlorophenyl) ethylamine (25 g, 0.16 mol) was added dropwise to the solution of ethyl 2-(1-bromo-2-ethoxy-2-oxoethyl)benzoate (25 g, 0.0793 mol) in acetonitrile (150 ml) at 0° C. under nitrogen atmosphere. Then the reaction mixture was stirred at room temperature for overnight. The reaction mixture was filtered, residue was washed with dichloromethane and combined filtrate was concentrated. The crude product was purified using 15% ethylacetate in petether as eluent to give the title compound (22 g, 80.8%) as white solid.

Preparation AA

### 2-[2-(4-chlorophenyl)ethyl]-3-oxoisoindoline-1-carboxylic acid

An aqueous solution of sodium hydroxide (3 g, in 50 ml water, 0.075 mol) was added to the well stirred solution of ethyl 2-[2-(4-chlorophenyl)ethyl]-3-oxoisoindoline-1-carboxylate (preparation Z above) (15 g, 0.043 mol) in ethanol (100 ml) at 0° C. Then the reaction mixture was allowed to stirred at room temperature for 2 h. The reaction mixture was concentrated under reduced pressure and diluted with water and extracted with ethylacetate. The aqueous layer was acidified with 2(N) HCl and extracted with ethylacetate. The ethylacetate layer was washed with brine, dried over anh. sodium sulfate and concentrated under reduced pressure to give the title compound (13 g, 94.8%).

Preparation AB

### (2-phenoxybenzyl)amine hydrochloride

### (i) 1-bromo-2-phenoxybenzene

A solution of sodium nitrite (9.2 g, 0.135 mol) in water (20 ml) was added dropwise to a solution of 2-phenoxyaniline (25 g, 0.135 mol) in 40% hydrobromic acid (50 ml) at 0° C. and stirred for another 10 minutes. Then the reaction mixture was added to the boiling mixture of cuprous bromide (21.3 g, 0.149 mol) in 40% hydrobromic acid (50 ml) and after addition it was allowed to reflux for another 30 minutes. Reaction mixture was cooled, diluted with water and extracted with diethyl ether. The diethyl ether layer was washed with 5% hydrochloric acid, 10% potassium hydroxide, water, brine, dried over sodium sulfate and concentrated to give the crude intermediate. The sub.-title compound (14.8 g, 45%) was obtained by column purification of the crude intermediate using pet ether as eluent

### (ii) 2-phenoxybenzonitrile

A solution of 1-bromo-2-phenoxybenzene from step (i) above (14.8 g, 0.0594 mol), Zn(CN)₂ (6.9 g, 0.0594 mol) and Pd (PPh₃)₄ (6.8 g, 0.00594 mol) in dimethylformamide (100 ml) was heated at 130° C. for overnight under nitrogen atmosphere. Reaction mixture was cooled to room temperature and diluted with water (200 ml). The reaction mixture was stirred with ethyl acetate (200 ml) for 15 minutes and filtered through celite. The ethyl acetate layer was separated and washed with water, brine, dried over anh. sodium sulfate and concentrated. The crude intermediate was purified by column chromatography using 4% ethyl acetate in pet ether to give the sub-title compound (10.1 g, 87.8%) as colorless liquid.

### (iii) (2-phenoxybenzyl)amine hydrochloride

A solution of 2-phenoxybenzonitrile (from step (ii) above (10.1 g, 0.0517 mol) in THF (50 ml) was added dropwise to the well-stirred suspension of LAH (4.9 g, 0.129 mol) in THF (50 ml) at 0° C. under nitrogen atmosphere and then allowed to stir at room temperature for overnight. The reaction mixture was quenched with 6(N) KOH (5 ml) 0° C. and stirred with THF (50 ml) for another 30 minutes. The reaction mixture was filtered and the residue was washed with ethyl acetate. The filtrate was concentrated to give the crude amine. Satd. HCl in diethyl ether (20 ml) was added to the solution of crude amine in diethyl ether (20 ml) and stirred for 2 h. Then the reaction mixture was filtered and residue was dried to give the title compound (10 g, 97.08

Preparation AC

### (dipyridin-3-ylmethyl)amine hydrochloride

### (i) dipyridin-3-ylmethanone

n-BuLi (71.3 ml, 1.6 M, 0.114 mol) was added dropwise to the solution of 3-bromo pyridine (15 g, 0.095 mol) in dry diethyl ether (200 ml) at −78° C. and stirred for 15 minutes. A solution of ethyl nicotinate (13 g, 0.095 mol) in dry diethyl ether (50 ml) was added dropwise to the reaction mixture at −78° C. and stirred for another 2 h at the same temperature. Then the reaction was quenched with satd. ammonium chloride and extracted with ethyl acetate. The organic layer was washed with satd. brine, dried over anh. sodium sulfate and concentrated. The crude product was purified on neutral alumina column using (methanol/dichloromethane) to give the sub-title compound (8.5 g, 49% as brown liquid.

### (ii) dipyridin-3-ylmethanone oxime

A solution of dipyridin-3-ylmethanone from step (i) above (8.5 g, 0.046 mol) in dry methanol (50 ml) was added to the well stirred solution of sodium acetate (9.6 g, 0.117 mol) and hydroxylamine hydrochloride (8.12 g, 0.117 mol) in dry methanol (50 ml). The reaction mixture was refluxed under nitrogen atmosphere for 2 h. Then the reaction mixture was concentrated, diluted with water and extracted with dichloromethane. The organic layer was washed with water, brine and dried over anh. sodium sulphate and concentrated to give the sub-title compound (9.5 g, 100%)

### (iii) (dipyridin-3-ylmethyl)amine hydrochloride

dipyridin-3-ylmethanone oxime (step (ii) above (9.5 g, 0.0477 mmol) and ammonium acetate (5.5 g, 0.0716 mmol) were dissolved in ethanol (00 ml), water (80 ml) and 40% NH3 (aq) (100 ml). The mixture was heated to 80° C. and zinc dust (15.5 g, 0.23 mmol) was added over a period of 1 h. The reaction was then stirred at 80° C. for overnight and was then cooled to rt, filtered and the filtrate was concentrated in vacuum. The remaining aqueous solution was basified with 10 M NaOH (aq), extracted with dichloromethane. The combined organic phases were washed with brine (40 ml) dried over anh. Sodium sulphate and concentrated. The concentrated yellow gummy mass was dissolved in ethyl acetate and diethyl ether (50 ml) satd. with HCl gas was added drop wise and stirred for 1 h. The reaction mixture was filtered and solid residue was dried to give the title compound (9.2 g, 65%) as off white solid

Preparation AD

### 3-(2-aminoethyl)benzonitrile trifluoroacetic acid salt

### (i) tert-butyl[2-(3-bromophenyl)ethyl]carbamate

BOC anhydride (12 ml, 0.054 mol) was added to the ice-cold solution of 3-Bromo phenethylamine (10 g, 0.049 mol) and triethylamine (10.4 ml, 0.10 mol) in dichloromethane (100 ml). After 1 h stirring at room temperature the reaction mixture was quenched with water and extracted with dichloromethane. Organic layer was washed with water and brine, dried over anh. sodium sulphate and concentrated to give the sub-title compound (15 g, 100%)

### (ii) tert-butyl[2-(3-cyanophenyl)ethyl]carbamate

A mixture tert-butyl tert-butyl[2-(3-bromophenyl)ethyl]carbamate (15 g, 0.05 mol), Zn(CN)₂ (5.87 g, 0.05 mol) and tetrakis triphenylphosphine palladium (0) (5.7 g, 0.005 mol) was heated at 140° C. for 5 h in dry DMF (150 ml) under nitrogen atmosphere. Then the reaction mixture was cooled to room temperature, quenched with water and filtered through celite pad. The reaction mixture was extracted with ethyl acetate and the ethyl acetate layer was washed with water, brine successively and dried over anh. sodium sulphate and concentrated. Then the crude material was purified by column chromatography using 20% ethyl acetate in pet ether as eluent to give the sub-title compound desired (7 g, 57%) as white solid

### (iii) 3-(2-aminoethyl)benzonitrile trifluoroacetic acid salt

Trifluoroacetic acid (5 ml) was added to a solution of tert-butyl[2-(3-cyanophenyl)ethyl]carbamate (10 g) in dichloromethane (20 ml) and stirred at room temperature for overnight. The solvent and excess trifluoroacetic acid was removed under reduced pressure to give gummy oil. On trituration of gummy oil with diethyl ether, white solid appeared. Diethyl ether was decanted and white solid was dried to get the title compound (6 g)

Preparation AE

### [2-fluoro-4-(methylsulfonyl)benzyl]amine

### (i) O-(4-cyano-3-fluorophenyl)dimethylthiocarbamate

A mixture of 2-fluoro-4-hydroxy benzonitrile (10 g, 0.0729 mol), DMAP (900 mg, 0.00729 mol), triethylamine (30 ml, 0.218 mol) and dimethylthiocarbamoyl chloride (11 g, 0.0875 mol) in dry dichloromethane (200 ml) was stirred at reflux under nitrogen atmosphere overnight. The reaction mixture was quenched with water and extracted with dichloromethane. The organic layer was washed with water, brine and dried over sodium sulfate. The solvent was evaporated under reduced pressure and the residue was triturated with pet ether. The product was filtered and dried under vacuum to give the sub-title compound (14 g, 85.8%) as yellow solid

### (ii) S-(4-cyano-3-fluorophenyl)dimethylthiocarbamate

O-(4-cyano-3-fluorophenyl) dimethylthiocarbamate from step (i) above (14 g) and diphenyl ether (200 ml) were mixed and stirred at 210° C. for 6 h. Then diphenyl ether was removed by distillation under reduced pressure and the crude mass was stirred with pet ether and filtered. The residue was washed with pet ether for several times and air dried to give the sub-title compound (13.4 g, 95.7%) as light brown solid .

### (iii) 2-fluoro-4-mercaptobenzonitrile

S-(4-cyano-3-fluorophenyl) dimethylthiocarbamate from step (ii) above (13.4 g, 0.059 mol) was taken in THF (150 ml) and to which a solution of KOH (6.7 g, 0.11 mol) in methanol (200 L) was added. The reaction mixture was stirred at RT for 3 h and then concentrated. The crude mass was dissolved in ethylacetate. The ethylacetate layer was acidified with 3(N) HCl to pH=2. The ethylacetate layer was washed with water and brine, dried over anh. sodium sulphate and concentrated. The crude intermediate (9 g) obtained was directly taken for the next step without further

### (iv) 2-fluoro-4-(methylthio)benzonitrile

Methyl iodide (12.5 g, 0.058 mol) was added dropwise to a solution of 2-fluoro-4-mercaptobenzonitrile (9 g, 0.058 mol) (from step (iii) above and potassium carbonate (12.1 g, 0.088 mol) in dry acetonitrile (150 ml) and stirred at room temperature for 3 h under nitrogen atmosphere. The reaction mixture was filtered and the filtrate was concentrated to give the sub-title compound (9.6 g, 100%)

### (v) 2-fluoro-4-(methylsulfonyl)benzonitrile

2-fluoro-4-(methylthio)benzonitrile (9.6 g, 0.0524 mol) from step (iv) above) was added to a mixture of glacial acetic acid/water/ethanol (140 ml, 2:2:3) and stirred for 10 min. The reaction mixture was cooled to 0° C. and oxone (40.4 g, 0.065 mol) was added in portions. The reaction mixture was stirred at RT for 2 h and diluted with dichloromethane. Filtered off inorganic and mother liquor was partitioned between water and dichloromethane. Organic layer was washed with water and brine and dried over sodium sulfate. Solvent evaporation under reduced pressure followed by recrystallization of the crude product using ethylacetate/petether afforded desired sulfone (9 g, 80%) .

### (vi) 2-fluoro-4-(methylsulfonyl)benzonitrile

To a solution of 2-fluoro-4-(methylthio)benzonitrile (9 g) in methanol (150 ml), Raney nickel (2 g) was added and then saturated with ammonia (g). The reaction mixture was hydrogenated in parr shaker under 3 kg hydrogen pressure over overnight. The reaction mixture was filtered and the filtrate was concentrated. The concentrated mass was dissolved in ethyl acetate and stirred with saturated HCl in ether for overnight under nitrogen atmosphere. The solid precipitates were filtered, washed with diethyl ether and dried to give the title compound (1.3 g, 14.3%) as pale yellow solid.

Preparation AF

### [2-chloro-4-(methylsulfonyl)benzyl]amine Hydrochloride

### (i) O-(3-chloro-4-cyanophenyl)dimethylthiocarbamate

A mixture of 2-chloro-4-hydroxy benzonitrile (25 g, 0.162 mol), DMAP (1.9 g, 0.162 mol), triethylamine (68 ml, 0.483 mol) and dimethylthiocarbamoyl chloride (24 g, 0.195 mol) in dry dichloromethane (500 ml) was stirred at reflux under nitrogen atmosphere overnight. The reaction mixture was quenched with water and extracted with dichloromethane. Organic layer was washed with water, brine and dried over sodium sulfate. Solvent evaporated under reduced pressure and the residue was triturated with pet ether. The product was filtered and dried under vacuum to give desired intermediate (38 g, 97%) as yellow solid

### (ii) S-(3-chloro-4-cyanophenyl)dimethylthiocarbamate

O-(3-chloro-4-cyanophenyl) dimethylthiocarbamate (38 g) and diphenyl ether (500 ml) were mixed and stirred at 210° C. for 6 h. Then diphenyl ether was removed by distillation under reduced pressure and the crude mass was stirred with pet ether and filtered. The residue was washed with pet ether for several times and air dried to give desired intermediate (37.8 g, 99.5%) as light brown solid.

### (iii) 2-chloro-4-mercaptobenzonitrile

S-(3-chloro-4-cyanophenyl) dimethylthiocarbamate (35 g, 0.145 mol) was taken in THF (400 ml) and to which a solution of KOH (163 g, 0.30 mol) in methanol (200 L) was added. The reaction mixture was stirred at RT for 3 h and then concentrated. The crude mass was dissolved in ethylacetate. The ethylacetate layer was acidified with 3(N) HCl to P_{H}=2. The ethylacetate layer was washed with water and brine, dried over anh. sodium sulphate and concentrated. The crude intermediate (25 g) obtained was directly taken for the next step without further purification.

### (iv) 2-chloro-4-(methylthio)benzonitrile

Methyl iodide (31.4 g, 0.22 mol) was added dropwise to a solution of 2-chloro-4-mercaptobenzonitrile (25 g, 0.147 mol) and potassium carbonate (20.4 g, 0.22 mol) in dry acetonitrile (300 ml) and stirred at room temperature for 3 h under nitrogen atmosphere. Then the reaction mixture was filtered and the filtrate was concentrated to give the desired intermediate (26 g, 96.2%).

### (v) 2-chloro-4-(methylsulfonyl)benzonitrile

2-chloro-4-(methylthio)benzonitrile (26 g, 0.145 mol) was added to a mixture of glacial acetic acid/water/ethanol (900 ml, 2:2:3) and stirred for 10 min. The reaction mixture was cooled to 0° C. and oxone (217 g, 0.353 mol) was added in portions. The reaction mixture was stirred at RT for overnight and diluted with dichloromethane. Filtered off inorganic and mother liquor was partitioned between water and dichloromethane. Organic layer was washed with water and brine and dried over sodium sulfate. Solvent evaporation under reduced pressure followed by recrystallization of the crude product using ethyl acetate/petether afforded the sub-title compound (18 g, 59%) as white solid .

### (vi) [2-chloro-4-(methylsulfonyl)benzyl]amine hydrochloride

To a solution of step 5 intermediate (8 g) in methanol (200 ml), Raney nickel (2.5 g) was added and then saturated with ammonia (g). Then the reaction mixture was hydrogenated in parr shaker under 3 kg hydrogen pressure over overnight. The reaction mixture was filtered and the filtrate was concentrated. The concentrated mass was dissolved in ethyl acetate and stirred with saturated HCl in ether for overnight under nitrogen atmosphere. The solid precipitates were filtered, washed with diethyl ether and dried to give the title compound (7 g, 86.4%) as a solid.

Preparation AG

### 2-(4-chlorophenyl)-2-methyl-propan-1-amine hydrochloride

### (i) 2-(4-chlorophenyl)-2-methylpropanenitrile

To a stirred solution of 2-(4-chlorophenyl)acetonitrile (10 g, 0.066 mol) in DMF, sodium hydride (3.95 g, 0.1649 mol) was added at 0° C. in portions. The reaction mixture was allowed to stir for 1 hour at room temperature. Then it was again cooled to 0° C. and methyl iodide (28.0 g, 0.198 mol) was added dropwise. The reaction mixture was allowed to stir at 40° C. for 4 hr. The reaction mixture was diluted with cold water and product was extracted with EtOAc. The organic layer was washed with water and brine. The organic layer was dried over anhydrous sodium sulfates and concentrated under vacuum. The residue was purified by silica gel chromatography using (EtOAc/pet ether) to give the sub-title compound (7.7 g, 65%) as colourless liquid

### (ii) 2-(4-chlorophenyl)-2-methyl-propan-1-amine hydrochloride

To a mixture of LiAlH₄ (4.06 g, 0.107 mol) in THF was added dropwise step 1 intermediate (7.7 g, 0.043 mol) in THF at 0° C. The reaction mixture was allowed to stir at room temperature overnight. The reaction mixture was cooled to 0° C. and quenched with 10 ml of 6M KOH solution. The reaction mixture was diluted with THF and allowed to stir at room temperature for 30 min. The reaction mixture was filtered and filtrate was concentrated. The residue was diluted with diethyl ether and HCl in ether was added at 0° C. The white solid was collected by filtration, washed with dry diethyl ether and dried under vacuum to afford desired product (6 g, 77%) of the title compound

Preparation AH

### 7-fluoro-3-hydroxy-2-benzofuran-1(3H)-one

### (i) 1-(dimethoxymethyl)-3-fluorobenzene

To a stirred solution of 3-fluorobenzaldehyde (17.5 g, 0.141 mol) in methanol (175 ml), trimethyl orthoformate (17.5 ml, 0.159 mol) and PTSA (175 mg) were added. The reaction mixture was stirred at RT overnight. The reaction mixture was quenched with 30 ml of 1% methanol KOH solution. The reaction mass was concentrated, then diluted with water and product was extracted with ethyl acetate. The organic layer was washed with water and brine. The organic layer was dried over anh. Sodium sulfates and concentrated under vacuum. The crude was fractionally distilled to get the desired intermediate (20 g, 83.6%) as colorless liquid

### (ii) 7-fluoro-3-hydroxy-2-benzofuran-1(3H)-one

To a solution of 1-(dimethoxymethyl)-3-fluorobenzene (20 g, 0.11 mol) in THF (200 ml) (from step (i) above was added drop wise sec-BuLi (220 ml, 1.4 M in cyclohexane) at −78° C. under nitrogen atmosphere and stirred for another 3 h at the same temperature. Then CO2 was purged until the red solution became yellow and slowly warmed up to room temperature. The reaction mixture was stirred for another 1 h and then quenched with 20 ml of HCl. Then the reaction mixture was concentrated. The crude reaction mass so obtained was heated at 80° C. for overnight with HCl in water (300 ml Conc. HCl, 200 ml water). Then the reaction mixture was cooled to room temperature and stirred with diethyl ether (100 ml). The diethyl ether layer was washed with water and brine. The organic layer was dried over anh. Sodium sulfates and concentrated under vacuum. The crude mass was submitted for prep HPLC to give the title compound (2.3 g, 11.7%) as a white solid

Preparation AI

### 1-(3′,4′-difluorobiphenyl-2-yl)methanamine

### (i) Tert-butyl[(3′,4′-difluorobiphenyl-2-yl)methyl]carbamate

Tert-butyl (2-bromobenzyl)carbamate (1.74 mmol, 0.50 g), Tetrakis(triphenylphosphine)palladium (0.087 mmol, 0.10 g) and (3,4-difluorophenyl)boronic acid (2.10 mmol, 0.33 g) were dissolved in (DME 10 ml) in a micro vial. Cesium carbonate (3.49 mmol, 1.14 g) was dissolved in 2 ml water and then added to the mixture. Ar (g) was bubbled through the mixture for 5 minutes. The crude was purified by flash chromatography (started with isocratic heptane/EtOAc 90/10 and then the EtOAc concentration was increased to 70%, (silica gel 60 0.004-0.063 mm). The product containing fractions was pooled and the solvent was removed by evaporation to give the title compound (3.00 g, 89.6%). 1H-NMR (500 MHz, CDCl₃): δ 7.46-7.42 (m, 1H); 7.41-7.36 (m, 1H); 7.36-7.30 (m, 1H); 7.25-7.18 (m, 2H); 7.17-7.11 (m, 1H); 7.06-7.01 (m, 1H); 4.75 (bs, 1H); 4.31-4.17 (m, 2H); 1.45 (s, 9H).

### (i) 1-(3′,4′-difluorobiphenyl-2-yl)methanamine

Tert-butyl[(3′,4′-difluorobiphenyl-2-yl)methyl]carbamate (9.39 mmol, 3.00 g) was dissolved in HCl saturated EtOAc and stirred at rt for 2 h. The solvent was removed by evaporation and the HCl-salt was purified by flash chromatography (started with isocratic heptane/DCM 50/50 and then the DCM concentration was increased to 100% then the product was eluated with 10% MeOH (saturated with NH3), (silica gel 60 0.004-0.063 mm). The product containing fractions was pooled and the solvent was removed by evaporation to give the title compound (2.00 g, 97.1%)

1H-NMR (500 MHz, CDCl₃): δ 7.55-7.50 (m, 1H); 7.42-7.35 (m, 1H); 7.32-7.15 (m, 4H); 7.14-7.06 (m, 1H); 4.82-4.79 (m, 2H); 3.78-3.73 (m, 2H).

Preparation AJ

### 1-(4,4′-difluorobiphenyl-2-yl)methanamine

### (i) Tert-butyl[(4,4′-difluorobiphenyl-2-yl)methyl]carbamate

1-bromo-4-fluorobenzene (11.15 mmol, 1.95 g), Tetrakis(triphenylphosphine)palladium (0.41 mmol, 0.47 g) and (2-{[(tert-butoxycarbonyl)amino]methyl}-4-fluorophenyl)boronic acid (9.29 mmol, 2.50 g) were dissolved in (DME 10 ml) in a micro vial. Cesium carbonate (18.58 mmol, 6.05 g) was dissolved in 2 ml water and then added to the mixture. N2 (g) was bubbled through the mixture for 5 minutes. The reaction was performed in a micro oven (20 min, 130 deg). The crude was purified by flash chromatography (started with isocratic heptane/EtOAc 95/5 and then the EtOAc concentration was increased to 60%, (silica gel 60 0.004-0.063 mm). The product containing fractions was pooled and the solvent was removed by evaporation to give the title compound (2.54 g, 85.6%). 1H-NMR (500 MHz, CDCl₃): δ 7.30-7.21 (m, 2H), 7.21-7.08 (m, 4H); 7.03-6.97 (m, 1H), 4.79 (bs, 1H), 4.29-4.13 (m, 2H); 1.45 (s, 9H).

### (ii) 1-(4,4′-difluorobiphenyl-2-yl)methanamine

Tert-butyl[(4,4′-difluorobiphenyl-2-yl)methyl]carbamate (7.95 mmol, 2.54 g) was dissolved in HCl saturated EtOAc and stirred at rt for 2 h. The solvent was removed by evaporation and the HCl-salt of the product was purified by flash chromatography (started with isocratic heptane/EtOAc 50/50 and then the EtOAc concentration was increased to 100% then the product was eluated with 10% MeOH (saturated with NH3), (silica gel 60 0.004-0.063 mm). The product containing fractions was pooled and the solvent was removed by evaporation to give the title compound (1.64 g, 94.1%). 1H-NMR (500 MHz, CD₃OD): δ 7.34-7.27 (m, 3H); 7.22-7.13 (m, 3H), 7.05-7.0 (m, 1H); 4.81 (s, 2H), 3.72 (s, 2H).

Preparation AK

The Following Amines were Made According to Preparation AI and AJ Above

[(4-fluorobiphenyl-2-yl)methyl]amine

[(5-fluorobiphenyl-2-yl)methyl]amine

[(4′,5-difluorobiphenyl-2-yl)methyl]amine

### Examples

### Example 1

### 2-[2-(4-chlorophenyl)propyl]-N-[(5-methylisoxazol-3-yl)methyl]-3-oxoisoindoline-1-carboxamide

A solution of 2-formyl-benzoic acid (1.23 g, 8.2 mmol) in methanol (15 ml) was treated with 2-(4-chloro-phenyl)-propylamine hydrochloride (1.69 g, 8.2 mmol) and triethylamine (1.14 ml). The mixture was stirred at room temperature for 30 min. The 3-Isocyanomethyl-5-methyl-isoxazole solution from Preparation L above was added and the mixture was stirred at room temperature for 16 hr. The mixture was concentrated, dissolved in 50 ml dichloromethane and washed with 100 ml saturated NaHCO₃ solution. The organic phase was separated, dried over MgSO₄ and evaporated. The remaining oil was purified using preparative HPLC giving the title compound (0.903 g, 26% yield).

[M+1] (ES) 424.10

¹H NMR (500 MHz, CDCl₃) δ 7.46-7.61 (m, 3H); 7.35-7.44 (m, 1H); 7.07-7.27 (m, 5H); 5.81 (s, 1H); 4.78 (s, 1H); 4.46-4.56 (m, 1H); 4.31-4.39 (m, 1H); 4.12-4.27 (m, 1H); 3.15-3.40 (m, 2H); 2.35 (s, 3H); 1.23 (d, 3H)

### Example 2

### 2-(biphenyl-2-ylmethyl)-N-(tert-butyl)-5-hydroxy-4-methyl-3-oxoisoindoline-1-carboxamide

### (i) N-(biphenyl-2-ylmethyl)-N42-(tert-butylamino)-1-(2-furyl)-2-oxoethyl]but-2-ynamide

(biphenyl-2-ylmethyl)amine (23.78 mmol, 4.36 g) was dissolved in MeOH and 2-furaldehyde (23.78 mmol, 2,29 g) and but-2-ynoic acid (23.78 mmol, 2.00 g) was added. The mixture was stirred at rt for 30 min. Tert-butyl isocyanide (23.78 mmol, 1.98 g) was added and the mixture was stirred at rt over night. The solvent was removed by evaporation. The product was taken on to the next step without further purification.

### (ii) 2-(biphenyl-2-ylmethyl)-N-(tert-butyl)-5-hydroxy-4-methyl-3-oxoisoindoline-1-carboxamide

N-(biphenyl-2-ylmethyl)-N-[2-(tert-butylamino)-1-(2-furyl)-2-oxoethyl]but-2-ynamide (23.0 mmol, 9.87 g) (from step (i) above was dissolved in Xylene (200 ml), ytterbium (III) trifluoromethansulfonate (2.30 mmol, 1.43 g) was added. The mixture was refluxed for 1.5 h and then no starting material was left. The solvent was removed by evaporation. The crude was purified by flashcromatography (started with isocratic heptane/EtOAc 80/20 and then the EtOAc concentration was increased to 100% (silica gel 60 0.004-0.063 mm). The product precipitated on the column and had to be eluated with 50% MeOH. Then the product was re-crystallized from MeOH to give the title compound (4.52 g, 45.9%).

¹H-NMR (500 MHz, DMSO-d₆): δ 7.96 (s, 1H), 7.45-7.28 (m, 7H), 7.26-7.20 (m, 1H), 7.20-7.14 (m, 1H), 7.06-7.01 (m, 1H), 6.97-6.92 (m, 1H), 5.07 (d, 1H), 4.63 (s, 1H), 3.90 (d, 1H), 2.45 (s, 3H), 1.11 (s, 9H); HRMS calculated for (C₂₇H₂₈N₂O₃+H)+, 429.5436; found (ES [M+H]+), 429.5454.

The synthetic sequence originated in a published procedure:

D. L. Wright, C. V. Robotham and K. Aboud, Tetrahedron Lett. 2002, 43, 943-946.

### Example 3

### (R or S) 2-(biphenyl-2-ylmethyl)-N-(tert-butyl)-5-hydroxy-4-methyl-3-oxoisoindoline-1-carboxamide

### (S or R) 2-(biphenyl-2-ylmethyl)-N-(tert-butyl)-5-hydroxy-4-methyl-3-oxoisoindoline-1-carboxamide

The enantiomers of 2-(biphenyl-2-ylmethyl)-N-(tert-butyl)-5-hydroxy-4-methyl-3-oxoisoindoline-1-carboxamide (example 2) (0.20 g, 0.47 mmol) were separated by preparative HPLC using a Reprosil 20×250 mm chiral column using 40% isopropyl alcohol in heptane as mobile phase which gave (+)-enantiomer (0.10 g) (E1) and (−)-enantiomer (0.10 g) of the title compound. (E2)

(+)-Enantiomer:

HRMS: calculated for (C₂₇H₂₈N₂O₃+H)⁺ 429.2178; found (ES [M+H]⁺) 429.2166.

(−)-Enantiomer:

HRMS: calculated for (C₂₇H₂₈N₂O₃+H)⁺ 429.2178; found (ES [M+H]⁺) 429.2147.

### Example 4

### Methyl (2R)-2-{1-[(tert-butylamino)carbonyl]-3-oxo-1,3-dihydro-2H-isoindol-2-yl}-3-(4-chlorophenyl)propanoate

2-Formyl-benzoic acid (0.30 g, 2 mmol), methyl 4-chloro-D-phenylalaninate hydrochloride (0.5 g, 2 mmol) and NEt3 (0.28 mL, 2 mmol) were dissolved in MeOH (5 mL) and stirred at ambient temperature for 30 mins. tert-Butyl-isocyanide (0.23 mL, 2 mmol) was added and the resulting mixture was stirred for 3 days at ambient temperature. The mixture was concentrated under reduced pressure and the residue was purified by preparative HPLC using a Waters HPLC system, equipped with a Kromasil C8 50.8×300 mm column using a MeCN/0.1 M NH4OAc buffer system with a gradient from 100% A (5% MeCN+95% 0.1 M NH4OAc) to 100% B (100% 0.1 M NH4OAc) as mobile phase. The product fraction was concentrated under reduced pressure and freeze-dried to give the title compound (0.22 g, 25%).

1H-NMR (500 MHz, CDCl3) δ 7.87-7.83 (m, 1H), 7.60-7.49 (m, 3H), 7.17-7.13 (m, 2H), 7.02-6.98 (m, 2H), 4.17-4.12 (m, 1H), 3.96 (s, 1H), 3.94 (s, 3H), 3.80-3.40 (m, 2H), 1.22 (s, 9H)

13C-NMR (125 MHz, CDCl3) δ 170.9, 169.5, 166.7, 142.1, 135.6, 133.4, 133.0, 130.5, 130.0, 129.4, 129.1, 123.8, 123.3, 67.4, 59.5, 53.6, 51.8, 34.0, 28.6

HRMS: calculated for (C23H25ClN2O4+H)+ 429.1581; found (ES [M+H]+) 429.1583

### Example 5

### N-(tert-butyl)-2-{(1R)-1-(4-chlorobenzyl)-2-oxo-2-[(4,4,4-trifluorobutyl)amino]ethyl}-3-oxoisoindoline-1-carboxamide

4,4,4-trifluorobutan-1-amine (0.087 g, 0.53 mmol) was mixed with dichloromethane (2 ml) under nitrogen atmosphere and Me₃Al (2M in heptane) was added. The resulting mixture was stirred for 1.5 h at ambient temperature and methyl (2R)-2-{1-[(tert-butylamino)carbonyl]-3-oxo-1,3-dihydro-2H-isoindol-2-yl}-3-(4-chlorophenyl)propanoate (Example 4 above) (0.11 g, 0.26 mmol) was added. The resulting mixture was stirred at ambient temperature for 20 h. The reaction was quenched by addition of methanol and the formed salts were removed by filtration. The filtrate was purified by silica flash chromatography using a Biotage Horizon apparatus with ethyl acetate/heptane as mobile phase. The product fraction was concentrated under reduced pressure to give the title compound (0.075 g, 54%).

HRMS: calculated for (C₂₆H₂₉ClF₃N₃O₃+H)⁺ 524.1927; found (ES [M+H]⁺) 523.1893.

### Example 6

### N-butyl-2-[2-(4-chlorophenyl)ethyl]-N-methyl-3-oxoisoindoline-1-carboxamide

2-[2-(4-chlorophenyl)ethyl]-3-oxoisoindoline-1-carboxylic acid (0.4 mmol) (Preparation AA) was dissolved in DCM (2 mL) and (3-dimethylamino-propyl)-ethyl-carbodiimide hydrochloride (0.4 mmol) was added as a solid. After 15 mins N-methylbutan-1-amine (0.4 mmol) was added and the reaction was then allowed to continue at 30 degrees for 2 days. The reaction mixture was purified by silica flash chromatography using a Biotage Horizone apparatus with ethyl acetate/heptane as mobile phase. The product fraction was concentrated under reduced pressure to give the title compound (0.007 g, 5%).

¹H-NMR (500 MHz, CDCl₃) δ 7.91-7.87 (m, 1H), 7.58-7.48 (m, 2H), 7.40-7.32 (m, 1H), 7.30-7.24 (m, 3H), 7.21-7.13 (m, 2H), 5.26-5.15 (m, 1H), 4.40-4.24 (m, 1H), 3.45-3.21 (m, 3H), 3.10-2.89 (m, 4H), 1.57-1.45 (m, 2H), 1.35-1.26 (m, 2H), 0.94 (t, 3H)

HRMS calculated for (C₂₂H₂₅ClN₂O₂+H)⁺ 385.1683; found (ES [M+H]⁺) 385.1670.

### Example 7

### 2-(biphenyl-2-ylmethyl)-N-(4-cyanobenzyl)-3-oxoisoindoline-1-carboxamide

2-formylbenzoic acid (9.09 mmol, 1.36 g) was dissolved in Methanol (20 ml) and 1-biphenyl-2-ylmethanamine (9.09 mmol, 1.66 g) was added and the mixture was stirred at rt for 30 minutes. Then 4-(isocyanomethyl)benzonitrile (9.09 mmol, 1.29 g) dissolved in Acetonitrile (5 ml) was added to the mixture. The reaction was stirred at rt over night. The crude was purified by flashcromatography (started with isocratic Toluene/EtOAc 100/0 and then the EtOAc concentration was increased to 50%, (silica gel 60 0.004-0.063 mm). The product containing fractions was pooled and the solvent was removed by evaporation to give the title compound (2.32 g, 55.8%).

¹H-NMR (500 MHz, CDCl₃): δ 7.63-7.56 (m, 3H), 7.50-7.45 (m, 2H); 7.45-7.39 (m, 3H); 7.37-7.24 (m, 4H); 7.22-7.16 (m, 3H); 7.00 (d, 2H); 6.59-6.54 (m,1H); 5.24 (d, 1H), 4.77 (s, 1H); 4.31 (d, 1H); 4.29-4.18 (m, 2H).

### Example 8

### N-[4-(aminomethyl)benzyl]-2-(biphenyl-2-ylmethyl)-3-oxoisoindoline-1-carboxamide

To a solution of 2-(biphenyl-2-ylmethyl)-N-(4-cyanobenzyl)-3-oxoisoindoline-1-carboxamide (Example 7) (0.43 mmol, 200 mg) in 100 mL MeOH (2M NH₃) was added Raney-Ni (prewashed with EtOH (abs). The resulting mixture was hydrogenated at 1.3 bar for 16 h. The solvent was removed by evaporation. The crude was purified by flashcromatography (started with isocratic toluene/MeOH 90/10 and then the MeOH concentration was increased to 50%, (silica gel 60 0.004-0.063 mm). The product containing fractions was pooled and the solvent was removed by evaporation to give the title compound (0.172 g, 85.2%).

¹H-NMR (300 MHz, CDCl₃): δ 7.69 (d, 1H), 7.55-7.35 (m, 3H); 7.35-7.13 (m, 11H); 7.08 (d, 2H); 4.72 (s, 1H); 4.19 (s, 2H); 3.74 (s, 2H).

### Example 9

### N-benzyl-6-chloro-3-oxo-2-[(1R)-1-phenylethyl]isoindoline-1-carboxamide

5-chloro-3-hydroxy-2-benzofuran-1(3H)-one (Preparation V) (3.25 mmol, 0.6 g) was dissolved in Methanol (10 ml) and [(1R)-1-phenylethyl]amine (3.25 mmol, 0.38 g) was added and stirred for 20 min. Then (isocyanomethyl)benzene (3.25 mmol, 0.39 g) was added to the mixture. The reaction was stirred at rt over night. The solvent was removed by evaporation. The crude was purified by flashcromatography (started with isocratic heptane/EtOAc 90/10 and then the EtOAc concentration was increased to 50%, (silicagel 60 0.004-0.063 mm). The product containing fractions was pooled and the solvent was removed by evaporation. The substance was not pure enough so it was purified by preparative HPLC (started with isocratic acetonitrile/buffer 20/80 and then the acetonitrile concentration was increased to 95%, the buffer was a mixture of acetonitrile/water 10/90 and ammonium acetate (0.1 M, column KR-100-7-C8, 50 mm×250 mm, flow 40 ml/min). The product containing fractions was pooled and the acetonitrile was removed by evaporation. The product was frees dried over night to give the title compound (mixture of diastereomeres) (0.345 g, 26.2%). ¹H-NMR (500 MHz, CDCl3): δ 7.59-6.9 (m, 12H), 6.88-6.8 (m, 1H), 5.68-5.44 (m, 1H), 5.16-4.33 (t, 2H), 4.22-3.46 (m, 1H), 1.78-159 (m, 3H); HRMS calculated for (C₂₄H₂₁ClN₂O₂+H)⁺, 405.1368; found (ES [M+H]⁺), 405.1370.

### Example 10

### N-butyl-2-[2-(4-fluorophenoxy)benzyl]-3-oxoisoindoline-1-carboxamide

2-formylbenzoic acid (0.33 mmol, 50 mg) was dissolved in Methanol (1 ml) and [2-(4-fluorophenoxy)benzyl]amine hydrochloride (0.33 mmol, 84 mg) and TEA (0.66 mmol, 64 mg) was added. Then 1-isocyanobutane (0.33 mmol, 28 mg) dissolved in acetonitrile was added to the mixture. The reaction was stirred at rt over night. The solvent was removed by evaporation. The crude was purified by flashcromatography (started with isocratic heptane/EtOAc 90/10 and then the EtOAc concentration was increased to 100%, (silica gel 60 0.004-0.063 mm). The product containing fractions was pooled and the EtOAc was removed by evaporation to give the title compound (47 mg, 32.6%).

¹H-NMR (500 MHz, CDCl3): δ 7.68-7.64 (d, 1H), 7.62-7.56 (d, 1H), 7.56-7.5 (t, 1H), 7.42-7.36 (t, 1H), 7.36-7.31 (d, 1H), 7.27-7.20 (t, 1H), 7.10-6.93 (m, 5h), 6.85-6.80 (d, 1H), 6.77-6.71 (m,1H), 5.33-5.21 (d, 1H), 4.98 (s, 1H), 4.59-4.51 (d, 1H), 3.33-3.22 (m, 1H), 3.16-3.05 (m, 1H), 1.44-1.14 (m, 5H), 0.94-0.75, (m 3H); HRMS calculated for (C₂₆H₂₅FN₂O₃+H)+, 433.5069; found (ES [M+H]+), 433.5061

### Example 11

### 2-(biphenyl-2-ylmethyl)-N-(tert-butyl)-5-(difluoromethoxy)-4-methyl-3-oxoisoindoline-1-carboxamide

2-(biphenyl-2-ylmethyl)-N-(tert-butyl)-5-hydroxy-4-methyl-3-oxoisoindoline-1-carboxamide (Example 2) (0.252 mmol, 108 mg) was dissolved in DCM (2 ml) in a 2 ml micro vial, tetrabutylammoniumhydrogensulphate (0.262 mmol, 89 mg) and sodiumhydroxide (0.831 mmol, 33 mg) was added and the tube was sealed. N2 (g) was bubbled through the mixture for 5 min. chlorodifluoromethane (g) was bubbled through the mixture for 30 sec. The mixture was stirred at rt for 2 h. The solvent was removed by evaporation and the reaction mixture was purified by flashcromatography (started with isocratic Heptane/EtOAc 95/5 and then the EtOAc concentration was increased to 50%, (silica gel 60 0.004-0.063 mm). The product containing fractions was pooled and the solvent was removed by evaporation to give the title compound (44 mg, 36.5%).

¹H-NMR (500 MHz, CDCl3): δ 7.45-7.32 (m, 7H), 7.32-7.25 (m, 4H), 6.53 (t, 1H), 5.42 (s, 1H), 5.17 (d, 1H), 4.47 (s, 1H), 4.44 (d, 1H), 2.64 (s, 3H), 1.09 (s, 9H); HRMS calculated for (C₂₈H₂₈F₂N₂O₃+H)+, 479.5515; found (ES [M+H]+), 479.5485.

### Example 12

### N-butyl-2-[2-(4-chlorophenyl)propyl]-6-fluoro-3-oxoisoindoline-1-carboxamide

4-fluoro-3-hydroxy-2-benzofuran-1(3H)-one (Preparation U) (11.89 mmol, 2.0 g) was dissolved in Methanol (10 ml) and [2-(4-chlorophenyl)propyl]amine hydrochloride (11.89 mmol, 2.452 g) and TEA (13.08 mmol, 1.324 g) was added and stirred for 20 min. Then 1-isocyanobutane (11.89 mmol, 0.98 g) was added to the mixture. The reaction was stirred at rt over night. The solvent was removed by evaporation. The crude was purified by flashcromatography (started with isocratic heptane/EtOAc 90/10 and then the EtOAc concentration was increased to 50%, (silica gel 60 0.004-0.063 mm). The product containing fractions was pooled and the solvent was removed by evaporation. When evaporating the product precipitated and the solid was filtered of and dried to give the title compound (1.05 g, 21.9%).

¹H-NMR (500 MHz, CDCl3): δ 7.42-6.90 (m, 7H), 4.95-4.43 (d, 1H, 230.16 Hz), 4.28-4.14 (m, 1H,), 3.38-3.11 (m, 4H), 1.45-1.34 (m, 2H), 1.34-1.18 (m, 5H), 0.92-0.82 (m, 3H); MS calculated for (C₂₂H₂₄ClFN₂O₂+H)+, 403.90; found (ES [M+H]+), 403.12

### Example 13

### (1S or 1R)—N-butyl-2-[(2S or 2R)-2-(4-chlorophenyl)propyl]-6-fluoro-3-oxoisoindoline-1-carboxamide, Isomer E1

### (1S or 1R)—N-butyl-2-[(2R or 2S)-2-(4-chlorophenyl)propyl]-6-fluoro-3-oxoisoindoline-1-carboxamide, Isomer E2

### (1R or 1S)—N-butyl-2-[(2R or 2S)-2-(4-chlorophenyl)propyl]-6-fluoro-3-oxoisoindoline-1-carboxamide, Isomer E3

### (1R or 1S)—N-butyl-2-[(2S or 2R)-2-(4-chlorophenyl)propyl]-6-fluoro-3-oxoisoindoline-1-carboxamide, Isomer E4

The four stereoisomers of N-butyl-2-[2-(4-chlorophenyl)propyl]-6-fluoro-3-oxoisoindoline-1-carboxamide (Example 12 above) (2.60 mmol, 1.05 g) were separated by a preparative HPLC system, equipped with a Chiralpak IA, 5μ, 250 mm×20 mm column, using MTBE/MeOH (95/5) as mobile phase, giving fraction 1 containing isomer E1, fraction 2 containing isomer E2 and fraction 3 containing isomer E3 and isomer E4.

Fraction 1 was concentrated in vacuo to give (0.219 g, 20.9%) of isomer E1: [α]_{D}²⁰=30 (c 1.0, MeCN), (ee=98.5%).

Fraction 2 was concentrated in vacuo to give (0.232 g, 22.1%) of isomer E2: [α]_{D}²⁰=156 (c 1.0, MeCN), (ee=99.3%).

Fraction 3 was concentrated and isomer E3 and isomer E4 were separated by a preparative HPLC system, equipped with a (R,R) Whelk-O1, 5μ, 250 mm×20 mm column, using Heptan/IPA (50/50) as mobile phase giving fraction 3 containing isomer E3, and fraction 4 containing isomer E4.

Fraction 3 was concentrated in vacuo to give (0.204 g, 19.4%) of isomer E3: [α]_{D}²⁰=−36 (c 1.0, MeCN), (ee=97.7%).

Fraction 4 was concentrated in vacuo to give (0.160 g, 15.2%) of isomer E4: [α]_{D}²⁰=−177 (c 1.0, MeCN), (ee=96.0%).

### Example 14

### 2-(biphenyl-2-ylmethyl)-N-(tert-butyl)-5-(fluoromethoxy)-4-methyl-3-oxoisoindoline-1-carboxamide

2-(biphenyl-2-ylmethyl)-N-(tert-butyl)-5-hydroxy-4-methyl-3-oxoisoindoline-1-carboxamide (Example 2) (0.233 mmol, 100 mg) was dissolved in Acetonitrile (2 ml) in a 2 ml micro vial and K₂CO₃ (0.256 mmol, 0.25 mg) was added and the tube were sealed. N2 (g) was bubbled through the mixture for 5 min. bromofluoromethane (g) was bubbled through the mixture for 30 sec. The reaction was performed in a micro oven (20 min, 140° C.). The solvent was removed by evaporation and the reaction mixture was purified by flashcromatography (started with isocratic Heptane/EtOAc 95/5 and then the EtOAc concentration was increased to 50%, (silica gel 60 0.004-0.063 mm). The product containing fractions was pooled and the solvent was removed by evaporation to give the title compound (68 mg, 63.3%).

¹H-NMR (500 MHz, CDCl3): δ 7.45-7.25 (m, 11H), 5.82-5.79 (m, 1H), 5.71-5.69 (m, 1H), 5.47 (s, 1H), 5.16 (d, 1H), 4.47 (s, 1H), 4.43 (d, 1H), 2.59 (s, 3H), 1.08 (s, 9H); HRMS calculated for (C₂₈H₂₉FN₂O₃+H)+, 461.5611; found (ES [M+H]+), 461.5596.

### Example 15

### 2-(biphenyl-2-ylmethyl)-1-∂(tert-butylamino)carbonyl]-4-methyl-3-oxo-2,3-dihydro-1H-isoindol-5-yl methanesulfonate

2-(biphenyl-2-ylmethyl)-N-(tert-butyl)-5-hydroxy-4-methyl-3-oxoisoindoline-1-carboxamide (Example 2) (0.24 mmol, 104 mg) was dissolved in DCM and mesylchloride (0.36 mmol, 42 mg) was added, the mixture was cooled to 0° C. and TEA was added drop wise. The solution was heated to rt and stirred for 2 h until no starting material remained (LCMS). The solvent was removed by evaporation and the reaction mixture was purified by flashcromatography (started with isocratic Heptane/EtOAc 95/5 and then the EtOAc concentration was increased to 50%, (silica gel 60 0.004-0.063 mm). The product containing fractions was pooled and the solvent was removed by evaporation to give the title compound (62 mg, 50.4%).

¹H-NMR (500 MHz, CDCl3): δ 7.47-7.42 (m, 8H), 7.30-7.25 (m, 3H), 5.39 (s, 1H), 5.16 (d, 1H), 4.47 (s, 1H), 4.43 (d, 1H), 2.36 (s, 3H), 2.70 (s, 3H), 1.08 (s, 9H); HRMS calculated for (C₂₈H₃₀N₂O₅S +H)+, 507.6335; found (ES [M+H]+), 507.6326.

### Example 16

### N-{4-[(acetylamino)methyl]benzyl}-2-(biphenyl-2-ylmethyl)-3-oxoisoindoline-1-carboxamide

Acetic acid (0.19 mmol, 12 mg) and o-(benzotriazol-1-yl)-n,n,n′,n′-tetramethyluronium tetrafluoroborate (0.26 mmol, 0.83 mg) was mixed at rt and stirred for 20 min. n-methylmorpholine (0.26 mmol, 0.26 mg) and N-[4-(aminomethyl)benzyl]-2-(biphenyl-2-ylmethyl)-3-oxoisoindoline-1-carboxamide (example 8) (0.13 mmol, 60 mg) was added and the reaction was stirred at rt over night. The solvent was removed by evaporation and the crude was purified by preparative HPLC (started with isocratic Acetonitrile/buffer 20/80 and then the Acetonitrile concentration was increased to 95%, the buffer was a mixture of Acetonitrile/water 10/90 and ammonium acetate (0.1 M, column KR-100-7-C8, 50 mm×250 mm, flow 40 ml/min). The product containing fractions was pooled and the product was freeze dried over night to give the title compound (10 mg, 15.3%). ¹H-NMR (500 MHz, CDCl3): δ 7.75-7.71 (m, 1H), 7.64-7.65 (m, 2H), 7.52-7.47 (m, 1H), 7.46-7.37 (m, 6H), 7.37-7.22 (m, 3H), 7.13 (d, 2H), 6.86 (d, 2H), 6.04-5.99 (m, 1H), 5.67 (brs, 1H), 5.27 (d, 1H), 4.74 (s, 1H), 4.39 (d, 2H), 4.31 (d, 1H), 4.22-4.09 (m, 2H), 2.03 (s, 3H); HRMS calculated for (C₃₂H₂₉N₃O₃+H)+, 504.6140; found (ES [M+H]+), 504.6145.

### Example 17

### 2-(biphenyl-2-ylmethyl)-N-(4-{[(difluoroacetyl)amino]methyl} benzyl)-3-oxoisoindoline-1-carboxamide

Difluoroacetic acid (0.19 mmol, 19 mg) and o-(benzotriazol-1-yl)-n,n,n′,n′-tetramethyluronium tetrafluoroborate (0.26 mmol, 0.83 mg) was mixed at rt and stirred for 20 min. n-methylmorpholine (0.26 mmol, 0.26 mg) and N-[4-(aminomethyl)benzyl]-2-(biphenyl-2-ylmethyl)-3-oxoisoindoline-1-carboxamide (Example 8) (0.13 mmol, 60 mg) was added and the reaction was stirred at rt over night. The solvent was removed by evaporation and the crude was purified by preparative HPLC (started with isocratic Acetonitrile/buffer 20/80 and then the Acetonitrile concentration was increased to 95%, the buffer was a mixture of Acetonitrile/water 10/90 and ammonium acetate (0.1 M, column KR-100-7-C8, 50 mm×250 mm, flow 40 ml/min). The product containing fractions was pooled and the product was freeze dried over night to give the title compound (12 mg, 17.0%). ¹H-NMR (500 MHz, CDCl3): δ 7.61-7.47 (m, 3H), 7.45-7.18 (m, 10H), 7.18-7.12 (m, 1H), 7.07 (d, 2H), 6.85 (d, 2H), 6.49 (brs, 1H), 6.27 (m, 1H), 5.90 (t, 1H), 5.20 (d, 1H), 4.69 (s, 1H), 4.41 (d, 2H), 4.29-4.21 (d, 1H), 4.21-4.05 (m, 2H); HRMS calculated for (C₃₂H₂₇F₂N₃O₃+H)+, 540.5948; found (ES [M+H]+), 540.5895.

### Example 18

### 2-(biphenyl-2-ylmethyl)-N-(tert-butyl)-4,7-difluoro-1-methyl-3-oxoisoindoline-1-carboxamide

2-acetyl-3,6-difluorobenzoic acid (0.24 mmol, 50 mg) was dissolved in Methanol (1 ml) and (biphenyl-2-ylmethyl)amine (0.24 mmol, 46 mg) was added. The mixture was stirred at 50° C. over night to form the imine. Then tert-butyl isocyanide (0.24 mmol, 21 mg) dissolved in Acetonitrile was added to mixture. The reaction was stirred at 50° C. for 170 h. The product precipitated and was filtered of and the solid was washed with EtOAc to give the title compound (23 mg, 20.5%).

¹H-NMR (500 MHz, CDCl3): δ 7.64-7.56 (m, 1H), 7.52-7.43 (m, 2H), 7.42-7.08 (m, 8H), 5.08 (s, 1H), 4.65 (dd, 2H), 1.37 (s, 3H), 0.94 (s, 9H); HRMS calculated for (C₂₇H₂₆F₂N₂O₂+H)+, 449.5250; found (ES [M+H]+), 449.5248

### Example 19

### 2-(biphenyl-2-ylmethyl)-6-fluoro-3-oxo-N-(4,4,4-trifluorobutyl)isoindoline-1-carboxamide

4-fluoro-3-hydroxy-2-benzofuran-1(3H)-one (Preparation X) ((0.87 mmol, 147 mg) was dissolved in Methanol (10 ml) and (biphenyl-2-ylmethyl)amine (0.87 mmol, 160 mg) was added and stirred for 20 min. Then 1,1,1-trifluoro-4-isocyanobutane (0.87 mmol, 120 mg) was added to the mixture. The reaction was stirred at rt over night. The solvent was removed by evaporation. The crude was purified by flashcromatography (started with isocratic heptane/Acetone 90/10 and then the Acetone concentration was increased to 50%, (silica gel 60 0.004-0.063 mm). The product containing fractions was pooled and the solvent was removed by evaporation to give the title compound (66 mg, 16%).

¹H-NMR (500 MHz, CDCl3): δ 7.50-7.02 (m, 12H), 7.02-6.92 (m, 1H), 5.18-5.08 (D, 1H), 4.62 (s, 1H), 4.28 (d, 1H), 3.25-2.93 (m, 2H), 2.00-1.82 (m,2H), 1.62-1.44 (m, 2H);HRMS calculated for (C₂₆H₂₂F₄N₂O₂+H)+, 471.4788; found (ES [M+H]+), 471.4789.

### Example 20

### 2-(biphenyl-2-ylmethyl)-1-[(tert-butylamino)carbonyl]-4-methyl-3-oxo-2,3-dihydro-1H-isoindol-5-yl trifluoromethanesulfonate

2-(biphenyl-2-ylmethyl)-N-(tert-butyl)-5-hydroxy-4-methyl-3-oxoisoindoline-1-carboxamide (Example 2) (1.40 mmol, 0.6 g) was dissolved in THF (10 ml), K2CO3 (4.20 mmol, 0.58 g) and n-phenyltrifluoromethanesulfonimide (1.54 mmol, 0.55 g) was added. The reaction was performed in a microwave oven (10 min, 120° C.).

The solvent was removed by evaporation and the crude was purified by flashcromatography (started with isocratic heptane/DCM 50/50 and then the DCM concentration was increased to 100%, then EtOAc added as eluent and the EtOAc concentration was increased to 30% (silica gel 60 0.004-0.063 mm). The product containing fractions was pooled and the solvent was removed by evaporation to give the title compound (0.655 g, 83.4%). ¹H-NMR (500 MHz, CDCl₃): δ 7.521 (d, 1H), 7.42-7.31 (m, 7H), 7.30-7.24 (m, 2H), 6.08 (s, 1H), 5.26 (d, 1H), 4.53 (s, 1H), 4.44 (d, 1H), 2.52 (s, 3H), 1.16 (s, 9H); HRMS calculated for (C₂₈H₂₇F₃N₂O₅S+H)+, 561.6048; found (ES [M+H]+), 561.6019.

### Example 21

### 2-(biphenyl-2-ylmethyl)-N-(4-{[(fluoroacetyl)amino]methyl}benzyl)-3-oxoisoindoline-1-carboxamide

Fluoroacetic acid (0.19 mmol, 15 mg) and o-(benzotriazol-1-yl)-n,n,n′,n′-tetramethyluronium tetrafluoroborate (0.26 mmol, 83 mg) was mixed at rt and stirred for 20 min. n-methylmorpholine (0.26 mmol, 26 mg) and N-[4-(aminomethyl)benzyl]-2-(biphenyl-2-ylmethyl)-3-oxoisoindoline-1-carboxamide (Example 8) (0.13 mmol, 60 mg) was added and the reaction was stirred at rt over night. The solvent was removed by evaporation and the crude was purified by preparative HPLC (started with isocratic Acetonitrile/buffer 20/80 and then the Acetonitrile concentration was increased to 95%, the buffer was a mixture of Acetonitrile/water 10/90 and ammonium acetate (0.1 M, column KR-100-7-C8, 50 mm×250 mm, flow 40 ml/min). The product containing fractions was pooled and the product was freeze dried over night to give the title compound (35 mg, 51.6%). ¹H-NMR (500 MHz, CDCl₃): δ 7.63-7.58 (m, 1H), 7.57-7.48 (m, 2H), 7.44-7.35 (m, 4H), 7.35-7.21 (m, 6H), 7.19-7.15 (m, 114), 7.11 (d, 2H), 6.91 (d, 2H), 6.63-6.58 (m, 1H), 6.55 (bs, 1H), 5.22 (d, 1H), 4.88 (s, 1H), 4.79 (s, 1H), 4.73 (s, 1H), 4.44 (d, 2H), 4.28 (d, 1H), 4.25-4.13 (m, 2H); HRMS calculated for (C₃₂H₂₈FN₃O₃+H)+, 522.6044; found (ES [M+H]+), 522.6043.

### Example 22

### N-(4,4-difluorobutyl)-2-(diphenylmethyl)-3-oxoisoindoline-1-carboxamide

2-formylbenzoic acid (6.66 mmol, 1.00 g) was dissolved in Methanol (10 ml) and (diphenylmethyl)amine (6.66 mmol, 1.22 g) was added and stirred for 20 min. Then 1,1-difluoro-4-isocyanobutane (6.66 mmol, 0.79 g) was added to the mixture. The reaction was stirred at rt over night. The solvent was removed by evaporation. The crude was purified by flashcromatography (started with isocratic heptane/EtOAc 90/10 and then the EtOAc concentration was increased to 50%, (silica gel 60 0.004-0.063 mm). The product containing fractions was pooled and the solvent was removed by evaporation. The substance was not pure enough so it was purified by preparative HPLC (started with isocratic acetonitrile/buffer 20/80 and then the acetonitrile concentration was increased to 95%, the buffer was a mixture of acetonitrile/water 10/90 and ammonium acetate (0.1 M, column KR-100-7-C8, 50 mm×250 mm, flow 40 ml/min). The product containing fractions was pooled and the acetonitrile was removed by evaporation. The product was freeze dried over night to give the title compound (140 mg, 32.3%).

¹H-NMR (500 MHz, CDCl₃): δ 7.68-7.49 (m, 3H), 7.49-7.18 (m, 9H), 7.06-6.93 (m, 2H), 6.78 (s, 1H), 6.25-6.12 (m, 1H), 7.78-5.30 (m, 1H), 5.24 (s, 1H), 2.89-2.70 (m, 1H), 2.63-2.43 (m, 1H), 1.54-1.30 (m, 2H), 1.25-1.05 (m, 2H); HRMS calculated for (C₂₆H₂₄F₂N₂O₂+H)⁺, 435.1884; found (ES [M+H]⁺), 435.1882.

### Example 23

### (1S or 1R)—N-(4,4-difluorobutyl)-2-(diphenylmethyl)-3-oxoisoindoline-1-carboxamide; Isomer E1

### (1R or 1S)—N-(4,4-difluorobutyl)-2-(diphenylmethyl)-3-oxoisoindoline-1-carboxamide; Isomer E2

N-(4,4-difluorobutyl)-2-(diphenylmethyl)-3-oxoisoindoline-1-carboxamide (Example 22) (1.63 mmol, 0.71 g) were separated by a preparative HPLC system, equipped with a ReproSil, 10μ, 250 mm×20 mm column, using Heptan/IPA (30/70) as mobile phase, giving fraction 1 containing isomer E1, fraction 2 containing isomer E2.

Fraction 1 was concentrated in vacuo to give (0.316 g, 44.5%) of isomer E1, ee=100%.

Fraction 2 was concentrated in vacuo to give (0.308 g, 43.4%) of isomer E2, ee=99.6%.

### Example 24

### N-benzyl-3-oxo-2-(1-phenylethyl)isoindoline-1-carboxamide

2-Formylbenzoic acid (2.72 g, 18.1 mmol) and 1-Phenylethanamine (2.30 mL, 18.1 mmol) was added to a flask, MeOH (15 ml) was then added and the mixture was left stirring in r.t. for 2 h. (Isocyanomethyl)benzene (2.12 g, 18.1 mmol) dissolved in MeCN (25 ml) was added to the mixture and the reaction was left stirring in r.t. over night. The reaction was finished the next morning and the solvent was evaporated. The crude was dissolved in DCM (20 mL) and extracted with water (10 mL), the organic phase was collected and most of the solvent was evaporated then EtOAc was added and the solvent was evaporated. The crude was purified by flash chromatography (SP1™ flash system from Biotage™, silica cartridge), using heptane and EtOAc as eluent, followed by concentration in vacuo afforded the title compound (4.16 g, 62%).

¹³C-NMR (500 MHz, CDCl₃) δ 170.73, 170.47, 169.09, 168.26, 142.14, 142.02, 140.45, 140.37, 137.34, 137.14, 132.79, 132.75, 131.09, 130.99, 129.38, 129.35, 129.21, 129.10, 128.96, 128.71, 128.50, 128.27, 128.15, 128.02, 127.96, 127.68, 127.58, 127.49, 124.32, 124.26, 122.93, 122.78, 63.75, 63.31, 52.56, 52.12, 43.89, 43.39, 18.19, 17.58; HRMS calculated for (C24 H22 N2 O2+H)⁺, 371.1760 found (ES [M+H]⁺), 371.1768 and 371.1771 for respectively diasteromer.

### Example 25

### (1S or 1R)—N-benzyl-3-oxo-2-[(1R or 1S)-1-phenylethyl]isoindoline-1-carboxamide (E1)

### (1S or 1R)—N-benzyl-3-oxo-2-[(1S or 1R)-1-phenylethyl]isoindoline-1-carboxamide (E2)

### (1R or 1S)—N-benzyl-3-oxo-2-[(1R or 1S)-1-phenylethyl]isoindoline-1-carboxamide (E3)

### (1R or 1S)—N-benzyl-3-oxo-2-[(1S or 1R)-1-phenylethyl]isoindoline-1-carboxamide (E4)

The four stereo isomers of N-benzyl-3-oxo-2-(1-phenylethyl)isoindoline-1-carboxamide (example 24) were separated by a preparative HPLC system, equipped with a Chiralpak AD-H 5μ, 250 mm×20 mm column, using Heptan/EtOH (85/15) as mobile phase, giving fraction 1 containing isomer 1, fraction 2 containing isomer 2 and isomer 3, and fraction 3 containing isomer 4. Fraction 1 was concentrated in vacuo to give (El) (0.602 g, 22.8%) of isomer 1: [α]_{D}²⁰=+121.9 (c 1.0, MeCN); HRMS calculated for (C24H22N2O2+H)⁺, 371.1759 found (ES [M+H]⁺), 371.1760

Fraction 3 was concentrated in vacuo to give (E4) (0.516 g, 19.5%) of isomer 4: [α]_{D}²⁰=−121.3 (c 1.0, MeCN); HRMS calculated for (C24 H22 N2 O2+H)⁺, 371.175 found (ES [M+H]⁺), 371.1779

Fraction 2 was concentrated and isomer 2 and isomer 3 were separated by a preparative HPLC system, equipped with a Chiralpak IA 5μ, 250 mm×20 mm column, using Heptan/EtOH (80/20) as mobile phase giving fraction 1 containing isomer 2, and fraction 2 containing isomer 3.

Fraction 1 was concentrated in vacuo to give (E2) (0.571 g, 21.6%) of isomer 2: [α]_{D}²⁰=+161.0 (c 1.0, MeCN); HRMS calculated for (C24 H22 N2 O2+H)⁺, 371.1759 found (ES [M+H]⁺), 371.1761

Fraction 2 was concentrated in vacuo to give (E3) (0.718 g, 27.1%) of isomer 3: [α]_{D}²⁰=−153.0 (c 1.0, MeCN); HRMS calculated for (C24 H22 N2 O2+H)⁺, 371.1759 found (ES [M+H]⁺), 371.1758

### Example 26

### N-(3-chlorobenzyl)-2-(3-hydroxy-2,2-dimethylpropyl)-3-oxoisoindoline-1-carboxamide

The 3-amino-2,2-dimethylpropan-1-ol (0.276 mmol) was dissolved in MeCN (1 mL) and added to 2-Formylbenzoic acid (0.276 mmol) dissolved in MeOH (1 mL), this was stirring for 30 min in r.t. 1-chloro-3-(isocyanomethyl)benzene (0.248 mmol) dissolved in MeCN (2 mL) was added to the mixture and the reaction was left stirring in r.t. over night. The reaction was finished the next morning and the solvent was evaporated. The crude was dissolved in DCM (4 mL) and extracted with water (3 mL), the organic phase was collected and the solvent was evaporated. The crude was dissolved in DMSO (1 ml), filtrated and purified by preparative HPLC using a Waters FractionLynx III HPLC system with a mass triggered fraction collector, equipped with an Xbridge Prep C18 150×19 mm column using a MeCN/0.2% NH₃ buffer system with a gradient from 100% A (5% MeCN+95% 0.2% NH₃) to 100% B (95% MeCN+5% 0.2% NH₃) as mobile phase, followed by concentration in vacuo afforded the title compound (0.025 g, 23.%).

¹H-NMR* (600 MHz, DMSO-d₆, DMSO*) δ 9.29-9.17 (m, 1H), 7.77-7.46 (m, 4H), 7.4-7.15 (m, 4H), 5.48-5.4 (s, 1H), 4.74-4.63 (m, 1H), 4.4-4 26 (m, 2H), 3.79-3.71 (d, 1H), 3.19-3.05 (m), 2.76-2.64 (d), 0.88-0.75 (s, 6H); HRMS calculated for (C21 H23 Cl N2 O3+H)⁺, 387.1475 found (ES [M+H]⁺), 387.1475

### Example 27

### 2-[2-(4-chlorophenyl)propyl]-N-[(5-methyl-2-phenyl-1,3-oxazol-4-yl)methyl]-3-oxoisoindoline-1-carboxamide

2-Formylbenzoic acid (0.060 g, 0.44 mmol), 2-(4-chlorophenyl)propan-1-amine hydrochloride (0.068 g, 0.40 mmol) and TEA (0.062 mL, 0.44 mmol) was added to a flask and dissolved in MeOH (2 mL), this was stirring for 2 h in r.t. 4-(isocyanomethyl)-5-methyl-2-phenyl-1,3-oxazole (0.079 g, 0.40 mmol) dissolved in MeCN (2 mL) was added to the mixture and the reaction was left stirring in r.t. for two days. The solvent was evaporated, the crude was dissolved in DCM (4 mL) and extracted with water (3 mL), the organic phase was collected and the solvent was evaporated. The crude was purified by flash chromatography (SP1™ flash system from Biotage™, silica cartridge), using heptane and EtOAc as eluent, followed by concentration in vacuo afforded the title compound (0.017 g, 8.5%). ¹H-NMR (500 MHz, CDCl₃) δ 8.22-7.34 (m, 9H), 7.21-7.01 (m, 4H), 6.56-6.34 (m, 1H), 4.64-3.98 (m, 3H), 3.42-3.12 (m, 2 H), 2.53-2.16 (m, 3H), 1.4-1.08 (m, 3H);

### Example 28

### N-(tert-butyl)-2-[(1-methyl-5-phenyl-1H-pyrazol-3-yl)methyl]-3-oxoisoindoline-1-carboxamide

1-(1-methyl-5-phenyl-1H-pyrazol-3-yl)methanamine (0.400 mmol) was dissolved in MeCN (1 mL) and added to 2-Formylbenzoic acid (0.400 mmol) dissolved in MeOH (1 mL), this was stirring for 1 h in r.t. tert-butyl isocyanide (0.400 mmol) was added to the mixture and the reaction was left stirring in r.t. for two days. The solvent was evaporated, the crude was dissolved in DCM (4 mL) and extracted with water (3 mL), the organic phase was collected and the solvent was evaporated. The crude was purified by flash chromatography (SP1™ flash system from Biotage™, silica cartridge), using heptane and EtOAc as eluent, followed by concentration in vacuo afforded the title compound (0.078 g, 48.4%). ¹H-NMR (500 MHz, CDCl₃) δ 7.94-7.36 (m, 9H), 6.39-6.28 (m, 1H), 5.28-5.07 (m, 2H), 4.39-4.23 (d, 1H), 3.92-3.79 (s, 3H), 1.44-1.2 (s, 9H); HRMS calculated for (C24 H26 N4 O2+H)⁺, 403.2134 found (ES [M+H]⁺), 403.2156

### Example 29

### 2-(biphenyl-2-ylmethyl)-6-bromo-N-(tert-butyl)-3-oxoisoindoline-1-carboxamide

1-biphenyl-2-ylmethanamine (0.225 g, 1.23 mmol) and 5-bromo-3-hydroxy-2-benzofuran-1(3H)-one (Preparation W) (0.283 g, 1.24 mmol) was added to a flask, MeOH (1 mL) was added an the mixture was left stirring in r.t. for 3 h. tert-butyl isocyanide (0.139 mL, 1.23 mmol) was added to the mixture and the reaction was left stirring in r.t. over night. The solvent was evaporated, the crude was dissolved in DCM (4 mL) and extracted with water (3 mL), the organic phase was collected and the solvent was evaporated. The crude was purified by flash chromatography (SP1™ flash system from Biotage™, silica cartridge), using heptane and EtOAc as eluent, followed by concentration in vacuo afforded the title compound (0.191 g, 32.4%). ¹³C-NMR (500 MHz, CDCl₃) δ 168.58, 166.16, 143.65, 142.49, 140.52, 133.37, 132.34, 130.62, 130.46, 129.15, 128.62, 128.35, 128.15, 127.99, 127.87, 127.28, 126.31, 125.21, 124.92, 62.87, 51.61, 42.93, 31.88, 28.99, 27.52, 27.24, 22.58, 13.28; HRMS calculated for (C26 H25 Br N2 O2+H)⁺, 477.1178 found (ES [M+H]⁺), 477.1173

### Example 30

### 2-(biphenyl-2-ylmethyl)-5-bromo-N-(tert-butyl)-3-oxoisoindoline-1-carboxamide

1-biphenyl-2-ylmethanamine (0.106 g, 0.578 mmol) and 5-bromo-2-formylbenzoic acid (commercially available) (0.132 g, 0.578 mmol) was added to a flask, MeOH (1 mL) was added an the mixture was left stirring in r.t. for 30 mins. .tert-butyl isocyanide (0.065 mL, 0.578 mmol) was added to the mixture and the reaction was left stirring in r.t. for 16 h. The solvent was evaporated, the crude was dissolved in DCM (4 mL) and extracted with water (3 mL), the organic phase was collected and the solvent was evaporated. The crude was purified by flash chromatography (SP1™ flash system from Biotage™, silica cartridge), using heptane and EtOAc as eluent, followed by concentration in vacuo afforded the title compound (0.143 g, 51.8%). ¹H-NMR (500 MHz, CDCl₃) δ 7.89-7.82 (m, 1H), 7.78-7.71 (m, 1H), 7.56-7.24 (m, 10H), 5.47-5.33 (d, 1H), 4.72-4.63 (s, 1H), 4.23-4.07 (d, 1H), 1.39-1.13 (s, 9H); HRMS calculated for (C26 H25 Br N2 O2+H)⁺, 477.1178 found (ES [M+H]⁺), 477.1184

### Example 31

### 2-(biphenyl-2-ylmethyl)-N-(4,4-difluorobutyl)-3-oxoisoindoline-1-carboxamide

The 1-biphenyl-2-ylmethanamine (0.400 mmol) was dissolved in MeCN (1 mL) and added to 2-Formylbenzoic acid (0.400 mmol) dissolved in MeOH (1 mL), this was stirring for 30 min in r.t 1,1-difluoro-4-isocyanobutane (preparation P) (0.400 mmol) dissolved in MeCN (2 mL) was added to the mixture and the reaction was left stirring in r.t. over night. The reaction was finished the next morning and the solvent was evaporated. The crude was dissolved in DCM (4 mL) and extracted with water (3 mL), the organic phase was collected and the solvent was evaporated. The crude was dissolved in DMSO (1 ml), filtrated and purified by preparative HPLC using a Waters FractionLynx I HPLC system with a mass triggered fraction collector, equipped with an Xbridge Prep C18 150×19 mm column using a MeCN/0.2% NH₃ buffer system with a gradient from 100% A (5% MeCN+95% 0.2% NH₃) to 100% B (95% MeCN+5% 0.2% NH₃) as mobile phase, followed by concentration in vacuo afforded the title compound (0.057 g, 33%). ¹H-NMR* (600 MHz, DMSO-d₆, DMSO*) δ 8.526-8.3 (m, 1H), 7.75-7.03 (m, 13H), 6.2-5.85 (m, 1H), 5.27-5.07 (d, 1H), 4.83-4.66 (s, 1H), 4.13-3.85 (d, 1H), 3.1-2.83 (m), 1.81-1.55 (m, 2H), 1.5-1.27 (m, 2H); HRMS calculated for (C26 H24 F N2 O2+H)⁺, 435.1884 found (ES [M+H]⁺), 435.1865

### Example 32

### 2-(biphenyl-2-ylmethyl)-N-[(2,2-difluoro-1,3-benzodioxol-5-yl)methyl]-6-fluoro-3-oxoisoindoline-1-carboxamide

The 1-biphenyl-2-ylmethanamine (0.370 mmol) was dissolved in MeCN (1 mL) and added to 5-fluoro-3-hydroxy-2-benzofuran-1(3H)-one (preparation X) (0.370 mmol) dissolved in MeOH (1 mL), this was stirring for 30 min in r.t. (2,2-difluoro-1,3-benzodioxol-5-yl)methyl isocyanide (preparation R) (0.370 mmol) dissolved in MeCN (2 mL) was added to the mixture and the reaction was left stirring in r.t. for 3 days. The solvent was evaporated and, the crude was dissolved in DCM (4 mL) and extracted with water (3 mL), the organic phase was collected and the solvent was evaporated. The crude was dissolved in DMSO (1 ml), filtrated and purified by preparative HPLC using a Waters FractionLynx I HPLC system with a mass triggered fraction collector, equipped with an Xbridge Prep C18 150×19 mm column using a MeCN/0.2% NH₃ buffer system with a gradient from 100% A (5% MeCN+95% 0.2% NH₃) to 100% B (95% MeCN+5% 0.2% NH₃) as mobile phase, followed by concentration in vacuo afforded the title compound (0.053 g, 26%). ¹H-NMR* (600 MHz, DMSO-d₆, DMSO*) δ 8.95-8.83 (m, 1H), 7.76-7.64 (m, 1H), 7.48-6.87 (m, 14H), 5.18-5.08 (d, 1H), 4.84-4.78 (s, 1H), 4.2-3.95 (m); HRMS calculated for (C30 H21 F3 N2 O4+H)⁺, 531.1532 found (ES [M+H]⁺), 531.1537

### Example 33

### 2-(biphenyl-2-ylmethyl)-N-tert-butyl-3-oxoisoindoline-1-carboxamide

A mixture of 2-formylbenzoic acid (2.50 g, 16.7 mmol) and 1-biphenyl-2-ylmethanamine (3.05 g, 16.7 mmol) in MeOH (55 ml) was stirred at room temperature for 1 h and then tert-butyl isocyanide (1.38 g, 16.7 mmol) was added. The resulting mixture was stirred at room temperature over night and concentrated under reduced pressure. Crystals were obtained by adding ethanol to the residue. The crystals (2.06 g) were filtered off and dried in vacuo. The filtrate was purified by flash chromatography (SP1™ flash system from Biotage™, silica cartridge), using ethyl acetate (gradient from 6 to 50%) in heptane as eluent. Removal of the solvent gave 2.05 g of a white solid. The products from crystallisation and chromatography were combined and triturated with methanol giving 2.88 g of a white solid. The remaining material was purified by preparative HPLC giving a second crop of 0.70 g.

¹H NMR (500 MHz, CD₃OD) δ 7.71 (dm, 1H), 7.56 (m, 1H), 7.50 (m, 1H), 7.42 (dm, 1H), 7.38-7.24 (m, 9H), 5.37 (d, 1H), 4.71 (s, 1H), 4.15 (d, 1H), 1.22 (s, 9H); HRMS calculated for (C₂₆H₂₆N₂O₂+H)⁺, 399.2066; found (ESI [M+H]⁺), 399.2073.

### Example 34

### (R or S)2-(biphenyl-2-ylmethyl)-N-tert-butyl-3-oxoisoindoline-1-carboxamide (isomer 1)

### (S or R)2-(biphenyl-2-ylmethyl)-N-tert-butyl-3-oxoisoindoline-1-carboxamide (isomer 2)

The first crop (2.88 g) of example 33 above was separated into its enantiomers on a Chiralpak IA using 25% 2-propanol in heptane. Concentration under reduced pressure gave 1.39 g of isomer 1 and 1.25 g of isomer 2.

### Example 35

### N-benzyl-6-cyano-3-oxo-2-[(1R)-1-phenylethyl]isoindoline-1-carboxamide

### (i) N-benzyl-6-bromo-3-oxo-2-[(1R)-1-phenylethyl]isoindoline-1-carboxamide

(R)-(+)-1-Phenylethylamine (0.12 g, 1.0 mmol) was added to a slurry of 5-bromo-3-hydroxy-2-benzofuran-1(3H)-one in MeOH (5 ml). The solution was stirred for 30 mins at room temperature. Benzyl isocyanide (0.12 g, 1.0 mmol) was added and the resulting mixture was stirred at room temperature over night and evaporated. Purification by flash chromatography (SP1™ flash system from Biotage™, silica cartridge), using ethyl acetate (gradient from 6 to 50%) in heptane as eluent, followed by concentration in vacuo afforded the title compound (0.33 g, 72%) as a white powder. ¹H NMR (500 MHz, CD₃OD) δ 7.68 (m, 2H), 7.52 (m, 1H), 7.44 (m, 1H), 7.36-7.20 (m, 8H), 7.68 (m, 1H), 5.61 and 5.32 (q, 1H, rotamers), 5.22 and 4.79 (s, 1H, rotamers), 4.37 (br s, 1H), 3.97 (m, 1H), 1.74 and 1.61 (d, 3H, rotamers); MS (ESI) m/z 449.0 ([M+H]⁺);

### (ii) N-benzyl-6-cyano-3-oxo-2-[(1R)-1-phenylethyl]isoindoline-1-carboxamide

A mixture of N-benzyl-6-bromo-3-oxo-2-[(1R)-1-phenylethyl]isoindoline-1-carboxamide (0.10 g, 0.22 mmol), zinc cyanide (0.040 g, 0.34 mmol), and Pd(PPh₃)₄ (0.026 g, 0.022 mmol) in DMF (3 ml) was degassed by argon-bubbling for 15 mins. The mixture was then heated at 200° C. for 30 mins in a microwave reactor and concentrated in vacuo. Purification by flash chromatography (SP1™ flash system from Biotage™, silica cartridge), using ethyl acetate (gradient from 9 to 76%) in heptane as eluent, followed by concentration in vacuo afforded the title compound (0.064 g, 72%) as a white solid. ¹H NMR (500 MHz, (CD₃)₂CO) δ 8.24 (br s, 1H), 7.94-7.81 (m, 3H), 7.52 (m, 1H), 7.36-7.19 (m, 8H), 7.13 (m, 1H), 5.63 and 5.21 (q, 1H), 5.41 and 5.04 (s, 1H), 4.43 (m, 1H), 4.15 (m, 1H), 1.84 and 1.67 (d, 3H, rotamers). MS (ESI) m/z 396.0 ([M+H]⁺); HRMS calculated for (C₂₆H₂₁N₃O₂+H)⁺, 396.1712; found (ESI [M+H]⁺), 396.1739.

### Example 36

### 2-(biphenyl-2-ylmethyl)-N-(tert-butyl)-6-cyano-3-oxoisoindoline-1-carboxamide

was synthesised according the procedure described in example 35 above using 1-biphenyl-2-ylmethanamine instead of (R)-(+)-1-phenylethylamine and tert-butyl isocyanide instead of benzyl isocyanide. HRMS calculated for (C₂₇H₂₅N₃O₂+H)⁺, 424.2025; found (ESI [M+H]⁺), 424.2010.

### Example 37

### 2-(2-bromobenzyl)-N-tert-butyl-5-hydroxy-4-methyl-3-oxoisoindoline-1-carboxamide

### (i) N-(2-bromobenzyl)-N-[2-(tert-butylamino)-1-(2-furyl)-2-oxoethyl]but-2-ynamide

A mixture of but-2-ynoic acid (0.42 g, 5.0 mmol), 1-(2-bromophenyl)methanamine (0.93 g, 5.0 mmol), and 2-furaldehyde (0.48 g, 5.0 mmol) in MeOH (20 ml) was stirred at room temperature for 1 h and then tert-butyl isocyanide (0.42 g, 5.0 mmol) was added. The resulting mixture was stirred at room temperature over night and concentrated under reduced pressure. The crude product was used in the next step without purification.

### (ii)2-[2-bromobenzyl)-N-tert-butyl-5-hydroxy-4-methyl-3-oxoisoindoline-1-carboxamide

A mixture of N-(2-bromobenzyl)-N-[2-(tert-butylamino)-1-(2-furyl)-2-oxoethyl]but-2-ynamide (2.10 g, 0.487 mmol) from step (i) above and ytterbium (III) trifluoromethanesulfonate (0.302 g, 0.487 mmol) in xylene (100 ml) was heated at reflux for 1.5 h. A white precipitate was filtered off giving 0.87 g of pure product. The filtrate was concentrated and purified by flash chromatography (SP1™ flash system from Biotage™, silica cartridge), using ethyl acetate (gradient from 30 to 70%) in heptane as eluent giving a second crop of 0.32 g making the total yield 1.19 g (57%). NMR (500 MHz, (CD₃OD) δ 7.60 (m, 1H), 7.36-7.27 (m, 2H), 7.21 (m, 1H), 7.08 (m, 1H), 6.95 (m, 1H) 5.23 (d, 1H), 4.66 (s, 1H), 4.37 (d, 1H), 2.55 (s, 3H), 1.30 (s, 9H).

### Example 38

### 2-(biphenyl-2-ylmethyl)-N-tert-butyl-4-methyl-5-1(methylsulfonyl)amino]-3-oxoisoindoline-1-carboxamide

### (i) N-(biphenyl-2-ylmethyl)-N-{2-(tert-butylamino)-1-[1-(methylsulfonyl)-1H-pyrrol-2-yl]-2-oxoethyl}but-2-ynamide

A mixture of but-2-ynoic acid (0.050 g, 0.60 mmol), 1-biphenyl-2-ylmethanamine (0.11 g, 0.60 mmol), and 1-(methylsulfonyl)-1H-pyrrole-2-carbaldehyde (J. Am. Chem. Soc., 1998, 1741.) (0.10 g, 0.60 mmol) in MeOH (6 ml) was stirred at room temperature for 1 h. and then tert-butyl isocyanide (0.050 g, 0.60 mmol) was added. The resulting mixture was stirred at room temperature for three days giving a white precipitate. The precipitate was filtered off and dried in vacou giving 0.23 g (76%) of a white powder which was used in the next step without further purification. MS (ESI) m/z 506.2 ([M+H]⁺).

### (ii)2-(biphenyl-2-ylmethyl)-N-tert-butyl-4-methyl-5-1(methylsulfonyl)amino]-3-oxoisoindoline-1-carboxamide

A solution of N-(biphenyl-2-ylmethyl)-N-{2-(tert-butylamino)-1-[1-(methylsulfonyl)-1H-pyrrol-2-yl]-2-oxoethyl}but-2-ynamide from step (i) above (0.050 g, 0.099 mol) in dioxane (4 ml) and 1-butyl-3-methyl-1H-imidazol-3-ium hexafluorophosphate (BMIMPF₆, 0.2 ml) was heated at 200° C. for 30 mins in a microwave reactor. The mixture was concentrated under reduced pressure and the residue was partitioned between ethyl acetate and water. The layers were separated and the aqueous phase was extracted with two portions of ethyl acetate. The combined organic layers were washed with two portions of water, dried over MgSO₄, and concentrated under reduced pressure. Purification by flash chromatography (SP1™ flash system from Biotage™, silica cartridge), using ethyl acetate (gradient from 12 to 100%) in heptane as eluent, followed by concentration in vacuo afforded the title compound (0.055 g, 56%) as a white solid. ¹H NMR (500 MHz, (CD₃)₂CO) δ 8.00 (br s, 1H), 7.53 (d, 1H), 7.37-7.20 (m, 10H), 5.32 (d, 1H), 4.62 (s, 1H), 4.10 (d, 1H), 2.97 (s, 3H), 2.67 (s, 3H), 1.21 (s, 9H); MS (ESI) m/z 506.2 ([M+H]⁺).

### Example 39

### 2-(biphenyl-2-ylmethyl)-N-(tert-butyl)-5-[(methylsulfonyl)amino]-3-oxoisoindoline-1-carboxamide

was synthesised according the procedure in example 38 described above using propiolic acid instead of but-2-ynoic acid. MS (ESI) m/z 492.1 ([M+H]⁺).

### Example 40

### N-(tert-butyl)-2-(4-chlorobenzyl)-5-hydroxy-3-oxoisoindoline-1-carboxamide

### (i) N-[2-(tert-butylamino)-1-(2-furyl)-2-oxoethyl]-N-(4-chlorobenzyl)-3-(trimethylsilyl)prop-2-ynamide

A mixture of 3-(trimethylsilyl)prop-2-ynoic acid (0.071 g, 0.50 mmol), 1-(4-chlorophenyl)methanamine (0.071 g, 0.50 mmol), and 2-furaldehyde (0.048 g, 0.50 mmol) in MeOH (2 ml) was stirred at room temperature for 1 h and then tert-butyl isocyanide (0.042 g, 0.50 mmol) was added. The resulting mixture was stirred at room temperature for 2 days and concentrated under reduced pressure. The crude product was used in the next step without purification; MS (ESI) m/z 444.9 ([M+H]⁺).

### (ii) N-(tert-butyl)-2-(4-chlorobenzyl)-5-hydroxy-3-oxoisoindoline-1-carboxamide

A mixture of N-[2-(tert-butylamino)-1-(2-furyl)-2-oxoethyl]-N-(4-chlorobenzyl)-3-(trimethylsilyl)prop-2-ynamide (0.223 g, 0.50 mmol) from step (i) above, ytterbium (III) trifluoromethanesulfonate (0.124 g, 0.40 mmol), and 1-butyl-3-methyl-1H-imidazol-3-ium hexafluorophosphate (BMIMPF₆, 0.3 ml) in dioxane (15 ml) was heated at 200° C. for 30 mins in a microwave reactor and concentrated under reduced pressure. Purification by flash chromatography (SP1™ flash system from Biotage™, silica cartridge), using ethyl acetate (gradient from 30 to 70%) in heptane as eluent, followed by concentration in vacuo afforded 0.032 g (17%) of the title compound. ¹H NMR (500 MHz, (CD₃)₂SO) δ 9.87 (s, 1H), 8.13 (s, 1H), 7.41 (dm, 2H), 7.26 (dm, 1H), 7.22 (dm, 2H), 7.03 (m, 1H), 6.98 (m, 1H), 5.07 (d, 1H), 4.78 (s, 1H), 3.97 (d, 1H), 1.24 (s, 9H); MS (ESI) m/z 373.0 ([M+H]⁺);

HRMS calculated for (C₂₀H₂₁ClN₂O₃+H)⁺, 373.1319; found (ESI [M+H]⁺), 373.1338.

### Example 41

### N-(tert-butyl)-2-(4-chlorobenzyl)-7-hydroxy-3-oxoisoindoline-1-carboxamide

The title compound was isolated as by-product in the synthesis of N-(tert-butyl)-2-(4-chlorobenzyl)-5-hydroxy-3-oxoisoindoline-1-carboxamide

HRMS calculated for (C₂₀H₂₁ClN₂O₃+H)⁺, 373.1319; found (ESI [M+H]⁺), 373.1324.

### Example 42

### 2-(biphenyl-2-ylmethyl)-N-(tert-butyl)-7-hydroxy-3-oxoisoindoline-1-carboxamide

The title compound was isolated as by-product in the synthesis of 2-(biphenyl-2-ylmethyl)-N-(tert-butyl)-5-hydroxy-3-oxoisoindoline-1-carboxamide,

HRMS calculated for (C₂₆H₂₆N₂O₃+H)⁺, 415.2022; found (ESI [M+H]⁺), 415.2005.

### Example 43

### 2-(biphenyl-4-ylmethyl)-N-(tert-butyl)-5-hydroxy-4-methyl-3-oxoisoindoline-1-carboxamide

### (i) N-(biphenyl-4-ylmethyl)-N-[2-(tert-butylamino)-1-(2-furyl)-2-oxoethyl]but-2-ynamide

A mixture of but-2-ynoic acid (0.025 g, 0.30 mmol), 1-biphenyl-4-ylmethanamine (0.055 g, 0.30 mmol), and 2-furaldehyde (0.029 g, 0.30 mmol) in MeOH (2 ml) was stirred at room temperature for 30 mins and then tert-butyl isocyanide (0.025 g, 0.30 mmol) was added. The resulting mixture was stirred at room temperature for 3 days and concentrated under reduced pressure giving 0.125 g (97%) of the title compound. The crude product was used in the next step without purification; MS (ESI) m/z 429.0 ([M+H]⁺).

### (ii) 2-(biphenyl-4-ylmethyl)-N-tert-butyl-5-hydroxy-4-methyl-3-oxoisoindoline-1-carboxamide

A mixture of N-(biphenyl-4-ylmethyl)-N-[2-(tert-butylamino)-1-(2-furyl)-2-oxoethyl]but-2-ynamide (0.125 g, 0.29 mmol) from step (i) above and ytterbium (III) trifluoromethanesulfonate (0.037 g, 0.06 mmol) in dioxane (20 ml) was heated at 200° C. for 30 mins in a microwave reactor and concentrated under reduced pressure. The residue was partitioned between DCM and saturated aqueous NaHCO₃ in a Phase Separator. The aqueous phase was extracted with two more portions of DCM and the combined organic layers were concentrated in vacuo. Purification by preparative HPLC using a FractionLynx I HPLC system equipped with a Gemini 5u C18 110A 21.2×100 mm column using a gradient of 5 to 95% CH₃CN in 0.2% NH₃ as mobile phase, followed by concentration in vacuo gave 0.073 g (57%) of the title compund. ¹H NMR (500 MHz, (CD₃)₂SO) δ 9.61 (s, 1H), 8.10 (s, 1H), 7.61 (m, 4H), 7.59 (m, 2H), 7.32 (m, 1H), 7.26 (dm, 2H), 7.06 (dm, 1H), 6.95 (dm, 1H), 5.12 (d, 1H), 4.73 (s, 1H), 3.91 (d, 1H), 1.23 (s, 9H); MS (ESI) m/z 429.2 ([M+H]⁺); HRMS calculated for (C₂₇H₂₈N₂O₃+H)⁺, 429.2178; found (ESI [M+H]⁺), 429.2144.

### Example 44

### N-(tert-butyl)-2-[(4′-fluorobiphenyl-2-yl)methyl]-5-hydroxy-4-methyl-3-oxoisoindoline-1-carboxamide

A mixture of 2-(2-bromobenzyl)-N-tert-butyl-5-hydroxy-4-methyl-3-oxoisoindoline-1-carboxamide (example 37 above) (0.078 g, 0.18 mmol), (4-fluorophenyl)boronic acid (0.030 g, 0.22 mmol), Pd(PPh₃)₄ (0.010 g, 0.009 mmol), and aqueous Cs₂CO₃ (0.117 g in 0.2 ml water) in 1,2-dimethoxyethane (1.0 ml) in a 2 ml microwave reactor vial was degassed by argon-bubbling for 5 mins. The mixture was then heated at 130° C. for 15 mins in a microwave reactor and concentrated in vacuo. Hydrochloric acid (2 ml of a 2 M solution) was added to the residue and the aqueous phase was extracted with three portions of DCM in a Phase Separator. The combined organic layers were concentrated under reduced pressure. Purification by preparative HPLC using a FractionLynx II HPLC system equipped with a Sunfire 5 μm C18 OBD 19×150 mm column using a gradient of 5 to 95% CH₃CN in 0.1 M HCO₂H as mobile phase, followed by concentration in vacuo gave 0.078 g (97%) of the title compund. ¹H NMR (500 MHz, (CD₃)₂SO) δ 9.61 (s, 1H), 7.90 (s, 1H), 7.32 (m, 4H), 7.20 (m, 4H), 7.00 (dm, 1H), 6.91 (dm, 1H), 5.07 (d, 1H), 4.54 (s, 1H), 3.84 (d, 1H), 2.41 (s, 3H), 1.08 (s, 9H); MS (ESI) m/z 447.4 ([M+H]⁺); HRMS calculated for (C₂₇H₂₇FN₂O₃+H)⁺, 447.2084; found (ESI [M+H]⁺), 447.2092.

### Example 45

### N-benzyl-2-[2-(4-chlorophenyl)ethyl]-1-hydroxy-3-oxoisoindoline-1-carboxamide

### (i) 2-[2-(4-chlorophenyl)ethyl]isoquinoline-1,3(2H,4H)-dione

1H-isochromene-1,3(4H)-dione (1.50 g, 6.94 mmol) and 2-(4-chlorophenyl)ethanamine (1.08 g, 6.94 mmol) were mixed in toluene (5 ml) under nitrogen atmosphere and the resulting mixture was refluxed for 20 hours. The mixture was cooled by dilution with 20 ml toluene which resulted in formation of a white solid, which was collected by filtration to give the title compound (1.40 g, 67%).

¹H-NMR (500 MHz, CDCl₃) δ 8.23 (d, 1H), 7.61 (t, 1H), 7.48 (t, 1H), 7.31-7.23 (m, 5H), 4.23-4.18 (m, 2H), 4.04 (s, 2H), 2.94-2.89 (m, 2H).

### (ii) 2-[2-(4-chlorophenyl)ethyl]isoquinoline-1,3,4(2H)-trione

2-[2-(4-chlorophenyl)ethyl]isoquinoline-1,3(2H,4H)-dione (0.50 g, 1.67 mmol) was mixed with SeO₂ (0.19 g, 1.67 mmol) in toluene (10 ml) and heated at reflux for 16 hours. Solid material was removed by filtration and the filtrate was concentrated under reduced pressure. The residue was filtered through a silica gel plug, eluting with DCM. The solvent was removed under reduced pressure to give the title compound (0.52 g, 99%). [M+H] (ES) 314.0

### (iii) N-benzyl-2-[2-(4-chlorophenyl)ethyl]-1-hydroxy-3-oxoisoindoline-1-carboxamide

2-[2-(4-chlorophenyl)ethyl]isoquinoline-1,3,4(2H)-trione (0.15 g, 0.47 mmol) and benzyl amine (0.076 g, 0.71 mmol) were mixed in toluene (2 ml) and the resulting mixture was heated at 60° C. for 16 hours. The solvent was removed under reduced pressure. The residue was purified by flash chromatography using a Biotage SP1 with ethyl acetate/heptane as mobile phase and further purified by reverse phase HPLC equipped with a Kromasil C8 column and MeCN/water (0.1M ammonium acetate) as mobile phase. The product fraction was freeze-dried to give the title compound (0.023 g, 12%).

HRMS: calculated for (C₂₄H₂₁ClN₂O₃+H)⁺ 421.1319; found (ES [M+H]⁺) 421.1336.

¹H-NMR (500 MHz, DMSO-d₆) δ 7.69-7.61 (m, 3H), 7.58-7.51 (m, 2H), 7.36-7.16 (m, 9H), 4.40-4.28 (m, 2H), 3.64-3.56 (m, 1H), 3.28-3.20 (m, 1H), 2.93-2.75 (m, 2H).

### Example 46

### N-(tert-butyl)-2-[(3′,4′-difluorobiphenyl-2-yl)methyl]-3-oxoisoindoline-1-carboxamide

2-formylbenzoic acid (9.12 mmol, 1.37 g) was dissolved in Methanol (10 ml) and 1-(3′,4′-difluorobiphenyl-2-yl)methanamine (9.12 mmol, 2.00 g) (prep AI) was added and stirred for 20 min. Then 2-isocyano-2-methylpropane (9.12 mmol, 0.76 g) was added to the mixture. The reaction was stirred at rt over night. The solvent was removed by evaporation. The crude was purified by flash chromatography (started with isocratic heptane/EtOAc 90/10 and then the EtOAc concentration was increased to 50%, (silica gel 60 0.004-0.063 mm). The product containing fractions was pooled and the solvent was removed by evaporation. The substance was not pure enough so it was purified by preparative HPLC (started with isocratic acetonitrile/buffer 20/80 and then the acetonitrile concentration was increased to 95%, the buffer was a mixture of H2O/ACN/FA (94.8/5/0.2) (0.1 M, column KR-100-7-C8, 50 mm×250 mm, flow 40 ml/min) The product containing fractions was pooled and the acetonitrile was removed by evaporation to give the title compound (2.10 g, 53.1%). 1H-NMR (500 MHz, CDCl₃): δ 7.60-7.54 (m, 2H); 7.54-7.48 (m,1H); 7.42-7.37 (m, 1H); 7.33-7.27 (m, 3H); 7.22-7.13 (m, 2H); 7.12-7.06 (m, 1H); 7.02-6.96 (m, 1H); 6.08 (s, 1H); 5.18 (d, 1H); 4.56 (s, 1H); 4.37-4.30 (d, 1H), 1.15 (s, 9H).

### Example 47

### (1R or 1S)—N-(tert-butyl)-2-[(3′,4′-difluorobiphenyl-2-yl)methyl]-3-oxoisoindoline-1-carboxamide

### (1S or 1R)—N-(tert-butyl)-2-[(3′,4′-difluorobiphenyl-2-yl)methyl]-3-oxoisoindoline-1-carboxamide

N-(tert-butyl)-2-[(3′,4′-difluorobiphenyl-2-yl)methyl]-3-oxoisoindoline-1-carboxamide (Example 46) (4.83 mmol, 2.10 g) were separated by a preparative HPLC system, equipped with a Chiralpak AD, 5μ, 250 mm×20 mm column, using Heptan/EtOH (75/25) as mobile phase, giving fraction 1 containing isomer E1, fraction 2 containing isomer E2.

Fraction 1 was concentrated in vacuo to give (0.903 g, 43.0%) of isomer E1, ee=99.9%.

HRMS calculated for (C26H24F2N2O2+H)+, 435.1884; found (ES [M+H]+), 435.1881.

Fraction 2 was concentrated in vacuo to give (0.908 g, 43.2%) of isomer E2, ee=99.9%.

HRMS calculated for (C26H24F2N2O2+H)+, 435.1884; found (ES [M+H]+), 435.1894.

### Example 48

### N-(tert-butyl)-2-[(4,4′-difluorobiphenyl-2-yl)methyl]-3-oxoisoindoline-1-carboxamide

2-formylbenzoic acid (0.5 mmol, 75 mg) was dissolved in Methanol (10 ml) and 1-(4,4′-difluorobiphenyl-2-yl)methanamine (0.50 mmol, 110 mg) was added and stirred for 20 min. Then 2-isocyano-2-methylpropane (0.50 mmol, 42 mg) was added to the mixture. The reaction was stirred at rt over night. The solvent was removed by evaporation. The crude was purified by preparative HPLC (started with isocratic acetonitrile/buffer 20/80 and then the acetonitrile concentration was increased to 9 5%, the buffer was a mixture of H2O/ACN/FA (94.8/5/0.2) (0.1 M, column KR-100-7-C8, 50 mm×250 mm, flow 40 ml/min). The product containing fractions was pooled and the acetonitrile was removed by evaporation to give the title compound (138 mg, 63.5%). 1H-NMR (500 MHz, CDCl₃): δ 7.76-7.72 (m, 1H); 7.60-7.52 (m, 2H); 7.49-7.44 (m, 1H), 7.29-7.18 (m, 3H); 7.14-7.08 (m, 2H); 7.06-6.98 (m, 2H); 5.62 (s, 1H); 5.11 (d, 1H); 4.59 (s, 1H); 4.35 (d, 1H); 1.10 (s, 9H). HRMS calculated for (C₂₆H₂₄F₂N₂O₂+H)+, 435.1884; found (ES [M+H]+), 435.1904.

### Example 49

### N-butyl-2-[2-(4-chlorophenyl)-2-methyl-propyl]-3-oxo-1H-isoindole-1-carboxamide

2-Formylbenzoic acid (0.060 g, 0.40 mmol), 2-(4-chlorophenyl)-2-methyl-propan-1-amine hydrochloride (0.088 g, 0.40 mmol) and NEt₃ (55 μL, 0.40 mmol) were mixed in MeOH (2 ml) and the resulting mixture was stirred at ambient temperature for 20 mins. 1-Isocyanobutane (42 μL, 0.40 mmol) was added and the resulting mixture was stirred at ambient temperature for 18 hours. The mixture was partitioned between dichloromethane (7 ml) and water (1 ml). The layers were separated in a phase separator and the organic layer was concentrated under reduced pressure. The residue was purified by reverse phase HPLC using a Sunfire prep C18 column and 5-95% MeCN in 0.1 M aqueous HCO₂H as mobile phase, which gave the title compound (0.087 g, 54%).

HRMS: calculated for (C₂₃H₂₇ClN₂O₂+H)⁺ 399.1839; found (ES [M+H]⁺) 399.1839.

¹H-NMR* (500 MHz, DMSO-d₆, DMSO*) δ 8.23-8.19 (m, 1H), 7.65-7.62 (m, 1H), 7.55-7.51 (m, 1H), 7.47-7.42 (m, 2H), 7.37-7.30 (m, 4H), 4.51 (s, 1H), 4.09 (d, 1H), 3.13 (d, 1H), 3.03-2.93 (m, 2H), 1.37-1.31 (m, 2H), 1.28-1.20 (m, 8H), 0.84 (t, 3H).

### Example 50

The following compounds were prepared, from appropriate intermediates (such as those described hereinbefore), according to or by analogy with methods described herein, and identified with accurate mass (HRMS) spectral data (Specified as HRMS calculated (M+H) and HRMS found (M+H). In some cases the ammonium adduct is detected, thus showing (M+NH4)):

N-benzyl-2-(1-methyl-1-phenylethyl)-3-oxoisoindoline-1-carboxamide (385.1916; 385.1902);

N-benzyl-3-oxo-2-(1-phenylpropyl)isoindoline-1-carboxamide (385.1916; 385.1899);

N-[3-(difluoromethoxy)benzyl]-3-oxo-2-(1-phenylethyl)isoindoline-1-carboxamide (437.1676; 437.1670);

N,2-dibenzyl-6-bromo-3-oxoisoindoline-1-carboxamide (435.0708; 435.0693);

6-bromo-2-(2-cyclopentylbenzyl)-N-(4,4-difluorobutyl)-3-oxoisoindoline-1-carboxamide (505.1302; 505.1307);

2-(2-cyclopentylbenzyl)-N-(4,4-difluorobutyl)-6-fluoro-3-oxoisoindoline-1-carboxamide (445.2103; 445.2104);

6-bromo-2-(2-cyclopentylbenzyl)-N-methyl-3-oxoisoindoline-1-carboxamide (427.1021; 427.1015);

2-(2-cyclopentylbenzyl)-6-fluoro-N-methyl-3-oxoisoindoline-1-carboxamide (367.1821; 367.1822);

6-chloro-2-(2-cyclopentylbenzyl)-N-(4,4-difluorobutyl)-3-oxoisoindoline-1-carboxamide (461.1807; 461.1797);

6-chloro-2-(2-cyclopentylbenzyl)-N-methyl-3-oxoisoindoline-1-carboxamide (383.1526; 383.1523);

2-(2-cyclopentylbenzyl)-N-(4,4-difluorobutyl)-3-oxoisoindoline-1-carboxamide (427.2197; 427.2200);

2-(2-cyclopentylbenzyl)-N-methyl-3-oxoisoindoline-1-carboxamide (349.1916; 349.1913);

N-benzyl-6-chloro-3-oxo-2-[(1S)-1-phenylethyl]isoindoline-1-carboxamide (405.1369; 405.1365);

6-chloro-N-(2-methoxyethyl)-3-oxo-2-[2,2,2-trifluoro-1-(3-fluorophenyl)ethyl]isoindoline-1-carboxamide (445.0942; 445.0936);

N-benzyl-6-chloro-2-(dipyridin-3-ylmethyl)-3-oxoisoindoline-1-carboxamide (469.1431; 469.1407);

6-chloro-N-methyl-3-oxo-2-[2-(trifluoromethyl)benzyl]isoindoline-1-carboxamide (383.0774; 383.0798);

2-[2-(4-chlorophenyl)propyl]-6-fluoro-N-methyl-3-oxoisoindoline-1-carboxamide (361.1119; 361.1130);

6-fluoro-N-methyl-3-oxo-2-[(1R)-1-phenylethyl]isoindoline-1-carboxamide (313.1352; 313.1359);

6-chloro-2-[2-(4-chlorophenyl)propyl]-N-methyl-3-oxoisoindoline-1-carboxamide (377.0823; 377.0821);

6-chloro-N-methyl-3-oxo-2-[(1R)-1-phenylethyl]isoindoline-1-carboxamide (329.1056; 329.1062);

2-[2-(4-chlorophenyl)propyl]-N-methyl-3-oxoisoindoline-1-carboxamide (343.1213; 343.1231);

6-chloro-N-ethyl-3-oxo-2-[(1R)-1-phenylethyl]isoindoline-1-carboxamide (343.1213; 343.1238);

N-benzyl-5-[(methylsulfonyl)amino]-3-oxo-2-[(1R)-1-phenylethyl]isoindoline-1-carboxamide (464.1644; 464.1651);

N-benzyl-4-methyl-5-[(methylsulfonyl)amino]-3-oxo-2-[(1R)-1-phenylethyl]isoindoline-1-carboxamide (478.1800; 478.1820);

N-benzyl-5-cyano-3-oxo-2-[(1R)-1-phenylethyl]isoindoline-1-carboxamide (396.1712; 396.1734);

N-benzyl-5-bromo-3-oxo-2-[(1R)-1-phenylethyl]isoindoline-1-carboxamide (449.0864; 449.0868);

N-benzyl-6-bromo-3-oxo-2-[(1R)-1-phenylethyl]isoindoline-1-carboxamide (449.0864; 449.0872);

N-[3-(difluoromethoxy)benzyl]-6-fluoro-2-(3-hydroxy-2,2-dimethylpropyl)-3-oxoisoindoline-1-carboxamide (437.1688; 437.1702);

N-(3,4-dichlorobenzyl)-6-fluoro-2-(3-hydroxy-2,2-dimethylpropyl)-3-oxoisoindoline-1-carboxamide (439.0991; 439.1017);

N-(3-chlorobenzyl)-6-fluoro-2-(3-hydroxy-2,2-dimethylpropyl)-3-oxoisoindoline-1-carboxamide (405.1381; 405.1379);

6-fluoro-2-(3-hydroxy-2,2-dimethylpropyl)-3-oxo-N-[3-(trifluoromethyl)benzyl]isoindoline-1-carboxamide (439.1644; 439.1643);

6-chloro-N-[3-(difluoromethoxy)benzyl]-2-(3-hydroxy-2,2-dimethylpropyl)-3-oxoisoindoline-1-carboxamide (453.1392; 453.1390);

6-chloro-N-(3,4-dichlorobenzyl)-2-(3-hydroxy-2,2-dimethylpropyl)-3-oxoisoindoline-1-carboxamide (455.0696; 455.0714);

6-chloro-N-(3-chlorobenzyl)-2-(3-hydroxy-2,2-dimethylpropyl)-3-oxoisoindoline-1-carboxamide (421.1085; 421.1044);

6-chloro-2-(3-hydroxy-2,2-dimethylpropyl)-3-oxo-N-[3-(trifluoromethyl)benzyl]isoindoline-1-carboxamide (455.1349; 455.1355);

N-(3,4-dichlorobenzyl)-2-(3-hydroxy-2,2-dimethylpropyl)-3-oxoisoindoline-1-carboxamide (421.1085; 421.1091);

N-[3-(difluoromethoxy)benzyl]-2-(3-hydroxy-2,2-dimethylpropyl)-3-oxoisoindoline-1-carboxamide (419.1782; 419.1767);

2-(3-hydroxy-2,2-dimethylpropyl)-3-oxo-N-[3-(trifluoromethyl)benzyl]isoindoline-1-carboxamide (421.1739; 421.1747);

N-(4,4-difluorobutyl)-6-fluoro-3-oxo-2-[2-(trifluoromethyl)benzyl]isoindoline-1-carboxamide (445.1350; 445.1346);

6-chloro-N-(4,4-difluorobutyl)-3-oxo-2-[2-(trifluoromethyl)benzyl]isoindoline-1-carboxamide (461.1055; 461.1014);

6-chloro-N-(4,4-difluorobutyl)-3-oxo-2-[(1R)-1-phenylethyl]isoindoline-1-carboxamide (407.1338; 407.1321);

N-(4,4-difluorobutyl)-3-oxo-2-[(1R)-1-phenylethyl]isoindoline-1-carboxamide (373.1727; 373.1748);

N-(tert-butyl)-6-fluoro-3-oxo-2-[2-(trifluoromethyl)benzyl]isoindoline-1-carboxamide (409.1539; 409.1524);

N-(tert-butyl)-3-oxo-2-[2-(trifluoromethyl)benzyl]isoindoline-1-carboxamide (391.1633; 391.1629);

N-(tert-butyl)-6-chloro-3-oxo-2-[2-(trifluoromethyl)benzyl]isoindoline-1-carboxamide (425.1243; 425.1239);

6-fluoro-3-oxo-N-(4,4,4-trifluorobutyl)-2-[2-(trifluoromethyl)benzyl]isoindoline-1-carboxamide (463.1256; 463.1240);

3-oxo-N-(4,4,4-trifluorobutyl)-2-[2-(trifluoromethyl)benzyl]isoindoline-1-carboxamide (445.1350; 445.1341);

6-chloro-3-oxo-N-(4,4,4-trifluorobutyl)-2-[2-(trifluoromethyl)benzyl]isoindoline-1-carboxamide (479.0961; 479.0967);

N-(tert-butyl)-6-fluoro-3-oxo-2-[(1R)-1-phenylethyl]isoindoline-1-carboxamide (355.1821; 355.1814);

6-fluoro-3-oxo-2-[(1R)-1-phenylethyl]-N-(4,4,4-trifluorobutyl)isoindoline-1-carboxamide (409.1539; 409.1566);

N-(tert-butyl)-6-chloro-3-oxo-2-[(1R)-1-phenylethyl]isoindoline-1-carboxamide (371.1526; 371.1530);

6-chloro-3-oxo-2-[(1R)-1-phenylethyl]-N-(4,4,4-trifluorobutyl)isoindoline-1-carboxamide (425.1243; 425.1255);

3-oxo-2-[(1R)-1-phenylethyl]-N-(4,4,4-trifluorobutyl)isoindoline-1-carboxamide (391.1633; 391.1649);

6-chloro-N-[4-(methylsulfonyl)benzyl]-3-oxo-2-[(1R)-1-phenylethyl]isoindoline-1-carboxamide (483.1145; 483.1145);

N-benzyl-3-oxo-2-[2-(trifluoromethyl)benzyl]isoindoline-1-carboxamide (425.1477; 425.1455);

N-[(4-amino-2-methylpyrimidin-5-yl)methyl]-6-chloro-2-[2-(4-chlorophenyl)propyl]-3-oxoisoindoline-1-carboxamide (484.1307; 484.1289);

2-(biphenyl-2-ylmethyl)-N-[(5-methylpyrazin-2-yl)methyl]-3-oxoisoindoline-1-carboxamide (449.1977; 449.1978);

2-(biphenyl-2-ylmethyl)-3-oxo-N-(pyridin-3-ylmethyl)isoindoline-1-carboxamide (434.1868; 434.1839);

N-[(4-amino-2-methylpyrimidin-5-yl)methyl]-2-(biphenyl-2-ylmethyl)-6-chloro-3-oxoisoindoline-1-carboxamide (498.1696; 498.1682);

N-[(4-amino-2-methylpyrimidin-5-yl)methyl]-2-(biphenyl-2-ylmethyl)-3-oxoisoindoline-1-carboxamide (464.2086; 464.2096);

N-butyl-2-[(4-fluorophenyl)(pyridin-3-yl)methyl]-3-oxoisoindoline-1-carboxamide (418.1930; 418.1921);

N-butyl-3-oxo-2-[phenyl(pyridin-2-yl)methyl]isoindoline-1-carboxamide (400.2025; 400.2018);

N-butyl-2-[(4-chlorophenyl)(pyridin-4-yl)methyl]-3-oxoisoindoline-1-carboxamide (434.1635; 434.1618);

2-[(4-chlorophenyl)(pyridin-4-yl)methyl]-N-(4-fluorobenzyl)-3-oxoisoindoline-1-carboxamide (486.1384; 486.1342);

6-chloro-2-(diphenylmethyl)-N-ethyl-3-oxoisoindoline-1-carboxamide (405.1369; 405.1332);

2-(biphenyl-2-ylmethyl)-6-chloro-N-ethyl-3-oxoisoindoline-1-carboxamide (405.1369; 405.1336);

2-(biphenyl-2-ylmethyl)-6-chloro-3-oxo-N-propylisoindoline-1-carboxamide (419.1526; 419.1521);

2-(diphenylmethyl)-N-ethyl-3-oxoisoindoline-1-carboxamide (371.1759; 371.1759);

2-(biphenyl-2-ylmethyl)-N-ethyl-3-oxoisoindoline-1-carboxamide (371.1759; 371.1758);

2-(biphenyl-2-ylmethyl)-6-fluoro-3-oxo-N-propylisoindoline-1-carboxamide (403.1821; 403.1805);

N-(4-fluorobenzyl)-2-[(4-fluorophenyl)(pyridin-3-yl)methyl]-3-oxoisoindoline-1-carboxamide (470.1680; 470.1664);

N-benzyl-2-[(4-fluorophenyl)(pyridin-3-yl)methyl]-3-oxoisoindoline-1-carboxamide (452.1774; 452.1761);

2-[2-(4-chlorophenyl)propyl]-N-[(5-methylpyrazin-2-yl)methyl]-3-oxoisoindoline-1-carboxamide (435.1587; 435.1592);

6-chloro-2-[2-(4-chlorophenyl)propyl]-N-[(5-methylpyrazin-2-yl)methyl]-3-oxoisoindoline-1-carboxamide (469.1198; 469.1177);

2-(biphenyl-2-ylmethyl)-6-chloro-N-[(5-methylpyrazin-2-yl)methyl]-3-oxoisoindoline-1-carboxamide (483.1587; 483.1582);

6-chloro-2-[2-(4-chlorophenyl)propyl]-3-oxo-N-(pyridin-3-ylmethyl)isoindoline-1-carboxamide (454.1089; 454.1069);

6-chloro-2-[2-(4-chlorophenyl)propyl]-3-oxo-N-{[6-(trifluoromethyl)pyridin-3-yl]methyl}isoindoline-1-carboxamide (522.0963; 522.0932);

N-[(4-amino-2-methylpyrimidin-5-yl)methyl]-2-[2-(4-chlorophenyl)propyl]-3-oxoisoindoline-1-carboxamide (450.1696; 450.1699);

2-(biphenyl-2-ylmethyl)-6-chloro-3-oxo-N-{[6-(trifluoromethyl)pyridin-3-yl]methyl}isoindoline-1-carboxamide (536.1352; 536.1326);

2-[2-(4-chlorophenyl)propyl]-3-oxo-N-{[6-(trifluoromethyl)pyridin-3-yl]methyl}isoindoline-1-carboxamide (488.1352; 488.1335);

2-(biphenyl-2-ylmethyl)-6-chloro-3-oxo-N-(pyridin-3-ylmethyl)isoindoline-1-carboxamide (468.1478; 468.1448);

2-(biphenyl-2-ylmethyl)-3-oxo-N-{[6-(trifluoromethyl)pyridin-3-yl]methyl}isoindoline-1-carboxamide (502.1742; 502.1722);

N-benzyl-6-fluoro-3-oxo-2-[(1R)-1-phenylethyl]isoindoline-1-carboxamide (389.1665; 389.1656);

N-[4-(methylsulfonyl)benzyl]-3-oxo-2-[(1R)-1-phenylethyl]isoindoline-1-carboxamide (449.1535; 449.1510);

6-fluoro-N-[4-(methylsulfonyl)benzyl]-3-oxo-2-[(1R)-1-phenylethyl]isoindoline-1-carboxamide (467.1440; 467.1443);

N-benzyl-6-chloro-3-oxo-2-[2-(trifluoromethyl)benzyl]isoindoline-1-carboxamide (459.1087; 459.1079);

N-benzyl-6-fluoro-3-oxo-2-[2-(trifluoromethyl)benzyl]isoindoline-1-carboxamide (443.1382;443.1373);

6-chloro-N-[4-(methylsulfonyl)benzyl]-3-oxo-2-[2-(trifluoromethyl)benzyl]isoindoline-1-carboxamide (537.0862; 537.0843);

6-chloro-N-[2-chloro-4-(methylsulfonyl)benzyl]-2-(diphenylmethyl)-3-oxoisoindoline-1-carboxamide (579.0912; 579.0919);

N-[2-chloro-4-(methylsulfonyl)benzyl]-2-(diphenylmethyl)-3-oxoisoindoline-1-carboxamide (545.1301; 545.1290);

6-chloro-2-(diphenylmethyl)-N-[2-fluoro-4-(methylsulfonyl)benzyl]-3-oxoisoindoline-1-carboxamide (563.1207; 563.1201);

2-(diphenylmethyl)-N-[2-fluoro-4-(methylsulfonyl)benzyl]-3-oxoisoindoline-1-carboxamide (529.1597; 529.1584);

6-chloro-2-(diphenylmethyl)-N-[4-(methylsulfonyl)benzyl]-3-oxoisoindoline-1-carboxamide (545.1301; 545.1316);

2-(biphenyl-2-ylmethyl)-6-chloro-3-oxo-N-(pyridin-4-ylmethyl)isoindoline-1-carboxamide (468.1478; 468.1479);

6-chloro-2-[2-(4-chlorophenyl)propyl]-3-oxo-N-(pyridin-4-ylmethyl)isoindoline-1-carboxamide (454.1089; 454.1075);

2-(biphenyl-2-ylmethyl)-3-oxo-N-(pyridin-4-ylmethyl)isoindoline-1-carboxamide (434.1868; 434.1853);

2-(biphenyl-2-ylmethyl)-N-(tert-butyl)-5-cyano-3-oxoisoindoline-1-carboxamide (424.2025; 424.2027);

6-chloro-2-(diphenylmethyl)-3-oxo-N-propylisoindoline-1-carboxamide (419.1526; 419.1544);

2-[2-(4-chlorophenyl)propyl]-N-ethyl-6-fluoro-3-oxoisoindoline-1-carboxamide (375.1275; 375.1263);

2-(diphenylmethyl)-N-ethyl-6-fluoro-3-oxoisoindoline-1-carboxamide (389.1665; 389.1671);

2-[2-(4-chlorophenyl)propyl]-N-ethyl-3-oxoisoindoline-1-carboxamide (357.1369; 357.1374);

2-[2-(4-chlorophenyl)propyl]-6-fluoro-3-oxo-N-propylisoindoline-1-carboxamide (389.1432; 389.1431);

2-(diphenylmethyl)-6-fluoro-3-oxo-N-propylisoindoline-1-carboxamide (403.1821; 403.1837);

2-(biphenyl-2-ylmethyl)-3-oxo-N-propylisoindoline-1-carboxamide (385.1916; 385.1903);

2-[2-(4-chlorophenyl)propyl]-3-oxo-N-propylisoindoline-1-carboxamide (371.1526; 371.1546);

2-(diphenylmethyl)-3-oxo-N-propylisoindoline-1-carboxamide (385.1916; 385.1930);

2-(diphenylmethyl)-6-fluoro-3-oxo-N-(4,4,4-trifluorobutyl)isoindoline-1-carboxamide (471.1695; 471.1715);

2-[2-(4-chlorophenyl)propyl]-6-fluoro-3-oxo-N-(4,4,4-trifluorobutyl)isoindoline-1-carboxamide (457.1306; 457.1325);

2-[2-(4-chlorophenyl)propyl]-3-oxo-N-(4,4,4-trifluorobutyl)isoindoline-1-carboxamide (439.1400; 439.1401);

2-(biphenyl-2-ylmethyl)-3-oxo-N-(4,4,4-trifluorobutyl)isoindoline-1-carboxamide (453.1790; 453.1784);

2-(diphenylmethyl)-3-oxo-N-(4,4,4-trifluorobutyl)isoindoline-1-carboxamide (453.1790; 453.1807);

N-(tert-butyl)-2-[(4-chlorophenyl)(pyridin-4-yl)methyl]-3-oxoisoindoline-1-carboxamide (434.1635; 434.1620);

N-(4-fluorobenzyl)-3-oxo-2-[phenyl(pyridin-2-yl)methyl]isoindoline-1-carboxamide (452.1774; 452.1789);

N-benzyl-2-[(4-chlorophenyl)(pyridin-4-yl)methyl]-3-oxoisoindoline-1-carboxamide (468.1478; 468.1465);

N-benzyl-3-oxo-2-[phenyl(pyridin-2-yl)methyl]isoindoline-1-carboxamide (434.1868; 434.1871);

2-(2,2-dimethylpropyl)-N-[(5-methyl-2-phenyl-1,3-oxazol-4-yl)methyl]-3-oxoisoindoline-1-carboxamide (418.2130; 418.2128);

2-(2,2-dimethylpropyl)-N-[(1-methyl-5-phenyl-1H-pyrazol-3-yl)methyl]-3-oxoisoindoline-1-carboxamide (417.2290; 417.2298);

N-benzyl-2-[(1-methyl-5-phenyl-1H-pyrazol-3-yl)methyl]-3-oxoisoindoline-1-carboxamide (437.1977; 437.1993);

N-benzyl-2-[(5-methyl-2-phenyl-1,3-oxazol-4-yl)methyl]-3-oxoisoindoline-1-carboxamide (438.1817; 438.1825);

N-benzyl-2-(biphenyl-2-ylmethyl)-5-hydroxy-4-methyl-3-oxoisoindoline-1-carboxamide (463.2021; 463.2019);

2-(biphenyl-2-ylmethyl)-5-hydroxy-4-methyl-3-oxo-N-(4,4,4-trifluorobutyl)isoindoline-1-carboxamide (483.1895; 483.1881);

2-(biphenyl-2-ylmethyl)-5-hydroxy-4-methyl-3-oxo-N-propylisoindoline-1-carboxamide (415.2021; 415.2033);

2-(biphenyl-2-ylmethyl)-N-butyl-5-hydroxy-4-methyl-3-oxoisoindoline-1-carboxamide (429.2178; 429.2196);

2-(biphenyl-2-ylmethyl)-N-ethyl-5-hydroxy-4-methyl-3-oxoisoindoline-1-carboxamide (401.1865; 401.1870);

N-(tert-butyl)-2-[(3′,4′-difluorobiphenyl-2-yl)methyl]-5-hydroxy-4-methyl-3-oxoisoindoline-1-carboxamide (465.1989; 465.2002);

N-(tert-butyl)-2-[(2′,4′-dichlorobiphenyl-2-yl)methyl]-5-hydroxy-4-methyl-3-oxoisoindoline-1-carboxamide (497.1398; 497.1393);

N-(tert-butyl)-2-[(3′,4′-dichlorobiphenyl-2-yl)methyl]-5-hydroxy-4-methyl-3-oxoisoindoline-1-carboxamide (497.1398; 497.1392);

N-(tert-butyl)-2-[(2′-chlorobiphenyl-2-yl)methyl]-5-hydroxy-4-methyl-3-oxoisoindoline-1-carboxamide (463.1788; 463.1804);

N-(tert-butyl)-2-[(3′-chloro-4′-fluorobiphenyl-2-yl)methyl]-5-hydroxy-4-methyl-3-oxoisoindoline-1-carboxamide (481.1694; 481.1681);

N-(tert-butyl)-2-[(4′-chlorobiphenyl-2-yl)methyl]-5-hydroxy-4-methyl-3-oxoisoindoline-1-carboxamide (463.1788; 463.1764);

N-(tert-butyl)-2-[(4′-fluoro-2′-methylbiphenyl-2-yl)methyl]-5-hydroxy-4-methyl-3-oxoisoindoline-1-carboxamide (461.2240; 461.2221);

N-(tert-butyl)-2-[(2′,4′-difluorobiphenyl-2-yl)methyl]-5-hydroxy-4-methyl-3-oxoisoindoline-1-carboxamide (465.1989; 465.1987);

N-(tert-butyl)-2-[(2′,5′-difluorobiphenyl-2-yl)methyl]-5-hydroxy-4-methyl-3-oxoisoindoline-1-carboxamide (465.1989; 465.1998);

N-(tert-butyl)-2-[(3′-fluorobiphenyl-2-yl)methyl]-5-hydroxy-4-methyl-3-oxoisoindoline-1-carboxamide (447.2084; 447.2097);

N-butyl-3-oxo-2-(2-phenoxybenzyl)isoindoline-1-carboxamide (415.2021; 415.2060);

N-[3-(difluoromethoxy)benzyl]-2-(3,3-dimethylbutyl)-3-oxoisoindoline-1-carboxamide (417.1989; 417.2034);

2-(3,3-dimethylbutyl)-3-oxo-N-[3-(trifluoromethoxy)benzyl]isoindoline-1-carboxamide (435.1895; 435.1855);

2-(2,2-dimethylpropyl)-3-oxo-N-[3-(trifluoromethoxy)benzyl]isoindoline-1-carboxamide (421.1739; 421.1717);

N-[3-(difluoromethoxy)benzyl]-2-(2,2-dimethylpropyl)-3-oxoisoindoline-1-carboxamide (403.1833; 403.1873);

6-chloro-2-(diphenylmethyl)-3-oxo-N-(4,4,4-trifluorobutyl)isoindoline-1-carboxamide (487.1400; 487.1445);

6-chloro-2-(diphenylmethyl)-N-(2-hydroxybenzyl)-3-oxoisoindoline-1-carboxamide (483.1475; 483.1472);

N-benzyl-6-chloro-2-(diphenylmethyl)-3-oxoisoindoline-1-carboxamide (467.1526; 467.1544);

N-(tert-butyl)-6-chloro-2-(diphenylmethyl)-3-oxoisoindoline-1-carboxamide (433.1682; 433.1709);

2-(biphenyl-2-ylmethyl)-6-chloro-3-oxo-N-(4,4,4-trifluorobutyl)isoindoline-1-carboxamide (487.1400; 487.1390);

2-(biphenyl-2-ylmethyl)-6-chloro-N-(3-cyanobenzyl)-3-oxoisoindoline-1-carboxamide (492.1478; 492.1513);

N-benzyl-2-(biphenyl-2-ylmethyl)-6-chloro-3-oxoisoindoline-1-carboxamide (467.1526; 467.1513);

2-(biphenyl-2-ylmethyl)-N-(tert-butyl)-6-chloro-3-oxoisoindoline-1-carboxamide (433.1682; 433.1723);

6-chloro-2-[2-(4-chlorophenyl)propyl]-3-oxo-N-(4,4,4-trifluorobutyl)isoindoline-1-carboxamide (473.1010; 473.1010);

6-chloro-2-[2-(4-chlorophenyl)propyl]-N-[4-(methylsulfonyl)benzyl]-3-oxoisoindoline-1-carboxamide (531.0912; 531.0937);

N-(tert-butyl)-6-chloro-2-[2-(4-chlorophenyl)propyl]-3-oxoisoindoline-1-carboxamide (419.1293; 419.1326);

N-benzyl-6-chloro-2-[2-(4-chlorophenyl)propyl]-3-oxoisoindoline-1-carboxamide (453.1136; 453.1156);

N-(1H-1,2,3-benzotriazol-1-ylmethyl)-4,5-dimethoxy-2-(2-methoxybenzyl)-3-oxoisoindoline-1-carboxamide (488.1934; 488.1938);

2-[1-(1,5-dimethyl-1H-pyrazol-4-yl)ethyl]-5,7-dimethoxy-3-oxo-N-[2-(trifluoromethyl)benzyl]isoindoline-1-carboxamide (517.2062; 517.2051);

5,7-dimethoxy-2-(2-methoxybenzyl)-3-oxo-N-[2-(trifluoromethyl)benzyl]isoindoline-1-carboxamide (515.1793; 515.1784);

2-(2-fluorobenzyl)-5,7-dimethoxy-3-oxo-N-[2-(trifluoromethyl)benzyl]isoindoline-1-carboxamide (503.1594; 503.1598);

N-(tert-butyl)-5,7-dimethoxy-2-(2-methoxybenzyl)-3-oxoisoindoline-1-carboxamide (413.2076; 413.2094);

3-oxo-2-(2-phenoxybenzyl)-N-(4,4,4-trifluorobutyl)isoindoline-1-carboxamide (469.1739; 469.1732);

2-[2-(4-chlorophenyl)propyl]-N-[(2,2-difluoro-1,3-benzodioxol-5-yl)methyl]-3-oxoisoindoline-1-carboxamide (499.1236; 499.1221);

N-(3,4-dichlorobenzyl)-2-(2,2-dimethylpropyl)-3-oxoisoindoline-1-carboxamide (405.1136; 405.1137);

2-(2,2-dimethylpropyl)-N-(1H-indol-3-ylmethyl)-3-oxoisoindoline-1-carboxamide (376.2025; 376.2009);

2-(2,2-dimethylpropyl)-3-oxo-N-{2-[3-(trifluoromethyl)phenyl]ethyl}isoindoline-1-carboxamide (419.1946; 419.1918);

2-(biphenyl-2-ylmethyl)-N-(tert-butyl)-6-fluoro-3-oxoisoindoline-1-carboxamide (417.1978; 417.1967);

N-benzyl-2-(biphenyl-2-ylmethyl)-6-fluoro-3-oxoisoindoline-1-carboxamide (451.1821; 451.1820);

2-[2-(4-chlorophenyl)ethyl]-N-[(2,2-difluoro-1,3-benzodioxol-5-yl)methyl]-6-fluoro-3-oxoisoindoline-1-carboxamide (503.0985; 503.0987);

2-[2-(4-chlorophenyl)ethyl]-6-fluoro-3-oxo-N-(4,4,4-trifluorobutyl)isoindoline-1-carboxamide (443.1149; 443.1151);

N-(tert-butyl)-2-[2-(4-chlorophenyl)ethyl]-6-fluoro-3-oxoisoindoline-1-carboxamide (389.1432; 389.1446);

N-benzyl-2-[2-(4-chlorophenyl)ethyl]-6-fluoro-3-oxoisoindoline-1-carboxamide (423.1275; 423.1266);

6-fluoro-2-[2-(4-fluorophenyl)ethyl]-3-oxo-N-(4,4,4-trifluorobutyl)isoindoline-1-carboxamide (427.1445; 427.1434);

N-benzyl-6-fluoro-2-[2-(4-fluorophenyl)ethyl]-3-oxoisoindoline-1-carboxamide (407.1571; 407.1566);

6-fluoro-2-[2-(4-fluorophenyl)propyl]-3-oxo-N-(4,4,4-trifluorobutyl)isoindoline-1-carboxamide (441.1601; 441.1592);

N-benzyl-6-fluoro-2-[2-(4-fluorophenyl)propyl]-3-oxoisoindoline-1-carboxamide (421.1727; 421.1709);

N-(tert-butyl)-6-fluoro-2-[2-(4-fluorophenyl)propyl]-3-oxoisoindoline-1-carboxamide (387.1884; 387.1868);

2-(biphenyl-2-ylmethyl)-1-methyl-N-[4-(methylsulfonyl)benzyl]-3-oxoisoindoline-1-carboxamide (525.1848; 525.1854);

2-[2-(4-chlorophenyl)propyl]-4,7-difluoro-1-methyl-N-[4-(methylsulfonyl)benzyl]-3-oxoisoindoline-1-carboxamide;

N-butyl-2-[2-(4-chlorophenyl)propyl]-1-methyl-3-oxoisoindoline-1-carboxamide (399.1839; 399.1825);

2-(biphenyl-2-ylmethyl)-N-(tert-butyl)-5,6-dimethoxy-3-oxoisoindoline-1-carboxamide (459.2283; 459.2263);

N-benzyl-2-(diphenylmethyl)-5-methoxy-3-oxoisoindoline-1-carboxamide (463.2021; 463.1992);

2-(diphenylmethyl)-3-oxo-N-[2-(trifluoromethyl)benzyl]isoindoline-1-carboxamide (501.1790; 501.1780);

N-(tert-butyl)-2-[2-(4-chlorophenyl)propyl]-1-methyl-3-oxoisoindoline-1-carboxamide (399.1839; 399.1826);

N-butyl-2-(diphenylmethyl)-3-oxoisoindoline-1-carboxamide (399.2072; 399.2089);

2-(biphenyl-2-ylmethyl)-N-(tert-butyl)-5-methoxy-4-methyl-3-oxoisoindoline-1-carboxamide (443.2334; 443.2317);

2-(biphenyl-2-ylmethyl)-1-[(tert-butylamino)carbonyl]-4-methyl-3-oxo-2,3-dihydro-1H-isoindol-5-yl dimethylcarbamate (500.2549; 500.2561);

2-(biphenyl-2-ylmethyl)-5-hydroxy-3-oxo-N-(2-phenylethyl)isoindoline-1-carboxamide (463.2021; 463.1998);

5-hydroxy-2-[2-(4-methoxyphenyl)ethyl]-3-oxo-N-(2-phenylethyl)isoindoline-1-carboxamide (431.1970; 431.1963);

2-(4-chlorobenzyl)-5-hydroxy-3-oxo-N-(2-phenylethyl)isoindoline-1-carboxamide (421.1319; 421.1311);

N-(4-fluorobenzyl)-5-hydroxy-2-[2-(4-methoxyphenyl)ethyl]-3-oxoisoindoline-1-carboxamide (435.1720; 435.1714);

2-[2-(3,4-dichlorophenyl)ethyl]-N-(4-fluorobenzyl)-5-hydroxy-3-oxoisoindoline-1-carboxamide (473.0835; 473.0795);

2-(4-chlorobenzyl)-N-(4-fluorobenzyl)-5-hydroxy-3-oxoisoindoline-1-carboxamide (425.1068; 425.1054);

2-(biphenyl-2-ylmethyl)-N-(4-fluorobenzyl)-5-hydroxy-3-oxoisoindoline-1-carboxamide (467.1771; 467.1772);

N-(tert-butyl)-2-[2-(3,4-dichlorophenyl)ethyl]-5-hydroxy-3-oxoisoindoline-1-carboxamide (421.1085; 421.1022);

N-(3,4-dichlorobenzyl)-2-isobutyl-3-oxoisoindoline-1-carboxamide (391.0980; 391.0989);

N-[2-(1H-indol-3-yl)ethyl]-2-isobutyl-3-oxoisoindoline-1-carboxamide (376.2025; 376.2027);

N-(3-chlorobenzyl)-2-isobutyl-3-oxoisoindoline-1-carboxamide (357.1369; 357.1352);

N-[4-(difluoromethoxy)benzyl]-2-isobutyl-3-oxoisoindoline-1-carboxamide (389.1676; 389.1673);

2-isobutyl-3-oxo-N-[3-(trifluoromethyl)benzyl]isoindoline-1-carboxamide (391.1633; 391.1613);

N-(1H-indol-3-ylmethyl)-2-isobutyl-3-oxoisoindoline-1-carboxamide (362.1868; 362.1859);

N-[2-(1,3-benzodioxol-5-yl)ethyl]-2-isobutyl-3-oxoisoindoline-1-carboxamide (381.1814; 381.1801);

N-[2-(3-fluorophenyl)ethyl]-2-isobutyl-3-oxoisoindoline-1-carboxamide (355.1821; 355.1813);

2-isobutyl-3-oxo-N-{2-[3-(trifluoromethyl)phenyl]ethyl}isoindoline-1-carboxamide (405.1790; 405.1775);

N-[2-(3,4-dichlorophenyl)ethyl]-2-isobutyl-3-oxoisoindoline-1-carboxamide (405.1136; 405.1135);

N-[2-(4-chlorophenyl)ethyl]-2-isobutyl-3-oxoisoindoline-1-carboxamide (371.1526; 371.1523);

N-[2-(3-chlorophenyl)ethyl]-2-isobutyl-3-oxoisoindoline-1-carboxamide (371.1526; 371.1520);

N-[2-(2-chlorophenyl)ethyl]-2-isobutyl-3-oxoisoindoline-1-carboxamide (371.1526; 371.1513);

N-[2-(2,4-dichlorophenyl)ethyl]-2-isobutyl-3-oxoisoindoline-1-carboxamide (405.1136; 405.1129);

N-[2-(2,6-dichlorophenyl)ethyl]-2-isobutyl-3-oxoisoindoline-1-carboxamide (405.1136; 405.1125);

2-(3,3-dimethylbutyl)-N-[2-(1H-indol-3-yl)ethyl]-3-oxoisoindoline-1-carboxamide (404.2338; 404.2336);

N-(3-chlorobenzyl)-2-(3,3-dimethylbutyl)-3-oxoisoindoline-1-carboxamide (385.1682; 385.1676);

N-(3,4-dichlorobenzyl)-2-(3,3-dimethylbutyl)-3-oxoisoindoline-1-carboxamide (419.1293; 419.1273);

2-(3,3-dimethylbutyl)-N-(1H-indol-3-ylmethyl)-3-oxoisoindoline-1-carboxamide (390.2181; 390.2174);

N-[4-(difluoromethoxy)benzyl]-2-(3,3-dimethylbutyl)-3-oxoisoindoline-1-carboxamide (417.1989; 417.1975);

2-(3,3-dimethylbutyl)-N-[2-(3-fluorophenyl)ethyl]-3-oxoisoindoline-1-carboxamide (383.2134; 383.2103);

N-[2-(1,3-benzodioxol-5-yl)ethyl]-2-(3,3-dimethylbutyl)-3-oxoisoindoline-1-carboxamide (409.2127; 409.2125);

N-[2-(3-cyanophenyl)ethyl]-2-(3,3-dimethylbutyl)-3-oxoisoindoline-1-carboxamide (390.2181; 390.2171);

2-(3,3-dimethylbutyl)-3-oxo-N-{2-[3-(trifluoromethyl)phenyl]ethyl}isoindoline-1-carboxamide (433.2103; 433.2092);

N-[2-(3-chlorophenyl)ethyl]-2-(3,3-dimethylbutyl)-3-oxoisoindoline-1-carboxamide (399.1839; 399.1830);

N-[2-(3,4-dichlorophenyl)ethyl]-2-(3,3-dimethylbutyl)-3-oxoisoindoline-1-carboxamide (433.1449; 433.1431);

N-[2-(4-chlorophenyl)ethyl]-2-(3,3-dimethylbutyl)-3-oxoisoindoline-1-carboxamide (399.1839; 399.1831);

N-[2-(2-chlorophenyl)ethyl]-2-(3,3-dimethylbutyl)-3-oxoisoindoline-1-carboxamide (399.1839; 399.1841);

N-[2-(2,4-dichlorophenyl)ethyl]-2-(3,3-dimethylbutyl)-3-oxoisoindoline-1-carboxamide (433.1449; 433.1428);

2-(2,2-dimethylpropyl)-N-[2-(1H-indol-3-yl)ethyl]-3-oxoisoindoline-1-carboxamide (390.2181; 390.2172);

N-(3-chloroenzyl)-2-(2,2-dimethylpropyl)-3-oxoisoindoline-1-carboxamide (371.1526; 371.1515);

N-[4-(difluoromethoxy)benzyl]-2-(2,2-dimethylpropyl)-3-oxoisoindoline-1-carboxamide (403.1833; 403.1833);

2-(2,2-dimethylpropyl)-3-oxo-N-[3-(trifluoromethyl)benzyl]isoindoline-1-carboxamide (405.1790; 405.1787);

N-[2-(1,3-benzodioxol-5-yl)ethyl]-2-(2,2-dimethylpropyl)-3-oxoisoindoline-1-carboxamide (395.1970; 395.1967);

2-(2,2-dimethylpropyl)-N-[2-(3-fluorophenyl)ethyl]-3-oxoisoindoline-1-carboxamide (369.1978; 369.1977);

N-[2-(3-cyanophenyl)ethyl]-2-(2,2-dimethylpropyl)-3-oxoisoindoline-1-carboxamide (376.2025; 376.2029);

N-[2-(2-chlorophenyl)ethyl]-2-(2,2-dimethylpropyl)-3-oxoisoindoline-1-carboxamide (385.1682; 385.1675);

N-[2-(3-chlorophenyl)ethyl]-2-(2,2-dimethylpropyl)-3-oxoisoindoline-1-carboxamide (385.1682; 385.1671);

N-[2-(2,4-dichlorophenyl)ethyl]-2-(2,2-dimethylpropyl)-3-oxoisoindoline-1-carboxamide (419.1293; 419.1290);

2-[2-(4-chlorophenyl)ethyl]-N-ethyl-3-oxo-N-(2-pyridin-2-ylethyl)isoindoline-1-carboxamide (448.1791; 448.1776);

2-[2-(4-chlorophenyl)ethyl]-3-(1,3-dihydro-2H-isoindol-2-ylcarbonyl)isoindolin-1-one (417.1369; 417.1382);

2-[2-(4-chlorophenyl)ethyl]-N-methyl-3-oxo-N-[2-(trifluoromethyl)benzyl]isoindoline-1-carboxamide (487.1400; 487.1352);

N-benzyl-2-[2-(4-chlorophenyl)ethyl]-N-ethyl-3-oxoisoindoline-1-carboxamide (433.1682; 433.1662);

N-benzyl-2-[2-(4-chlorophenyl)ethyl]-N-methyl-3-oxoisoindoline-1-carboxamide (419.1526; 419.1516);

N-(tert-butyl)-3-oxo-2-{2-8 4-(trifluoromethyl)phenyl]ethyl}isoindoline-1-carboxamide (405.1790; 405.1766);

N-butyl-3-oxo-2-{2-8 4-(trifluoromethyl)phenyl]ethyl}isoindoline-1-carboxamide (405.1790; 405.1771);

N-benzyl-3-oxo-2-{2-[4-(trifluoromethyl)phenyl]ethyl}isoindoline-1-carboxamide (439.1633; 439.1621);

N-(tert-butyl)-5-hydroxy-2-[2-(1H-indol-3-yl)ethyl]-4-methyl-3-oxoisoindoline-1-carboxamide (406.2130; 406.2102);

N-(tert-butyl)-2-[2-(4-fluorophenyl)propyl]-5-hydroxy-4-methyl-3-oxoisoindoline-1-carboxamide (399.2084; 399.2061);

2-[3,5-bis(trifluoromethyl)benzyl]-N-(tert-butyl)-5-hydroxy-4-methyl-3-oxoisoindoline-1-carboxamide (489.1613; 489.1611);

N-(tert-butyl)-2-(2,2-diphenylethyl)-5-hydroxy-4-methyl-3-oxoisoindoline-1-carboxamide (443.2334; 443.2317);

N-(tert-butyl)-2-(diphenylmethyl)-5-hydroxy-4-methyl-3-oxoisoindoline-1-carboxamide (429.2178; 429.2160);

N-(tert-butyl)-2-(9H-fluoren-9-yl)-5-hydroxy-4-methyl-3-oxoisoindoline-1-carboxamide (427.2021; 427.2007);

N-(tert-butyl)-5-hydroxy-4-methyl-3-oxo-2-{2-[4-(trifluoromethyl)phenoxy]benzyl}isoindoline-1-carboxamide (513.2001; 513.1967);

2-(biphenyl-3-ylmethyl)-N-(tert-butyl)-5-hydroxy-4-methyl-3-oxoisoindoline-1-carboxamide (429.2178; 429.2139);

N-butyl-2-[2-(4-fluorophenyl)propyl]-3-oxoisoindoline-1-carboxamide (369.1978; 369.1956);

N-butyl-2-[2-(4-chlorophenyl)ethyl]-3-oxoisoindoline-1-carboxamide (371.1526; 371.1507);

N-(tert-butyl)-2-[2-(4-chlorophenyl)ethyl]-3-oxoisoindoline-1-carboxamide (371.1526; 371.1534);

N-(tert-butyl)-2-[2-(4-fluorophenyl)propyl]-3-oxoisoindoline-1-carboxamide (369.1978; 369.1954);

N-benzyl-2-[2-(4-fluorophenyl)propyl]-3-oxoisoindoline-1-carboxamide (403.1821; 403.1789);

N-benzyl-2-[2-(4-fluorophenyl)ethyl]-3-oxoisoindoline-1-carboxamide (389.1665; 389.1668);

N-benzyl-2-[2-(4-chlorophenyl)ethyl]-3-oxoisoindoline-1-carboxamide (405.1369; 405.1367);

N-[2-(1H-indol-3-yl)ethyl]-3-oxo-2-[4-(piperidin-1-ylcarbonyl)benzyl]isoindoline-1-carboxamide (521.2552; 521.2518);

2-(biphenyl-2-ylmethyl)-N-(2,4-difluorobenzyl)-3-oxoisoindoline-1-carboxamide (469.1727; 469.1689);

2-(biphenyl-2-ylmethyl)-N-(4-cyano-2,6-difluorobenzyl)-3-oxoisoindoline-1-carboxamide (494.1680; 494.1667);

N-(2,4-difluorobenzyl)-2-(diphenylmethyl)-3-oxoisoindoline-1-carboxamide (469.1727; 469.1695);

N-(2-chlorobenzyl)-2-(diphenylmethyl)-3-oxoisoindoline-1-carboxamide (467.1526; 467.1486);

2-(diphenylmethyl)-N-[2-(4-fluorophenyl)ethyl]-3-oxoisoindoline-1-carboxamide (465.1978; 465.1948);

2-(biphenyl-2-ylmethyl)-N-[2-(4-fluorophenyl)ethyl]-3-oxoisoindoline-1-carboxamide (465.1978; 465.1957);

2-(diphenylmethyl)-N-(4-fluorobenzyl)-3-oxoisoindoline-1-carboxamide (451.1821; 451.1785);

N-(2,4-difluorobenzyl)-3-oxo-2-(2-pyridin-3-ylbenzyl)isoindoline-1-carboxamide (470.1680; 470.1645);

N-(2-chlorobenzyl)-3-oxo-2-(2-pyridin-3-ylbenzyl)isoindoline-1-carboxamide (468.1478; 468.1437);

N-[2-(4-fluorophenyl)ethyl]-3-oxo-2-(2-pyridin-3-ylbenzyl)isoindoline-1-carboxamide (466.1930; 466.1917);

N-benzyl-3-oxo-2-(2-pyridin-3-ylbenzyl)isoindoline-1-carboxamide (434.1868; 434.1839);

N-(4-fluorobenzyl)-3-oxo-2-(2-pyridin-3-ylbenzyl)isoindoline-1-carboxamide (452.1774; 452.1760);

N-butyl-5-methoxy-2-(2-methyl-2-phenylpropyl)-3-oxoisoindoline-1-carboxamide (395.2334; 395.2298);

2-(biphenyl-2-ylmethyl)-N-butyl-5-methoxy-3-oxoisoindoline-1-carboxamide (429.2178; 429.2147);

N-butyl-2-[2-(4-fluorophenyl)propyl]-5-methoxy-3-oxoisoindoline-1-carboxamide (399.2084; 399.2057);

N-butyl-2-[2-(4-chlorophenyl)propyl]-5-methoxy-3-oxoisoindoline-1-carboxamide (415.1788; 415.1772);

N-(tert-butyl)-5-methoxy-2-[2-(1-naphthyl)propyl]-3-oxoisoindoline-1-carboxamide (431.2334; 431.2338);

N-(tert-butyl)-2-[2-(4-fluorophenyl)propyl]-5-methoxy-3-oxoisoindoline-1-carboxamide (399.2084; 399.2060);

N-(tert-butyl)-2-[2-(4-chlorophenyl)propyl]-5-methoxy-3-oxoisoindoline-1-carboxamide (415.1788; 415.1774);

2-(biphenyl-2-ylmethyl)-N-(tert-butyl)-5-methoxy-3-oxoisoindoline-1-carboxamide (429.2178; 429.2162);

N-benzyl-5-methoxy--[2-(1-naphthyl)propyl]-3-oxoisoindoline-1-carboxamide (465.2178; 465.2154);

N-benzyl-5-methoxy-2-(2-methyl-2-phenylpropyl)-3-oxoisoindoline-1-carboxamide (429.2178; 429.2183);

N-benzyl-2-(biphenyl-2-ylmethyl)-5-methoxy-3-oxoisoindoline-1-carboxamide (463.2021; 463.1988);

N-butyl-5,6-dimethoxy-2-(2-methyl-2-phenylpropyl)-3-oxoisoindoline-1-carboxamide (425.2440; 425.2408);

N-benzyl-2-[2-(4-chlorophenyl)propyl]-5-methoxy-3-oxoisoindoline-1-carboxamide (449.1632; 449.1597);

N-butyl-5,6-dimethoxy-2-[2-(1-naphthyl)propyl]-3-oxoisoindoline-1-carboxamide (461.2440; 461.2424);

N-butyl-2-[2-(4-fluorophenyl)propyl]-5,6-dimethoxy-3-oxoisoindoline-1-carboxamide (429.2189; 429.2148);

2-(biphenyl-2-ylmethyl)-N-butyl-5,6-dimethoxy-3-oxoisoindoline-1-carboxamide (459.2283; 459.2237);

N-butyl-2-[2-(4-chlorophenyl)propyl]-5,6-dimethoxy-3-oxoisoindoline-1-carboxamide (445.1894; 445.1857);

N-(tert-butyl)-5,6-dimethoxy-2-[2-(1-naphthyl)propyl]-3-oxoisoindoline-1-carboxamide (461.2440; 461.2417);

N-benzyl-5,6-dimethoxy-2-[2-(1-naphthyl)propyl]-3-oxoisoindoline-1-carboxamide (495.2283; 495.2259);

N-benzyl-5,6-dimethoxy-2-(2-methyl-2-phenylpropyl)-3-oxoisoindoline-1-carboxamide (459.2283; 459.2267);

N-benzyl-2-[2-(4-fluorophenyl)propyl]-5,6-dimethoxy-3-oxoisoindoline-1-carboxamide (463.2033; 463.2008);

N-benzyl-2-(biphenyl-2-ylmethyl)-5,6-dimethoxy-3-oxoisoindoline-1-carboxamide (493.2127; 493.2099);

N-benzyl-2-(diphenylmethyl)-5,6-dimethoxy-3-oxoisoindoline-1-carboxamide (493.2127; 493.2092);

N-benzyl-2-[2-(4-chlorophenyl)propyl]-5,6-dimethoxy-3-oxoisoindoline-1-carboxamide (479.1737; 479.1711);

2-(biphenyl-2-ylmethyl)-N-(tert-butyl)-1-methyl-3-oxoisoindoline-1-carboxamide (413.2229; 413.2210);

2-(biphenyl-2-ylmethyl)-N-butyl-1-methyl-3-oxoisoindoline-1-carboxamide (413.2229; 413.2204);

ethyl N-benzyl-N-({-2-[2-(4-chlorophenyl)ethyl]-3-oxo-2,3-dihydro-1H-isoindol-1-yl}carbonyl)glycinate (491.1737; 491.1708);

2-[2-(4-chlorophenyl)ethyl]-N-methyl-3-oxo-N-(2-phenylethyl)isoindoline-1-carboxamide (433.1682; 433.1641);

2-[2-(4-chlorophenyl)ethyl]-N,N-diethyl-3-oxoisoindoline-1-carboxamide (371.1526; 371.1500);

N-benzyl-N-butyl-2-[2-(4-chlorophenyl)ethyl]-3-oxoisoindoline-1-carboxamide (461.1995; 461.1977);

N-[2-(2,6-dichlorophenyl)ethyl]-2-(3,3-dimethylbutyl)-3-oxoisoindoline-1-carboxamide (433.1449; 433.1440);

N-[2-(4-chlorophenyl)ethyl]-2-(2,2-dimethylpropyl)-3-oxoisoindoline-1-carboxamide (385.1682; 385.1677);

N-[2-(2,6-dichlorophenyl)ethyl]-2-(2,2-dimethylpropyl)-3-oxoisoindoline-1-carboxamide (419.1293; 419.1270);

N-butyl-5-methoxy-2-[2-(1-naphthyl)propyl]-3-oxoisoindoline-1-carboxamide (431.2334; 431.2332);

N-benzyl-2-[2-(4-fluorophenyl)propyl]-5-methoxy-3-oxoisoindoline-1-carboxamide (433.1927; 433.1907);

N-(tert-butyl)-2-[2-(4-chlorophenyl)propyl]-5,6-dimethoxy-3-oxoisoindoline-1-carboxamide (445.1894; 445.1870);

N-(tert-butyl)-2-[(4′-fluoro-2′-methylbiphenyl-2-yl)methyl]-3-oxoisoindoline-1-carboxamide (431.2134; 431.2122);

N-(tert-butyl)-2-[(4′-methylbiphenyl-2-yl)methyl]-3-oxoisoindoline-1-carboxamide (413.2229; 413.2214);

N-(tert-butyl)-2-[(4′-methoxybiphenyl-2-yl)methyl]-3-oxoisoindoline-1-carboxamide (429.2178; 429.2179);

N-(tert-butyl)-2-[(4′-fluorobiphenyl-2-yl)methyl]-3-oxoisoindoline-1-carboxamide (417.1978; 417.1974);

methyl N-({2-[(3′,4′-difluorobiphenyl-2-yl)methyl]-3-oxo-2,3-dihydro-1H-isoindol-1-yl}carbonyl)glycinate (451.1469; 451.1465);

methyl N-({2-[(4′-fluoro-2′-methylbiphenyl-2-yl)methyl]-3-oxo-2,3-dihydro-1H-isoindol-1-yl}carbonyl)glycinate (447.1720; 447.1724);

methyl N-({2-[(4′-fluorobiphenyl-2-yl)methyl]-3-oxo-2,3-dihydro-1H-isoindol-1-yl}carbonyl)glycinate (433.1563; 433.1558);

methyl N-({2-[(4′-methylbiphenyl-2-yl)methyl]-3-oxo-2,3-dihydro-1H-isoindol-1-yl}carbonyl)glycinate (429.1814; 429.1809);

2-[(3′,4′-difluorobiphenyl-2-yl)methyl]-N-[4-(methylsulfonyl)benzyl]-3-oxoisoindoline-1-carboxamide (547.1503; 547.1497);

2-[(4′-fluoro-2′-methylbiphenyl-2-yl)methyl]-N-[4-(methylsulfonyl)benzyl]-3-oxoisoindoline-1-carboxamide (543.1753; 543.1778);

2-[(4′-methoxybiphenyl-2-yl)methyl]-N-[4-(methylsulfonyl)benzyl]-3-oxoisoindoline-1-carboxamide (541.1797; 541.1797);

2-[(4′-fluorobiphenyl-2-yl)methyl]-N-[4-(methylsulfonyl)benzyl]-3-oxoisoindoline-1-carboxamide (529.1597; 529.1594);

2-[(4′-methylbiphenyl-2-yl)methyl]-N-[4-(methylsulfonyl)benzyl]-3-oxoisoindoline-1-carboxamide (525.1848; 525.1854);

N-(tert-butyl)-2-(4-chlorobenzyl)-5-hydroxy-4-methyl-3-oxoisoindoline-1-carboxamide (387.1475; 387.1486);

N-(tert-butyl)-5-hydroxy-2-[2-(4-methoxyphenyl)ethyl]-3-oxoisoindoline-1-carboxamide (383.1970; 383.2007);

2-[2-(4-chlorophenyl)propyl]-N-[2-(1H-indol-3-yl)ethyl]-3-oxoisoindoline-1-carboxamide (472.1791; 472.1816);

N-(tert-butyl)-7-hydroxy-2-[2-(4-methoxyphenyl)ethyl]-3-oxoisoindoline-1-carboxamide (383.1970; 383.1978);

2-(biphenyl-2-ylmethyl)-N-(tert-butyl)-5-hydroxy-3-oxoisoindoline-1-carboxamide (415.2021; 415.2018);

2-[2-(3,4-dichlorophenyl)ethyl]-5-hydroxy-3-oxo-N-(2-phenylethyl)isoindoline-1-carboxamide (469.1085; 469.1005);

N-(3,4-difluorobenzyl)-2-(4-hydroxybenzyl)-3-oxoisoindoline-1-carboxamide (409.1363; 409.1375);

N-(3-chlorobenzyl)-2-(4-hydroxybenzyl)-3-oxoisoindoline-1-carboxamide (407.1162; 407.1162);

2-(4-hydroxybenzyl)-3-oxo-N-[4-(trifluoromethyl)benzyl]isoindoline-1-carboxamide (441.1426; 441.1477);

N-[3,5-bis(trifluoromethyl)benzyl]-2-(4-hydroxybenzyl)-3-oxoisoindoline-1-carboxamide (509.1300; 509.1384);

N-(3-chlorobenzyl)-2-(3-cyanobenzyl)-3-oxoisoindoline-1-carboxamide (416.1165; 416.1188);

N-[3,5-bis(trifluoromethyl)benzyl]-2-(3-cyanobenzyl)-3-oxoisoindoline-1-carboxamide (518.1303; 518.1320);

2-(3-cyanobenzyl)-N-(3,4-difluorobenzyl)-3-oxoisoindoline-1-carboxamide (418.1367; 418.1381);

2-(3-cyanobenzyl)-3-oxo-N-[4-(trifluoromethyl)benzyl]isoindoline-1-carboxamide (450.1429; 450.1442);

N-[4-(aminocarbonyl)benzyl]-2-[2-(4-chlorophenyl)propyl]-3-oxoisoindoline-1-carboxamide (462.1584; 462.1599);

N-[4-(aminocarbonyl)benzyl]-2-(biphenyl-2-ylmethyl)-3-oxoisoindoline-1-carboxamide (476.1974; 476.1927);

2-(3,4-difluorobenzyl)-N-{4-[(dimethylamino)methyl]benzyl}-3-oxoisoindoline-1-carboxamide;

2-(3-chlorobenzyl)-N-{4-[(dimethylamino)methyl]benzyl}-3-oxoisoindoline-1-carboxamide;

2-[3,5-bis(trifluoromethyl)benzyl]-N-{4-[(dimethylamino)methyl]benzyl}-3-oxoisoindoline-1-carboxamide

2-(3,4-difluorobenzyl)-N-(4-hydroxybenzyl)-3-oxoisoindoline-1-carboxamide (409.1363; 409.1383);

2-(3-chlorobenzyl)-N-(4-hydroxybenzyl)-3-oxoisoindoline-1-carboxamide (407.1162; 407.1158);

2-[3,5-bis(trifluoromethyl)benzyl]-(4-hydroxybenzyl)-3-oxoisoindoline-1-carboxamide (509.1300; 509.1281);

2-(3-chlorobenzyl)-N-(3-cyanobenzyl)-3-oxoisoindoline-1-carboxamide (416.1165; 416.1172);

N-(3-cyanobenzyl)-2-(3,4-difluorobenzyl)-3-oxoisoindoline-1-carboxamide (418.1367; 418.1353);

2-[2-(4-chlorophenyl)propyl]-N-(4-cyanobenzyl)-3-oxoisoindoline-1-carboxamide (444.1478; 444.1504);

2-[3,5-bis(trifluoromethyl)benzyl]-N-(3-cyanobenzyl)-3-oxoisoindoline-1-carboxamide (518.1303; 518.1278);

N-(tert-butyl)-2-[2-(4-chlorophenyl)propyl]-5-hydroxy-3-oxoisoindoline-1-carboxamide (401.1632; 401.1666);

N-{4-[(dimethylamino)methyl]benzyl}-3-oxo-2-[4-(trifluoromethyl)benzyl]isoindoline-1-carboxamide (482.2055; 482.2060);

N-(4-hydroxybenzyl)-3-oxo-2-[4-(trifluoromethyl)benzyl]isoindoline-1-carboxamide (441.1426; 441.1414);

N-(3-cyanobenzyl)-3-oxo-2-[4-(trifluoromethyl)benzyl]isoindoline-1-carboxamide (450.1429; 450.1461);

2-(biphenyl-3-ylmethyl)-N-(4-cyanobenzyl)-3-oxoisoindoline-1-carboxamide (458.1868; 458.1872);

2-(biphenyl-4-ylmethyl)-N-(4-cyanobenzyl)-3-oxoisoindoline-1-carboxamide (458.1868; 458.1894);

N-butyl-3-oxo-2-[2-(2-phenoxyphenyl)ethyl]isoindoline-1-carboxamide (429.2178; 429.2170);

N-butyl-2-(2-{4-[(diethylamino)carbonyl]phenyl}ethyl)-3-oxoisoindoline-1-carboxamide (436.2600; 436.2588);

N-butyl-2-[2-(3-fluorophenyl)ethyl]-3-oxoisoindoline-1-carboxamide (355.1821; 355.1839);

N-butyl-3-oxo-2-{2-[2-(trifluoromethoxy)phenyl]ethyl}isoindoline-1-carboxamide (421.1739; 421.1722);

2-(2-biphenyl-4-ylethyl)-N-butyl-3-oxoisoindoline-1-carboxamide (413.2229; 413.2253);

N-butyl-2-[2-(4-fluorophenyl)ethyl]-3-oxoisoindoline-1-carboxamide (355.1821; 355.1834);

N-butyl-2-[2-(3,5-dimethoxyphenyl)ethyl]-3-oxoisoindoline-1-carboxamide (397.2127; 397.2129);

N-butyl-3-oxo-2-[2-(4-phenoxyphenyl)ethyl]isoindoline-1-carboxamide (429.2178; 429.2179);

N-butyl-2-[2-(2-ethoxyphenyl)ethyl]-3-oxoisoindoline-1-carboxamide (381.2178; 381.2173);

2-[2-(1,3-benzodioxol-5-yl)ethyl]-N-butyl-3-oxoisoindoline-1-carboxamide (381.1814; 381.1814);

N-(tert-butyl)-3-oxo-2-[2-(2-phenoxyphenyl)ethyl]isoindoline-1-carboxamide (429.2178; 429.2170);

N-(tert-butyl)-3-oxo-2-{2-[2-(trifluoromethoxy)phenyl]ethyl}isoindoline-1-carboxamide (421.1739; 421.1741);

2-(2-biphenyl-4-ylethyl)-N-(tert-butyl)-3-oxoisoindoline-1-carboxamide (413.2229; 413.2261);

N-(tert-butyl)-2-[2-(3,5-dimethoxyphenyl)ethyl]-3-oxoisoindoline-1-carboxamide (397.2127; 397.2129);

N-(tert-butyl)-3-oxo-2-[2-(4-phenoxyphenyl)ethyl]isoindoline-1-carboxamide (429.2178; 429.2147);

N-(tert-butyl)-2-[2-(2-ethoxyphenyl)ethyl]-3-oxoisoindoline-1-carboxamide (381.2178; 381.2169);

2-[2-(1,3-benzodioxol-5-yl)ethyl]-N-(tert-butyl)-3-oxoisoindoline-1-carboxamide (381.1814; 381.1810);

N-[2-(1H-indol-3-yl)ethyl]-3-oxo-2-[2-(2-phenoxyphenyl)ethyl]isoindoline-1-carboxamide (516.2287; 516.2333);

2-(2-{4-[(diethylamino)carbonyl]phenyl}ethyl)-N-[2-(1H-indol-3-yl)ethyl]-3-oxoisoindoline-1-carboxamide (523.2709; 523.2714);

2-[2-(3-fluorophenyl)ethyl]-N-[2-(1H-indol-3-yl)ethyl]-3-oxoisoindoline-1-carboxamide (442.1930; 442.1936);

N-[2-(1H-indol-3-yl)ethyl]-3-oxo-2-{2-[2-(trifluoromethoxy)phenyl]ethyl}isoindoline-1-carboxamide (508.1848; 508.1853);

2-[2-(4-fluorophenyl)ethyl]-N-[2-(1H-indol-3-yl)ethyl]-3-oxoisoindoline-1-carboxamide (442.1930; 442.1929);

2-[2-(3,5-dimethoxyphenyl)ethyl]-N-[2-(1H-indol-3-yl)ethyl]-3-oxoisoindoline-1-carboxamide (484.2236; 484.2237);

2-(2-biphenyl-4-ylethyl)-N-[2-(1H-indol-3-yl)ethyl]-3-oxoisoindoline-1-carboxamide (500.2338; 500.2371);

N-[2-(1H-indol-3-yl)ethyl]-3-oxo-2-[2-(4-phenoxyphenyl)ethyl]isoindoline-1-carboxamide (516.2287; 516.2280);

2-[2-(2-ethoxyphenyl)ethyl]-N-[2-(1H-indol-3-yl)ethyl]-3-oxoisoindoline-1-carboxamide (468.2287; 468.2276);

2-[2-(1,3-benzodioxol-5-yl)ethyl]-N-[2-(1H-indol-3-yl)ethyl]-3-oxoisoindoline-1-carboxamide (468.1923; 468.1899);

(1R)-2-[(1S)-1-(4-fluorophenyl)ethyl]-N-[4-(methylsulfonyl)benzyl]-3-oxoisoindoline-1-carboxamide;

N-[4-(dimethylamino)benzyl]-2-[2-(dimethylamino)-2-phenylethyl]-3-oxoisoindoline-1-carboxamide (457.2603; 457.2610);

N-[4-(dimethylamino)benzyl]-2-(2,2-diphenylethyl)-3-oxoisoindoline-1-carboxamide (490.2494; 490.2497);

2-(1-benzyl-2-fluoroethyl)-N-[(5-methylisoxazol-3-yl)methyl]-3-oxoisoindoline-1-carboxamide (408.1723; 408.1700);

N-(tert-butyl)-2-(2-chloro-4-fluorobenzyl)-3-oxoisoindoline-1-carboxamide (375.1275; 375.1276);

2-(3,4-difluorobenzyl)-N-(4-fluorobenzyl)-5-hydroxy-4-methyl-3-oxoisoindoline-1-carboxamide (441.1426; 441.1414);

2-(3,4-difluorobenzyl)-5-hydroxy-4-methyl-3-oxo-N-(pyridin-3-ylmethyl)isoindoline-1-carboxamide (424.1472; 424.1458);

2-(3,4-difluorobenzyl)-5-hydroxy-4-methyl-3-oxo-N-(2-phenylethyl)isoindoline-1-carboxamide (437.1676; 437.1667);

2-(3,4-difluorobenzyl)-5-hydroxy-3-oxo-4-phenyl-N-(2-phenylethyl)isoindoline-1-carboxamide (499.1833; 499.1788);

N-(2-chlorobenzyl)-2-(3,4-difluorobenzyl)-5-hydroxy-4-methyl-3-oxoisoindoline-1-carboxamide (457.1130; 457.1088);

2-(3,4-difluorobenzyl)-5-hydroxy-4-methyl-3-oxo-N-[2-(trifluoromethyl)benzyl]isoindoline-1-carboxamide (491.1394; 491.1378);

N-(tert-butyl)-2-(2-chlorobenzyl)-5-hydroxy-3-oxo-4-(trimethylsilyl)isoindoline-1-carboxamide;

N-(tert-butyl)-2-(2-chlorobenzyl)-5-hydroxy-4-methyl-3-oxoisoindoline-1-carboxamide (387.1475; 387.1461);

2-(biphenyl-2-ylmethyl)-N-(tert-butyl)-5-hydroxy-3-oxo-4-phenylisoindoline-1-carboxamide (491.2334; 491.2325);

2-[3,5-bis(trifluoromethyl)benzyl]-N-(tert-butyl)-5-hydroxy-3-oxo-4-phenylisoindoline-1-carboxamide (551.1769; 551.1776);

2-[2-(4-chlorophenyl)propyl]-N-{3-[(dimethylamino)carbonyl]-4-fluorobenzyl}-3-oxoisoindoline-1-carboxamide;

2-[3,5-bis(trifluoromethyl)benzyl]-N-(4-cyanophenyl)-3-oxoisoindoline-1-carboxamide;

2-(1-benzyl-2-fluoroethyl)-N-butyl-3-oxoisoindoline-1-carboxamide (369.1978; 369.1972);

2-(1-benzyl-2-fluoroethyl)-N-(4-fluorobenzyl)-3-oxoisoindoline-1-carboxamide (421.1727; 421.1689);

2-(1-benzyl-2-fluoroethyl)-N-[4-(dimethylamino)benzyl]-3-oxoisoindoline-1-carboxamide (446.2243; 446.2229);

N-[4-(aminomethyl)phenyl]-2-[2-(4-chlorophenyl)propyl]-3-oxoisoindoline-1-carboxamide (434.1635; 434.1641);

2-[2-(4-chlorophenyl)propyl]-N-(4-{[(difluoroacetyl)amino]methyl}phenyl)-3-oxoisoindoline-1-carboxamide (512.1552; 512.1567);

N-[4-(aminocarbonyl)phenyl]-2-[2-(4-chlorophenyl)propyl]-3-oxoisoindoline-1-carboxamide (448.1428; 448.1418);

N-(tert-butyl)-2-[2-(4-chlorophenyl)propyl]-5-(fluoromethoxy)-4-methyl-3-oxoisoindoline-1-carboxamide (447.1850; 447.1835);

N-[4-(dimethylamino)benzyl]-3-oxo-2-(4-phenylbutyl)isoindoline-1-carboxamide (442.2494; 442.2494);

N-[4-(dimethylamino)benzyl]-2-(2-hydroxy-2-phenylethyl)-3-oxoisoindoline-1-carboxamide (430.2130; 430.2120);

N-[4-(dimethylamino)benzyl]-3-oxo-2-[2-(1H-pyrazol-1-yl)benzyl]isoindoline-1-carboxamide (466.2243; 466.2235);

N-[4-(dimethylamino)benzyl]-3-oxo-2-(4-phenoxybenzyl)isoindoline-1-carboxamide;

N-[4-(dimethylamino)benzyl]-3-oxo-2-[(1-phenyl-1H-pyrazol-4-yl)methyl]isoindoline-1-carboxamide (466.2243; 466.2254);

N-[4-(dimethylamino)benzyl]-2-[1-(4-fluorophenyl)ethyl]-3-oxoisoindoline-1-carboxamide;

N-[4-(dimethylamino)benzyl]-2-(diphenylmethyl)-3-oxoisoindoline-1-carboxamide (476.2338; 476.2325);

2-(2-chloro-4-fluorobenzyl)-N-[4-(methylsulfonyl)benzyl]-3-oxoisoindoline-1-carboxamide (487.0894; 487.0908);

N-[4-(dimethylamino)benzyl]-3-oxo-2-(1-phenylpropyl)isoindoline-1-carboxamide (428.2338; 428.2318);

N-[4-(methylsulfonyl)benzyl]-3-oxo-2-[2-(1H-pyrazol-1-yl)benzyl]isoindoline-1-carboxamide (501.1596; 501.1581);

2-(2-hydroxy-2-phenylethyl)-N-[4-(methylsulfonyl)benzyl]-3-oxoisoindoline-1-carboxamide (465.1484; 465.1487);

2-(2,2-diphenylethyl)-N-[4-(methylsulfonyl)benzyl]-3-oxoisoindoline-1-carboxamide (525.1848; 525.1848);

2-(diphenylmethyl)-N-[4-(methylsulfonyl)benzyl]-3-oxoisoindoline-1-carboxamide (511.1691; 511.1691);

2-[2-(4-chlorophenyl)propyl]-3-oxo-N-(pyridin-4-ylmethyl)isoindoline-1-carboxamide (420.1478; 420.1482);

N-[4-(methylsulfonyl)benzyl]-3-oxo-2-(1,2,3,4-tetrahydronaphthalen-1-yl)isoindoline-1-carboxamide (475.1691; 475.1682);

2-(2-chloro-4-fluorobenzyl)-3-oxo-N-(pyridin-4-ylmethyl)isoindoline-1-carboxamide (410.1071; 410.1057);

2-[1-(4-fluorophenyl)ethyl]-3-oxo-N-(pyridin-4-ylmethyl)isoindoline-1-carboxamide (390.1617; 390.1623);

(1S)-2-[(2R)-2-(4-chlorophenyl)propyl]-N-(3-methoxypropyl)-1-methyl-3-oxoisoindoline-1-carboxamide (415.1788; 415.1790);

(1R)-2-[(2R)-2-(4-chlorophenyl)propyl]-N-(3-methoxypropyl)-1-methyl-3-oxoisoindoline-1-carboxamide (415.1788; 415.1797);

2-[2-(4-chlorophenyl)propyl]-N-(3-methoxypropyl)-1-methyl-3-oxoisoindoline-1-carboxamide (415.1788; 415.1776);

N-(tert-butyl)-2-[2-(4-chlorophenyl)propyl]-5-hydroxy-3-oxo-4-(trimethylsilyl)isoindoline-1-carboxamide (473.2027; 473.2016);

N-(tert-butyl)-2-(3,4-difluorobenzyl)-5-hydroxy-3-oxo-4-(trimethylsilyl)isoindoline-1-carboxamide (447.1915; 447.1912);

N-(tert-butyl)-2-(3,4-difluorobenzyl)-5-hydroxy-4-methyl-3-oxoisoindoline-1-carboxamide (389.1676; 389.1661);

N-(tert-butyl)-2-(3,4-difluorobenzyl)-5-hydroxy-3-oxo-4-phenylisoindoline-1-carboxamide (451.1833; 451.1846);

N-(tert-butyl)-2-[2-(4-chlorophenyl)propyl]-5-hydroxy-4-methyl-3-oxoisoindoline-1-carboxamide (415.1788; 415.1793);

2-(2-chloro-4-fluorobenzyl)-N-(3-cyano-4-fluorobenzyl)-3-oxoisoindoline-1-carboxamide (452.0977; 452.0974);

2-[3,5-bis(trifluoromethyl)benzyl]-N-(3-cyano-4-fluorobenzyl)-3-oxoisoindoline-1-carboxamide;

N,2-bis(3-cyano-4-fluorobenzyl)-3-oxoisoindoline-1-carboxamide (443.1319; 443.1297);

N-(3-cyano-4-fluorobenzyl)-3-oxo-2-(2-phenylethyl)isoindoline-1-carboxamide (414.1617; 414.1617);

N-(3-cyano-4-fluorobenzyl)-2-(4-fluorobenzyl)-3-oxoisoindoline-1-carboxamide (418.1367; 418.1362);

2-benzyl-N-(3-cyano-4-fluorobenzyl)-3-oxoisoindoline-1-carboxamide (400.1461; 400.1465);

N-(3-cyano-4-fluorobenzyl)-2-(3,4-difluorobenzyl)-3-oxoisoindoline-1-carboxamide (436.1273; 436.1272);

2-(biphenyl-2-ylmethyl)-N-(3-cyano-4-fluorobenzyl)-3-oxoisoindoline-1-carboxamide (476.1774; 476.1775);

2-[2-(4-chlorophenyl)propyl]-N-(3-cyano-4-fluorobenzyl)-3-oxoisoindoline-1-carboxamide (462.1384; 462.1371);

2-(2-chloro-4-fluorobenzyl)-N-(3-{[(difluoroacetyl)amino]methyl}-4-fluorobenzyl)-3-oxoisoindoline-1-carboxamide (534.1207; 534.1210);

2-[2-(4-chlorophenyl)propyl]-N-(3-[(difluoroacetyl)amino]methyl)-4-fluorobenzyl)-3-oxoisoindoline-1-carboxamide;

N-[3-(aminomethyl)-4-fluorobenzyl]-2-[2-(4-chlorophenyl)propyl]-3-oxoisoindoline-1-carboxamide (466.1697; 466.1700);

N-[3-(aminocarbonyl)-4-fluorobenzyl]-2-[2-(4-chlorophenyl)propyl]-3-oxoisoindoline-1-carboxamide (480.1490; 480.1479);

N-[3-(aminocarbonyl)-4-fluorobenzyl]-2-(2-chloro-4-fluorobenzyl)-3-oxoisoindoline-1-carboxamide (470.1083; 470.1077);

N-(tert-butyl)-3-oxo-2-(4-phenylbutyl)isoindoline-1-carboxamide (365.2229; 365.2231);

N-(tert-butyl)-3-oxo-2-(1,2,3,4-tetrahydronaphthalen-1-yl)isoindoline-1-carboxamide (363.2072; 363.2064);

N-(tert-butyl)-3-oxo-2-(4-phenoxybenzyl)isoindoline-1-carboxamide (415.2021; 415.2055);

N-(tert-butyl)-3-oxo-2-[2-(1H-pyrazol-1-yl)benzyl]isoindoline-1-carboxamide (389.1977; 389.1992);

N-(tert-butyl)-2-(2,2-diphenylethyl)-3-oxoisoindoline-1-carboxamide (413.2229; 413.2237);

N-(tert-butyl)-2-(diphenylmethyl)-3-oxoisoindoline-1-carboxamide (399.2072; 399.2099);

N-(1,3-benzodioxol-5-ylmethyl)-2-(2,2-dimethylpropyl)-3-oxoisoindoline-1-carboxamide (381.1814; 381.1772);

2-(2-chloro-4-fluorobenzyl)-N-[(1-methyl-1H-pyrrol-2-yl)methyl]-3-oxoisoindoline-1-carboxamide (412.1228; 412.1213);

N-(1,3-benzodioxol-5-ylmethyl)-2-(2-chloro-4-fluorobenzyl)-3-oxoisoindoline-1-carboxamide (453.1017; 453.0986);

2-(2-chloro-4-fluorobenzyl)-N-[4-(difluoromethoxy)benzyl]-3-oxoisoindoline-1-carboxamide (475.1036; 475.1006);

2-[3,5-bis(trifluoromethyl)benzyl]-3-oxo-N-(2-pyridin-4-ylethyl)isoindoline-1-carboxamide (508.1459; 508.1463);

2-[2-(4-chlorophenyl)propyl]-3-oxo-N-(2-pyridin-4-ylethyl)isoindoline-1-carboxamide (434.1635; 434.1630);

2-[3,5-bis(trifluoromethyl)benzyl]-N-[(1-methyl-1H-pyrrol-2-yl)methyl]-3-oxoisoindoline-1-carboxamide (496.1459; 496.1419);

2-[2-(4-chlorophenyl)propyl]-N-[(1-methyl-1H-pyrrol-2-yl)methyl]-3-oxoisoindoline-1-carboxamide (422.1635; 422.1634);

2-[2-(4-chlorophenyl)propyl]-N-[4-(difluoromethoxy)benzyl]-3-oxoisoindoline-1-carboxamide (485.1443; 485.1420);

2-[3,5-bis(trifluoromethyl)benzyl]-N-[4-(difluoromethoxy)benzyl]-3-oxoisoindoline-1-carboxamide;

N-(1,3-benzodioxol-5-ylmethyl)-2-[3,5-bis(trifluoromethyl)benzyl]-3-oxoisoindoline-1-carboxamide;

2-(2-chloro-4-fluorobenzyl)-N-[4-(dimethylamino)benzyl]-3-oxoisoindoline-1-carboxamide;

N-(1-benzylpyrrolidin-3-yl)-2-(2-chloro-4-fluorobenzyl)-3-oxoisoindoline-1-carboxamide (478.1697; 478.1697);

N-(1-benzylpyrrolidin-3-yl)-2-[2-(4-chlorophenyl)propyl]-3-oxoisoindoline-1-carboxamide (488.2104; 488.2104);

2-(2-chloro-4-fluorobenzyl)-N-[{5-(2-furyl)isoxazol-3-yl]methyl}-3-oxoisoindoline-1-carboxamide (466.0970; 466.0988);

2-(2,2-dimethylpropyl)-N-{[5-(2-furyl)isoxazol-3-yl]methyl}-3-oxoisoindoline-1-carboxamide;

2-[3,5-bis(trifluoromethyl)benzyl]-N-{[5-(2-furyl)isoxazol-3-yl]methyl}-3-oxoisoindoline-1-carboxamide (550.1201; 550.1180);

2-[2-(4-chlorophenyl)propyl]-N-[3-(1H-imidazol-1-yl)propyl]-3-oxoisoindoline-1-carboxamide (437.1744; 437.1733);

2-[3,5-bis(trifluoromethyl)benzyl]-N-[4-(dimethylamino)benzyl]-3-oxoisoindoline-1-carboxamide (536.1772; 536.1783);

2-[2-(4-chlorophenyl)propyl]-N-[4-(dimethylamino)benzyl]-3-oxoisoindoline-1-carboxamide (462.1948; 462.1909);

N-(1-benzylpyrrolidin-3-yl)-2-[3,5-bis(trifluoromethyl)benzyl]-3-oxoisoindoline-1-carboxamide (562.1929; 562.1935);

2-[2-(4-chlorophenyl)propyl]-N-[4-(methylsulfonyl)benzyl]-3-oxoisoindoline-1-carboxamide (497.1301; 497.1281);

N-(tert-butyl)-3-oxo-2-[(1-phenyl-1H-tetrazol-5-yl)methyl]isoindoline-1-carboxamide (391.1882; 391.1883);

2-[2-(4-chlorophenyl)propyl]-N-[3-(dimethylamino)propyl]-3-oxoisoindoline-1-carboxamide;

2-[2-(4-chlorophenyl)propyl]-N-[2-(dimethylamino)ethyl]-3-oxoisoindoline-1-carboxamide (400.1791; 400.1766);

2-[2-(4-chlorophenyl)propyl]-3-oxo-N-(pyridin-3-ylmethyl)isoindoline-1-carboxamide (420.1478; 420.1465);

N-[2-(4-benzoylpiperazin-1-yl)ethyl]-2-[2-(4-chlorophenyl)propyl]-3-oxoisoindoline-1-carboxamide;

2-[2-(4-chlorophenyl)propyl]-3-oxo-N-(1-pyridin-3-ylethyl)isoindoline-1-carboxamide (434.1635; 434.1627);

2-[2-(4-chlorophenyl)propyl]-N-(3-methoxyphenyl)-3-oxoisoindoline-1-carboxamide (435.1475; 435.1462);

2-[2-(4-chlorophenyl)propyl]-3-oxo-N-(1-pyridin-4-ylethyl)isoindoline-1-carboxamide (434.1635; 434.1620);

2-[2-(4-chlorophenyl)propyl]-N-(4-cyanophenyl)-3-oxoisoindoline-1-carboxamide (430.1322; 430.1315);

2-[2-(4-chlorophenyl)propyl]-N-(3-methoxypropyl)-3-oxoisoindoline-1-carboxamide (401.1632; 401.1633);

N-(1,3-benzodioxol-5-ylmethyl)-2-[2-(4-chlorophenyl)propyl]-3-oxoisoindoline-1-carboxamide (463.1424; 463.1411);

2-[2-(4-chlorophenyl)propyl]-N-(3,4-dimethoxybenzyl)-3-oxoisoindoline-1-carboxamide (479.1737; 479.1748);

2-[2-(4-chlorophenyl)propyl]-N-[(3-methyl-5-phenylisoxazol-4-yl)methyl]-3-oxoisoindoline-1-carboxamide (500.1741; 500.1745);

N-butyl-2-[2-(4-chlorophenyl)propyl]-7-fluoro-3-oxoisoindoline-1-carboxamide (403.1588; 403.1577);

N-(tert-butyl)-2-[2-(4-chlorophenyl)propyl]-7-fluoro-3-oxoisoindoline-1-carboxamide (403.1588; 403.1569);

N-(tert-butyl)-3-oxo-2-[2-(phenylsulfonyl)ethyl]isoindoline-1-carboxamide;

N-(tert-butyl)-2-[2-(4-fluorophenoxy)propyl]-3-oxoisoindoline-1-carboxamide (385.1927; 385.1899);

N-(tert-butyl)-3-oxo-2-(2-phenoxypropyl)isoindoline-1-carboxamide (367.2021; 367.2033);

N-benzyl-2-[(5-methylisoxazol-3-yl)methyl]-3-oxoisoindoline-1-carboxamide;

N-benzyl-2-[4-(methylsulfonyl)benzyl]-3-oxoisoindoline-1-carboxamide;

N-benzyl-3-oxo-2-[2-(phenylsulfonyl)ethyl]isoindoline-1-carboxamide (435.1378; 435.1366);

N-benzyl-3-oxo-2-(2-phenoxyethyl)isoindoline-1-carboxamide (387.1708; 387.1705);

N-benzyl-3-oxo-2-(2-phenoxypropyl)isoindoline-1-carboxamide (401.1865; 401.1888);

N-benzyl-2-[2-(4-fluorophenoxy)propyl]-3-oxoisoindoline-1-carboxamide (419.1771; 419.1798);

N-benzyl-2-[(1-benzyl-1H-pyrazol-4-yl)methyl]-3-oxoisoindoline-1-carboxamide (437.1977; 437.1988);

N-benzyl-2-[(5-methyl-3-phenylisoxazol-4-yl)methyl]-3-oxoisoindoline-1-carboxamide (438.1817; 438.1818);

N-benzyl-3-oxo-2-[(3-phenylisoxazol-5-yl)methyl]isoindoline-1-carboxamide (424.1661; 424.1669);

N-(tert-butyl)-5,6-dichloro-2-(4-cyanobenzyl)-3-oxoisoindoline-1-carboxamide;

N-(tert-butyl)-5,6-dichloro-2-(4-fluorobenzyl)-3-oxoisoindoline-1-carboxamide (409.0886; 409.0912);

N-(tert-butyl)-5,6-dichloro-2-(2-methoxybenzyl)-3-oxoisoindoline-1-carboxamide (421.1085; 421.1078);

N-(tert-butyl)-5,6-dichloro-2-[4-(difluoromethoxy)benzyl]-3-oxoisoindoline-1-carboxamide (457.0897; 457.0864);

N-(tert-butyl)-5-fluoro-2-(2-methoxybenzyl)-3-oxoisoindoline-1-carboxamide (371.1771; 371.1768);

N-(4-fluorobenzyl)-3-oxo-2-(2-pyridin-4-ylethyl)isoindoline-1-carboxamide (390.1617; 390.1628);

2-[(1-methyl-1H-pyrrol-2-yl)methyl]-3-oxo-N-[3-(trifluoromethyl)benzyl]isoindoline-1-carboxamide (428.1586; 428.1586);

N-(2-furylmethyl)-3-oxo-2-(2-phenylpropyl)isoindoline-1-carboxamide (375.1708; 375.1698);

2-[2-(4-chlorophenyl)ethyl]-N-[(5-methyl-2-furyl)methyl]-3-oxoisoindoline-1-carboxamide (409.1319; 409.1317);

N-(4-fluorobenzyl)-2-[(1-methyl-1H-pyrrol-2-yl)methyl]-3-oxoisoindoline-1-carboxamide (378.1617; 378.1609);

N-(2-chlorobenzyl)-2-[2-(1H-indol-3-yl)-1-methylethyl]-3-oxoisoindoline-1-carboxamide (458.1635; 458.1631);

N-(tert-butyl)-5,6-dichloro-2-[2-(4-chloro-2-methylphenyl)-2,2-difluoroethyl]-3-oxoisoindoline-1-carboxamide;

N-(tert-butyl)-5,6-dichloro-2-[2-(4-chlorophenyl)propyl]-3-oxoisoindoline-1-carboxamide (453.0903; 453.0917);

N-(tert-butyl)-2-[2-(4-chloro-2-methylphenyl)-2,2-difluoroethyl]-3-oxoisoindoline-1-carboxamide (421.1494; 421.1518);

N-benzyl-2-[2-(4-chloro-2-methylphenyl)-2,2-difluoroethyl]-3-oxoisoindoline-1-carboxamide (455.1338; 455.1351);

2-[2-(4-chlorophenyl)propyl]-3-oxo-N-[2-(trifluoromethyl)benzyl]isoindoline-1-carboxamide (487.1400; 487.1414);

2-[3-(difluoromethoxy)benzyl]-3-oxo-N-[(trimethylsilyl)methyl]isoindoline-1-carboxamide (419.1602; 419.1599);

N-(2-chlorobenzyl)-2-[2-(4-chlorophenyl)propyl]-3-oxoisoindoline-1-carboxamide (453.1136; 453.1123);

2-[4-(difluoromethoxy)benzyl]-3-oxo-N-[(trimethylsilyl)methyl]isoindoline-1-carboxamide (419.1602; 419.1601);

N-(2-chlorobenzyl)-2-(2,5-dimethylbenzyl)-3-oxoisoindoline-1-carboxamide (419.1526; 419.1513);

2-(biphenyl-2-ylmethyl)-N-(4-fluorobenzyl)-3-oxoisoindoline-1-carboxamide (451.1821; 451.1804);

N-(2-chlorobenzyl)-2-[(1R)-1-(4-methoxyphenyl)ethyl]-3-oxoisoindoline-1-carboxamide (435.1475; 435.1466);

N-(2-chlorobenzyl)-2-[(1R)-1-(3-methoxyphenyl)ethyl]-3-oxoisoindoline-1-carboxamide (435.1475; 435.1469);

N-(2-chlorobenzyl)-2-[(1S)-1-(1-naphythyl)ethyl]-3-oxoisoindoline-1-carboxamide (455.1526; 455.1518);

N-benzyl-3-oxo-2-(4-phenoxybenzyl)isoindoline-1-carboxamide (449.1865; 449.1849);

N-(2-chlorobenzyl)-3-oxo-2-(3-phenylpropyl)isoindoline-1-carboxamide (419.1526; 419.1524);

N-(2-chlorobenzyl)-3-oxo-2-(2-phenylethyl)isoindoline-1-carboxamide (405.1369; 405.1336);

N-(2-chlorobenzyl)-3-oxo-2-(1-phenylpropyl)isoindoline-1-carboxamide (419.1526; 419.1520);

N-(2-chlorobenzyl)-2-(1-methyl-3-phenylpropyl)-3-oxoisoindoline-1-carboxamide (433.1682; 433.1693);

N-(2-chlorobenzyl)-3-oxo-2-(2-phenylpropyl)isoindoline-1-carboxamide (419.1526; 419.1516);

2-(biphenyl-2-ylmethyl)-3-oxo-N-[2-(trifluoromethyl)benzyl]isoindoline-1-carboxamide (501.1790; 501.1790);

2-(biphenyl-2-ylmethyl)-N-(2-chlorobenzyl)-3-oxoisoindoline-1-carboxamide (467.1526; 467.1514);

3-oxo-2-(1-phenylpropyl)-N-[2-(trifluoromethyl)benzyl]isoindoline-1-carboxamide (453.1790; 453.1809);

3-oxo-2-(2-phenylpropyl)-N-[2-(trifluoromethyl)benzyl]isoindoline-1-carboxamide (453.1790; 453.1777);

2-(1-methyl-3-phenylpropyl)-3-oxo-N-[2-(trifluoromethyl)benzyl]isoindoline-1-carboxamide (467.1946; 467.1926);

3-oxo-2-(2-phenylethyl)-N-[2-(trifluoromethyl)benzyl]isoindoline-1-carboxamide (439.1633; 439.1626);

N-benzyl-2-[2-(5-bromo-2-methoxyphenyl)ethyl]-3-oxoisoindoline-1-carboxamide (479.0970; 479.0968);

3-oxo-2-(3-phenylpropyl)-N-[2-(trifluoromethyl)benzyl]isoindoline-1-carboxamide (453.1790; 453.1769);

N-benzyl-2-[2-(3-bromo-4-methoxyphenyl)ethyl]-3-oxoisoindoline-1-carboxamide (479.0970; 479.0972);

2-(2-methylbutyl)-3-oxo-N-[2-(trifluoromethyl)benzyl]isoindoline-1-carboxamide (405.1790; 405.1786);

N-benzyl-2-(2,4-difluorobenzyl)-3-oxoisoindoline-1-carboxamide (393.1414; 393.1432);

2-(cyclohexylmethyl)-3-oxo-N-[2-(trifluoromethyl)benzyl]isoindoline-1-carboxamide (431.1946; 431.1945);

2-(3-fluorobenzyl)-3-oxo-N-[2-(trifluoromethyl)benzyl]isoindoline-1-carboxamide (443.1382; 443.1384);

2-(2-ethoxybenzyl)-3-oxo-N-[2-(trifluoromethyl)benzyl]isoindoline-1-carboxamide (469.1739; 469.1753);

3-oxo-2-[4-(trifluoromethoxy)benzyl]-N-[2-(trifluoromethyl)benzyl]isoindoline-1-carboxamide;

2-[2-(4-chlorophenyl)propyl]-N-[2-(3,4-dimethoxyphenyl)ethyl]-3-oxoisoindoline-1-carboxamide (493.1894; 493.1895);

2-[2-(4-chlorophenyl)propyl]-3-oxo-N-[2-(2-thienyl)ethyl]isoindoline-1-carboxamide (439.1247; 439.1242);

2-[2-(4-chlorophenyl)propyl]-N-[2-(4-methoxyphenyl)ethyl]-3-oxoisoindoline-1-carboxamide (463.1788; 463.1794);

2-[2-(4-chlorophenyl)propyl]-3-oxo-N-(2-phenylethyl)isoindoline-1-carboxamide (433.1682; 433.1679);

2-[2-(4-chlorophenyl)propyl]-N-(2-methoxyethyl)-3-oxoisoindoline-1-carboxamide (387.1475; 387.1483);

2-[2-(4-chlorophenyl)propyl]-N-(4-fluorobenzyl)-3-oxoisoindoline-1-carboxamide (437.1432; 437.1448);

N-benzyl-2-[4-(difluoromethoxy)benzyl]-3-oxoisoindoline-1-carboxamide (423.1520; 423.1511);

N-benzyl-2-[3-(difluoromethoxy)benzyl]-3-oxoisoindoline-1-carboxamide (423.1520; 423.1518);

N-butyl-2-(1-naphthylmethyl)-3-oxoisoindoline-1-carboxamide (373.1916; 373.1883);

N-benzyl-2-cycloheptyl-3-oxoisoindoline-1-carboxamide (363.2072; 363.2062);

N-(1H-1,2,3-benzotriazol-1-ylmethyl)-2-[2-(4-bromophenyl)ethyl]-3-oxoisoindoline-1-carboxamide;

N-(1H-1,2,3-benzotriazol-1-ylmethyl)-2-(1-ethylpropyl)-3-oxoisoindoline-1-carboxamide;

N-benzyl-3-oxo-2-[3-(1H-pyrrol-1-yl)benzyl]isoindoline-1-carboxamide (422.1868; 422.1874);

N-benzyl-2-(3-fluorobenzyl)-3-oxoisoindoline-1-carboxamide (375.1508; 375.1501);

N-benzyl-2-[2-(2-methoxyphenyl)ethyl]-3-oxoisoindoline-1-carboxamide (401.1865; 401.1854);

N-benzyl-2-(2-ethoxybenzyl)-3-oxoisoindoline-1-carboxamide (401.1865; 401.1869);

N-(1H-1,2,3-benzotriazol-1-ylmethyl)-3-oxo-2-(2-phenylpropyl)isoindoline-1-carboxamide;

N-benzyl-2-(4-cyanobenzyl)-3-oxoisoindoline-1-carboxamide (382.1555; 382.1558);

N-benzyl-2-(3,5-dimethoxybenzyl)-3-oxoisoindoline-1-carboxamide (417.181; 417.1806);

N-benzyl-2-[1-(1-naphythyl)ethyl]-3-oxoisoindoline-1-carboxamide (421.1916; 421.1899);

N-benzyl-3-oxo-2-[4-(trifluoromethyl)benzyl]isoindoline-1-carboxamide (425.1477; 425.1482);

ethyl N-({2-[3,5-bis(trifluoromethyl)benzyl]-3-oxo-2,3-dihydro-1H-isoindol-1-yl}carbonyl)-beta-alaninate (503.1405; 503.1403);

2-(1-naphthylmethyl)-3-oxo-N-[(trimethylsilyl)methyl]isoindoline-1-carboxamide (403.1841; 403.1844);

ethyl N-({-2-[2-(3,4-dichlorophenyl)ethyl]-3-oxo-2,3-dihydro-1H-isoindol-1-yl}carbonyl)-beta-alaninate (449.1035; 449.1038);

methyl 4-({1-[(benzylamino)carbonyl]-3-oxo-1,3-dihydro-2H-isoindol-2-yl}methyl)benzoate (415.1657; 415.1665);

3-oxo-2-[3-(trifluoromethyl)benzyl]-N-[(trimethylsilyl)methyl]isoindoline-1-carboxamide (421.1559; 421.1552);

2-[1-(1-naphthyl)ethyl]-3-oxo-N-[(trimethylsilyl)methyl]isoindoline-1-carboxamide (417.1998; 417.1983);

3-oxo-2-[4-(trifluoromethyl)benzyl]-N-[(trimethylsilyl)methyl]isoindoline-1-carboxamide (421.1559; 421.1546);

2-[2-(4-bromophenyl)ethyl]-3-oxo-N-[(trimethylsilyl)methyl]isoindoline-1-carboxamide (445.0947; 445.0931);

2-(2-chloro-6-phenoxybenzyl)-3-oxo-N-[(trimethylsilyl)methyl]isoindoline-1-carboxamide (479.1557; 479.1568);

2-(3,4-dichlorobenzyl)-3-oxo-N-[(trimethylsilyl)methyl]isoindoline-1-carboxamide (421.0906; 421.0896);

N-benzyl-2-(1-benzylpyrrolidin-3-yl)-3-oxoisoindoline-1-carboxamide (426.2181; 426.2169);

N-benzyl-2-(1-benzylpyrrolidin-3-yl)-4,5-dimethoxy-3-oxoisoindoline-1-carboxamide (486.2392; 486.2405);

N-benzyl-2-(3,4-difluorobenzyl)-4,5-dimethoxy-3-oxoisoindoline-1-carboxamide (453.1626; 453.1620);

N-benzyl-2-[2-(4-chlorophenyl)propyl]-4,5-dimethoxy-3-oxoisoindoline-1-carboxamide (479.1737; 479.1729);

N-benzyl-4,5-dimethoxy-2-(1-methyl-3-phenylpropyl)-3-oxoisoindoline-1-carboxamide;

N-benzyl-2-[(1-methyl-1H-pyrrol-2-yl)methyl]-3-oxoisoindoline-1-carboxamide (360.1712; 360.1697);

N-benzyl-3-oxo-2-(1-phenyl-2-pyrrolidin-1-ylethyl)isoindoline-1-carboxamide (440.2338; 440.2336);

N-benzyl-2-[2-(4-methoxyphenyl)-2-oxoethyl]-3-oxoisoindoline-1-carboxamide (415.1657; 415.1674);

N-benzyl-2-[(1R)-1-(4-methoxyphenyl)ethyl]-3-oxoisoindoline-1-carboxamide (401.1865; 401.1883);

N-benzyl-2-(3,4-difluorobenzyl)-3-oxoisoindoline-1-carboxamide (393.1414; 393.1403);

N-benzyl-2-[(1R)-1-(3-methoxyphenyl)ethyl]-3-oxoisoindoline-1-carboxamide (401.1865; 401.1859);

N-benzyl-2-(2,5-dimethylbenzyl)-3-oxoisoindoline-1-carboxamide (385.1916; 385.1924);

N-benzyl-2-(1-methyl-3-phenylpropyl)-3-oxoisoindoline-1-carboxamide (399.2072; 399.2062);

N-benzyl-3-oxo-2-{2-[3-(trifluoromethyl)phenyl]ethyl}isoindoline-1-carboxamide (439.1633; 439.1634);

N-benzyl-2-[3,5-bis(trifluoromethyl)benzyl]-3-oxoisoindoline-1-carboxamide (493.1350; 493.1341);

N-benzyl-2-[2-(6-chloro-1H-indol-3-yl)ethyl]-3-oxoisoindoline-1-carboxamide (444.1478; 444.1484);

N,2-dibenzyl-3-oxoisoindoline-1-carboxamide (357.1603; 357.1613);

N-benzyl-2-(cyclohexylmethyl)-3-oxoisoindoline-1-carboxamide (363.2072; 363.2079);

N-benzyl-3-oxo-2-(2-thienylmethyl)isoindoline-1-carboxamide (363.1167; 363.1162);

2-(1,3-benzodioxol-5-ylmethyl)-N-cyclohexyl-3-oxoisoindoline-1-carboxamide (393.1814; 393.1807);

2-(2-methoxybenzyl)-N-(4-methylcyclohexyl)-3-oxoisoindoline-1-carboxamide (393.2178; 393.2169);

tert-butyl N-{[3-oxo-2-(3-pyrrolidin-1-ylpropyl)-2,3-dihydro-1H-isoindol-1-yl]carbonyl}glycinate (402.2392; 402.2388);

N-(tert-butyl)-2-(2-methoxybenzyl)-3-oxoisoindoline-1-carboxamide (353.1865; 353.1861);

N-(1H-1,2,3-benzotriazol-1-ylmethyl)-2-(2-bromobenzyl)-3-oxoisoindoline-1-carboxamide;

2-[2-(4-chlorophenyl)ethyl]-N-cyclohexyl-3-oxoisoindoline-1-carboxamide (397.1682; 397.1648);

N-(2,3-dimethylcyclohexyl)-3-oxo-2-(2-thienylmethyl)isoindoline-1-carboxamide (383.1793; 383.1778);

N-(1H-1,2,3-benzotriazol-1-ylmethyl)-2-(biphenyl-2-ylmethyl)-3-oxoisoindoline-1-carboxamide (474.1930; 474.1937);

2-(2-chlorobenzyl)-N-(4-methylcyclohexyl)-3-oxoisoindoline-1-carboxamide (397.1682; 397.1671);

N-butyl-2-(2-cyclohex-1-en-1-ylethyl)-3-oxoisoindoline-1-carboxamide (341.2229; 341.2213);

N-benzyl-2-[(2R)-2-hydroxy-1,2-diphenylethyl]-3-oxoisoindoline-1-carboxamide (463.2021; 463.2000);

2-(biphenyl-2-ylmethyl)-N-butyl-3-oxoisoindoline-1-carboxamide (399.2072; 399.2090);

2-(biphenyl-2-ylmethyl)-N-isopropyl-3-oxoisoindoline-1-carboxamide (385.1916; 385.1915);

tert-butyl N-{[2-(2-bromobenzyl)-3-oxo-2,3-dihydro-1H-isoindol-1-yl]carbonyl}glycinate (459.0919; 459.0904);

2-[2-(4-chlorophenyl)propyl]-N-isopropyl-3-oxoisoindoline-1-carboxamide (371.1526; 371.1545);

methyl N-{[3-oxo-2-(2-phenylethyl)-2,3-dihydro-1H-isoindol-1-yl]carbonyl}glycinate (353.1501; 353.1496);

N-(tert-butyl)-2-[1-(2-naphthyl)ethyl]-3-oxoisoindoline-1-carboxamide (387.2072; 387.2083);

N-(1H-1,2,3-benzotriazol-1-ylmethyl)-2-[1-(2-naphthyl)ethyl]-3-oxoisoindoline-1-carboxamide (462.1930; 462.1919);

2-[2-(3,4-diethoxyphenyl)ethyl]-3-oxo-N-(2-phenylethyl)isoindoline-1-carboxamide (473.2440; 473.2461);

2-benzyl-3-oxo-N-(2-phenylethyl)isoindoline-1-carboxamide (371.1759; 371.1755);

N-(1H-1,2,3-benzotriazol-1-ylmethyl)-2-[4-(dimethylamino)benzyl]-3-oxoisoindoline-1-carboxamide (441.2039; 441.2030);

N-benzyl-2-(3-methoxybenzyl)-3-oxoisoindoline-1-carboxamide (387.1708; 387.1705);

2-(2-chloro-4-fluorobenzyl)-N-cyclopentyl-3-oxoisoindoline-1-carboxamide (387.1275; 387.1291);

2-(2-chloro-4-fluorobenzyl)-3-oxo-N-(pyridin-3-ylmethyl)isoindoline-1-carboxamide (410.1071; 410.1057);

2-(2,5-dimethoxybenzyl)-3-oxo-N-(2-phenylethyl)isoindoline-1-carboxamide (431.1970; 431.1964);

N-(sec-butyl)-2-[2-(4-chlorophenyl)ethyl]-3-oxoisoindoline-1-carboxamide (371.1526; 371.1496);

N-benzyl-2-(2,3-difluorobenzyl)-3-oxoisoindoline-1-carboxamide (393.1414; 393.1390);

2-(2-chloro-4-fluorobenzyl)-3-oxo-N-(2-phenylethyl)isoindoline-1-carboxamide (423.1275; 423.1260);

2-[2-(4-chlorophenyl)ethyl]-3-oxo-N-(2-phenylethyl)isoindoline-1-carboxamide (419.1526; 419.1539);

N-(1H-1,2,3-benzotriazol-1-ylmethyl)-2-(2-methylbenzyl)-3-oxoisoindoline-1-carboxamide (412.1773; 412.1747);

N-(tert-butyl)-2-(cyclohexylmethyl)-3-oxoisoindoline-1-carboxamide (329.2229; 329.2238);

2-(1-methyl-3-phenylpropyl)-3-oxo-N-(2-phenylethyl)isoindoline-1-carboxamide (413.2229; 413.2227);

N-benzyl-2-cyclohexyl-3-oxoisoindoline-1-carboxamide (349.1916; 349.1906);

N-(tert-butyl)-2-(2-cyclohex-1-en-1-ylethyl)-3-oxoisoindoline-1-carboxamide (341.2229; 341.2216);

N-(tert-butyl)-3-oxo-2-(1-phenylethyl)isoindoline-1-carboxamide;

tert-butyl N-{[2-(cyclohexylmethyl)-3-oxo-2,3-dihydro-1H-isoindol-1-yl]carbonyl}glycinate (387.2283; 387.2270);

N-(1H-1,2,3-benzotriazol-1-ylmethyl)-2-(2-cyclohex-1-en-1-ylethyl)-3-oxoisoindoline-1-carboxamide (416.2086; 416.2081);

tert-butyl N-{[2-(biphenyl-2-ylmethyl)-3-oxo-2,3-dihydro-1H-isoindol-1-yl]carbonyl}glycinate (457.2127; 457.2120);

N-benzyl-2-(2,2-dimethylpropyl)-3-oxoisoindoline-1-carboxamide (337.1916; 337.1917);

N-benzyl-2-(3-methylbutyl)-3-oxoisoindoline-1-carboxamide (337.1916; 337.1907);

N-benzyl-3-oxo-2-[2-(2-thienyl)ethyl]isoindoline-1-carboxamide (377.1323; 377.1326);

N-(1H-1,2,3-benzotriazol-1-ylmethyl)-3-oxo-2-(2-phenylethyl)isoindoline-1-carboxamide (412.1773; 412.1770);

N-benzyl-2-[2-(4-chlorophenyl)propyl]-3-oxoisoindoline-1-carboxamide (419.1526; 419.1497);

N-(tert-butyl)-2-[2-(4-chlorophenyl)propyl]-3-oxoisoindoline-1-carboxamide (385.1682; 385.1663);

N-benzyl-2-(2-methylbenzyl)-3-oxoisoindoline-1-carboxamide (371.1759; 371.1779);

N-benzyl-2-(2-methoxybenzyl)-3-oxoisoindoline-1-carboxamide (387.1708; 387.1711);

N-benzyl-3-oxo-2-(2-phenylethyl)isoindoline-1-carboxamide (371.1759; 371.1746);

N-benzyl-2-[1-(2-naphythyl)ethyl]-3-oxoisoindoline-1-carboxamide (421.1916; 421.1919);

methyl N-({-2-[2-(4-chlorophenyl)propyl]-3-oxo-2,3-dihydro-1H-isoindol-1-yl}carbonyl)glycinate (401.1268; 401.1258);

tert-butyl N-({2-[1-(2-naphythyl)ethyl]-3-oxo-2,3-dihydro-1H-isoindol-1-yl}carbonyl)glycinate (445.2127; 445.2119);

N-(1H-1,2,3-benzotriazol-1-ylmethyl)-2-[2-(4-chlorophenyl)propyl]-3-oxoisoindoline-1-carboxamide (460.1540; 460.1520);

N-butyl-2-[1-(2-naphythyl)ethyl]-3-oxoisoindoline-1-carboxamide (387.2072; 387.2103);

N-benzyl-2-(2-bromobenzyl)-3-oxoisoindoline-1-carboxamide (435.0708; 435.0700);

N-benzyl-2-(2-cyclohex-1-en-1-ylethyl)-3-oxoisoindoline-1-carboxamide (375.2072; 375.2065);

N-benzyl-2-(biphenyl-2-ylmethyl)-3-oxoisoindoline-1-carboxamide (433.1916; 433.1916);

N-butyl-2-[2-(4-chlorophenyl)propyl]-3-oxoisoindoline-1-carboxamide (385.1682; 385.1676);

2-[2-(4-chlorophenyl)-2-methylpropyl]-N-[(1-isopropyl-5-oxopyrrolidin-3-yl)methyl]-3-oxoisoindoline-1-carboxamide (482.221; 482.218);

2-[2-(4-chlorophenyl)propyl]-N-[(1-isopropyl-5-oxopyrrolidin-3-yl)methyl]-3-oxoisoindoline-1-carboxamide;

1H-isoindole-1-carboxamide, 2-[2-(4-chlorophenyl)propyl]-2,3-dihydro-N-[2-[(1-methylethyl)amino]-2-oxoethyl]-3-oxo-(428.1741; 428.174);

N-(tert-butyl)-2-[(4′,5-difluorobiphenyl-2-yl)methyl]-3-oxoisoindoline-1-carboxamide (435.1884; 435.1892);

N-(tert-butyl)-2-[(5-fluorobiphenyl-2-yl)methyl]-3-oxoisoindoline-1-carboxamide (417.1978; 417.1976);

N-(tert-butyl)-2-[(4-fluorobiphenyl-2-yl)methyl]-3-oxoisoindoline-1-carboxamide (417.1978; 417.1978);

2-[2-(4-chlorophenyl)-2-methylpropyl]-N-[(1-isopropyl-5-oxopyrrolidin-3-yl)methyl]-3-oxoisoindoline-1-carboxamide;

2-[2-(4-chlorophenyl)propyl]-N-[(1-isopropyl-5-oxopyrrolidin-3-yl)methyl]-3-oxoisoindoline-1-carboxamide;

1H-isoindole-1-carboxamide, 2-[2-(4-chlorophenyl)propyl]-2,3-dihydro-N-[2-[(1-methylethyl)amino]-2-oxoethyl]-3-oxo-;

2-(2,2-dimethylpropyl)-6-fluoro-3-oxo-N-(1-phenylethyl)isoindoline-1-carboxamide;

2-(2,2-dimethylpropyl)-6-fluoro-N-(3-fluorobenzyl)-3-oxoisoindoline-1-carboxamide;

2-(2,2-dimethylpropyl)-6-fluoro-N-(2-fluorobenzyl)-3-oxoisoindoline-1-carboxamide;

2-(2,2-dimethylpropyl)-N-(3-fluorobenzyl)-3-oxoisoindoline-1-carboxamide;

2-(2,2-dimethylpropyl)-N-(2-fluorobenzyl)-3-oxoisoindoline-1-carboxamide;

2-(1-methyl-1-phenylethyl)-3-oxo-N-(1-phenylethyl)isoindoline-1-carboxamide;

6-fluoro-3-oxo-N,2-bis(1-phenylethyl)isoindoline-1-carboxamide;

N-(1-methyl-1-phenylethyl)-3-oxo-2-(1-phenylethyl)isoindoline-1-carboxamide;

3-oxo-N,2-bis(1-phenylethyl)isoindoline-1-carboxamide;

N-(3-fluorobenzyl)-3-oxo-2-(1-phenylethyl)isoindoline-1-carboxamide;

N-(2-fluorobenzyl)-3-oxo-2-(1-phenylethyl)isoindoline-1-carboxamide;

(1R or 1S)-2-[(2S or 2R)-2-(4-chlorophenyl)propyl]-3-oxo-N-propylisoindoline-1-carboxamide;

(1S or 1R)-2-[(2R or 2S)-2-(4-chlorophenyl)propyl]-3-oxo-N-propylisoindoline-1-carboxamide;

(1R or 1S)-2-[(2R or 2S)-2-(4-chlorophenyl)propyl]-3-oxo-N-propylisoindoline-1-carboxamide;

(R or S)2-(biphenyl-2-ylmethyl)-6-chloro-N-ethyl-3-oxoisoindoline-1-carboxamide;

(S or R)2-(biphenyl-2-ylmethyl)-6-chloro-N-ethyl-3-oxoisoindoline-1-carboxamide;

N-(4-fluorobenzyl)-2-[1-(4-fluorophenyl)ethyl]-3-oxoisoindoline-1-carboxamide;

2-[1-(4-chlorophenyl)ethyl]-N-(4-fluorobenzyl)-3-oxoisoindoline-1-carboxamide;

N-benzyl-2-[1-(4-fluorophenyl)ethyl]-3-oxoisoindoline-1-carboxamide;

2-[1-(4-chlorophenyl)-1-methylethyl]-N-(4-fluorobenzyl)-3-oxoisoindoline-1-carboxamide;

N-benzyl-6-fluoro-2-[1-(4-fluorophenyl)-1-methylethyl]-3-oxoisoindoline-1-carboxamide;

2-[1-(4-chlorophenyl)-1-methylethyl]-3-oxo-N-(4,4,4-trifluorobutyl)isoindoline-1-carboxamide;

N-(4-fluorobenzyl)-2-[1-(4-fluorophenyl)-1-methylethyl]-3-oxoisoindoline-1-carboxamide;

N-benzyl-6-fluoro-2-(1-methyl-1-phenylethyl)-3-oxoisoindoline-1-carboxamide;

2-[1-(4-fluorophenyl)-1-methylethyl]-3-oxo-N-(4,4,4-trifluorobutyl)isoindoline-1-carboxamide;

N-(4-fluorobenzyl)-2-(1-methyl-1-phenylethyl)-3-oxoisoindoline-1-carboxamide;

2-(1-methyl-1-phenylethyl)-3-oxo-N-(4,4,4-trifluorobutyl)isoindoline-1-carboxamide;

2-[2-(4-chlorophenyl)propyl]-N-(5-cyanopentyl)-6-fluoro-3-oxoisoindoline-1-carboxamide;

N-benzyl-6-bromo-3-oxo-2-(1-phenylpropyl)isoindoline-1-carboxamide;

N-benzyl-2-[2-(4-chlorophenyl)propyl]-4-fluoro-3-oxoisoindoline-1-carboxamide;

2-[2-(4-chlorophenyl)propyl]-N-(4,4-difluorobutyl)-4-fluoro-3-oxoisoindoline-1-carboxamide;

2-[2-(4-chlorophenyl)propyl]-4-fluoro-3-oxo-N-propylisoindoline-1-carboxamide;

2-[2-(4-chlorophenyl)propyl]-N-ethyl-4-fluoro-3-oxoisoindoline-1-carboxamide;

N-benzyl-2-(biphenyl-2-ylmethyl)-4-fluoro-3-oxoisoindoline-1-carboxamide;

2-(biphenyl-2-ylmethyl)-N-(4,4-difluorobutyl)-4-fluoro-3-oxoisoindoline-1-carboxamide;

2-(biphenyl-2-ylmethyl)-4-fluoro-3-oxo-N-propylisoindoline-1-carboxamide;

2-(biphenyl-2-ylmethyl)-N-ethyl-4-fluoro-3-oxoisoindoline-1-carboxamide;

N-benzyl-4-fluoro-3-oxo-2-[(1R)-1-phenylethyl]isoindoline-1-carboxamide.

1. A compound of Formula I 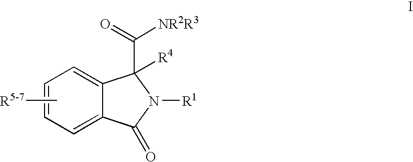
or a pharmaceutically acceptable salt thereof, wherein
R¹ represents C₁-C₁₂ alkyl (which alkyl group is optionally substituted by one or more groups selected from halogen, C₂-C₆ alkenyl, C₃-C₈ cycloalkyl, cyano, oxo, —OR⁸, —COR⁹, —SR¹⁰, —COXR¹¹, —N(R^{12a})(R^{12b}), —N(R^{13a})C(O)OR^{13b}, —OC(O)N(R^{14a})(R^{14b}), —SO₂R¹⁵, aryl and Het¹); further R¹ represents aryl or Het²;
R⁸ to R¹¹, R^{13a}, R^{13b}, R¹⁵ independently represent, at each occurrence, hydrogen, C₁-C₆ alkyl, aryl or Het⁹ (which C₁-C₆ alkyl, aryl and Het⁹ groups are optionally substituted with one or more substituents selected from —OH, halogen, cyano, nitro, C₁-C₆ alkyl, aryl and Het¹⁰);
R^{12a} and R^{12b} independently represent, at each occurrence, hydrogen, C₁-C₆ alkyl, aryl or Het¹¹ (which C₁-C₆ alkyl, aryl and Het¹¹ groups are optionally substituted with one or more substituents selected from —OH, halogen, cyano, nitro, C₁-C₆ alkyl, aryl and Het¹²), or together represent C₃-C₆ alkylene, optionally interrupted by an O atom;
R^{14a} and R^{14b} independently represent, at each occurrence, hydrogen, C₁-C₆ alkyl, aryl or Het¹³ (which C₁-C₆ alkyl, aryl and Het¹³ groups are optionally substituted with one or more substituents selected from —OH, halogen, cyano, nitro, C₁-C₆ alkyl, aryl and Het¹⁴), or together represent C₃-C₆ alkylene, optionally interrupted by an O atom;
R² represents C₁-C₁₂ alkyl (which alkyl group is optionally substituted by one or more groups selected from halogen, —OR¹⁶, —COR¹⁷, C₂-C₆ alkenyl, C₃-C₈ cycloalkyl, cyano, trialkylsilyl, —COXR¹⁸, aryl and Het³); further R² represents —(CH₂)ₖN(R^{19a})(R^{19b}), —(CH₂)ₖNR^{20a}C(O)N(R^{20b})(R^{20c}), —(CH₂)ₙNR^{21a}SO₂R^{21b}, —(CH₂)SO₂R²², —(CH₂)ₖN(R^{23a})C(O)OR^{23b}, —OC(O)N(R^{24a})(R^{24b}), C₃-C₈ cycloalkyl, aryl or Het⁴;
R¹⁶ to R¹⁸, R²¹, R²², R^{23a}, R^{23b} independently represent, at each occurrence, hydrogen, C₁-C₆ alkyl, aryl or Het¹⁵ (which C₁-C₆ alkyl, aryl and Het¹⁵ groups are optionally substituted with one or more substituents selected from —OH, halogen, cyano, nitro, C₁-C₆ alkyl, aryl and Het¹⁶);
R^{19a} and R^{19b} independently represent, at each occurrence, hydrogen, C₁-C₆ alkyl, aryl or Het¹⁹ (which C₁-C₆ alkyl, aryl and Het¹⁹ groups are optionally substituted with one or more substituents selected from —OH, halogen, cyano, nitro, C₁-C₆ alkyl, aryl and Het²⁰) or together represent C₃-C₆ alkylene, optionally interrupted by an O atom;
R^{20a}, R^{20b} and R^{20c} independently represent, at each occurrence, hydrogen, C₁-C₆ alkyl, aryl or Het²¹ (which C₁-C₆ alkyl, aryl and Het²¹ groups are optionally substituted with one or more substituents selected from —OH, halogen, cyano, nitro, C₁-C₆ alkyl, aryl and Het²²); or R^{20b} and R^{20c} may together represent C₃-C₆ alkylene, optionally interrupted by an O atom;
R³ represents hydrogen, C₁-C₁₂ alkyl (which alkyl group is optionally substituted by one or more groups selected from halogen, —OR²⁵, —COR²⁶, C₂-C₆ alkenyl, C₃-C₈ cycloalkyl, trialkylsilyl, —COXR²⁷, aryl and Het⁵); further R³ represents —(CH₂)ₖN(R^{28a})(R^{28b}), —(CH₂)ₖN(R^{29a})C(O)N(R^{29b})(R^{29c}), —(CH₂)ₙNR^{30a}SO₂R^{30b, —(CH}₂)ₙSO₂R³¹, —(CH₂)ₖN(R^{32a})C(O)OR^{32b}, —OC(O)N(R^{33a})(R^{33b}), C₃-C₈ cycloalkyl, aryl or Het⁶;
R²⁵ to R²⁷, R³⁰, R³¹, R^{32a}, R^{32b} independently represent, at each occurrence, hydrogen, C₁-C₆ alkyl, aryl or Het²³ (which C₁-C₆ alkyl, aryl and Het²³ groups are optionally substituted with one or more substituents selected from —OH, halogen, cyano, nitro, C₁-C₆ alkyl, aryl and Het²⁴);
R^{28a} and R^{28b} independently represent, at each occurrence, hydrogen, C₁-C₆ alkyl, aryl or Het²⁵ (which C₁-C₆ alkyl, aryl and Het²⁵ groups are optionally substituted with one or more substituents selected from —OH, halogen, cyano, nitro, C₁-C₆ alkyl, aryl and Het²⁶), or together represent C₃-C₆ alkylene, optionally interrupted by an O atom;
R^{33a} and R^{33b} independently represent, at each occurrence, hydrogen, C₁-C₆ alkyl, aryl or Het²⁷ (which C₁-C₆ alkyl, aryl and Het²⁷ groups are optionally substituted with one or more substituents selected from —OH, halogen, cyano, nitro, C₁-C₆ alkyl, aryl and Het²⁸) or together represent C₃-C₆ alkylene, optionally interrupted by an O atom;
R^{29a}, R^{29b}, and R^{29c} independently represent, at each occurrence, hydrogen, C₁-C₆ alkyl, aryl or Het²⁹ (which C₁-C₆ alkyl, aryl and Het²⁹ groups are optionally substituted with one or more substituents selected from —OH, halogen, cyano, nitro, C₁-C₆ alkyl, aryl and Het³⁰); or R^{29b} and R^{29c} may together represent C₃-C₆ alkylene, optionally interrupted by an O atom;
R⁴ represents hydrogen, —OH, aryl, C₁-C₆ alkyl (which alkyl group is optionally substituted by one or more groups selected from halogen, hydroxy, C₂-C₄ alkenyl, and trialkylsilyl), —OR³⁴, or —(CH₂)ₘR³⁵;
R³⁴ independently represent, at each occurrence, hydrogen, C₁-C₆ alkyl, aryl or Het³¹ (which C₁-C₆ alkyl, aryl and Het³¹ groups are optionally substituted with one or more substituents selected from —OH, halogen, cyano, nitro, C₁-C₆ alkyl, aryl and Het³²);
R³⁵ independently represent aryl or Het³³ (which aryl and Het³³ groups are optionally substituted with one or more substituents selected from —OH, halogen, cyano, nitro, C₁-C₆ alkyl, aryl and Het³⁴);
R⁵ to R⁷ independently represent, at each occurrence, hydrogen, —OH, halogen, cyano, nitro, C₁₋₆ alkyl, —OR³⁶, —N(R^{37a})(R^{37b}), —C(O)R³⁸, —C(O)OR³⁹, —C(O)N(R^{40a})(R^{40b}), —NC(O)OR⁴¹, —OC(O)N(R^{42a})(R^{42b}), —N(R^{43a})C(O)R^{43b}, —N(R^{44a})S(O)₂R^{44b}, —S(O)₂R⁴⁵, —OS(O)₂R⁴⁶, —(CH₂)ₙN(R^{47a})(R^{47b}), —(CH₂)ₙNR^{48a}C(O)N(R^{48b})(R^{48c}), —(CH₂)ₙNR^{49a}SO₂R^{49b}, trialkylsilyl, aryl or Het⁷;
R³⁶, R³⁸, R³⁹, R⁴¹, R⁴³, R^{44a}, R^{44b}, R⁴⁵, R⁴⁶, R^{49a} and R^{49b} independently represent, at each occurrence, hydrogen, C₁-C₆ alkyl, aryl or Het³⁵ (which C₁-C₆ alkyl, aryl and Het³⁵ groups are optionally substituted with one or more substituents selected from —OH, halogen, cyano, nitro, C₁-C₆ alkyl, aryl and Het³⁶);
R^{37a} and R^{37b} independently represent, at each occurrence, hydrogen, C₁-C₆ alkyl, aryl or Het³⁷ (which C₁-C₆ alkyl, aryl and Het³⁷ groups are optionally substituted with one or more substituents selected from —OH, halogen, cyano, nitro, C₁-C₆ alkyl, aryl and Het³⁸), or together represent C₃-C₆ alkylene, optionally interrupted by an O atom;
R^{40a} and R^{40b} independently represent, at each occurrence, hydrogen, C₁-C₆ alkyl, aryl or Het³⁹ (which C₁-C₆ alkyl, aryl and Het³⁹ groups are optionally substituted with one or more substituents selected from —OH, halogen, cyano, nitro, C₁-C₆ alkyl, aryl and Het⁴⁰), or together represent C₃-C₆ alkylene, optionally interrupted by an O atom;
R^{42a} and R^{42b} independently represent, at each occurrence, hydrogen, C₁-C₆ alkyl, aryl or Het⁴¹ (which C₁-C₆ alkyl, aryl and Het⁴¹ groups are optionally substituted with one or more substituents selected from —OH, halogen, cyano, nitro, C₁-C₆ alkyl, aryl and Het⁴²), or together represent C₃-C₆ alkylene, optionally interrupted by an O atom;
R^{47a} and R^{47b} independently represent, at each occurrence, hydrogen, C₁-C₆ alkyl, aryl or Het⁴³ (which C₁-C₆ alkyl, aryl and Het⁴³ groups are optionally substituted with one or more substituents selected from —OH, halogen, cyano, nitro, C₁-C₆ alkyl, aryl and Het⁴⁴), or together represent C₃-C₆ alkylene, optionally interrupted by an O atom;
R^{48a}, R^{48b} and R^{48c} independently represent, at each occurrence, hydrogen, C₁-C₆ alkyl, aryl or Het⁴⁵ (which C₁-C₆ alkyl, aryl and Het⁴⁵ groups are optionally substituted with one or more substituents selected from —OH, halogen, cyano, nitro, C₁-C₆ alkyl, aryl and Het⁴⁶); or R^{48b} and R^{48c} may together represent C₃-C₆ alkylene, optionally interrupted by an O atom;
aryl is independently, at each occurrence, optionally substituted by —OH, halogen, cyano, nitro, C₁-C₆ alkyl, C₃-C₈ cycloalkyl, C₂-C₆ alkenyl, phenyl, Het⁸, —OR⁵⁰, —(CH₂)ₘR⁵¹, —SR⁵², —C(O)R⁵³, —COXR⁵⁴, —N(R^{55a})(R^{55b}), —SO₂R⁵⁶, —OS(O)₂R⁵⁷, —(CH₂)ₘN(R^{58a})(R^{58b}), —CH₂)ₘNR^{59a}C(O)N(R^{59b})(R^{59c}), —C(O)OR⁶⁰, —C(O)N(R^{61a})(R^{61b}), —N(R^{62a}C(O)R^{62b}, —N(R^{63a})C(O)OR^{63b}, —OC(O)N(R^{64a})(R^{64b}), —N(R^{65a})S(O)₂R^{65b} and OC(O)R⁶⁶;
R⁵⁰ to R⁵⁴, R⁵⁶, R⁵⁷, R⁶⁰, R^{62a}, R^{62b}, R^{63a}, R^{63b}, R^{65a}, R^{65b} and R⁶⁶ independently represent, at each occurrence, hydrogen, C₁-C₆ alkyl, aryl or Het⁴⁷ (which C₁-C₆ alkyl, aryl and Het⁴⁷ groups are optionally substituted with one or more substituents selected from —OH, halogen, cyano, nitro, C₁-C₆ alkyl, aryl and Het⁴⁸);
R⁵¹ independently represent aryl or Het⁴⁹ (which aryl and Het⁴⁹ groups are optionally substituted with one or more substituents selected from —OH, halogen, cyano, nitro, C₁-C₆ alkyl, aryl and Het⁵⁰);
R^{55a} and R^{55b} independently represent, at each occurrence, hydrogen, C₁-C₆ alkyl, aryl or Het⁵¹ (which C₁-C₆ alkyl, aryl and Het⁵¹ groups are optionally substituted with one or more substituents selected from —OH, halogen, cyano, nitro, C₁-C₆ alkyl, aryl and Het⁵²), or together represent C₃-C₆ alkylene, optionally interrupted by an O atom;
R^{58a} and R^{58b} independently represent, at each occurrence, hydrogen, C₁-C₆ alkyl, aryl or Het⁵³ (which C₁-C₆ alkyl, aryl and Het⁵³ groups are optionally substituted with one or more substituents selected from —OH, halogen, cyano, nitro, C₁-C₆ alkyl, aryl and Het⁵⁴), or together represent C₃-C₆ alkylene, optionally interrupted by an O atom;
R^{59a}, independently represent, at each occurrence, hydrogen, C₁-C₆ alkyl, aryl or Het⁵⁵ (which C₁-C₆ alkyl, aryl and Het⁵⁵ groups are optionally substituted with one or more substituents selected from —OH, halogen, cyano, nitro, C₁-C₆ alkyl, aryl and Het⁵⁶); or R^{59b} and R^{59c} may together represent C₃-C₆ alkylene, optionally interrupted by an O atom;
R^{61a} and R^{61b} independently represent, at each occurrence, hydrogen, C₁-C₆ alkyl, aryl or Het⁵⁷ (which C₁-C₆ alkyl, aryl and Het⁵⁷ groups are optionally substituted with one or more substituents selected from —OH, halogen, cyano, nitro, C₁-C₆ alkyl, aryl and Het⁵⁸); or together represent C₃-C₆ alkylene, optionally interrupted by an O atom;
R^{64a} and R^{64b} independently represent, at each occurrence, hydrogen, C₁-C₆ alkyl, aryl or Het⁵⁹ (which C₁-C₆ alkyl, aryl and Het⁵⁹ groups are optionally substituted with one or more substituents selected from —OH, halogen, cyano, nitro, C₁-C₆ alkyl, aryl and Het⁶⁰);
Het¹ to Het⁶⁰ independently represent, at each occurrence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₂₋₆ alkenyl, aryl, a further Het, —OR⁶⁷, —(CH₂)ₘR⁶⁸, —SR⁶⁹, —COXR⁷⁰, —N(R^{71a})(R^{71b}), —SO₂R⁷², —(CH₂)ₘN(R^{73a})(R^{73b}), —(CH₂)ₘNR^{74a}C(O)N(R^{74b})(R^{74c}), —C(O)R⁷⁵, —C(O)OR⁷⁶, —C(O)N(R^{77a})(R^{77b}), —N(R^{78a})C(O)R^{78b}, —N(R^{79a})S(O)₂R^{79b}, OC(O)R⁸⁰, —NC(O)OR⁸¹, and —OC(O)N(R^{82a})(R^{82b});
R⁶⁷, R⁶⁹, R⁷⁰, R⁷², R⁷⁵, R⁷⁶, R^{78a}, R^{78b}, R^{79a}, R^{79b}, R⁸⁰ and R⁸¹ independently represent, at each occurrence, hydrogen, C₁-C₆ alkyl, aryl or Het⁶¹ (which C₁-C₆ alkyl, aryl and Het⁶¹ groups are optionally substituted with one or more substituents selected from —OH, halogen, cyano, nitro, C₁-C₆ alkyl, aryl and Het⁶²);
R⁶⁸ represents aryl or Het⁶³ (which aryl and Het⁶³ groups are optionally substituted with one or more substituents selected from —OH, halogen, cyano, nitro, C₁-C₆ alkyl, aryl and Het⁶⁴);
R^{71a} and R^{71b} independently represent, at each occurrence, hydrogen, C₁-C₆ alkyl, aryl or Het⁶⁵ (which C₁-C₆ alkyl, aryl and Het⁶⁵ groups are optionally substituted with one or more substituents selected from —OH, halogen, cyano, nitro, C₁-C₆ alkyl, aryl and Het⁶⁶), or together represent C₃-C₆ alkylene, optionally interrupted by an O atom;
R^{73a} and R^{73b} independently represent, at each occurrence, hydrogen, C₁-C₆ alkyl, aryl or Het⁶⁷ (which C₁-C₆ alkyl, aryl and Het⁶⁷ groups are optionally substituted with one or more substituents selected from —OH, halogen, cyano, nitro, C₁-C₆ alkyl, aryl and Het⁶⁸); or together represent C₃-C₆ alkylene, optionally interrupted by an O atom;
R^{74a}, R^{74b} and R^{74c} independently represent, at each occurrence, hydrogen, C₁-C₆ alkyl, aryl or Het⁶⁹ (which C₁-C₆ alkyl, aryl and Het⁶⁹ groups are optionally substituted with one or more substituents selected from —OH, halogen, cyano, nitro, C₁-C₆ alkyl, aryl and Het⁷⁰); or R^{74b} and R^{74c} may together represent C₃-C₆ alkylene, optionally interrupted by an O atom;
R^{77a}, and R^{77b} independently represent, at each occurrence, hydrogen, C₁-C₆ alkyl, aryl or Het⁷¹ (which C₁-C₆ alkyl, aryl and Het⁷¹ groups are optionally substituted with one or more substituents selected from —OH, halogen, cyano, nitro, C₁-C₆ alkyl, aryl and Het⁷²); or together represent C₃-C₆ alkylene, optionally interrupted by an O atom;
R^{82a}, and R^{82b} independently represent, at each occurrence, hydrogen, C₁-C₆ alkyl, aryl or Het⁷³ (which C₁-C₆ alkyl, aryl and Het⁷³ groups are optionally substituted with one or more substituents selected from —OH, halogen, cyano, nitro, C₁-C₆ alkyl, aryl and Het⁷⁴) or together represent C₃-C₆ alkylene, optionally interrupted by an O atom;
Het⁶¹ to Het⁷⁴ independently represent, at each occurrence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, and C₁₋₆ alkyl;
X represents a nitrogen or oxygen atom;
m is an integer of 0 to 10;
n is an integer of 0 to 4;
k is an integer of 1 to 5;
provided that
a) R² or R³ do not represent a fragment of formula 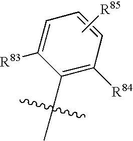
wherein
R⁸³ and R⁸⁴ represent independently, at each occurrence, halogen, C₁-C₁₂ alkyl, C₁-C₁₂ alkoxy, C₁-C₁₂ haloalkyl, C₁-C₁₂ haloalkoxy, cyano, —SR⁸⁶, —N(R^{87a})R^{87b}, C₂-C₆ alkynyl, aryl or Het⁷⁵;
R⁸⁵ represents hydrogen, C₁-C₁₂ alkyl group or C₁-C₁₂ alkoxy group (which C₁-C₁₂ alkyl and C₁-C₁₂ alkoxy groups are optionally substituted by one or more groups selected from halogen, C₂-C₆ alkenyl, C₂-C₆ alkynyl, cyano, oxo, aryl, Het⁷⁶, —OR⁸⁸, —SR⁸⁹, —COXR⁹⁰, —N(R^{91a})R^{91b}, —SO₂R⁹²);
Het⁷⁵ to Het⁷⁶ independently represent, at each occurrence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₁₋₆ alkoxy, aryl, aryloxy, —N(R^{93a}) R^{93b}, —C(O)R^{93c}, —C(O)OR^{93d}, —C(O)N(R^{93e})R^{93f}, —N(R^{93g})C(O)R^{93h} and —N(R⁹³ⁱ)S(O)₂R^{93j}, OC(O)R^{93k} and a further Het; and
R⁸⁶ to R⁹³ represent independently, at each occurrence, hydrogen or C₁₋₆ alkyl;
b) the compound is not:
2-(4-nitrophenyl)-3-(pyrrolidin-1-ylcarbonyl)isoindolin-1-one;
N,2-dibenzyl-3-oxoisoindoline-1-carboxamide;
N,2-diethyl-3-oxoisoindoline-1-carboxamide;
N,2-dibutyl-3-oxoisoindoline-1-carboxamide;
N,2-didodecyl-3-oxoisoindoline-1-carboxamide;
N,2-bis(4-methoxybenzyl)-3-oxoisoindoline-1-carboxamide;
3-oxo-N,2-dipropylisoindoline-1-carboxamide;
N,2-diheptyl-3-oxoisoindoline-1-carboxamide;
3-oxo-N,2-diphenylisoindoline-1-carboxamide;
N-(tert-butyl)-3-oxo-2-propylisoindoline-1-carboxamide;
N-(tert-butyl)-1-methyl-3-oxo-2-propyl-isoindoline-1-carboxamide;
N,1-dimethyl-3-oxo-2-propylisoindoline-1-carboxamide;
N-cyclohexyl-3-oxo-2-propylisoindoline-1-carboxamide;
N-(phenyl)-3-oxo-2-propylisoindoline-1-carboxamide;
2-benzyl-N-tert-butyl-3-oxoisoindoline-1-carboxamide:
2-benzyl-N,1-dimethyl-3-oxoisoindoline-1-carboxamide;
2-benzyl-N-tert-butyl-1-methyl-3-oxoisoindoline-1-carboxamide;
2-benzyl-N,1-dimethyl-3-oxoisoindoline-1-carboxamide;
tert-butyl(4-{1-[(tert-butylamino)carbonyl]-3-oxo-1,3-dihydro-2H-isoindol-2-yl}butyl)carbamate;
2-benzyl-3-oxo-N-(2-phenylethyl)isoindoline-1-carboxamide;
2-benzyl-N-butyl-3-oxoisoindoline-1-carboxamide;
2-benzyl-N-(2-methoxyethyl)-3-oxoisoindoline-1-carboxamide;
2-(2-hydroxyethyl)-3-oxo-N-(2-phenylethyl)isoindoline-1-carboxamide;
N-butyl-2-(2-hydroxyethyl)-3-oxoisoindoline-1-carboxamide;
2-(2-hydroxyethyl)-N-(2-methoxyethyl)-3-oxoisoindoline-1-carboxamide;
2-(3-(1H-imidazol-1-yl)propyl)-3-oxo-N-(2-phenylethyl)isoindoline-1-carboxamide;
N-butyl-2-[3-(1H-imidazol-1-yl)propyl]-3-oxoisoindoline-1-carboxamide;
2-[3-(1H-imidazol-1-yl)propyl]-N-(2-methoxyethyl)-3-oxoisoindoline-1-carboxamide
2-(cyclohexyl)-3-oxo-N-(2-phenylethyl)isoindoline-1-carboxamide;
N-butyl-2-cyclohexyl-3-oxoisoindoline-1-carboxamide;
2-cyclohexyl-N-(2-methoxyethyl)-3-oxoisoindoline-1-carboxamide;
N,2-dibenzyl-5-hydroxy-4-methyl-3-oxoisoindoline-1-carboxamide;
N-benzyl-2-tert-butyl-5-hydroxy-3-oxo-4-phenylisoindoline-1-carboxamide;
N-benzyl-2-tert-butyl-5-hydroxy-4-methyl-3-oxoisoindoline-1-carboxamide;
N,2-dibenzyl-5-hydroxy-3-oxo-4-phenylisoindoline-1-carboxamide;
N-benzyl-2-tert-butyl-5-hydroxy-3-oxoisoindoline-1-carboxamide;
2-cyclohexyl-N-hexyl-3-oxoisoindoline-1-carboxamide;
N,2-dihexyl-3-oxoisoindoline-1-carboxamide;
N-hexyl-(2-hydroxyethyl)-3-oxoisoindoline-1-carboxamide;
N-hexyl-2-(4-hydroxybutyl)-3-oxoisoindoline-1-carboxamide;
N,2-dicyclohexyl-3-oxoisoindoline-1-carboxamide;
N-cyclohexyl-2-hexyl-3-oxoisoindoline-1-carboxamide;
N-cyclohexyl-2-(2-hydroxyethyl)-3-oxoisoindoline-1-carboxamide;
N-cyclohexyl-2-(4-hydroxybutyl)-3-oxoisoindoline-1-carboxamide;
tert-butyl (4-{1-[(cyclohexylamino)carbonyl]-3-oxo-1,3-dihydro-2H-isoindol-2-yl}butyl)carbamate;
N-adamantan-1-yl-2-cyclohexyl-3-oxoisoindoline-1-carboxamide;
N-adamantan-1-yl-2-hexyl-3-oxoisoindoline-1-carboxamide;
N-adamantan-1-yl-2-(2-hydroxyethyl)-3-oxoisoindoline-1-carboxamide;
N-adamantan-1-yl-2-(2-morpholin-4-ylethyl)-3-oxoisoindoline-1-carboxamide;
N,2-dibenzyl-5-{[(2-nitrophenyl)sulfonyl]amino}-3-oxoisoindoline-1-carboxamide;
ethyl [1-(tert-butylcarbamoyl)-3-oxo-1,3-dihydro-2H-isoindol-2-yl]acetate;
N-[2-(3,4-dimethoxyphenyl)ethyl]-3-oxo-2-(1-phenylethyl)isoindoline-1-carboxamide
N-cyclopentyl-2-(3-methoxybenzyl)-3-oxoisoindoline-1-carboxamide;
2-(1,3-benzodioxol-5-ylmethyl)-N-{[(4-methylphenyl)sulfonyl]methyl}-3-oxoisoindoline-1-carboxamide;
N-cyclohexyl-3-oxo-2-(2-thienylmethyl)isoindoline-1-carboxamide;
2-benzyl-N-cyclohexyl-3-oxoisoindoline-1-carboxamide;
N-{[(4-methylphenyl)sulfonyl]methyl}-3-oxo-2-(2-thienylmethyl)isoindoline-1-carboxamide;
2-(4-chlorobenzyl)-N-{[(4-methylphenyl)sulfonyl]methyl}-3-oxoisoindoline-1-carboxamide;
N-cyclohexyl-2-(2-furylmethyl)-3-oxoisoindoline-1-carboxamide;
2-(4-chlorobenzyl)-N-cyclohexyl-3-oxoisoindoline-1-carboxamide;
tert-butyl{1-benzyl-2-hydroxy-3-[(2-hydroxy-3-{[(3-oxo-2,3-dihydro-1H-isoindol-1-yl)carbonyl]amino}-4-phenylbutyl)amino]propyl}carbamate;
1-hydroxy-2-methyl-3-oxo-N-(pyridin-2-ylmethyl)isoindoline-1-carboxamide;
N-[3-(dimethylamino)propyl]-1-hydroxy-2-(2-hydroxyethyl)-3-oxoisoindoline-1-carboxamide;
N-(3-azepan-1-ylpropyl)-1-hydroxy-3-oxo-2-phenylisoindoline-1-carboxamide;
2-benzoyl-1-hydroxy-3-oxo-N-phenylisoindoline-1-carboxamide;
3-oxo-N,2-diphenylisoindoline-1-carboxamide;
6-{[(1-methyl-2-octyl-3-oxo-2,3-dihydro-1H-isoindol-1-yl)carbonyl]amino}hexanoic acid;
N-(methyl)-2-benzoyl-1-hydroxy-3-oxoindoline-1-carboxamide;
N-(phenyl)-2-benzoyl-1-hydroxy-3-oxoisoindoline-1-carboxamide;
6-[(2-allyl-1-methyl-3-oxoisoindoline-1-carbonyl)-amino]-hexanoic acid;
N-{(1S,2R)-1-(3,5-difluorobenzyl)-3-[(3-ethylbenzyl)amino]-2-hydroxypropyl}-2-ethyl-3-oxoisoindoline-1-carboxamide;
N1-cyclopentyl-N4-(2,6-difluorophenyl)-2-(2,4-dimethylphenyl)-5-methyl-3-oxoisoindoline-1,4-dicarboxamide;
methyl[1-(tert-butylcarbamoyl)-3-oxo-1,3-dihydro-2H-isoindol-2-yl]acetate;
1-hydroxy-2-methyl-3-oxoisoindoline-1-carbohydrazide;
1-hydroxy-3-oxo-phenylisoindoline-1-carbohydrazide;
or a pharmaceutically acceptable derivative thereof;
c) the compound is not:
2-(2-ethoxyethyl)-N-isopropyl-3-oxoisoindoline-1-carboxamide;
N-(tert-butyl)-3-oxo-2-(3-pyrrolidin-1-ylpropyl)isoindoline-1-carboxamide;
N-(tert-butyl)-3-oxo-2-(tetrahydrofuran-2-ylmethyl)isoindoline-1-carboxamide;
2-[1-(hydroxymethyl)butyl]-N-isopropyl-3-oxoisoindoline-1-carboxamide;
N-isopropyl-2-(3-methylbutyl)-3-oxoisoindoline-1-carboxamide;
N-(tert-butyl)-2-cyclohexyl-3-oxoisoindoline-1-carboxamide;
N-(tert-butyl)-2-(3-methylbutyl)-3-oxoisoindoline-1-carboxamide;
methyl N-{[2-(3-methylbutyl)-3-oxo-2,3-dihydro-1H-isoindol-1-yl]carbonyl}glycinate;
tert-butyl N-({2-[1-(hydroxymethyl)butyl]-3-oxo-2,3-dihydro-1H-isoindol-1-yl}carbonyl)glycinate;
tert-butyl N-{[2-(3-methylbutyl)-3-oxo-2,3-dihydro-1H-isoindol-1-yl]carbonyl}glycinate;
N-(tert-butyl)-2-[1-(methoxymethyl)propyl]-3-oxoisoindoline-1-carboxamide;
N-(tert-butyl)-2-[2-(diethylamino)ethyl]-3-oxoisoindoline-1-carboxamide;
N-(tert-butyl)-2-[1-(hydroxymethyl)butyl]-3-oxoisoindoline-1-carboxamide;
tert-butyl N-{[3-oxo-2-(2-thienylmethyl)-2,3-dihydro-1H-isoindol-1-yl]carbonyl}glycinate;
tert-butyl N-({-2-[2-(methylthio)ethyl]-3-oxo-2,3-dihydro-1H-isoindol-1-yl}carbonyl)glycinate;
methyl N-{[2-(cyclopropylmethyl)-3-oxo-2,3-dihydro-1H-isoindol-1-yl]carbonyl}glycinate; or
2-(2,2-dimethylpropyl)-3-oxo-N-(4,4,4-trifluorobutyl)isoindoline-1-carboxamide.

2. A compound according to claim 1 wherein R¹ represents C₁-C₇ alkyl (which alkyl group is optionally substituted by one or more groups selected from halogen, C₂-C₆ alkenyl, C₃-C₈ cycloalkyl, cyano, oxo, —OR⁸, —COXR¹⁰, aryl and Het¹); further R¹ represents Het².

3. A compound according to claim 1 wherein R¹ represents C₁-C₇ alkyl (which alkyl group is optionally substituted by one or more groups selected from halogen, C₂-C₆ alkenyl, C₃-C₈ cycloalkyl, cyano, oxo, —OR⁸, —COXR¹⁰, phenyl, naphthalene and Het¹).

4. A compound according to claim 1 wherein R¹ represents
(1-benzylpyrrolidin-3-yl); (1-fluoro-3-phenyl-propan-2-yl); (1-methyl-5-phenyl-pyrazol-3-yl)methyl; (1-methylpyrrol-2-yl)methyl; (2,3-difluorophenyl)methyl; (2,4-difluorophenyl)methyl; (2,5-dimethoxyphenyl)methyl; (2,5-dimethylphenyl)methyl; (2-bromophenyl)methyl; (2-chloro-4-fluoro-phenyl)methyl; (2-chloro-6-phenoxy-phenyl)methyl; (2-chlorophenyl)methyl; (2-dimethylamino-2-phenyl-ethyl); (2-ethoxyphenyl)methyl; (2-fluorophenyl)methyl; (2-methoxyphenyl)methyl; (2-methyl-2-phenyl-propyl); (2-methylphenyl)methyl; (2-phenoxyphenyl)methyl; (2-phenylphenyl)methyl; (2-pyridin-3-ylphenyl)methyl; (3,4-dichlorophenyl)methyl; (3,4-difluorophenyl)methyl; (3,5-dimethoxyphenyl)methyl; (3-chlorophenyl)methyl; (3-cyano-4-fluoro-phenyl)methyl; (3-cyanophenyl)methyl; (3-fluorophenyl)methyl; (3-hydroxy-2,2-dimethyl-propyl); (3-methoxyphenyl)methyl; (3-phenyl1,2-oxazol-5-yl)methyl; (3-phenylphenyl)methyl; (3-pyrrol-1-ylphenyl)methyl; (4-chlorophenyl)methyl; (4-dimethylaminophenyl)methyl; (4-fluorophenyl)methyl; (4-hydroxyphenyl)methyl; (4-methoxycarbonylphenyl)methyl; (4-phenoxyphenyl)methyl; (4-phenylphenyl)methyl; (5-methyl-2-phenyl-1,3-oxazol-4-yl)methyl; (5-methyl-3-phenyl-1,2-oxazol-4-yl)methyl; (phenyl-pyridin-2-yl-methyl); [(1R)-1-(4-methoxyphenyl)ethyl]; [(1S)-1-phenylethyl]; [(1R)-1-phenylethyl]; [(1R)-2-(4-chlorophenyl)-1-(4,4,4-trifluorobutylcarbamoyl)ethyl]; [(1R)-2-(4-chlorophenyl)-1-methoxycarbonyl-ethyl]; [(1S)-1-naphthalen-1-ylethyl]; [(2R)-2-(4-chlorophenyl)propyl]; [(2S)-2-(4-chlorophenyl)propyl]; [(4-chlorophenyl)-pyridin-4-yl-methyl]; [(4-fluorophenyl)-pyridin-3-yl-methyl]; [(4-fluorophenyl)-pyridin-3-yl-methyl]; [(4-fluorophenyl)-pyridin-3-yl-methyl]; [2-(2,4-dichlorophenyl)phenyl]methyl; [2-(2,4-difluorophenyl)phenyl]methyl; [2-(2,5-difluorophenyl)phenyl]methyl; [2-(2-chlorophenyl)phenyl]methyl; [2-(3,4-dichlorophenyl)phenyl]methyl; [2-(3,4-difluorophenyl)phenyl]methyl; [2-(3-chloro-4-fluoro-phenyl)phenyl]methyl; [2-(3-fluorophenyl)phenyl]methyl; [2-(4-chloro-2-methyl-phenyl)-2,2-difluoro-ethyl]; [2-(4-chloro-2-methyl-phenyl)-2,2-difluoro-ethyl]; [2-(4-chlorophenyl)phenyl]methyl; [2-(4-fluoro-2-methyl-phenyl)phenyl]methyl; [2-(4-fluorophenoxy)phenyl]methyl; [2-(4-fluorophenyl)phenyl]methyl; [2-(4-methoxyphenyl)-2-oxo-ethyl]; [2-(4-methoxyphenyl)phenyl]methyl; [2-(4-methylphenyl)phenyl]methyl; [2-(trifluoromethyl)phenyl]methyl; [2-[4-(trifluoromethyl)phenoxy]phenyl]methyl; [3-(difluoromethoxy)phenyl]methyl; [3,5-bis(trifluoromethyl)phenyl]methyl; [4-(difluoromethoxy)phenyl]methyl; [4-(trifluoromethyl)phenyl]methyl; 1-(1H-indol-3-yl)propan-2-yl; 1-(4-fluorophenyl)ethyl; 1-naphthalen-1-ylethyl; 1 naphthalen-2-ylethyl; 1-phenylethyl; 1-phenylpropyl; 2-(1-cyclohexenyl)ethyl; 2-(2-ethoxyphenyl)ethyl; 2-(2-methoxyphenyl)ethyl; 2-(2-phenoxyphenyl)ethyl; 2-(3,4-dichlorophenyl)ethyl; 2-(3,5-dimethoxyphenyl)ethyl; 2-(3-bromo-4-methoxy-phenyl)ethyl; 2-(3-fluorophenyl)ethyl; 2-(4-bromophenyl)ethyl; 2-(4-chlorophenyl)ethyl; 2-(4-chlorophenyl)propyl; 2-(4-fluorophenoxy)propyl; 2-(4-fluorophenyl)ethyl; 2-(4-fluorophenyl)propyl; 2-(4-phenoxyphenyl)ethyl; 2-(4-methoxyphenyl)ethyl; 2-(4-methoxyphenyl)ethyl; 2-(4-phenylphenyl)ethyl; 2-(5-bromo-2-methoxy-phenyl)ethyl; 2-(6-chloro-1H-indol-3-yl)ethyl; 2,2-dimethylpropyl; 2,2-diphenylethyl; 2-[2-(trifluoromethoxy)phenyl]ethyl; 2-[3-(trifluoromethyl)phenyl]ethyl; 2-[4-(diethylcarbamoyl)phenyl]ethyl; 2-[4-(trifluoromethyl)phenyl]ethyl; 2-benzo[1,3]dioxol-5-ylethyl; 2-methylbutyl; 2-methylpropyl; 2-naphthalen-1-ylpropyl; 2-phenoxypropyl; 2-phenylpropyl; 2-thiophen-2-ylethyl; 3,3-dimethylbutyl; 3-phenylpropyl; 3-pyrrolidin-1-ylpropyl; 4-phenylbutan-2-yl; 4-phenylbutyl; 9H-fluoren-9-yl; benzhydryl; benzyl; cycloheptyl; cyclohexyl; cyclohexylmethyl; naphthalen-1-ylmethyl; pentan-3-yl; phenethyl; thiophen-2-ylmethyl; 2-phenylpropan-2-yl; 1-phenylpropyl; [2-(4-chlorophenyl)-2-methyl-propyl]; [4-fluoro-2-(4-fluorophenyl)phenyl]methyl; (4-fluoro-2-phenyl-phenyl)methyl; [5-fluoro-2-(4-fluorophenyl)phenyl]methyl; (5-fluoro-2-phenyl-phenyl)methyl; 1-(4 fluorophenyl)ethyl; 2-(4-chlorophenyl)propan-2-yl; 2-(4-fluorophenyl)propan-2-yl; or 1-(4-chlorophenyl)ethyl.

5. A compound according to claim 1 wherein R² represents C₁-C₆ alkyl (which alkyl group is optionally substituted by one or more groups selected from fluoro, C₂-C₆ alkenyl, C₃-C₈ cycloalkyl, —COR¹⁷, trimethylsilyl, —COXR¹⁸, aryl and Het³); further R² is aryl or Het⁴.

6. A compound according to claim 1 wherein R² represents (1-benzylpyrrolidin-3-yl); (1-methylpyrrol-2-yl)methyl; (2,2-difluorobenzo[1,3]dioxol-5-yl)methyl; (2,3-dimethylcyclohexyl); (2,4-difluorophenyl)methyl; (2-chloro-4-methylsulfonyl-phenyl)methyl; (2-chlorophenyl)methyl; (2-fluoro-4-methylsulfonyl-phenyl)methyl; (2-hydroxyphenyl)methyl; (2-methylpropan-2-yl)oxycarbonylmethyl; (3,4-dichlorophenyl)methyl; (3,4-difluorophenyl)methyl; (3,4-dimethoxyphenyl)methyl; (3-carbamoyl-4-fluoro-phenyl)methyl; (3-chlorophenyl)methyl; (3-cyano-4-fluoro-phenyl)methyl; (3-cyanophenyl)methyl; (3-methoxyphenyl); (3-methyl-5-phenyl-1,2-oxazol-4-yl)methyl; (4-amino-2-methyl-pyrimidin-5-yl)methyl; (4-carbamoylphenyl); (4-carbamoylphenyl)methyl; (4-cyano-2,6-difluoro-phenyl)methyl; (4-cyanophenyl); (4-cyanophenyl)methyl; (4-dimethylaminophenyl)methyl; (4-fluorophenyl)methyl; (4-hydroxyphenyl)methyl; (4-methylcyclohexyl); (4-methylsulfonylphenyl)methyl; (5-methyl-1,2-oxazol-3-yl)methyl; (5-methyl-2-furyl)methyl; (5-methyl-2-phenyl-1,3-oxazol-4-yl)methyl; (5-methylpyrazin-2-yl)methyl; [2-(trifluoromethyl)phenyl]methyl; [3-(aminomethyl)-4-fluoro-phenyl]methyl; [3-(difluoromethoxy)phenyl]methyl; [3-(dimethylcarbamoyl)-4-fluoro-phenyl]methyl; [3-(trifluoromethyl)phenyl]methyl; [3,5-bis(trifluoromethyl)phenyl]methyl; [3-[[(2,2-difluoroacetyl)amino]methyl]-4-fluoro-phenyl]methyl; [4-(acetamidomethyl)phenyl]methyl; [4-(aminomethyl)phenyl]; [4-(difluoromethoxy)phenyl]methyl; [4-(trifluoromethyl)phenyl]methyl; [4-[[(2,2-difluoroacetyl)amino]methyl]phenyl]; [4-[[(2-fluoroacetyl)amino]methyl]phenyl]methyl; [5-(2-furyl)1,2-oxazol-3-yl]methyl; [6-(trifluoromethyl)pyridin-3-yl]methyl; 1H-indol-3-ylmethyl; 1-pyridin-4-ylethyl; 2-(1H-indol-3-yl)ethyl; 2-(2,4-dichlorophenyl)ethyl; 2-(2,6-dichlorophenyl)ethyl; 2-(2-chlorophenyl)ethyl; 2-(3,4-dichlorophenyl)ethyl; 2-(3,4-dimethoxyphenyl)ethyl; 2-(3-chlorophenyl)ethyl; 2-(3-fluorophenyl)ethyl; 2-(4-benzoylpiperazin-1-yl)ethyl; 2-(4-chlorophenyl)ethyl; 2-(4-fluorophenyl)ethyl; 2-(4-methoxyphenyl)ethyl; 2-[3-(trifluoromethyl)phenyl]ethyl; 2-benzo[1,3]dioxol-5-ylethyl; 2-ethoxycarbonylethyl; 2-furylmethyl; 2-methoxyethyl; 2-pyridin-2-ylethyl; 2-pyridin-4-ylethyl; 2-thiophen-2-ylethyl; 3-imidazol-1-ylpropyl; 3-methoxypropyl; 4,4,4-trifluorobutyl; 4,4-difluorobutyl; benzo[1,3]dioxol-5-ylmethyl; benzotriazol-1-ylmethyl; benzyl; butyl; cyclohexyl; ethyl; methoxycarbonylmethyl; phenethyl; propan-2-yl; propyl; pyridin-3-ylmethyl; pyridin-4-ylmethyl; tert-butyl; trimethylsilylmethyl; (5-oxo-1-propan-2-yl-pyrrolidin-3-yl)methyl; propan-2-ylcarbamoylmethyl; (2-fluorophenyl)methyl; (3-fluorophenyl)methyl; 1-phenylethyl; 2-phenylpropan-2-yl or 5-cyanopentyl.

7. A compound according to claim 1 wherein
R¹ represents C₁-C₇ alkyl (which alkyl group is optionally substituted by one or more groups selected from fluoro, C₂-C₆ alkenyl, C₃-C₈ cycloalkyl, cyano, oxo, —OR⁸, —COXR¹¹, aryl and Het¹); further R¹ represents Het²; and
R² represents C₁-C₆ alkyl (which alkyl group is optionally substituted by one or more groups selected from fluoro, C₂-C₆ alkenyl, C₃-C₇ cycloalkyl, —COR¹⁷, trimethylsilyl, —COXR¹⁸, aryl and Het³); further R² represents aryl or Het⁴.

8. A compound according to claim 1 wherein R³ represents hydrogen or C₁-C₄ alkyl (which alkyl group is optionally substituted by one or more groups selected from fluoro, C₂-C₆ alkenyl, trialkylsilyl, —COXR²⁷, aryl and Het⁵).

9. A compound according to claim 1 wherein R³ represents hydrogen.

10. A compound according to claim 1 wherein R⁴ represents hydrogen.

11. A compound according to claim 1 wherein R⁵ to R⁷ independently represent, at each occurrence, hydrogen, —OH, halogen, cyano, C₁₋₆ alkyl, —OR³⁶, —C(O)N(R^{40a})(R^{40b}), or —N(R^{44a})S(O)₂R^{44b}.

12. A compound according to claim 1 wherein aryl is independently, at each occurrence, optionally substituted by —OH, halogen, cyano, nitro, C₁-C₆ alkyl, —OR⁵⁰, C₂-C₆ alkenyl, aryl, or Het⁸; wherein R⁵⁰ represents C₁-C₆ alkyl or phenyl.

13. A compound according to claim 1 wherein aryl is, at each occurrence, phenyl.

14. A compound according to claim 1 wherein the compound of formula I is 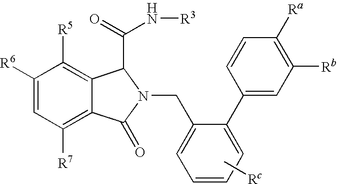
in which
R^{a} is hydrogen or fluoro;
R^{b} is hydrogen or fluoro;
R^{c} is hydrogen or fluoro;
R³ is C₁₋₄ alkyl optionally terminally substituted by 1, 2 or 3 fluoro;
R⁵ is hydrogen or C₁₋₄ alkyl,
R⁶ is hydrogen, OH, halo or C₁₋₄alkoxy; and
R⁷ is hydrogen or halo.

15. A compound according to claim 1 wherein the compound of formula I is 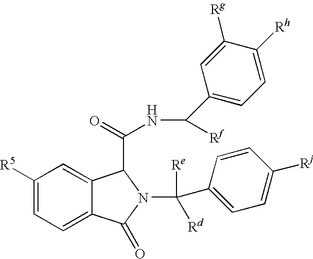
in which
R^{d} is hydrogen or C₁₋₄ alkyl;
R^{e} is hydrogen or C₁₋₄ alkyl;
R^{f} is hydrogen or C₁₋₄ alkyl;
R^{g} is hydrogen or halo;
R^{h} is hydrogen or halo;
R^{j} is hydrogen or halo; and
R⁵ is hydrogen or halo.

16. A compound according to claim 1 wherein the compound of formula I is 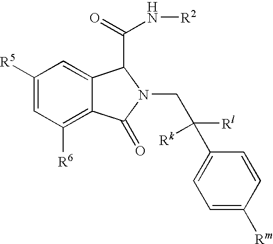
in which
R^{k} is hydrogen or C₁₋₄ alkyl;
R^{l} is hydrogen or C₁₋₄ alkyl;
R^{m} is hydrogen or halo;
R² is C₃₋₆ alkyl;
R⁵ is hydrogen or halo; and
R⁶ is hydrogen or halo.

17. A compound according to claim 1 wherein the compound of formula I is 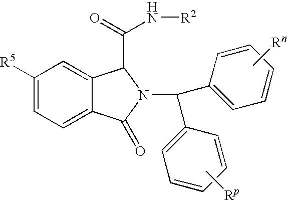
in which
Rⁿ is hydrogen or halo;
R^{p} is hydrogen or halo;
R² is C₃₋₆ alkyl or benzyl, optionally substituted by halo in the phenyl ring; and
R⁵ is hydrogen or halo.

18. A compound according to claim 1 wherein
R¹ is (2-phenylphenyl)methyl, optionally substituted by one to three fluoro;
R² is selected from ethyl, propyl, n-butyl, tert-butyl, 4,4,4-trifluorobutyl, 4,4-difluorobutyl, 4-fluorobutyl, benzo[1,3]dioxol-5-yl-methyl, (2,2-difluorobenzo[1,3]dioxol-5-yl)methyl, benzyl, (2-chlorophenyl)methyl, (4-fluorophenyl)methyl, (2-trifluoromethylphenyl)methyl, (3-cyanophenyl)methyl, (4-cyanophenyl)methyl, (3-cyano-4-fluorophenyl)methyl, (4-carbamoylphenyl)methyl, (5-methylpyrazin-2-yl)methyl, pyridin-3-ylmethyl, (4-amino-2-methyl-pyrimidin-5-yl)methyl, [6-(trifluoromethyl)pyridin-3-yl]methyl, pyridin-3-ylmethyl, [6-(trifluoromethyl)pyridin-3-yl]methyl, pyridin-4-ylmethyl, [4-[[(2,2-difluoroacetyl)amino]methyl]phenyl]methyl, [4-(acetamidomethyl)phenyl]methyl, [4-[[(2-fluoroacetyl)amino]methyl]phenyl]methyl, 2-phenylethyl and 2-(4-fluorophenyl)ethyl;
R⁵ to R⁷ are independently selected from —OH, methyl, methoxy, chloro, fluoro, cyano, methylsulfonylamino, fluoromethoxy, difluoromethoxy, and trifluoromethanesulfonate;
or an enantiomer thereof.

19. A compound according to claim 1 wherein
R¹ is benzhydryl, optionally substituted by one or more substituents selected from fluoro and chloro;
R² is selected from ethyl, propyl, butyl, tert-butyl, 4,4-difluorobutyl, 4,4,4-trifluorobutyl, benzyl, (2-chloro-4-methylsulfonyl-phenyl)methyl, (4-methylsulfonylphenyl)methyl, (2-fluoro-4-methylsulfonyl-phenyl)methyl, (4-methylsulfonylphenyl)methyl, (2-hydroxyphenyl)methyl, [2-(trifluoromethyl)phenyl]methyl, (2,4-difluorophenyl)methyl, (2-chlorophenyl)methyl of and 2-(4-fluorophenyl)ethyl;
R³ is hydrogen;
R⁴ is hydrogen;
R⁵ to R⁷ are independently selected from hydrogen, —OH, methyl, methoxy, fluoro and chloro;
or an enantiomer thereof.

20. A compound according to claim 1 wherein
R¹ is 4-phenylbutan-2-yl, optionally substituted by one or more substituents selected from fluoro and chloro;
R² is selected from (2-chlorophenyl)methyl, [2-(trifluoromethyl)phenyl]methyl, benzyl, and 2-phenylethyl;
R³ is hydrogen;
R⁴ is hydrogen;
R⁵ to R⁷ are independently selected from hydrogen, —OH, methyl, methoxy, fluoro of and chloro;
or an enantiomer thereof.

21. A compound according to claim 1 wherein
R¹ is 3,3-dimethylbutyl;
R² is selected from [3-(difluoromethoxy)phenyl]methyl, [3-(trifluoromethoxy)phenyl]methyl, 2-(1H-indol-3-yl)ethyl, 1H-indol-3-ylmethyl, (3-chlorophenyl)methyl, (3,4-dichlorophenyl)methyl, [4-(difluoromethoxy)phenyl]methyl, 2-(3-fluorophenyl)ethyl, 2-benzo[1,3]dioxol-5-ylethyl, 2-[3-(trifluoromethyl)phenyl]ethyl, 2-(3,4-dichlorophenyl)ethyl, 2-(2,4-dichlorophenyl)ethyl, 2-(2,6-dichlorophenyl)ethyl, 2-(4-chlorophenyl)ethyl, 2-(3-chlorophenyl)ethyl of and 2-(2-chlorophenyl)ethyl;
R³ is hydrogen;
R⁴ is hydrogen;
R⁵ to R⁷ are independently selected from hydrogen, —OH, methyl, methoxy, fluoro and chloro;
or an enantiomer thereof.

22. A compound according to claim 1 wherein
R¹ is benzyl, optionally substituted by one or more substituents selected from fluoro, chloro, and cyano;
R² is selected from ethyl, n-propyl, isopropyl, n-butyl, sec-butyl, tert-butyl, benzo[1,3]dioxol-5-yl-methyl, benzyl, 1-phenylethyl, 2-phenylethyl, and cyclopentyl, which groups are optionally substituted by one or more substituents selected from fluoro, chloro, cyano, and trifluoromethyl;
further R² represents pyridin-3-ylmethyl, pyridin-4-ylmethyl, [3-[[(2,2-difluoroacetyl)amino]methyl]-4-fluoro-phenyl]methyl, [4-(difluoromethoxy)phenyl]methyl, (4-dimethylaminophenyl)methyl, [5-(2-furyl)1,2-oxazol-3-yl]methyl, [5-(2-furyl)1,2-oxazol-3-yl]methyl, 2-(3,4-dimethoxyphenyl)ethyl, butan-2-yl, cyclopentyl, (2,3-dimethylcyclohexyl), (4-hydroxyphenyl)methyl, or [2-(trifluoromethyl)phenyl]methyl;
R³ is hydrogen;
R⁴ is hydrogen;
R⁵ to R⁷ are independently selected from hydrogen, —OH, methyl, methoxy, bromo, chloro, fluoro, and trimethylsilyl;
or an enantiomer thereof.

23. A compound according to claim 1 wherein
R¹ is (2-cyclopentylphenyl)methyl;
R² is 4,4-difluorobutyl, or methyl;
R³ is hydrogen;
R⁴ is hydrogen;
R⁵ to R⁷ are independently selected from bromo, fluoro, chloro and cyano;
or an enantiomer thereof.

24. A compound according to claim 1 wherein
R¹ is 1-phenylethyl, optionally substituted by one or more substituents selected from fluoro, chloro, cyano, and methoxy;
R² is selected from ethyl, propyl, tert-butyl, 4,4-difluorobutyl, 4,4,4-trifluorobutyl, 4-methylsulfonyl, and benzyl, which benzyl group is optionally substituted by one or more substituents selected from fluoro, chloro, and cyano; further R² represents pyridinmethyl, ((2,2-difluoroacetyl)amino)methyl, difluoromethoxy, dimethylamino, 5-(2-furyl)1,2-oxazol-3-yl-methyl, cyclopentyl;
R³ is hydrogen;
R⁴ is hydrogen;
R⁵ to R⁷ are independently selected from bromo, fluoro, chloro Of and cyano;
or an enantiomer thereof.

25. A compound according to claim 1 wherein
R¹ is 3-hydroxy-2,2-dimethylpropyl;
R² is selected from [3-(difluoromethoxy)phenyl]methyl, (3,4-dichlorophenyl)methyl, (3-chlorophenyl)methyl, and [3-(trifluoromethyl)phenyl]methyl;
R³ is hydrogen;
R⁴ is hydrogen;
R⁵ to R⁷ are independently selected from hydrogen, fluoro, and chloro;
or an enantiomer thereof.

26. A compound according to claim 1 wherein
R¹ is 2-(4-chlorophenyl)propyl;
R² is selected from methyl, ethyl, n-propyl, propan-2-yl, butyl, (4-amino-2-methyl-pyrimidin-5-yl)methyl, (5-methylpyrazin-2-yl)methyl, pyridin-3-ylmethyl, [6-(trifluoromethyl)pyridin-3-yl]methyl, (4-amino-2-methyl-pyrimidin-5-yl)methyl, [6-(trifluoromethyl)pyridin-3-yl]methyl, (5-methyl-2-phenyl-1,3-oxazol-4-yl)methyl, 4,4,4-trifluorobutyl, (4-methylsulfonylphenyl)methyl, benzyl, (2,2-difluorobenzo[1,3]dioxol-5-yl)methyl, (4-methylsulfonylphenyl)methyl, butyl, 2-(1H-indol-3-yl)ethyl; (4-carbamoylphenyl)methyl, (4-cyanophenyl)methyl, [3-(dimethylcarbamoyl)-4-fluoro-phenyl]methyl, [3-(dimethylcarbamoyl)-4-fluoro-phenyl]methyl, [4-(aminomethyl)phenyl], [4-[[(2,2-difluoroacetyl)amino]methyl]phenyl], (4-carbamoylphenyl), pyridin-4-ylmethyl, 3-methoxypropyl, (3-cyano-4-fluoro-phenyl)methyl, [3-[[(2,2-difluoroacetyl)amino]methyl]-4-fluoro-phenyl]methyl, [3-(aminomethyl)-4-fluoro-phenyl]methyl, (3-carbamoyl-4-fluoro-phenyl)methyl, 2-pyridin-4-ylethyl, (1-methylpyrrol-2-yl)methyl, [4-(difluoromethoxy)phenyl]methyl, (1-benzylpyrrolidin-3-yl), 3-imidazol-1-ylpropyl, (4-dimethylaminophenyl)methyl, (4-methylsulfonylphenyl)methyl, 3-dimethylaminopropyl, 1-pyridin-3-ylethyl, (3-methoxyphenyl), 1-pyridin-4-ylethyl, (4-cyanophenyl), 3-methoxypropyl, benzo[1,3]dioxol-5-ylmethyl, (3,4-dimethoxyphenyl)methyl, (3-methyl-5-phenyl-1,2-oxazol-4-yl)methyl, (5-methyl 1,2-oxazol-3-yl)methyl, [2-(trifluoromethyl)phenyl]methyl, (2-chlorophenyl)methyl, 2-(3,4-dimethoxyphenyl)ethyl, 2-thiophen-2-ylethyl, 2-(4-methoxyphenyl)ethyl, phenethyl, 2-methoxyethyl, (4-fluorophenyl)methyl, methoxycarbonylmethyl, and benzotriazol-1-ylmethyl;
R³ is hydrogen;
R⁴ is hydrogen;
R⁵ to R⁷ are independently selected from hydrogen, bromo, fluoro, and chloro;
or an enantiomer thereof.

27. A compound according to claim 1 wherein
R¹ is 2-(4-chlorophenyl)propyl;
R² is tert-butyl;
R³ is hydrogen;
R⁴ is hydrogen;
R⁵-R⁷ are independently selected from hydrogen, —OH, bromo, fluoro, chloro, methyl, —OCH₃, —OCH₂F, and trimethylsilyl;
or an enantiomer thereof.

28. A compound according to claim 1 wherein
R¹ is 2-(4-fluorophenyl)propyl;
R² is 4,4,4-trifluorobutyl, benzyl, tert-butyl, or butyl,
R³ is hydrogen;
R⁴ is hydrogen;
R⁵ to R⁷ are independently selected from hydrogen, —OH, bromo, fluoro, chloro, and methoxy;
or an enantiomer thereof.

29. A compound according to claim 1 wherein
R¹ is 2,2-dimethylpropyl;
R² is [3-(trifluoromethoxy)phenyl]methyl, [3-(difluoromethoxy)phenyl]methyl, (3,4-dichlorophenyl)methyl, 2-[3-(trifluoromethyl)phenyl]ethyl, 2-(1H-indol-3-yl)ethyl, (3-chlorophenyl)methyl, [4-(difluoromethoxy)phenyl]methyl, [3-(trifluoromethyl)phenyl]methyl, 2-(3-fluorophenyl)ethyl, 2-(2-chlorophenyl)ethyl, 2-(3-chlorophenyl)ethyl, 2-(2,4-dichlorophenyl)ethyl, 2-(4-chlorophenyl)ethyl, 2-(2,6-dichlorophenyl)ethyl, benzo[1,3]dioxol-5-ylmethyl, or phenylmethyl;
R³ is hydrogen;
R⁴ is hydrogen;
R⁵ to R⁷ are independently selected from hydrogen, bromo, fluoro, and chloro, or an enantiomer thereof.

30. A compound according to claim 1 wherein
R¹ is 2-phenylpropan-2-yl;
R² is benzyl, 1-phenylethyl, or (4-fluorophenyl)methyl,4,4,4-trifluorobutyl;
R³ is hydrogen;
R⁴ is hydrogen;
R⁵ to R⁷ are independently selected from hydrogen, bromo, fluoro, and chloro, or an enantiomer thereof.

31. A compound according to claim 1 wherein
R¹ is 1-phenylpropyl;
R² is benzyl, (2-chlorophenyl)methyl, [2-(trifluoromethyl)phenyl]methyl or (4-dimethylaminophenyl)methyl;
R³ is hydrogen;
R⁴ is hydrogen;
R⁵ to R⁷ are independently selected from hydrogen, bromo, fluoro, and chloro;
or an enantiomer thereof.

32. A compound according to claim 1 wherein
R¹ is [2-(4-chlorophenyl)-2-methyl-propyl];
R² is n-butyl;
R³ is hydrogen;
R⁴ is hydrogen;
R⁵ to R⁷ are independently selected from hydrogen, bromo, fluoro and chloro;
or an enantiomer thereof.

33. A compound according to claim 1 wherein
R² is (2-chlorophenyl)methyl;
R¹ is benzhydryl, (2-pyridin-3-ylphenyl)methyl, (3,4-difluorophenyl)methyl 1-(1H-indol-3-yl)propan-2-yl, 2-(4-chlorophenyl)propyl, (2,5-dimethylphenyl)methyl, [(1R)-1-(4-methoxyphenyl)ethyl], 2-(1H-indol-3-yl)propyl, [(1R)-1-(3-methoxyphenyl)ethyl], [(1S)-1-naphthalen-1-ylethyl], 1-phenylpropyl, 2-phenylpropyl, 3-phenylpropyl, 2-phenethyl, 4-phenylbutan-2-yl, or (2-phenylphenyl)methyl;
R³ is hydrogen;
R⁴ is hydrogen;
R⁵ to R⁷ are independently selected from hydrogen, bromo, fluoro and chloro;
or an enantiomer thereof.

34. A compound according to claim 1 wherein
R² is (3,4-dichlorophenyl)methyl;
R¹ is (3-hydroxy-2,2-dimethyl-propyl), 2,2-dimethylpropyl, 2-methylpropyl or 3,3-dimethylbutyl;
R³ is hydrogen;
R⁴ is hydrogen;
R⁵ to R⁷ are independently selected from hydrogen, bromo, fluoro and chloro;
or an enantiomer thereof.

35. A compound according to claim 1 wherein
R² is (3-chlorophenyl)methyl;
R¹ is (3-hydroxy-2,2-dimethyl-propyl), 2-methylpropyl, 2,2-dimethylpropyl, 3,3-dimethylbutyl, (4-hydroxyphenyl)methyl or (3-cyanophenyl)methyl;
R³ is hydrogen;
R⁴ is hydrogen;
R⁵ to R⁷ are independently selected from hydrogen, bromo, fluoro and chloro;
or an enantiomer thereof.

36. A compound according to claim 1 wherein
R² is (3-cyano-4-fluoro-phenyl)methyl;
R¹ is (2-chloro-4-fluoro-phenyl)methyl, [3,5-bis(trifluoromethyl)phenyl]methyl, (3-cyano-4-fluoro-phenyl)methyl, 2-phenylethyl, benzyl, (3,4-difluorophenyl)methyl, (2-phenylphenyl)methyl or 2-(4-chlorophenyl)propyl;
R³ is hydrogen;
R⁴ is hydrogen;
R⁵ to R⁷ are independently selected from hydrogen, bromo, fluoro and chloro;
or an enantiomer thereof.

37. A compound according to claim 1 wherein
R² is (3-cyanophenyl)methyl;
R¹ is (2-phenylphenyl)methyl, (3-chlorophenyl)methyl, (3,4-difluorophenyl)methyl, [3,5-bis(trifluoromethyl)phenyl]methyl or [4-(trifluoromethyl)phenyl]methyl;
R³ is hydrogen;
R⁴ is hydrogen;
R⁵ to R⁷ are independently selected from hydrogen, bromo, fluoro and chloro;
or an enantiomer thereof.

38. A compound according to claim 1 wherein
R² is (4-fluorophenyl)methyl;
R¹ is [(4-chlorophenyl)-pyridin-4-yl-methyl], [(4-fluorophenyl)-pyridin-3-yl-methyl], (phenyl-pyridin-2-yl-methyl), 2-(4-methoxyphenyl)ethyl, (4-chlorophenyl)methyl (2-phenylphenyl)methyl, benzhydryl, (2-pyridin-3-ylphenyl)methyl, (3,4-difluorophenyl)methyl, (1-fluoro-3-phenyl-propan-2-yl), (1-methylpyrrol-2-yl)methyl, (2-phenylphenyl)methyl, 2-(4-chlorophenyl)propyl, 1-(4-chlorophenyl)ethyl, 2-(4-chlorophenyl)propan-2-yl, 2-(4-fluorophenyl)propan-2-yl, or 2-phenylpropan-2-yl;
R³ is hydrogen;
R⁴ is hydrogen;
R⁵ to R⁷ are independently selected from hydrogen, bromo, fluoro and chloro;
or an enantiomer thereof.

39. A compound according to wherein
R² is (4-hydroxyphenyl)methyl;
R¹ represents (3,4-difluorophenyl)methyl, (3-chlorophenyl)methyl, [3,5-bis(trifluoromethyl)phenyl]methyl or [4-(trifluoromethyl)phenyl]methyl;
R³ is hydrogen;
R⁴ is hydrogen;
R⁵ to R⁷ are independently selected from hydrogen, bromo, fluoro and chloro;
or an enantiomer thereof.

40. A compound according to claim 1 wherein
R² is [(2-trifluoromethyl)phenyl]methyl;
R¹ is (2-methoxyphenyl)methyl, (2-fluorophenyl)methyl, benzhydryl, 2-(4-chlorophenyl)ethyl, [4-(piperidine-1-carbonyl)phenyl]methyl, 2-(4-chlorophenyl)propyl, (2-phenylphenyl)methyl, 1-phenylpropyl, 2-phenylpropyl, 4-phenylbutan-2-yl, 2-phenylethyl, 3-phenylpropyl, 2-methylbutyl, cyclohexylmethyl, (3-fluorophenyl)methyl, (2-ethoxyphenyl)methyl, [4-(trifluoromethoxy)phenyl]methyl or (3,4-difluorophenyl)methyl
R³ is hydrogen;
R⁴ is hydrogen;
R⁵ to R⁷ are independently selected from hydrogen, bromo, fluoro, chloro, methoxy e.g. and methyl;
or an enantiomer thereof.

41. A compound according to claim 1 wherein
R² is [3-difluoromethoxy)phenyl]methyl;
R¹ is 1-phenylethyl, (3-hydroxy-2,2-dimethyl-propyl), 3,3-dimethylbutyl or 2,2-dimethylpropyl;
R³ is hydrogen;
R⁴ is hydrogen;
R⁵-R⁷ are independently selected from hydrogen, bromo, fluoro and chloro;
or an enantiomer thereof.

42. A compound according to claim 1 wherein
R² is [3-trifluoromethoxy)phenyl]methyl;
R¹ represents 3,3-dimethylbutyl or 2,2-dimethylpropyl;
R³ is hydrogen;
R⁴ is hydrogen;
R⁵ to R⁷ are independently selected from hydrogen, bromo, fluoro and chloro;
or an enantiomer thereof.

43. A compound according to claim 1 wherein
R² is [3-trifluoromethyl)phenyl]methyl;
R¹is (3-hydroxy-2,2-dimethyl-propyl), 2-methylpropyl, 3,3-dimethylbutyl, 2,2-dimethylpropyl or (1-methylpyrrol-2-yl)methyl;
R³ is hydrogen;
R⁴ is hydrogen;
R⁵ to R⁷ are independently selected from hydrogen, bromo, fluoro and chloro;
or an enantiomer thereof.

44. A compound according to claim 1 wherein
R² is [4-difluoromethoxy)phenyl]methyl;
R¹ is 2-methylpropyl, 3,3-dimethylbutyl, 2,2-dimethylpropyl, (2-chloro-4-fluoro-phenyl)methyl, 2-(4-chlorophenyl)propyl or [3,5-bis(trifluoromethyl)phenyl]methyl;
R³ is hydrogen;
R⁴ is hydrogen;
R⁵ to R⁷ are independently selected from hydrogen, bromo, fluoro and chloro;
or an enantiomer thereof.

45. A compound according to claim 1 wherein
R² is [6-(trifluoromethyl)pyridin-3-yl]methyl;
R¹ is 2-(4-chlorophenyl)propyl or (2-phenylphenyl)methyl;
R³ is hydrogen;
R⁴ is hydrogen;
R⁵ to R⁷ are independently selected from hydrogen, bromo, fluoro and chloro;
or an enantiomer thereof.

46. A compound according to claim 1 wherein
R² is 2-(1H-indol-3-yl)ethyl;
R¹ is 2-(4-chlorophenyl)propyl, 2-(2-phenoxyphenyl)ethyl, 2-[4-(diethylcarbamoyl)phenyl]ethyl, 2-(3-fluorophenyl)ethyl, 2-[2-(trifluoromethoxy)phenyl]ethyl, 2-(4-fluorophenyl)ethyl, 2-(3,5-dimethoxyphenyl)ethyl, 2-(4-phenylphenyl)ethyl, 2-(4-phenoxyphenyl)ethyl, 2-(2-ethoxyphenyl)ethyl or 2-benzo[1,3]dioxol-5-ylethyl, 2,2-dimethylpropyl; or 3,3-dimethylbutyl;
R³ is hydrogen;
R⁴ is hydrogen;
R⁵ to R⁷ are independently selected from hydrogen, bromo, fluoro and chloro;
or an enantiomer thereof.

47. A compound according to claim 1 wherein
R² is 2-(2,4-dichlorophenyl)ethyl;
R¹ is 2-methylpropyl, 3,3-dimethylbutyl or 2,2-dimethylpropyl;
R³ is hydrogen;
R⁴ is hydrogen;
R⁵ to R⁷ are independently selected from hydrogen, bromo, fluoro and chloro;
or an enantiomer thereof.

48. A compound according to claim 1 wherein
R² is 2-(2,6-dichlorophenyl)ethyl;
R¹ is 2-methylpropyl, 3,3-dimethylbutyl or 2,2-dimethylpropyl;
R³ is hydrogen;
R⁴ is hydrogen;
R⁵ to R⁷ are independently selected from hydrogen, bromo, fluoro and chloro;
or an enantiomer thereof.

49. A compound according to claim 1 wherein
R² is 4,4,4-trifluorobutyl;
R¹ is [2-(trifluoromethyl)phenyl]methyl, [(1R)-1-phenylethyl], benzhydryl, 2-(4-chlorophenyl)propyl, (2-phenylphenyl)methyl, (2-phenoxyphenyl)methyl, (2-phenylphenyl)methyl, 2-(4-chlorophenyl)ethyl, 2-(4-fluorophenyl)ethyl, 2-(4-fluorophenyl)propyl, 2-(4-chlorophenyl)propan-2-yl, 2-(4-fluorophenyl)propan-2-yl or 2-phenylpropan-2-yl;
R³ is hydrogen;
R⁴ is hydrogen;
R⁵ to R⁷ are independently selected from hydrogen, —OH, methyl, bromo, fluoro and chloro;
or an enantiomer thereof.

50. A compound according to claim 1 wherein
R² is 4,4-difluorobutyl;
R¹ is (2-cyclopentylphenyl)methyl, [2-(trifluoromethyl)phenyl]methyl, [(1R)-1-phenylethyl], (2-phenylphenyl)methyl, benzhydryl, 2-(4-chlorophenyl)propyl or (2-phenylphenyl)methyl;
R³ is hydrogen;
R⁴ is hydrogen;
R⁵ to R⁷ are independently selected from hydrogen, bromo, fluoro and chloro;
or an enantiomer thereof.

51. A compound according to claim 1 wherein
R² is benzyl;
R¹ is benzyl, benzhydryl, [2-(trifluoromethyl)phenyl]methyl, (2-pyridin-3-ylphenyl)methyl, (4-phenoxyphenyl)methyl, (2,4-difluorophenyl)methyl, [4-(difluoromethoxy)phenyl]methyl, [3-(difluoromethoxy)phenyl]methyl, (3-pyrrol-1-ylphenyl)methyl, (3-fluorophenyl)methyl, (4-cyanophenyl)methyl, (3,5-dimethoxyphenyl)methyl, (2-methoxyphenyl)methyl, (2-ethoxyphenyl)methyl, [4-(trifluoromethyl)phenyl]methyl, (3,4-difluorophenyl)methyl, (2,5-dimethylphenyl)methyl, [3,5-bis(trifluoromethyl)phenyl]methyl, (2-methylphenyl)methyl, (2,3-difluorophenyl)methyl, (2-bromophenyl)methyl, [(4-fluorophenyl)-pyridin-3-yl-methyl], [(4-chlorophenyl)-pyridin-4-yl-methyl], (phenyl-pyridin-2-yl-methyl), (1-methyl-5-phenyl-pyrazol-3-yl)methyl, (5-methyl-2-phenyl-1,3-oxazol-4-yl)methyl, (5-methyl-3-phenyl-1,2-oxazol-4-yl)methyl, (3-phenyl1,2-oxazol-5-yl)methyl, 2-(4-chlorophenyl)ethyl, 2-(4-fluorophenyl)ethyl, 2-[4-(trifluoromethyl)phenyl]ethyl, 2-(5-bromo-2-methoxy-phenyl)ethyl, 2-(3-bromo-4-methoxy-phenyl)ethyl, 2-(4-fluorophenyl)propyl, 2-(4-chlorophenyl)propyl, 4-phenylbutan-2-yl, [2-(4-chloro-2-methyl-phenyl)-2,2-difluoro-ethyl], 2-naphthalen-1-ylpropyl, (2-methyl-2-phenyl-propyl), 2-phenoxypropyl, 2-(4-fluorophenoxy)propyl, 2-phenylpropan-2-yl, cycloheptyl, 2-(2-methoxyphenyl)ethyl, 1-naphthalen-1-ylethyl, 2-[3-(trifluoromethyl)phenyl]ethyl, 2-(6-chloro-1H-indol-3-yl)ethyl, 2-(4-chlorophenyl)propyl, [(1R)-1-(4-methoxyphenyl)ethyl], [(1R)-1-(3-methoxyphenyl)ethyl], 4-phenylbutan-2-yl, 1-phenylethyl, 2-phenylethyl, 1-naphthalen-2-ylethyl, 2-(1-cyclohexenyl)ethyl, 1-(4-fluorophenyl)ethyl, 2-(4-fluorophenyl)propan-2-yl, 2-phenylpropan-2-yl, 1-phenylpropyl or (2-phenylphenyl)methyl;
R³ is hydrogen;
R⁴ is hydrogen;
R⁵ to R⁷ are independently selected from hydrogen, bromo, fluoro and chloro, —OH, methyl, and methoxy;
or an enantiomer thereof.

52. A compound according to claim 1 wherein
R² is n-butyl;
R¹ is (2-phenylphenyl)methyl, (2-phenoxyphenyl)methyl, [2-(4-fluorophenoxy)phenyl]methyl, 2-(3-fluorophenyl)ethyl, 2-(4-fluorophenyl)ethyl, 2-(4-chlorophenyl)ethyl, 2-[4-(trifluoromethyl)phenyl]ethyl, 2-(4-chlorophenyl)propyl, 2-(4-fluorophenyl)propyl, (2-phenylphenyl)methyl, 2-(4-phenylphenyl)ethyl, 2-naphthalen-1-ylpropyl, 2-(2-ethoxyphenyl)ethyl, 2-(2-phenoxyphenyl)ethyl, 2-(4-phenoxyphenyl)ethyl, 2-[2-(trifluoromethoxy)phenyl]ethyl, 2-(3,5-dimethoxyphenyl)ethyl, 2-benzo[1,3]dioxol-5-ylethyl, (1-fluoro-3-phenyl-propan-2-yl), 2-(4-chlorophenyl)propyl, naphthalen-1-ylmethyl, 1-naphthalen-2-ylethyl, (2-phenylphenyl)methyl, or [2-(4-chlorophenyl)-2-methyl-propyl];
R³ is hydrogen;
R⁴ is hydrogen;
R⁵ to R⁷ are independently selected from hydrogen, bromo, fluoro, chloro, —OH, methyl, and methoxy;
or an enantiomer thereof.

53. A compound according to claim 1 wherein
R² is ethyl;
R¹ is benzhydryl, (2-phenylphenyl)methyl or 2-(4-chlorophenyl)propyl
R³ is hydrogen;
R⁴ is hydrogen;
R⁵ to R⁷ are independently selected from hydrogen, bromo, fluoro, chloro, —OH, methyl, and methoxy;
or an enantiomer thereof.

54. A compound according to claim 1 wherein
R² is 2-phenylethyl;
R¹ is (2-phenylphenyl)methyl, 2-(4-methoxyphenyl)ethyl, (4-chlorophenyl)methyl, 2-(4-chlorophenyl)ethyl, 2-(3,4-dichlorophenyl)ethyl, (3,4-difluorophenyl)methyl, 2-(4-chlorophenyl)propyl, (2-chloro-4-fluoro-phenyl)methyl or 4-phenylbutan-2-yl;
R³ is hydrogen;
R⁴ is hydrogen;
R⁵ to R⁷ are independently selected from hydrogen, bromo, fluoro, chloro, —OH, methyl, and methoxy;
or an enantiomer thereof.

55. A compound according to claim 1 wherein
R² is propyl;
R¹ is (2-phenylphenyl)methyl, benzhydryl or 2-(4-chlorophenyl)propyl;
R³ is hydrogen;
R⁴ is hydrogen;
R⁵ to Ware independently selected from hydrogen, bromo, fluoro, chloro, —OH, methyl, and methoxy;
or an enantiomer thereof.

56. A compound according to claim 1 wherein
R² is pyridin-3-ylmethyl or pyridin-4-ylmethyl;
R¹ is (2-phenylphenyl)methyl, 2-(4-chlorophenyl)propyl, (3,4-difluorophenyl)methyl, (2-chloro-4-fluoro-phenyl)methyl or 1-(4-fluorophenyl)ethyl;
R³ is hydrogen;
R⁴ is hydrogen;
R⁵ to R⁷ are independently selected from hydrogen, bromo, fluoro, chloro, and —OH;
or an enantiomer thereof.

57. A compound according to claim 1 wherein
R² is tert-butyl;
R¹ is (2-phenylphenyl)methyl, [2-(trifluoromethyl)phenyl]methyl, [4-(difluoromethoxy)phenyl]methyl, (2-chlorophenyl)methyl, (2-methoxyphenyl)methyl, (3,4-difluorophenyl)methyl, (3,4-difluorophenyl)methyl, (4-phenoxyphenyl)methyl, [3,5-bis(trifluoromethyl)phenyl]methyl, (4-fluoro-2-phenyl-phenyl)methyl, (5-fluoro-2-phenyl-phenyl)methyl, 1-phenylethyl, 2-(4-chlorophenyl)ethyl, 2-(2-phenoxyphenyl)ethyl, 2-[2-(trifluoromethoxy)phenyl]ethyl, 2,2-diphenylethyl, 2-(4-fluorophenyl)propyl, 2-(4-chlorophenyl)propyl, (2-phenylphenyl)methyl, 2-(4-phenylphenyl)ethyl, [2-(3-fluorophenyl)phenyl]methyl, [2-(4-fluorophenyl)phenyl]methyl, [2-(3,4-difluorophenyl)phenyl]methyl, [2-(2,4-difluorophenyl)phenyl]methyl, [2-(2,5-difluorophenyl)phenyl]methyl, [2-(2,4-dichlorophenyl)phenyl]methyl, [2-(3,4-dichlorophenyl)phenyl]methyl, [2-(2-chlorophenyl)phenyl]methyl, [2-(4-chlorophenyl)phenyl]methyl, [2-(4-methylphenyl)phenyl]methyl, [2-(4-fluoro-2-methyl-phenyl)phenyl]methyl, [2-(4-methoxyphenyl)phenyl]methyl, [4-fluoro-2-(4-fluorophenyl)phenyl]methyl, [2-(3-chloro-4-fluoro-phenyl)phenyl]methyl, [2-(4-fluoro-2-methyl-phenyl)phenyl]methyl, [5-fluoro-2-(4-fluorophenyl)phenyl]methyl, benzhydryl, [(1R)-2-(4-chlorophenyl)-1-(4,4,4-trifluorobutylcarbamoyl)ethyl], [3,5-bis(trifluoromethyl)phenyl]methyl, 9H-fluoren-9-yl, [2-[4-(trifluoromethyl)phenoxy]phenyl]methyl, 2-naphthalen-1-ylpropyl, [(1R)-2-(4-chlorophenyl)-1-methoxycarbonyl-ethyl], (1-methyl-5-phenyl-pyrazol-3-yl)methyl or [2-(4-chloro-2-methyl-phenyl)-2,2-difluoro-ethyl], (3-phenylphenyl)methyl, (4-fluorophenyl)methyl, (4-phenylphenyl)methyl, [(4-chlorophenyl)-pyridin-4-yl-methyl], 2-(4-fluorophenyl)propyl or 2-(4-phenoxyphenyl)ethyl;
R³ is hydrogen;
R⁴ is hydrogen;
R⁵ to R⁷ are independently —OH, bromo, chloro, fluoro, methyl, methoxy methylsulfonylamino, trimethylsilyl, cyano, —OCHF₂, —OCH₂F, or —OSO₂CF₃, or an enantiomer thereof.

58. A compound according to claim 1 wherein
R² is trimethylsilylmethyl;
R¹ is [3-(difluoromethoxy)phenyl]methyl, [4-(difluoromethoxy)phenyl]methyl, naphthalen-1-ylmethyl, 1-naphthalen-1-ylethyl, 2-(4-bromophenyl)ethyl, (2-chloro-6-phenoxy-phenyl)methyl or (3,4-dichlorophenyl)methyl;
R³ is hydrogen;
R⁴ is hydrogen;
R⁵ to R⁷ are independently selected from hydrogen, bromo, fluoro, and chloro;
or an enantiomer thereof.

59. A compound selected from one or more of the following:
(1R or 1S)—N-(4,4-difluorobutyl)-2-(diphenylmethyl)-3-oxoisoindoline-1-carboxamide (E2);
(1S or 1R)—N-(4,4-difluorobutyl)-2-(diphenylmethyl)-3-oxoisoindoline-1-carboxamide (E1);
(1R or 1S)—N-benzyl-3-oxo-2-[(1S or 1R)-1-phenylethyl]isoindoline-1-carboxamide (E4);
(1S or 1R)—N-benzyl-3-oxo-2-[(1S or 1R)-1-phenylethyl]isoindoline-1-carboxamide (E3);
(1R or 1S)—N-benzyl-3-oxo-2-[(1R or 1S)-1-phenylethyl]isoindoline-1-carboxamide (E2);
(1S or 1R)—N-benzyl-3-oxo-2-[(1R or 1S)-1-phenylethyl]isoindoline-1-carboxamide (E1);
N-benzyl-6-cyano-3-oxo-2-[(1R)-1-phenylethyl]isoindoline-1-carboxamide;
2-(biphenyl-2-ylmethyl)-N-(tert-butyl)-5-[(methylsulfonyl)amino]-3-oxoisoindoline-1-carboxamide;
2-(biphenyl-2-ylmethyl)-N-(tert-butyl)-4-methyl-5-[(methylsulfonyl)amino]-3-oxoisoindoline-1-carboxamide;
N-(3-chlorobenzyl)-2-(3-hydroxy-2,2-dimethylpropyl)-3-oxoisoindoline-1-carboxamide;
2-(biphenyl-2-ylmethyl)-N-(tert-butyl)-6-cyano-3-oxoisoindoline-1-carboxamide;
N-benzyl-6-chloro-3-oxo-2-[(1R)-1-phenylethyl]isoindoline-1-carboxamide;
N-benzyl-2-[2-(4-chlorophenyl)ethyl]-1-hydroxy-3-oxoisoindoline-1-carboxamide;
2-[2-(4-chlorophenyl)propyl]-N-[(5-methyl-2-phenyl-1,3-oxazol-4-yl)methyl]-3-oxoisoindoline-1-carboxamide;
N-(tert-butyl)-2-[(1-methyl-5-phenyl-1H-pyrazol-3-yl)methyl]-3-oxoisoindoline-1-carboxamide;
2-(biphenyl-2-ylmethyl)-6-bromo-N-(tert-butyl)-3-oxoisoindoline-1-carboxamide;
N-(tert-butyl)-2-[(4′-fluorobiphenyl-2-yl)methyl]-5-hydroxy-4-methyl-3-oxoisoindoline-1-carboxamide;
2-(biphenyl-2-ylmethyl)-5-bromo-N-(tert-butyl)-3-oxoisoindoline-1-carboxamide;
N-butyl-2-[2-(4-fluorophenoxy)benzyl]-3-oxoisoindoline-1-carboxamide;
N-(4,4-difluorobutyl)-2-(diphenylmethyl)-3-oxoisoindoline-1-carboxamide;
2-(biphenyl-2-ylmethyl)-N-(4,4-difluorobutyl)-3-oxoisoindoline-1-carboxamide;
(R or S)2-(biphenyl-2-ylmethyl)-N-(tert-butyl)-5-hydroxy-4-methyl-3-oxoisoindoline-1-carboxamide (E1);
(S or R) 2-(biphenyl-2-ylmethyl)-N-(tert-butyl)-5-hydroxy-4-methyl-3-oxoisoindoline-1-carboxamide (E2);
2-(biphenyl-2-ylmethyl)-N-[(2,2-difluoro-1,3-benzodioxol-5-yl)methyl]-6-fluoro-3-oxoisoindoline-1-carboxamide;
2-(biphenyl-2-ylmethyl)-6-fluoro-3-oxo-N-(4,4,4-trifluorobutyl)isoindoline-1-carboxamide;
2-(biphenyl-2-ylmethyl)-1-[(tert-butylamino)carbonyl]-4-methyl-3-oxo-2,3-dihydro-1H-isoindol-5-yl methanesulfonate;
2-(biphenyl-2-ylmethyl)-N-(tert-butyl)-5-(difluoromethoxy)-4-methyl-3-oxoisoindoline-1-carboxamide;
2-(biphenyl-2-ylmethyl)-N-(tert-butyl)-5-(fluoromethoxy)-4-methyl-3-oxoisoindoline-1-carboxamide;
2-(biphenyl-2-ylmethyl)-1-[(tert-butylamino)carbonyl]-4-methyl-3-oxo-2,3-dihydro-1H-isoindol-5-yl trifluoromethanesulfonate;
N-(tert-butyl)-2-{(1R)-1-(4-chlorobenzyl)-2-oxo-2-[(4,4,4-trifluorobutyl)amino]ethyl}-3-oxoisoindoline-1-carboxamide;
N-butyl-2-[2-(4-chlorophenyl)ethyl]-N-methyl-3-oxoisoindoline-1-carboxamide;
2-(biphenyl-2-ylmethyl)-N-(tert-butyl)-4,7-difluoro-1-methyl-3-oxoisoindoline-1-carboxamide;
2-(biphenyl-4-ylmethyl)-N-(tert-butyl)-5-hydroxy-4-methyl-3-oxoisoindoline-1-carboxamide;
2-(biphenyl-2-ylmethyl)-N-(tert-butyl)-7-hydroxy-3-oxoisoindoline-1-carboxamide;
N-(tert-butyl)-2-(4-chlorobenzyl)-7-hydroxy-3-oxoisoindoline-1-carboxamide;
N-(tert-butyl)-2-(4-chlorobenzyl)-5-hydroxy-3-oxoisoindoline-1-carboxamide;
2-(biphenyl-2-ylmethyl)-N-(4-{[(difluoroacetyl)amino]methyl}benzyl)-3-oxoisoindoline-1-carboxamide;
N-{4-[(acetylamino)methyl]benzyl}-2-(biphenyl-2-ylmethyl)-3-oxoisoindoline-1-carboxamide;
2-(biphenyl-2-ylmethyl)-N-(4-{[(fluoroacetyl)amino]methyl}benzyl)-3-oxoisoindoline-1-carboxamide;
N-[4-(aminomethyl)benzyl]-2-(biphenyl-2-ylmethyl)-3-oxoisoindoline-1-carboxamide;
methyl (2R)-2-{1-[(tert-butylamino)carbonyl]-3-oxo-1,3-dihydro-2H-isoindol-2-yl}-3-(4-chlorophenyl)propanoate;
2-(biphenyl-2-ylmethyl)-N-(4-cyanobenzyl)-3-oxoisoindoline-1-carboxamide;
2-(biphenyl-2-ylmethyl)-N-(tert-butyl)-5-hydroxy-4-methyl-3-oxoisoindoline-1-carboxamide;
2-[2-(4-chlorophenyl)propyl]-N-[(5-methylisoxazol-3-yl)methyl]-3-oxoisoindoline-1-carboxamide;
(1S or 1R)—N-butyl-2-[(2R or 2S)-2-(4-chlorophenyl)propyl]-6-fluoro-3-oxoisoindoline-1-carboxamide (E2);
(1R or 1S)—N-butyl-2-[(2S or 2R)-2-(4-chlorophenyl)propyl]-6-fluoro-3-oxoisoindoline-1-carboxamide (E4);
(1R or 1S)—N-butyl-2-[(2R or 2S)-2-(4-chlorophenyl)propyl]-6-fluoro-3-oxoisoindoline-1-carboxamide (E3);
(1S or 1R)—N-butyl-2-[(2S or 2R)-2-(4-chlorophenyl)propyl]-6-fluoro-3-oxoisoindoline-1-carboxamide (E1);
N-butyl-2-[2-(4-chlorophenyl)propyl]-6-fluoro-3-oxoisoindoline-1-carboxamide;
(S or R) 2-(biphenyl-2-ylmethyl)-N-(tert-butyl)-3-oxoisoindoline-1-carboxamide;
(R or S) 2-(biphenyl-2-ylmethyl)-N-(tert-butyl)-3-oxoisoindoline-1-carboxamide;
N-benzyl-3-oxo-2-(1-phenylethyl)isoindoline-1-carboxamide;
2-(biphenyl-2-ylmethyl)-N-(tert-butyl)-3-oxoisoindoline-1-carboxamide;
2-(2-bromobenzyl)-N-tert-butyl-5-hydroxy-4-methyl-3-oxoisoindoline-1-carboxamide;
N-benzyl-2-(1-methyl-1-phenylethyl)-3-oxoisoindoline-1-carboxamide;
N-benzyl-3-oxo-2-(1-phenylpropyl)isoindoline-1-carboxamide;
N-[3-(difluoromethoxy)benzyl]-3-oxo-2-(1-phenylethyl)isoindoline-1-carboxamide;
N,2-dibenzyl-6-bromo-3-oxoisoindoline-1-carboxamide;
6-bromo-2-(2-cyclopentylbenzyl)-N-(4,4-difluorobutyl)-3-oxoisoindoline-1-carboxamide;
2-(2-cyclopentylbenzyl)-N-(4,4-difluorobutyl)-6-fluoro-3-oxoisoindoline-1-carboxamide;
6-bromo-2-(2-cyclopentylbenzyl)-N-methyl-3-oxoisoindoline-1-carboxamide;
2-(2-cyclopentylbenzyl)-6-fluoro-N-methyl-3-oxoisoindoline-1-carboxamide;
6-chloro-2-(2-cyclopentylbenzyl)-N-(4,4-difluorobutyl)-3-oxoisoindoline-1-carboxamide;
6-chloro-2-(2-cyclopentylbenzyl)-N-methyl-3-oxoisoindoline-1-carboxamide;
2-(2-cyclopentylbenzyl)-N-(4,4-difluorobutyl)-3-oxoisoindoline-1-carboxamide;
2-(2-cyclopentylbenzyl)-N-methyl-3-oxoisoindoline-1-carboxamide;
N-benzyl-6-chloro-3-oxo-2-[(1S)-1-phenylethyl]isoindoline-1-carboxamide;
6-chloro-N-(2-methoxyethyl)-3-oxo-2-[2,2,2-trifluoro-1-(3-fluorophenyl)ethyl]isoindoline-1-carboxamide;
N-benzyl-6-chloro-2-(dipyridin-3-ylmethyl)-3-oxoisoindoline-1-carboxamide;
6-chloro-N-methyl-3-oxo-2-[2-(trifluoromethyl)benzyl]isoindoline-1-carboxamide;
2-[2-(4-chlorophenyl)propyl]-6-fluoro-N-methyl-3-oxoisoindoline-1-carboxamide;
6-fluoro-N-methyl-3-oxo-2-[(1R)-1-phenylethyl]isoindoline-1-carboxamide;
6-chloro-2-[2-(4-chlorophenyl)propyl]-N-methyl-3-oxoisoindoline-1-carboxamide;
6-chloro-N-methyl-3-oxo-2-[(1R)-1-phenylethyl]isoindoline-1-carboxamide;
2-[2-(4-chlorophenyl)propyl]-N-methyl-3-oxoisoindoline-1-carboxamide;
6-chloro-N-ethyl-3-oxo-2-[(1R)-1-phenylethyl]isoindoline-1-carboxamide;
N-benzyl-5-[(methylsulfonyl)amino]-3-oxo-2-[(1R)-1-phenylethyl]isoindoline-1-carboxamide;
N-benzyl-4-methyl-5-[(methylsulfonyl)amino]-3-oxo-2-[(1R)-1-phenylethyl]isoindoline-1-carboxamide;
N-benzyl-5-cyano-3-oxo-2-[(1R)-1-phenylethyl]isoindoline-1-carboxamide;
N-benzyl-5-bromo-3-oxo-2-[(1R)-1-phenylethyl]isoindoline-1-carboxamide;
N-benzyl-6-bromo-3-oxo-2-[(1R)-1-phenylethyl]isoindoline-1-carboxamide;
N-[3-(difluoromethoxy)benzyl]-6-fluoro-2-(3-hydroxy-2,2-dimethylpropyl)-3-oxoisoindoline-1-carboxamide;
N-(3,4-dichlorobenzyl)-6-fluoro-2-(3-hydroxy-2,2-dimethylpropyl)-3-oxoisoindoline-1-carboxamide;
N-(3-chlorobenzyl)-6-fluoro-2-(3-hydroxy-2,2-dimethylpropyl)-3-oxoisoindoline-1-carboxamide;
6-fluoro-2-(3-hydroxy-2,2-dimethylpropyl)-3-oxo-N-[3-(trifluoromethyl)benzyl]isoindoline-1-carboxamide;
6-chloro-N-[3-(difluoromethoxy)benzyl]-2-(3-hydroxy-2,2-dimethylpropyl)-3-oxoisoindoline-1-carboxamide;
6-chloro-N-(3,4-dichlorobenzyl)-2-(3-hydroxy-2,2-dimethylpropyl)-3-oxoisoindoline-1-carboxamide;
6-chloro-N-(3-chlorobenzyl)-2-(3-hydroxy-2,2-dimethylpropyl)-3-oxoisoindoline-1-carboxamide;
6-chloro-2-(3-hydroxy-2,2-dimethylpropyl)-3-oxo-N-[3-(trifluoromethyl)benzyl]isoindoline-1-carboxamide;
N-(3,4-dichlorobenzyl)-2-(3-hydroxy-2,2-dimethylpropyl)-3-oxoisoindoline-1-carboxamide;
N-[3-(difluoromethoxy)benzyl]-2-(3-hydroxy-2,2-dimethylpropyl)-3-oxoisoindoline-1-carboxamide;
2-(3-hydroxy-2,2-dimethylpropyl)-3-oxo-N43-(trifluoromethyl)benzyl]isoindoline-1-carboxamide;
N-(4,4-difluorobutyl)-6-fluoro-3-oxo-2-[2-(trifluoromethyl)benzyl]isoindoline-1-carboxamide;
6-chloro-N-(4,4-difluorobutyl)-3-oxo-2-[2-(trifluoromethyl)benzyl]isoindoline-1-carboxamide;
6-chloro-N-(4,4-difluorobutyl)-3-oxo-2-[(1R)-1-phenylethyl]isoindoline-1-carboxamide;
N-(4,4-difluorobutyl)-3-oxo-2-[(1R)-1-phenylethyl]isoindoline-1-carboxamide;
N-(tert-butyl)-6-fluoro-3-oxo-2-[2-(trifluoromethyl)benzyl]isoindoline-1-carboxamide;
N-(tert-butyl)-3-oxo-2-[2-(trifluoromethyl)benzyl]isoindoline-1-carboxamide;
N-(tert-butyl)-6-chloro-3-oxo-2-[2-(trifluoromethyl)benzyl]isoindoline-1-carboxamide;
6-fluoro-3-oxo-N-(4,4,4-trifluorobutyl)-2-[2-(trifluoromethyl)benzyl]isoindoline-1-carboxamide;
3-oxo-N-(4,4,4-trifluorobutyl)-2-[2-(trifluoromethyl)benzyl]isoindoline-1-carboxamide;
6-chloro-3-oxo-N-(4,4,4-trifluorobutyl)-2-[2-(trifluoromethyl)benzyl]isoindoline-1-carboxamide;
N-(tert-butyl)-6-fluoro-3-oxo-2-[(1R)-1-phenylethyl]isoindoline-1-carboxamide;
6-fluoro-3-oxo-2-[(1R)-1-phenylethyl]-N-(4,4,4-trifluorobutyl)isoindoline-1-carboxamide;
N-(tert-butyl)-6-chloro-3-oxo-2-[(1R)-1-phenylethyl]isoindoline-1-carboxamide;
6-chloro-3-oxo-2-[(1R)-1-phenylethyl]-N-(4,4,4-trifluorobutyl)isoindoline-1-carboxamide;
3-oxo-2-[(1R)-1-phenylethyl]-N-(4,4,4-trifluorobutyl)isoindoline-1-carboxamide;
6-chloro-N-[4-(methylsulfonyl)benzyl]-3-oxo-2[(1R)-1-phenylethyl]isoindoline-1-carboxamide;
N-benzyl-3-oxo-2-[2-(trifluoromethyl)benzyl]isoindoline-1-carboxamide;
N-[(4-amino-2-methylpyrimidin-5-yl)methyl]-6-chloro-2-[2-(4-chlorophenyl)propyl]-3-oxoisoindoline-1-carboxamide;
2-(biphenyl-2-ylmethyl)-N-[(5-methylpyrazin-2-yl)methyl]-3-oxoisoindoline-1-carboxamide;
2-(biphenyl-2-ylmethyl)-3-oxo-N-(pyridin-3-ylmethypisoindoline-1-carboxamide;
N-[(4-amino-2-methylpyrimidin-5-yl)methyl]-2-(biphenyl-2-ylmethyl)-6-chloro-3-oxoisoindoline-1-carboxamide;
N-[(4-amino-2-methylpyrimidin-5-yl)methyl]-2-(biphenyl-2-ylmethyl)-3-oxoisoindoline-1-carboxamide;
N-butyl-2-[4-fluorophenyl)(pyridin-3-yl)methyl]-3-oxoisoindoline-1-carboxamide;
N-butyl-3-oxo-2-[phenyl(pyridin-2-yl)methyl]isoindoline-1-carboxamide;
N-butyl-2-[4-chlorophenyl)(pyridin-4-yl)methyl]-3-oxoisoindoline-1-carboxamide;
2-[(4-chlorophenyl)(pyridin-4-yl)methyl]-N-(4-fluorobenzyl)-3-oxoisoindoline-1-carboxamide;
6-chloro-2-(diphenylmethyl)-N-ethyl-3-oxoisoindoline-1-carboxamide;
2-(biphenyl-2-ylmethyl)-6-chloro-N-ethyl-3-oxoisoindoline-1-carboxamide;
2-(biphenyl-2-ylmethyl)-6-chloro-3-oxo-N-propylisoindoline-1-carboxamide;
2-(diphenylmethyl)-N-ethyl-3-oxoisoindoline-1-carboxamide;
2-(biphenyl-2-ylmethyl)-N-ethyl-3-oxoisoindoline-1-carboxamide;
2-(biphenyl-2-ylmethyl)-6-fluoro-3-oxo-N-propylisoindoline-1-carboxamide;
N-(4-fluorobenzyl)-2-[4-fluorophenyl)(pyridin-3-yl)methyl]-3-oxoisoindoline-1-carboxamide;
N-benzyl-2-[(4-fluorophenyl)(pyridin-3-yl)methyl]-3-oxoisoindoline-1-carboxamide;
2-[2-(4-chlorophenyl)propyl]-N-[(5-methylpyrazin-2-yl)methyl]-3-oxoisoindoline-1-carboxamide;
6-chloro-2-[2-(4-chlorophenyl)propyl]-N-[(5-methylpyrazin-2-yl)methyl]-3-oxoisoindoline-1-carboxamide;
2-(biphenyl-2-ylmethyl)-6-chloro-N-[(5-methylpyrazin-2-yl)methyl]-3-oxoisoindoline-1-carboxamide;
6-chloro-2-[2-(4-chlorophenyl)propyl]-3-oxo-N-(pyridin-3-ylmethyl)isoindoline-1-carboxamide;
6-chloro-2-[2-(4-chlorophenyl)propyl]-3-oxo-N-{[6-(trifluoromethyl)pyridin-3-yl]methyl}isoindoline-1-carboxamide;
N-[(4-amino-2-methylpyrimidin-5-yl)methyl]-2-[2-(4-chlorophenyl)propyl]-3-oxoisoindoline-1-carboxamide;
2-(biphenyl-2-ylmethyl)-6-chloro-3-oxo-N-{[6-(trifluoromethyl)pyridin-3-yl]methyl}isoindoline-1-carboxamide;
2-[2-(4-chlorophenyl)propyl]-3-oxo-N-{[6-(trifluoromethyl)pyridin-3-yl]methyl}isoindoline-1-carboxamide;
2-(biphenyl-2-ylmethyl)-6-chloro-3-oxo-N-(pyridin-3-ylmethyl)isoindoline-1-carboxamide;
2-(biphenyl-2-ylmethyl)-3-oxo-N-{[6-(trifluoromethyl)pyridin-3-yl]methyl}isoindoline-1-carboxamide;
N-benzyl-6-fluoro-3-oxo-2-[(1R)-1-phenylethyl]isoindoline-1-carboxamide;
N-[4-(methylsulfonyl)benzyl]-3-oxo-2-[(1R)-1-phenylethyl]isoindoline-1-carboxamide;
6-fluoro-N-[4-(methylsulfonyl)benzyl]-3-oxo-2-[(1R)-1-phenylethyl]isoindoline-1-carboxamide;
N-benzyl-6-chloro-3-oxo-2-[2-(trifluoromethyl)benzyl]isoindoline-1-carboxamide;
N-benzyl-6-fluoro-3-oxo-2-[2-(trifluoromethyl)benzyl]isoindoline-1-carboxamide;
6-chloro-N-[4-(methylsulfonyl)benzyl]-3-oxo-2-[2-(trifluoromethyl)benzyl]isoindoline-1-carboxamide;
6-chloro-N-[2-chloro-4-(methylsulfonyl)benzyl]-2-(diphenylmethyl)-3-oxoisoindoline-1-carboxamide;
N-[2-chloro-4-(methylsulfonyl)benzyl]-2-(diphenylmethyl)-3-oxoisoindoline-1-carboxamide;
6-chloro-2-(diphenylmethyl)-N-[2-fluoro-4-(methylsulfonyl)benzyl]-3-oxoisoindoline-1-carboxamide;
2-(diphenylmethyl)-N-[2-fluoro-4-(methylsulfonyl)benzyl]-3-oxoisoindoline-1-carboxamide;
6-chloro-2-(diphenylmethyl)-N-[4-(methylsulfonyl)benzyl]-3-oxoisoindoline-1-carboxamide;
2-(biphenyl-2-ylmethyl)-6-chloro-3-oxo-N-(pyridin-4-ylmethyl)isoindoline-1-carboxamide;
6-chloro-2-[2-(4-chlorophenyl)propyl]-3-oxo-N-(pyridin-4-ylmethypisoindoline-1-carboxamide;
2-(biphenyl-2-ylmethyl)-3-oxo-N-(pyridin-4-ylmethyl)isoindoline-1-carboxamide;
2-(biphenyl-2-ylmethyl)-N-(tert-butyl)-5-cyano-3-oxoisoindoline-1-carboxamide;
6-chloro-2-(diphenylmethyl)-3-oxo-N-propylisoindoline-1-carboxamide;
2-[2-(4-chlorophenyl)propyl]-N-ethyl-6-fluoro-3-oxoisoindoline-1-carboxamide;
2-(diphenylmethyl)-N-ethyl-6-fluoro-3-oxoisoindoline-1-carboxamide;
2-[2-(4-chlorophenyl)propyl]-N-ethyl-3-oxoisoindoline-1-carboxamide;
2-[2-(4-chlorophenyl)propyl]-6-fluoro-3-oxo-N-propylisoindoline-1-carboxamide;
2-(diphenylmethyl)-6-fluoro-3-oxo-N-propylisoindoline-1-carboxamide;
2-(biphenyl-2-ylmethyl)-3-oxo-N-propylisoindoline-1-carboxamide;
2-[2-(4-chlorophenyl)propyl]-3-oxo-N-propylisoindoline-1-carboxamide;
2-(diphenylmethyl)-3-oxo-N-propylisoindoline-1-carboxamide;
2-(diphenylmethyl)-6-fluoro-3-oxo-N-(4,4,4-trifluorobutyl)isoindoline-1-carboxamide;
2-[2-(4-chlorophenyl)propyl]-6-fluoro-3-oxo-N-(4,4,4-trifluorobutyl)isoindoline-1-carboxamide;
2-[2-(4-chlorophenyl)propyl]-3-oxo-N-(4,4,4-trifluorobutyl)isoindoline-1-carboxamide;
2-(biphenyl-2-ylmethyl)-3-oxo-N-(4,4,4-trifluorobutyl)isoindoline-1-carboxamide;
2-(diphenylmethyl)-3-oxo-N-(4,4,4-trifluorobutyl)isoindoline-1-carboxamide;
N-(tert-butyl)-2-[4-chlorophenyl)(pyridin-4-yl)methyl]-3-oxoisoindoline-1-carboxamide;
N-(4-fluorobenzyl)-3-oxo-2-[phenyl(pyridin-2-yl)methyl]isoindoline-1-carboxamide;
N-benzyl-2-[4-chlorophenyl)(pyridin-4-yl)methyl]-3-oxoisoindoline-1-carboxamide;
N-benzyl-3-oxo-2-[phenyl(pyridin-2-yl)methyl]isoindoline-1-carboxamide;
2-(2,2-dimethylpropyl)-N-[(5-methyl-2-phenyl-1,3-oxazol-4-yl)methyl]-3-oxoisoindoline-1-carboxamide;
2-(2,2-dimethylpropyl)-N-[(1-methyl-5-phenyl-1H-pyrazol-3-yl)methyl]-3-oxoisoindoline-1-carboxamide;
N-butyl-2-[(5-methyl-2-phenyl-1,3-oxazol-4-yl)methyl]-3-oxoisoindoline-1-carboxamide;
N-benzyl-2-[(1-methyl-5-phenyl-1H-pyrazol-3-yl)methyl]-3-oxoisoindoline-1-carboxamide;
N-benzyl-2-[(5-methyl-2-phenyl-1,3-oxazol-4-yl)methyl]-3-oxoisoindoline-1-carboxamide;
N-benzyl-2-(biphenyl-2-ylmethyl)-5-hydroxy-4-methyl-3-oxoisoindoline-1-carboxamide;
2-(biphenyl-2-ylmethyl)-5-hydroxy-4-methyl-3-oxo-N-(4,4,4-trifluorobutyl)isoindoline-1-carboxamide;
2-(biphenyl-2ylmethyl)-5-hydroxy-4-methyl-3-oxo-N-propylisoindoline-1-carboxamide;
2-(biphenyl-2-ylmethyl)-N-butyl-5-hydroxy-4-methyl-3-oxoisoindoline-1-carboxamide;
2-(biphenyl-2-ylmethyl)-N-ethyl-5-hydroxy-4-methyl-3-oxoisoindoline-1-carboxamide;
N-(tert-butyl)-2-[(3′,4′-difluorobiphenyl-2-yl)methyl]-5-hydroxy-4-methyl-3-oxoisoindoline-1-carboxamide;
N-(tert-butyl)-2-[(2′,4′-dichlorobiphenyl-2-yl)methyl]-5-hydroxy-4-methyl-3-oxoisoindoline-1-carboxamide;
N-(tert-butyl)-2-[(3′,4′-dichlorobiphenyl-2-yl)methyl]-5-hydroxy-4-methyl-3-oxoisoindoline-1-carboxamide;
N-(tert-butyl)-2-[(2′-chlorobiphenyl-2-yl)methyl]-5-hydroxy-4-methyl-3-oxoisoindoline-1-carboxamide;
N-(tert-butyl)-2-[(3′-chloro-4′-fluorobiphenyl-2-yl)methyl]-5-hydroxy-4-methyl-3-oxoisoindoline-1-carboxamide;
N-(tert-butyl)-2-[(4′-chlorobiphenyl-2-yl)methyl]-5-hydroxy-4-methyl-3-oxoisoindoline-1-carboxamide;
N-(tert-butyl)-2-[(4′-fluoro-2′-methylbiphenyl-2-yl)methyl]-5-hydroxy-4-methyl-3-oxoisoindoline-1-carboxamide;
N-(tert-butyl)-2-[(2′,4′-difluorobiphenyl-2-yl)methyl]-5-hydroxy-4-methyl-3-oxoisoindoline-1-carboxamide;
N-(tert-butyl)-2-[(2′,5′-difluorobiphenyl-2-yl)methyl]-5-hydroxy-4-methyl-3-oxoisoindoline-1-carboxamide;
N-(tert-butyl)-2-[(3′-fluorobiphenyl-2-yl)methyl]-5-hydroxy-4-methyl-3-oxoisoindoline-1-carboxamide;
N-butyl-3-oxo-2-(2-phenoxybenzyl)isoindoline-1-carboxamide;
N-[3-(difluoromethoxy)benzyl]-2-(3,3-dimethylbutyl)-3-oxoisoindoline-1-carboxamide;
2-(3,3-dimethylbutyl)-3-oxo-N-[3-(trifluoromethoxy)benzyl]isoindoline-1-carboxamide;
2-(2,2-dimethylpropyl)-3-oxo-N-[3-(trifluoromethoxy)benzyl]isoindoline-1-carboxamide;
N-[3-(difluoromethoxy)benzyl]-2-(2,2-dimethylpropyl)-3-oxoisoindoline-1-carboxamide;
6-chloro-2-(diphenylmethyl)-3-oxo-N-(4,4,4-trifluorobutyl)isoindoline-1-carboxamide;
6-chloro-2-(diphenylmethyl)-N-(2-hydroxybenzyl)-3-oxoisoindoline-1-carboxamide;
N-benzyl-6-chloro-2-(diphenylmethyl)-3-oxoisoindoline-1-carboxamide;
N-(tert-butyl)-6-chloro-2-(diphenylmethyl)-3-oxoisoindoline-1-carboxamide;
2-(biphenyl-2-ylmethyl)-6-chloro-3-oxo-N-(4,4,4-trifluorobutyl)isoindoline-1-carboxamide;
2-(biphenyl-2-ylmethyl)-6-chloro-N-(3-cyanobenzyl)-3-oxoisoindoline-1-carboxamide;
N-benzyl-2-(biphenyl-2-ylmethyl)-6-chloro-3-oxoisoindoline-1-carboxamide;
2-(biphenyl-2-ylmethyl)-N-(tert-butyl)-6-chloro-3-oxoisoindoline-1-carboxamide;
6-chloro-2-[2-(4-chlorophenyl)propyl]-3-oxo-N-(4,4,4-trifluorobutyl)isoindoline-1-carboxamide;
6-chloro-2-[2-(4-chlorophenyl)propyl]-N-[4-(methylsulfonyl)benzyl]-3-oxoisoindoline-1-carboxamide;
N-(tert-butyl)-6-chloro-2-[2-(4-chlorophenyl)propyl]-3-oxoisoindoline-1-carboxamide;
N-benzyl-6-chloro-2-[2-(4-chlorophenyl)propyl]-3-oxoisoindoline-1-carboxamide;
N-(1H-1,2,3-benzotriazol-1-ylmethyl)-4,5-dimethoxy-2-(2-methoxybenzyl)-3-oxoisoindoline-1-carboxamide;
2-[1-(1,5-dimethyl-1H-pyrazol-4-yl)ethyl]-5,7-dimethoxy-3-oxo-N-[2-(trifluoromethyl)benzyl]isoindoline-1-carboxamide;
5,7-dimethoxy-2-(2-methoxybenzyl)-3-oxo-N-[2-(trifluoromethyl)benzyl]isoindoline-1-carboxamide;
2-(2-fluorobenzyl)-5,7-dimethoxy-3-oxo-N-[2-(trifluoromethyl)benzyl]isoindoline-1-carboxamide;
N-(tert-butyl)-5,7-dimethoxy-2-(2-methoxybenzyl)-3-oxoisoindoline-1-carboxamide;
3-oxo-2-(2-phenoxybenzyl)-N-(4,4,4-trifluorobutyl)isoindoline-1-carboxamide;
2-[2-(4-chlorophenyl)propyl]-N-[(2,2-difluoro-1,3-benzodioxol-5-yl)methyl]-3-oxoisoindoline-1-carboxamide;
N-(3,4-dichlorobenzyl)-2-(2,2-dimethylpropyl)-3-oxoisoindoline-1-carboxamide;
2-(2,2-dimethylpropyl)-[1H-indol-3-ylmethyl)-3-oxoisoindoline-1-carboxamide;
2-(2,2-dimethylpropyl)-3-oxo-N-{2-[3-(trifluoromethyl)phenyl]ethyl}isoindoline-1-carboxamide;
2-(biphenyl-2-ylmethyl)-N-(tert-butyl)-6-fluoro-3-oxoisoindoline-1-carboxamide;
N-benzyl-2-(biphenyl-2-ylmethyl)-6-fluoro-3-oxoisoindoline-1-carboxamide;
2-[2-(4-chlorophenyl)ethyl]-N-[(2,2-difluoro-1,3-benzo dioxol-5-yl)methyl]-6-fluoro-3-oxoisoindoline-1-carboxamide;
2-[2-(4-chlorophenyl)ethyl]-6-fluoro-3-oxo-N-(4,4,4-trifluorobutyl)isoindoline-1-carboxamide;
N-(tert-butyl)-2-[2-(4-chlorophenyl)ethyl]-6-fluoro-3-oxoisoindoline-1-carboxamide;
N-benzyl-2-[2-(4-chlorophenyl)ethyl]-6-fluoro-3-oxoisoindoline-1-carboxamide;
6-fluoro-2-[2-(4-fluorophenyl)ethyl]-3-oxo-N-(4,4,4-trifluorobutyl)isoindoline-1-carboxamide;
N-benzyl-6-fluoro-2-[2-(4-fluorophenyl)ethyl]-3-oxoisoindoline-1-carboxamide;
N-(tert-butyl)-6-fluoro-2-[2-(4-fluorophenyl)ethyl]-3-oxoisoindoline-1-carboxamide;
6-fluoro-2-[2-(4-fluorophenyl)propyl]-3-oxo-N-(4,4,4-trifluorobutyl)isoindoline-1-carboxamide;
N-benzyl-6-fluoro-2-[2-(4-fluorophenyl)propyl]-3-oxoisoindoline-1-carboxamide;
N-(tert-butyl)-6-fluoro-2-[2-(4-fluorophenyl)propyl]-3-oxoisoindoline-1-carboxamide;
2-(biphenyl-2-ylmethyl)-1-methyl-N-[4-(methylsulfonyl)benzyl]-3-oxoisoindoline-1-carboxamide;
2-[2-(4-chlorophenyl)propyl]-4,7-difluoro-1-methyl-N-[4-(methylsulfonyl)benzyl]-3-oxoisoindoline-1-carboxamide;
N-butyl-2-[2-(4-chlorophenyl)propyl]-1-methyl-3-oxoisoindoline-1-carboxamide;
2-(biphenyl-2-ylmethyl)-N-(tert-butyl)-5,6-dimethoxy-3-oxoisoindoline-1-carboxamide;
N-benzyl-2-(diphenylmethyl)-5-methoxy-3-oxoisoindoline-1-carboxamide;
2-(diphenylmethyl)-3-oxo-N-[2-(trifluoromethyl)benzyl]isoindoline-1-carboxamide;
N-(tert-butyl)-2-[2-(4-chlorophenyl)propyl]-1-methyl-3-oxoisoindoline-1-carboxamide;
N-butyl-2-(diphenylmethyl)-3-oxoisoindoline-1-carboxamide;
2-(biphenyl-2-ylmethyl)-N-(tert-butyl)-5-methoxy-4-methyl-3-oxoisoindoline-1-carboxamide;
2-(biphenyl-2-ylmethyl)-1-[(tert-butylamino)carbonyl]-4-methyl-3-oxo-2,3-dihydro-1H-isoindol-5-yl dimethylcarbamate;
2-(biphenyl-2-ylmethyl)-5-hydroxy-3-oxo-N-(2-phenylethyl)isoindoline-1-carboxamide;
5-hydroxy-2-[2-(4-methoxyphenyl)ethyl]-3-oxo-N-(2-phenylethyl)isoindoline-1-carboxamide;
2-(4-chlorobenzyl)-5-hydroxy-3-oxo-N-(2-phenylethyl)isoindoline-1-carboxamide;
N-(4-fluorobenzyl)-5-hydroxy-2-[2-(4-methoxyphenyl)ethyl]-3-oxoisoindoline-1-carboxamide;
2-[2-(3,4-dichlorophenyl)ethyl]-N-(4-fluorobenzyl)-5-hydroxy-3-oxoisoindoline-1-carboxamide;
2-(4-chlorobenzyl)-N-(4-fluorobenzyl)-5-hydroxy-3-oxoisoindoline-1-carboxamide;
2-(biphenyl-2-ylmethyl)-N-(4-fluorobenzyl)-5-hydroxy-3-oxoisoindoline-1-carboxamide;
N-(tert-butyl)-2-[2-(3,4-dichlorophenyl)ethyl]-5-hydroxy-3-oxoisoindoline-1-carboxamide;
N-(3,4-dichlorobenzyl)-2-isobutyl-3-oxoisoindoline-1-carboxamide;
N-[2-(1H-indol-3-yl)ethyl]-2-isobutyl-3-oxoisoindoline-1-carboxamide;
N-(3-chlorobenzyl)-2-isobutyl-3-oxoisoindoline-1-carboxamide;
N-[4-(difluoromethoxy)benzyl]-2-isobutyl-3-oxoisoindoline-1-carboxamide;
2-isobutyl-3-oxo-N-[3-(trifluoromethyl)benzyl]isoindoline-1-carboxamide;
N-(1H-indol-3-ylmethyl)-2-isobutyl-3-oxoisoindoline-1-carboxamide;
N-[2-(1,3-benzodioxol-5-yl)ethyl]-2-isobutyl-3-oxoisoindoline-1-carboxamide;
N-[2-(3-fluorophenyl)ethyl]-2-isobutyl-3-oxoisoindoline-1-carboxamide;
2-isobutyl-3-oxo-N-{2-[3-(trifluoromethyl)phenyl]ethyl}isoindoline-1-carboxamide;
N-[2-(3,4-dichlorophenyl)ethyl]-2-isobutyl-3-oxoisoindoline-1-carboxamide;
N-[2-(4-chlorophenyl)ethyl]-2-isobutyl-3-oxoisoindoline-1-carboxamide;
N-[2-(3-chlorophenyl)ethyl]-2-isobutyl-3-oxoisoindoline-1-carboxamide;
N-[2-(2-chlorophenyl)ethyl]-2-isobutyl-3-oxoisoindoline-1-carboxamide;
N-[2-(2,4-dichlorophenyl)ethyl]-2-isobutyl-3-oxoisoindoline-1-carboxamide;
N-[2-(2,6-dichlorophenyl)ethyl]-2-isobutyl-3-oxoisoindoline-1-carboxamide;
2-(3,3-dimethylbutyl)-N-[2-(1H-indol-3-yl)ethyl]-3-oxoisoindoline-1-carboxamide;
N-(3-chlorobenzyl)-2-(3,3-dimethylbutyl)-3-oxoisoindoline-1-carboxamide;
N-(3,4-dichlorobenzyl)-2-(3,3-dimethylbutyl)-3-oxoisoindoline-1-carboxamide;
2-(3,3-dimethylbutyl)-N-(1H-indol-3-ylmethyl)-3-oxoisoindoline-1-carboxamide;
N-[4-(difluoromethoxy)benzyl]-2-(3,3-dimethylbutyl)-3-oxoisoindoline-1-carboxamide;
2-(3,3-dimethylbutyl)-N-[2-(3-fluorophenyl)ethyl]-3-oxoisoindoline-1-carboxamide;
N-[2-(1,3-benzodioxol-5-yl)ethyl]-2-(3,3-dimethylbutyl)-3-oxoisoindoline-1-carboxamide;
N-[2-(3-cyanophenyl)ethyl]-2-(3,3-dimethylbutyl)-3-oxoisoindoline-1-carboxamide;
2-(3,3-dimethylbutyl)-3-oxo-N-{2-[3-(trifluoromethyl)phenyl]ethyl}isoindoline-1-carboxamide;
N-[2-(3-chlorophenyl)ethyl]-2-(3,3-dimethylbutyl)-3-oxoisoindoline-1-carboxamide;
N-[2-(3,4-dichlorophenyl)ethyl]-2-(3,3-dimethylbutyl)-3-oxoisoindoline-1-carboxamide;
N-[2-(4-chlorophenyl)ethyl]-2-(3,3-dimethylbutyl)-3-oxoisoindoline-1-carboxamide;
N-[2-(2-chlorophenyl)ethyl]-2-(3,3-dimethylbutyl)-3-oxoisoindoline-1-carboxamide;
N-[2-(2,4-dichlorophenyl)ethyl]-2-(3,3-dimethylbutyl)-3-oxoisoindoline-1-carboxamide;
2-(2,2-dimethylpropyl)-N-[2-(1H-indol-3-yl)ethyl]-3-oxoisoindoline-1-carboxamide;
N-(3-chlorobenzyl)-2-(2,2-dimethylpropyl)-3-oxoisoindoline-1-carboxamide;
N-[4-(difluoromethoxy)benzyl]-2-(2,2-dimethylpropyl)-3-oxoisoindoline-1-carboxamide;
2-(2,2-dimethylpropyl)-3-oxo--[3-(trifluoromethyl)benzyl]isoindoline-1-carboxamide;
N-[2-(1,3-benzodioxol-5-yl)ethyl]-2-(2,2-dimethylpropyl)-3-oxoisoindoline-1-carboxamide;
2-(2,2-dimethylpropyl)-N-[2-(3-fluorophenyl)ethyl]-3-oxoisoindoline-1-carboxamide;
N-[2-(3-cyanophenyl)ethyl]-2-(2,2-dimethylpropyl)-3-oxoisoindoline-1-carboxamide;
N-[2-(2-chlorophenyl)ethyl]-2-(2,2-dimethylpropyl)-3-oxoisoindoline-1-carboxamide;
N-[2-(3-chlorophenyl)ethyl]-2-(2,2-dimethylpropyl)-3-oxoisoindoline-1-carboxamide;
N-[2-(2,4-dichlorophenyl)ethyl]-2-(2,2-dimethylpropyl)-3-oxoisoindoline-1-carboxamide;
2-[2-(4-chlorophenyl)ethyl]-N-ethyl-3-oxo-N-(2-pyridin-2-ylethyl)isoindoline-1-carboxamide;
2-[2-(4-chlorophenyl)ethyl]-3-(1,3-dihydro-2H-isoindol-2-ylcarbonyl)isoindolin-1-one;
2-[2-(4-chlorophenyl)ethyl]-N-methyl-3-oxo-N-[2-(trifluoromethyl)benzyl]isoindoline-1-carboxamide;
N-benzyl-2-[2-(4-chlorophenyl)ethyl]-N-ethyl-3-oxoisoindoline-1-carboxamide;
N-benzyl-2-[2-(4-chlorophenyl)ethyl]-N-methyl-3-oxoisoindoline-1-carboxamide;
N-(tert-butyl)-3-oxo-2-{2-[4-(trifluoromethyl)phenyl]ethyl}isoindoline-1-carboxamide;
N-butyl-3-oxo-2-{2-[4-(trifluoromethyl)phenyl]ethyl}isoindoline-1-carboxamide;
N-benzyl-3-oxo-2-{2-[4-(trifluoromethyl)phenyl]ethyl}isoindoline-1-carboxamide;
N-(tert-butyl)-5-hydroxy-2-[2-(1H-indol-3-yl)ethyl]-4-methyl-3-oxoisoindoline-1-carboxamide;
N-(tert-butyl)-2-[2-(4-fluorophenyl)propyl]-5-hydroxy-4-methyl-3-oxoisoindoline-1-carboxamide;
2-[3,5-bis(trifluoromethyl)benzyl]-N-(tert-butyl)-5-hydroxy-4-methyl-3-oxoisoindoline-1-carboxamide;
N-(tert-butyl)-2-(2,2-diphenylethyl)-5-hydroxy-4-methyl-3-oxoisoindoline-1-carboxamide;
N-(tert-butyl)-2-(diphenylmethyl)-5-hydroxy-4-methyl-3-oxoisoindoline-1-carboxamide;
N-(tert-butyl)-2-(9H-fluoren-9-yl)-5-hydroxy-4-methyl-3-oxoisoindoline-1-carboxamide;
N-(tert-butyl)-5-hydroxy-4-methyl-3-oxo-2-{2-[4-(trifluoromethyl)phenoxy]benzyl}isoindoline-1-carboxamide;
2-(biphenyl-3-ylmethyl)-N-(tert-butyl)-5-hydroxy-4-methyl-3-oxoisoindoline-1-carboxamide;
N-butyl-2-[2-(4-fluorophenyl)propyl]-3-oxoisoindoline-1-carboxamide;
N-butyl-2-[2-(4-chlorophenyl)ethyl]-3-oxoisoindoline-1-carboxamide;
N-(tert-butyl)-2-[2-(4-chlorophenyl)ethyl]-3-oxoisoindoline-1-carboxamide;
N-(tert-butyl)-2-[2-(4-fluorophenyl)propyl]-3-oxoisoindoline-1-carboxamide;
N-benzyl-2-[2-(4-fluorophenyl)propyl]-3-oxoisoindoline-1-carboxamide;
N-benzyl-2-[2-(4-fluorophenyl)ethyl]-3-oxoisoindoline-1-carboxamide;
N-benzyl-2-[2-(4-chlorophenyl)ethyl]-3-oxoisoindoline-1-carboxamide;
N-[2-(1H-indol-3-yl)ethyl]-3-oxo-2-[4-(piperidin-1-ylcarbonyl)benzyl]isoindoline-1-carboxamide;
2-(biphenyl-2-ylmethyl)-N-(2,4-difluorobenzyl)-3-oxoisoindoline-1-carboxamide;
2-(biphenyl-2-ylmethyl)-N-(4-cyano-2,6-difluorobenzyl)-3-oxoisoindoline-1-carboxamide;
N-(2,4-difluorobenzyl)-2-(diphenylmethyl)-3-oxoisoindoline-1-carboxamide;
N-(2-chlorobenzyl)-2-(diphenylmethyl)-3-oxoisoindoline-1-carboxamide;
2-(diphenylmethyl)-N-[2-(4-fluorophenyl)ethyl]-3-oxoisoindoline-1-carboxamide;
2-(biphenyl-2-ylmethyl)-N-[2-(4-fluorophenyl)ethyl]-3-oxoisoindoline-1-carboxamide;
2-(diphenylmethyl)-N-(4-fluorobenzyl)-3-oxoisoindoline-1-carboxamide;
N-(2,4-difluorobenzyl)-3-oxo-2-(2-pyridin-3-ylbenyzl)isoindoline-1-carboxamide;
N-(2-chlorobenzyl)-3-oxo-2-(2-pyridin-3-ylbenyzl)isoindoline-1-carboxamide;
N-[2-(4-fluorophenyl)ethyl]-3-oxo-2-(2-pyridin-3-ylbenzyl)isoindoline-1-carboxamide;
N-benzyl-3-oxo-2-(2-pyridin-3-ylbenzyl)isoindoline-1-carboxamide;
N-(4-fluorobenzyl)-3-oxo-2-(2-pyridin-3-ylbenzyl)isoindoline-1-carboxamide;
N-butyl-5-methoxy-2-(2-methyl-2-phenylpropyl)-3-oxoisoindoline-1-carboxamide;
2-(biphenyl-2-ylmethyl)-N-butyl-5-methoxy-3-oxoisoindoline-1-carboxamide;
N-butyl-2-[2-(4-fluorophenyl)propyl]-5-methoxy-3-oxoisoindoline-1-carboxamide;
N-butyl-2-[2-(4-chlorophenyl)propyl]-5-methoxy-3-oxoisoindoline-1-carboxamide;
N-(tert-butyl)-5-methoxy-2-[2-(1-naphthyl)propyl]-3-oxoisoindoline-1-carboxamide;
N-(tert-butyl)-2-[2-(4-fluorophenyl)propyl]-5-methoxy-3-oxoisoindoline-1-carboxamide;
N-(tert-butyl)-2-[2-(4-chlorophenyl)propyl]-5-methoxy-3-oxoisoindoline-1-carboxamide;
2-(biphenyl-2-ylmethyl)-N-(tert-butyl)-5-methoxy-3-oxoisoindoline-1-carboxamide;
N-benzyl-5-methoxy-2-[2-(1-naphthyl)propyl]-3-oxoisoindoline-1-carboxamide;
N-benzyl-5-methoxy-2-(2-methyl-2-phenylpropyl)-3-oxoisoindoline-1-carboxamide;
N-benzyl-2-(biphenyl-2-ylmethyl)-5-methoxy-3-oxoisoindoline-1-carboxamide;
N-butyl-5,6-dimethoxy-2-(2-methyl-2-phenylpropyl)-3-oxoisoindoline-1-carboxamide;
N-benzyl-2-[2-(4-chlorophenyl)propyl]-5-methoxy-3-oxoisoindoline-1-carboxamide;
N-butyl-5,6-dimethoxy-2-[2-(1-naphthyl)propyl]-3-oxoisoindoline-1-carboxamide;
N-butyl-2-[2-(4-fluorophenyl)propyl]-5,6-dimethoxy-3-oxoisoindoline-1-carboxamide;
2-(biphenyl-2-ylmethyl)-N-butyl-5,6-dimethoxy-3-oxoisoindoline-1-carboxamide;
N-butyl-2-[2-(4-chlorophenyl)propyl]-5,6-dimethoxy-3-oxoisoindoline-1-carboxamide;
N-(tert-butyl)-5,6-dimethoxy-2-[2-(1-naphthyl)propyl]-3-oxoisoindoline-1-carboxamide;
N-benzyl-5,6-dimethoxy-2-[2-(1-naphthyl)propyl]-3-oxoisoindoline-1-carboxamide;
N-benzyl-5,6-dimethoxy-2-(2-methyl-2-phenylpropyl)-3-oxoisoindoline-1-carboxamide;
N-benzyl-2-[2-(4-fluorophenyl)propyl]-5,6-dimethoxy-3-oxoisoindoline-1-carboxamide;
N-benzyl-2-(biphenyl-2-ylmethyl)-5,6-dimethoxy-3-oxoisoindoline-1-carboxamide;
N-benzyl-2-(diphenylmethyl)-5,6-dimethoxy-3-oxoisoindoline-1-carboxamide;
N-benzyl-2-[2-(4-chlorophenyl)propyl]-5,6-dimethoxy-3-oxoisoindoline-1-carboxamide;
2-(biphenyl-2-ylmethyl)-N-(tert-butyl)-1-methyl-3-oxoisoindoline-1-carboxamide;
2-(biphenyl-2-ylmethyl)-N-butyl-1-methyl-3-oxoisoindoline-1-carboxamide;
ethyl N-benzyl-N-(}2-[2-(4-chlorophenyl)ethyl]-3-oxo-2,3-dihydro-1H-isoindol-1-yl}carbonyl)glycinate;
2-[2-(4-chlorophenyl)ethyl]-N-methyl-3-oxo-N-(2-phenylethyl)isoindoline-1-carboxamide;
2-[2-(4-chlorophenyl)ethyl]-N,N-diethyl-3-oxoisoindoline-1-carboxamide;
N-benzyl-N-butyl-2-[2-(4-chlorophenyl)ethyl]-3-oxoisoindoline-1-carboxamide;
N-[2-(2,6-dichlorophenyl)ethyl]-2-(3,3-dimethylbutyl)-3-oxoisoindoline-1-carboxamide;
N-[2-(4-chlorophenyl)ethyl]-2-(2,2-dimethylpropyl)-3-oxoisoindoline-1-carboxamide;
N-[2-(2,6-dichlorophenyl)ethyl]-2-(2,2-dimethylpropyl)-3-oxoisoindoline-1-carboxamide;
N-butyl-5-methoxy-2-[2-(1-naphthyl)propyl]-3-oxoisoindoline-1-carboxamide;
N-benzyl-2-[2-(4-fluorophenyl)propyl]-5-methoxy-3-oxoisoindoline-1-carboxamide;
N-(tert-butyl)-2-[2-(4-chlorophenyl)propyl]-5,6-dimethoxy-3-oxoisoindoline-1-carboxamide;
N-(tert-butyl)-2-[(3′,4′-difluorobiphenyl-2-yl)methyl]-3-oxoisoindoline-1-carboxamide;
N-(tert-butyl)-2-[(4′-fluoro-2′-methylbiphenyl-2-yl)methyl]-3-oxoisoindoline-1-carboxamide;
N-(tert-butyl)-2-[(4′-methylbiphenyl-2-yl)methyl]-3-oxoisoindoline-1-carboxamide;
N-(tert-butyl)-2-[(4′-methoxybiphenyl-2-yl)methyl]-3-oxoisoindoline-1-carboxamide;
N-(tert-butyl)-2-[(4′-fluorobiphenyl-2-yl)methyl]-3-oxoisoindoline-1-carboxamide;
methyl N-({2-[(3′,4′-difluorobiphenyl-2-yl)methyl]-3-oxo-2,3-dihydro-1H-isoindol-1-yl}carbonyl)glycinate;
methyl N-({2-[(4′-fluoro-2′-methylbiphenyl-2-yl)methyl]-3-oxo-2,3-dihydro-1H-isoindol-1-yl}carbonyl)glycinate;
methyl N-({2-[(4′-fluorobiphenyl-2-yl)methyl]-3-oxo-2,3-dihydro-1H-isoindol-1-yl}carbonyl)glycinate;
methyl N-({2-[(4′-methylbiphenyl-2-yl)methyl]-3-oxo-2,3-dihydro-1H-isoindol-1-yl}carbonyl)glycinate;
2-[(3′,4′-difluorobiphenyl-2-yl)methyl]-N-[4-(methylsulfonyl)benzyl]-3-oxoisoindoline-1-carboxamide;
2-[(4′-fluoro-2′-methylbiphenyl-2-yl)methyl]-N-[4-(methylsulfonyl)benzyl]-3-oxoisoindoline-1-carboxamide;
2-[(4′-methoxybiphenyl-2-yl)methyl]-N-[4-(methylsulfonyl)benzyl]-3-oxoisoindoline-1-carboxamide;
2-[(4′-fluorobiphenyl-2-yl)methyl]-N-[4-(methylsulfonyl)benzyl]-3-oxoisoindoline-1-carboxamide;
2-[(4′-methylbiphenyl-2-yl)methyl]-N-[4-(methylsulfonyl)benzyl]-3-oxoisoindoline-1-carboxamide;
N-(tert-butyl)-2-(4-chlorobenzyl)-5-hydroxy-4-methyl-3-oxoisoindoline-1-carboxamide;
N-(tert-butyl)-5-hydroxy-2-[2-(4-methoxyphenyl)ethyl]-3-oxoisoindoline-1-carboxamide;
2-[2-(4-chlorophenyl)propyl]-N-[2-(1H-indol-3-yl)ethyl]-3-oxoisoindoline-1-carboxamide;
N-(tert-butyl)-7-hydroxy-2-[2-(4-methoxyphenyl)ethyl]-3-oxoisoindoline-1-carboxamide;
2-(biphenyl-2-ylmethyl)-N-(tert-butyl)-5-hydroxy-3-oxoisoindoline-1-carboxamide;
2-[2-(3,4-dichlorophenyl)ethyl]-5-hydroxy-3-oxo-N-(2-phenylethyl)isoindoline-1-carboxamide;
N-(3,4-difluorobenzyl)-2-(4-hydroxybenzyl)-3-oxoisoindoline-1-carboxamide;
N-(3-chlorobenzyl)-2-(4-hydroxybenzyl)-3-oxoisoindoline-1-carboxamide;
2-(4-hydroxybenzyl)-3-oxo-N-[4-(trifluoromethyl)benzyl]isoindoline-1-carboxamide;
N-[3,5-bis(trifluoromethyl)benzyl]-2-(4-hydroxybenzyl)-3-oxoisoindoline-1-carboxamide;
N-(3-chlorobenzyl)-2-(3-cyanobenzyl)-3-oxoisoindoline-1-carboxamide;
N-[3,5-bis(trifluoromethyl)benzyl]-2-(3-cyanobenzyl)-3-oxoisoindoline-1-carboxamide;
2-(3-cyanobenzyl)-N-(3,4-difluorobenzyl)-3-oxoisoindoline-1-carboxamide;
2-(3-cyanobenzyl)-3-oxo-N-[4-(trifluoromethyl)benzyl]isoindoline-1-carboxamide;
N-[4-(aminocarbonyl)benzyl]-2-[2-(4-chlorophenyl)propyl]-3-oxoisoindoline-1-carboxamide;
N-[4-(aminocarbonyl)benzyl]-2-(biphenyl-2-ylmethyl)-3-oxoisoindoline-1-carboxamide;
2-(3,4-difluorobenzyl)-N-{4-[(dimethylamino)methyl]benzyl}-3-oxoisoindoline-1-carboxamide;
2-(3-chlorobenzyl)-N-{4-[(dimethylamino)methyl]benzyl}-3-oxoisoindoline-1-carboxamide;
2-[3,5-bis(trifluoromethyl)benzyl]-N-{4-[(dimethylamino)methyl]benzyl}-3-oxoisoindoline-1-carboxamide;
2-(3,4-difluorobenzyl)-N-(4-hydroxybenzyl)-3-oxoisoindoline-1-carboxamide;
2-(3-chlorobenzyl)-N-(4-hydroxybenzyl)-3-oxoisoindoline-1-carboxamide;
2-[3,5-bis(trifluoromethyl)benzyl]-N-(4-hydroxybenzyl)-3-oxoisoindoline-1-carboxamide;
2-(3-chlorobenzyl)-N-(3-cyanobenzyl)-3-oxoisoindoline-1-carboxamide;
N-(3-cyanobenzyl)-2-(3,4-difluorobenzyl)-3-oxoisoindoline-1-carboxamide;
2-[2-(4-chlorophenyl)propyl]-N-(4-cyanobenzyl)-3-oxoisoindoline-1-carboxamide;
2-[3,5-bis(trifluoromethyl)benzyl]-N-(3-cyanobenzyl)-3-oxoisoindoline-1-carboxamide;
N-(tert-butyl)-2-[2-(4-chlorophenyl)propyl]-5-hydroxy-3-oxoisoindoline-1-carboxamide;
N-{4-[(dimethylamino)methyl]benzyl}-3-oxo-2-[4-(trifluoromethyl)benzyl]isoindoline-1-carboxamide;
N-(4-hydroxybenzyl)-3-oxo-2-[4-(trifluoromethyl)benzyl]isoindoline-1-carboxamide;
N-(3-cyanobenzyl)-3-oxo-2-[4-(trifluoromethyl)benzyl]isoindoline-1-carboxamide;
2-(biphenyl-3-ylmethyl)-N-(4-cyanobenzyl)-3-oxoisoindoline-1-carboxamide;
2-(biphenyl-4-ylmethyl)-N-(4-cyanobenzyl)-3-oxoisoindoline-1-carboxamide;
N-butyl-3-oxo-2-[2-(2-phenoxyphenyl)ethyl]isoindoline-1-carboxamide;
N-butyl-2-(2-{4-[(diethylamino)carbonyl]phenyl}ethyl)-3-oxoisoindoline-1-carboxamide;
N-butyl-2-[2-(3-fluorophenyl)ethyl]-3-oxoisoindoline-1-carboxamide;
N-butyl-3-oxo-2-{2-[2-(trifluoromethoxy)phenyl]ethyl}isoindoline-1-carboxamide;
2-(2-biphenyl-4-ylethyl)-N-butyl-3-oxoisoindoline-1-carboxamide;
N-butyl-2-[2-(4-fluorophenyl)ethyl]-3-oxoisoindoline-1-carboxamide;
N-butyl-2-[2-(3,5-dimethoxyphenyl)ethyl]-3-oxoisoindoline-1-carboxamide;
N-butyl-3-oxo-2-[2-(4-phenoxyphenyl)ethyl]isoindoline-1-carboxamide;
N-butyl-2-[2-(2-ethoxyphenyl)ethyl]-3-oxoisoindoline-1-carboxamide;
2-[2-(1,3-benzodioxol-5-yl)ethyl]-N-butyl-3-oxoisoindoline-1-carboxamide;
N-(tert-butyl)-3-oxo-2-[2-(2-phenoxyphenyl)ethyl]isoindoline-1-carboxamide
N-(tert-butyl)-3-oxo-2-{2-[2-(trifluoromethoxy)phenyl]ethyl}isoindoline-1-carboxamide;
2-(2-biphenyl-4-ylethyl)-N-(tert-butyl)-3-oxoisoindoline-1-carboxamide;
N-(tert-butyl)-2-[2-(3,5-dimethoxyphenyl)ethyl]-3-oxoisoindoline-1-carboxamide;
N-(tert-butyl)-3-oxo-2-[2-(4-phenoxyphenyl)ethyl]isoindoline-1-carboxamide;
N-(tert-butyl)-2-[2-(2-ethoxyphenyl)ethyl]-3-oxoisoindoline-1-carboxamide;
2-[2-(1,3-benzodioxol-5-yl)ethyl]-N-(tert-butyl)-3-oxoisoindoline-1-carboxamide;
N-[2-(1H-indol-3-yl)ethyl]-3-oxo-2-[2-(2-phenoxyphenyl)ethyl]isoindoline-1-carboxamide;
2-(2-{4-[(diethylamino)carbonyl]phenyl}ethyl)-N-[2-(1H-indol-3-yl)ethyl]-3-oxoisoindoline-1-carboxamide;
2-[2-(3-fluorophenyl)ethyl]-N-[2-(1H-indol-3-yl)ethyl]-3-oxoisoindoline-1-carboxamide;
N-[2-(1H-indol-3-yl)ethyl]-3-oxo-2-{2-[2-(trifluoromethoxy)phenyl]ethyl}isoindoline-1-carboxamide;
2-[2-(4-fluorophenyl)ethyl]-N-[2-(1H-indol-3-yl)ethyl]-3-oxoisoindoline-1-carboxamide;
2-[2-(3,5-dimethoxyphenyl)ethyl]-N-[2-(1H-indol-3-yl)ethyl]-3-oxoisoindoline-1-carboxamide;
2-(2-biphenyl-4-ylethyl)-N-[2-(1H-indol-3-yl)ethyl]-3-oxoisoindoline-1-carboxamide;
N-[2-(1H-indol-3-yl)ethyl]-3-oxo-2-[2-(4-phenoxyphenyl)ethyl]isoindoline-1-carboxamide;
2-[2-(2-ethoxyphenyl)ethyl]-N-[2-(1H-indol-3-yl)ethyl]-3-oxoisoindoline-1-carboxamide;
2-[2-(1,3-benzodioxol-5-yl)ethyl]-N-[2-(1H-indol-3-yl)ethyl]-3-oxoisoindoline-1-carboxamide;
(1R)-2-[(1S)-1-(4-fluorophenyl)ethyl]-N-[4-(methylsulfonyl)benzyl]-3-oxoisoindoline-1-carboxamide;
N-[4-(dimethylamino)benzyl]-2-[2-(dimethylamino)-2-phenylethyl]-3-oxoisoindoline-1-carboxamide;
N-[4-(dimethylamino)benzyl]-2-(2,2-diphenylethyl)-3-oxoisoindoline-1-carboxamide;
2-(1-benzyl-2-fluoroethyl)-N-[(5-methylisoxazol-3-yl)methyl]-3-oxoisoindoline-1-carboxamide;
N-(tert-butyl)-2-(2-chloro-4-fluorobenzyl)-3-oxoisoindoline-1-carboxamide;
2-(3,4-difluorobenzyl)-N-(4-fluorobenzyl)-5-hydroxy-4-methyl-3-oxoisoindoline-1-carboxamide;
2-(3,4-difluorobenzyl)-5-hydroxy-4-methyl-3-oxo-N-(pyridin-3-ylmethyl)isoindoline-1-carboxamide;
2-(3,4-difluorobenzyl)-5-hydroxy-4-methyl-3-oxo-N-(2-phenylethyl)isoindoline-1-carboxamide;
2-(3,4-difluorobenzyl)-5-hydroxy-3-oxo-4-phenyl-N-(2-phenylethyl)isoindoline-1-carboxamide;
N-(2-chlorobenzyl)-2-(3,4-difluorobenzyl)-5-hydroxy-4-methyl-3-oxoisoindoline-1-carboxamide;
2-(3,4-difluorobenzyl)-5-hydroxy-4-methyl-3-oxo-N-[2-(trifluoromethyl)benzyl]isoindoline-1-carboxamide;
N-(tert-butyl)-2-(2-chlorobenzyl)-5-hydroxy-3-oxo-4-(trimethylsilyl)isoindoline-1-carboxamide;
N-(tert-butyl)-2-(2-chlorobenzyl)-5-hydroxy-4-methyl-3-oxoisoindoline-1-carboxamide;
2-(biphenyl-2-ylmethyl)-N-(tert-butyl)-5-hydroxy-3-oxo-4-phenylisoindoline-1-carboxamide;
2-[3,5-bis(trifluoromethyl)benzyl]-N-(tert-butyl)-5-hydroxy-3-oxo-4-phenylisoindoline-1-carboxamide;
2-[2-(4-chlorophenyl)propyl]-N-{3-[(dimethylamino)carbonyl]-4-fluorobenzyl 1-3-oxoisoindoline-1-carboxamide;
2-[3,5-bis(trifluoromethyl)benzyl]-N-(4-cyanophenyl)-3-oxoisoindoline-1-carboxamide;
2-(1-benzyl-2-fluoroethyl)-N-butyl-3-oxoisoindoline-1-carboxamide;
2-(1-benzyl-2-fluoroethyl)-N-(4-fluorobenzyl)-3-oxoisoindoline-1-carboxamide;
2-(1-benzyl-2-fluoroethyl)-N-[4-(dimethylamino)benzyl]-3-oxoisoindoline-1-carboxamide;
N-[4-(aminomethyl)phenyl]-2-[2-(4-chlorophenyl)propyl]-3-oxoisoindoline-1-carboxamide;
2-[2-(4-chlorophenyl)propyl]-N-(4-{[(difluoroacetyl)amino]methyl}phenyl)-3-oxoisoindoline-1-carboxamide;
N-[4-(aminocarbonyl)phenyl]-2-[2-(4-chlorophenyl)propyl]-3-oxoisoindoline-1-carboxamide;
N-(tert-butyl)-2-[2-(4-chlorophenyl)propyl]-5-(fluoromethoxy)-4-methyl-3-oxoisoindoline-1-carboxamide;
N-[4-(dimethylamino)benzyl]-3-oxo-2-(4-phenylbutyl)isoindoline-1-carboxamide;
N-[4-(dimethylamino)benzyl]-2-(2-hydroxy-2-phenylethyl)-3-oxoisoindoline-1-carboxamide;
N-[4-(dimethylamino)benzyl]-3-oxo-2-[2-(1H-pyrazol-1-yl)benzyl]isoindoline-1-carboxamide;
N-[4-(dimethylamino)benzyl]-3-oxo-2-(4-phenoxybenyzl)isoindoline-1-carboxamide;
N-[4-(dimethylamino)benzyl]-3-oxo-24(1-phenyl-1H-pyrazol-4-yl)methyl]isoindoline-1-carboxamide;
N-[4-(dimethylamino)benzyl]-2-[1-(4-fluorophenyl)ethyl]-3-oxoisoindoline-1-carboxamide;
N-[4-(dimethylamino)benzyl]-2-(diphenylmethyl)-3-oxoisoindoline-1-carboxamide;
2-(2-chloro-4-fluorobenzyl)-N-[4-(methylsulfonyl)benzyl]-3-oxoisoindoline-1-carboxamide;
N-[4-(dimethylamino)benzyl]-3-oxo-2-(1-phenylpropyl)isoindoline-1-carboxamide;
N[4-(methylsulfonyl)benzyl]-3-oxo-2-[2-(1H-pyrazol-1-yl)benzyl]isoindoline-1-carboxamide;
2-(2-hydroxy-2-phenylethyl)-N-[4-(methylsulfonyl)benzyl]-3-oxoisoindoline-1-carboxamide;
2-(2,2-diphenylethyl)-N-[4-(methylsulfonyl)benzyl]-3-oxoisoindoline-1-carboxamide;
2-(diphenylmethyl)-N-[4-(methylsulfonyl)benzyl]-3-oxoisoindoline-1-carboxamide;
2-[2-(4-chlorophenyl)propyl]-3-oxo-N-(pyridin-4-ylmethyl)isoindoline-1-carboxamide;
N-[4-(methylsulfonyl)benzyl]-3-oxo-2-(1,2,3,4-tetrahydronaphthalen-1-yl)isoindoline-1-carboxamide;
2-(2-chloro-4-fluorobenzyl)-3-oxo-N-(pyridin-4-ylmethyl)isoindoline-1-carboxamide;
2-[1-(4-fluorophenyl)ethyl]-3-oxo-N-(pyridin-4-ylmethyl)isoindoline-1-carboxamide;
(1S)-2-[(2R)-2-(4-chlorophenyl)propyl]-N-(3-methoxypropyl)-1-methyl-3-oxoisoindoline-1-carboxamide;
(1R)-2-[(2R)-2-(4-chlorophenyl)propyl]-N-(3-methoxypropyl)-1-methyl-3-oxoisoindoline-1-carboxamide;
2-[2-(4-chlorophenyl)propyl]-N-(3-methoxypropyl)-1-methyl-3-oxoisoindoline-1-carboxamide;
N-(tert-butyl)-2-[2-(4-chlorophenyl)propyl]-5-hydroxy-3-oxo-4-(trimethylsilyl)isoindoline-1-carboxamide;
N-(tert-butyl)-2-(3,4-difluorobenzyl)-5-hydroxy-3-oxo-4-(trimethylsilyl)isoindoline-1-carboxamide;
N-(tert-butyl)-2-(3,4-difluorobenzyl)-5-hydroxy-4-methyl-3-oxoisoindoline-1-carboxamide;
N-(tert-butyl)-2-(3,4-difluorobenzyl)-5-hydroxy-3-oxo-4-phenylisoindoline-1-carboxamide;
N-(tert-butyl)-2-[2-(4-chlorophenyl)propyl]-5-hydroxy-4-methyl-3-oxoisoindoline-1-carboxamide;
2-(2-chloro-4-fluorobenzyl)-N-(3-cyano-4-fluorobenzyl)-3-oxoisoindoline-1-carboxamide;
2-[3,5-bis(trifluoromethyl)benzyl]-N-(3-cyano-4-fluorobenzyl)-3-oxoisoindoline-1-carboxamide;
N,2-bis(3-cyano-4-fluorobenzyl)-3-oxoisoindoline-1-carboxamide;
N-(3-cyano-4-fluorobenzyl)-3-oxo-2-(2-phenylethyl)isoindoline-1-carboxamide;
N-(3-cyano-4-fluorobenzyl)-2-(4-fluorobenzyl)-3-oxoisoindoline-1-carboxamide;
2-benzyl-N-(3-cyano-4-fluorobenzyl)-3-oxoisoindoline-1-carboxamide;
N-(3-cyano-4-fluorobenzyl)-2-(3,4-difluorobenzyl)-3-oxoisoindoline-1-carboxamide;
2-(biphenyl-2-ylmethyl)-N-(3-cyano-4-fluorobenzyl)-3-oxoisoindoline-1-carboxamide;
2-[2-(4-chlorophenyl)propyl]-N-(3-cyano-4-fluorobenzyl)-3-oxoisoindoline-1-carboxamide;
2-(2-chloro-4-fluorobenzyl)-N-(3-{[(difluoroacetyl)amino]methyl}-4-fluorobenzyl)-3-oxoisoindoline-1-carboxamide;
2-[2-(4-chlorophenyl)propyl]-N-(3-{[(difluoroacetyl)amino]methyl}-4-fluorobenzyl)-3-oxoisoindoline-1-carboxamide;
N-[3-(aminomethyl)-4-fluorobenzyl]-2-[2-(4-chlorophenyl)propyl]-3-oxoisoindoline-1-carboxamide;
N-[3-(aminocarbonyl)-4-fluorobenzyl]-2-[2-(4-chlorophenyl)propyl]-3-oxoisoindoline-1-carboxamide;
N-[3-(aminocarbonyl)-4-fluorobenzyl]-2-(2-chloro-4-fluorobenzyl)-3-oxoisoindoline-1-carboxamide;
N-(tert-butyl)-3-oxo-2-(4-phenylbutyl)isoindoline-1-carboxamide;
N-(tert-butyl)-3-oxo-2-(1,2,3,4-tetrahydronaphthalen-1-yl)isoindoline-1-carboxamide;
N-(tert-butyl)-3-oxo-2-(4-phenoxybenyzl)isoindoline-1-carboxamide;
N-(tert-butyl)-3-oxo-2-[2-(1H-pyrazol-1-yl)benzyl]isoindoline-1-carboxamide;
N-(tert-butyl)-2-(2,2-diphenylethyl)-3-oxoisoindoline-1-carboxamide;
N-(tert-butyl)-2-(diphenylmethyl)-3-oxoisoindoline-1-carboxamide;
N-(1,3-benzodioxol-5-ylmethyl)-2-(2,2-dimethylpropyl)-3-oxoisoindoline-1-carboxamide;
2-(2-chloro-4-fluorobenzyl)-N-[(1-methyl-1H-pyrrol-2-yl)methyl]-3-oxoisoindoline-1-carboxamide;
N-(1,3-benzodioxol-5-ylmethyl)-2-(2-chloro-4-fluorobenzyl)-3-oxoisoindoline-1-carboxamide;
2-(2-chloro-4-fluorobenzyl)-N-[4-(difluoromethoxy)benzyl]-3-oxoisoindoline-1-carboxamide;
2-[3,5-bis(trifluoromethyl)benzyl]-3-oxo-N-(2-pyridin-4-ylethyl)isoindoline-1-carboxamide;
2-[2-(4-chlorophenyl)propyl]-3-oxo-N-(2-pyridin-4-ylethyl)isoindoline-1-carboxamide;
2-[3,5-bis(trifluoromethyl)benzyl]-N-[(1-methyl-1H-pyrrol-2-yl)methyl]-3-oxoisoindoline-1-carboxamide;
2-[2-(4-chlorophenyl)propyl]-N-[1-methyl-1H-pyrrol-2-yl)methyl]-3-oxoisoindoline-1-carboxamide;
2-[2-(4-chlorophenyl)propyl]-N-[4-(difluoromethoxy)benzyl]-3-oxoisoindoline-1-carboxamide;
2-[3,5-bis(trifluoromethyl)benzyl]-N-[4-(difluoromethoxy)benzyl]-3-oxoisoindoline-1-carboxamide;
N-(1,3-benzodioxol-5-ylmethyl)-2-[3,5-bis(trifluoromethyl)benzyl]-3-oxoisoindoline-1-carboxamide;
2-(2-chloro-4-fluorobenzyl)-N-[4-(dimethylamino)benzyl]-3-oxoisoindoline-1-carboxamide;
N-(1-benzylpyrrolidin-3-yl)-2-(2-chloro-4-fluorobenzyl)-3-oxoisoindoline-1-carboxamide;
N-(1-benzylpyrrolidin-3-yl)-2-[2-(4-chlorophenyl)propyl]-3-oxoisoindoline-1-carboxamide;
2-(2-chloro-4-fluorobenzyl)-N-{[5-(2-furyl)isoxazol-3-yl]methyl}-3-oxoisoindoline-1-carboxamide;
2-(2,2-dimethylpropyl)-N-{[5-(2-furyl)isoxazol-3-yl]methyl}-3-oxoisoindoline-1-carboxamide;
2-[3,5-bis(trifluoromethyl)benzyl]-N-{[5-(2-furyl)isoxazol-3-yl]methyl}-3-oxoisoindoline-1-carboxamide;
2-[2-(4-chlorophenyl)propyl]-N-[3-(1H-imidazol-1-yl)propyl]-3-oxoisoindoline-1-carboxamide;
2-[3,5-bis(trifluoromethyl)benzyl]-N-[4-(dimethylamino)benzyl]-3-oxoisoindoline-1-carboxamide;
2-[2-(4-chlorophenyl)propyl]-N-[4-(dimethylamino)benzyl]-3-oxoisoindoline-1-carboxamide;
N-(1-benzylpyrrolidin-3-yl)-2-[3,5-bis(trifluoromethyl)benzyl]-3-oxoisoindoline-1-carboxamide;
2-[2-(4-chlorophenyl)propyl]-N-[4-(methylsulfonyl)benzyl]-3-oxoisoindoline-1-carboxamide;
N-(tert-butyl)-3-oxo-2-[(1-phenyl-1H-tetrazol-5-yl)methyl]isoindoline-1-carboxamide;
2-[2-(4-chlorophenyl)propyl]-N-[3-(dimethylamino)propyl]-3-oxoisoindoline-1-carboxamide;
2-[2-(4-chlorophenyl)propyl]-N-[2-(dimethylamino)ethyl]-3-oxoisoindoline-1-carboxamide;
2-[2-(4-chlorophenyl)propyl]-3-oxo-N-(pyridin-3-ylmethyl)isoindoline-1-carboxamide;
N-[2-(4-benzoylpiperazin-1-yl)ethyl]-2-[2-(4-chlorophenyl)propyl]-3-oxoisoindoline-1-carboxamide;
2-[2-(4-chlorophenyl)propyl]-3-oxo-N-(1-pyridin-3-ylethyl)isoindoline-1-carboxamide;
2-[2-(4-chlorophenyl)propyl]-N-(3-methoxyphenyl)-3-oxoisoindoline-1-carboxamide;
2-[2-(4-chlorophenyl)propyl]-3-oxo-N-(1-pyridin-4-ylethyl)isoindoline-1-carboxamide;
2-[2-(4-chlorophenyl)propyl]-N-(4-cyanophenyl)-3-oxoisoindoline-1-carboxamide;
2-[2-(4-chlorophenyl)propyl]-N-(3-methoxypropyl)-3-oxoisoindoline-1-carboxamide;
N-(1,3-benzodioxol-5-ylmethyl)-2-[2-(4-chlorophenyl)propyl]-3-oxoisoindoline-1-carboxamide;
2-[2-(4-chlorophenyl)propyl]-N-(3,4-dimethoxybenzyl)-3-oxoisoindoline-1-carboxamide;
2-[2-(4-chlorophenyl)propyl]-N-[(3-methyl-5-phenylisoxazol-4-yl)methyl]-3-oxoisoindoline-1-carboxamide;
N-butyl-2-[2-(4-chlorophenyl)propyl]-7-fluoro-3-oxoisoindoline-1-carboxamide;
N-(tert-butyl)-2-[2-(4-chlorophenyl)propyl]-7-fluoro-3-oxoisoindoline-1-carboxamide;
N-(tert-butyl)-3-oxo-2-[2-(phenylsulfonyl)ethyl]isoindoline-1-carboxamide;
N-(tert-butyl)-2-[2-(4-fluorophenoxy)propyl]-3-oxoisoindoline-1-carboxamide;
N-(tert-butyl)-3-oxo-2-(2-phenoxypropyl)isoindoline-1-carboxamide;
N-benzyl-2-[(5-methylisoxazol-3-yl)methyl]-3-oxoisoindoline-1-carboxamide;
N-benzyl-2-[4-(methylsulfonyl)benzyl]-3-oxoisoindoline-1-carboxamide;
N-benzyl-3-oxo-2-[2-(phenylsulfonyl)ethyl]isoindoline-1-carboxamide;
N-benzyl-3-oxo-2-(2-phenoxyethyl)isoindoline-1-carboxamide;
N-benzyl-3-oxo-2-(2-phenoxypropyl)isoindoline-1-carboxamide;
N-benzyl-2-[2-(4-fluorophenoxy)propyl]-3-oxoisoindoline-1-carboxamide;
N-benzyl-2-[(1-benzyl-1H-pyrazol-4-yl)methyl]-3-oxoisoindoline-1-carboxamide;
N-benzyl-2-[(5-methyl-3-phenylisoxazol-4-yl)methyl]-3-oxoisoindoline-1-carboxamide;
N-benzyl-3-oxo-2-[(3-phenylisoxazol-5-yl)methyl]isoindoline-1-carboxamide;
N-(tert-butyl)-5,6-dichloro-2-(4-cyanobenzyl)-3-oxoisoindoline-1-carboxamide;
N-(tert-butyl)-5,6-dichloro-2-(4-fluorobenzyl)-3-oxoisoindoline-1-carboxamide;
N-(tert-butyl)-5,6-dichloro-2-(2-methoxybenzyl)-3-oxoisoindoline-1-carboxamide;
N-(tert-butyl)-5,6-dichloro-2-[4-(difluoromethoxy)benzyl]-3-oxoisoindoline-1-carboxamide;
N-(tert-butyl)-5-fluoro-2-(2-methoxybenzyl)-3-oxoisoindoline-1-carboxamide;
N-(4-fluorobenzyl)-3-oxo-2-(2-pyridin-4-ylethyl)isoindoline-1-carboxamide;
2-[(1-methyl-1H-pyrrol-2-yl)methyl]-3-oxo-N-[3-(trifluoromethyl)benzyl]isoindoline-1-carboxamide;
N-(2-furylmethyl)-3-oxo-2-(2-phenylpropyl)isoindoline-1-carboxamide;
2-[2-(4-chlorophenyl)ethyl]-N-[(5-methyl-2-furyl)methyl]-3-oxoisoindoline-1-carboxamide;
N-(4-fluorobenzyl)-2-[1-methyl-1H-pyrrol-2-yl)methyl]-3-oxoisoindoline-1-carboxamide;
N-(2-chlorobenzyl)-2-[2-(1H-indol-3-yl)-1-methylethyl]-3-oxoisoindoline-1-carboxamide;
N-(tert-butyl)-5,6-dichloro-2-[2-(4-chloro-2-methylphenyl)-2,2-difluoroethyl]-3-oxoisoindoline-1-carboxamide;
N-(tert-butyl)-5,6-dichloro-2-[2-(4-chlorophenyl)propyl]-3-oxoisoindoline-1-carboxamide;
N-(tert-butyl)-2-[2-(4-chloro-2-methylphenyl)-2,2-difluoro ethyl]-3-oxoisoindoline-1-carboxamide;
N-benzyl-2-[2-(4-chloro-2-methylphenyl)-2,2-difluoroethyl]-3-oxoisoindoline-1-carboxamide;
2-[2-(4-chlorophenyl)propyl]-3-oxo-N-[2-(trifluoromethyl)benzyl]isoindoline-1-carboxamide;
2-[3-(difluoromethoxy)benzyl]-3-oxo-N-[(trimethylsilyl)methyl]isoindoline-1-carboxamide;
N-(2-chlorobenzyl)-2-[2-(4-chlorophenyl)propyl]-3-oxoisoindoline-1-carboxamide;
2-[4-(difluoromethoxy)benzyl]-3-oxo-N-[(trimethylsilyl)methyl]isoindoline-1-carboxamide;
N-(2-chlorobenzyl)-2-(2,5-dimethylbenzyl)-3-oxoisoindoline-1-carboxamide;
2-(biphenyl-2-ylmethyl)-N-(4-fluorobenzyl)-3-oxoisoindoline-1-carboxamide;
N-(2-chlorobenzyl)-2-[(1R)-1-(4-methoxyphenyl)ethyl]-3-oxoisoindoline-1-carboxamide;
N-(2-chlorobenzyl)-2-[(1R)-1-(3-methoxyphenyl)ethyl]-3-oxoisoindoline-1-carboxamide;
N-(2-chlorobenzyl)-2-[(1S)-1-(1-naphthyl)ethyl]-3-oxoisoindoline-1-carboxamide;
N-benzyl-3-oxo-2-(4-phenoxybenzyl)isoindoline-1-carboxamide;
N-(2-chlorobenzyl)-3-oxo-2-(3-phenylpropyl)isoindoline-1-carboxamide;
N-(2-chlorobenzyl)-3-oxo-2-(2-phenylethyl)isoindoline-1-carboxamide;
N-(2-chlorobenzyl)-3-oxo-2-(1-phenylpropyl)isoindoline-1-carboxamide;
N-(2-chlorobenzyl)-2-(1-methyl-3-phenylpropyl)-3-oxoisoindoline-1-carboxamide;
N-(2-chlorobenzyl)-3-oxo-2-(2-phenylpropyl)isoindoline-1-carboxamide;
2-(biphenyl-2-ylmethyl)-3-oxo-N-[2-(trifluoromethyl)benzyl]isoindoline-1-carboxamide;
2-(biphenyl-2-ylmethyl)-N-(2-chlorobenzyl)-3-oxoisoindoline-1-carboxamide;
3-oxo-2-(1-phenylpropyl)-N-[2-(trifluoromethyl)benzyl]isoindoline-1-carboxamide;
3-oxo-2-(2-phenylpropyl)-N-[2-(trifluoromethyl)benzyl]isoindoline-1-carboxamide;
2-(1-methyl-3-phenylpropyl)-3-oxo-N-[2-(trifluoromethyl)benzyl]isoindoline-1-carboxamide;
3-oxo-2-(2-phenylethyl)-N-[2-(trifluoromethyl)benzyl]isoindoline-1-carboxamide;
N-benzyl-2-[2-(5-bromo-2-methoxyphenyl)ethyl]-3-oxoisoindoline-1-carboxamide;
3-oxo-2-(3-phenylpropyl)-N-[2-(trifluoromethyl)benzyl]isoindoline-1-carboxamide;
N-benzyl-2-[2-(3-bromo-4-methoxyphenyl)ethyl]-3-oxoisoindoline-1-carboxamide;
2-(2-methylbutyl)-3-oxo-N-[2-(trifluoromethyl)benzyl]isoindoline-1-carboxamide;
N-benzyl-2-(2,4-difluorobenzyl)-3-oxoisoindoline-1-carboxamide;
2-(cyclohexylmethyl)-3-oxo-N-[2-(trifluoromethyl)benzyl]isoindoline-1-carboxamide;
2-(3-fluorobenzyl)-3-oxo-N-[2-(trifluoromethyl)benzyl]isoindoline-1-carboxamide;
2-(2-ethoxybenzyl)-3-oxo-N-[2-(trifluoromethyl)benzyl]isoindoline-1-carboxamide;
3-oxo-2-[4-(trifluoromethoxy)benzyl]-N-[2-(trifluoromethyl)benzyl]isoindoline-1-carboxamide;
2-[2-(4-chlorophenyl)propyl]-N-[2-(3,4-dimethoxyphenyl)ethyl]-3-oxoisoindoline-1-carboxamide;
2-[2-(4-chlorophenyl)propyl]-3-oxo-N-[2-(2-thienyl)ethyl]isoindoline-1-carboxamide;
2-[2-(4-chlorophenyl)propyl]-N-[2-(4-methoxyphenyl)ethyl]-3-oxoisoindoline-1-carboxamide;
2-[2-(4-chlorophenyl)propyl]-3-oxo-N-(2-phenylethyl)isoindoline-1-carboxamide;
2-[2-(4-chlorophenyl)propyl]-N-(2-methoxyethyl)-3-oxoisoindoline-1-carboxamide;
2-[2-(4-chlorophenyl)propyl]-N-(4-fluorobenzyl)-3-oxoisoindoline-1-carboxamide;
N-benzyl-2-[4-(difluoromethoxy)benzyl]-3-oxoisoindoline-1-carboxamide;
N-benzyl-2-[3-(difluoromethoxy)benzyl]-3-oxoisoindoline-1-carboxamide;
N-butyl-24 1-naphthylmethyl)-3-oxoisoindoline-1-carboxamide;
N-benzyl-2-cycloheptyl-3-oxoisoindoline-1-carboxamide;
N-(1H-1,2,3-benzotriazol-1-ylmethyl)-2-[2-(4-bromophenyl)ethyl]-3-oxoisoindoline-1-carboxamide;
N-(1H-1,2,3-benzotriazol-1-ylmethyl)-2-(1-ethylpropyl)-3-oxoisoindoline-1-carboxamide;
N-benzyl-3-oxo-2-[3-(1H-pyrrol-1-yl)benzyl]isoindoline-1-carboxamide;
N-benzyl-2-(3-fluorobenzyl)-3-oxoisoindoline-1-carboxamide;
N-benzyl-2-[2-(2-methoxyphenyl)ethyl]-3-oxoisoindoline-1-carboxamide;
N-benzyl-2-(2-ethoxybenzyl)-3-oxoisoindoline-1-carboxamide;
N-(1H-1,2,3-benzotriazol-1-ylmethyl)-3-oxo-2-(2-phenylpropyl)isoindoline-1-carboxamide;
N-benzyl-2-(4-cyanobenzyl)-3-oxoisoindoline-1-carboxamide;
N-benzyl-2-(3,5-dimethoxybenzyl)-3-oxoisoindoline-1-carboxamide;
N-benzyl-24 1-(1-naphthyl)ethyl]-3-oxoisoindoline-1-carboxamide;
N-benzyl-3-oxo-2-[4-(trifluoromethyl)benzyl]isoindoline-1-carboxamide;
ethyl N-({2-[3,5-bis(trifluoromethyl)benzyl]-3-oxo-2,3-dihydro-1H-isoindol-1-yl}carbonyl)-beta-alaninate;
2-(1-naphthylmethyl)-3-oxo-N-[(trimethylsilyl)methyl]isoindoline-1-carboxamide;
ethyl N-({2-[2-(3,4-dichlorophenyl)ethyl]-3-oxo-2,3-dihydro-1H-isoindol-1-yl}carbonyl)-beta-alaninate;
methyl 4-({1-[(benzylamino)carbonyl]-3-oxo-1,3-dihydro-2H-isoindol-2-yl}methyl)benzoate;
3-oxo-2-[3-(trifluoromethyl)benzyl]-N-[(trimethylsilyl)methyl]isoindoline-1-carboxamide;
2-[1-(1-naphthyl)ethyl]-3-oxo-N-[(trimethylsilyl)methyl]isoindoline-1-carboxamide;
3-oxo-2-[4-(trifluoromethyl)benzyl]-N-[(trimethylsilyl)methyl]isoindoline-1-carboxamide;
2-[2-(4-bromophenyl)ethyl]-3-oxo-N-[(trimethylsilyl)methyl]isoindoline-1-carboxamide;
2-(2-chloro-6-phenoxybenzyl)-3-oxo-N-[(trimethylsilyl)methyl]isoindoline-1-carboxamide;
2-(3,4-dichlorobenzyl)-3-oxo-N-[(trimethylsilyl)methyl]isoindoline-1-carboxamide;
N-benzyl-24 1-benzylpyrrolidin-3-yl)-3-oxoisoindoline-1-carboxamide;
N-benzyl-2-(1-benzylpyrrolidin-3-yl)-4,5-dimethoxy-3-oxoisoindoline-1-carboxamide;
N-benzyl-2-(3,4-difluorobenzyl)-4,5-dimethoxy-3-oxoisoindoline-1-carboxamide;
N-benzyl-2-[2-(4-chlorophenyl)propyl]-4,5-dimethoxy-3-oxoisoindoline-1-carboxamide;
N-benzyl-4,5-dimethoxy-2-(1-methyl-3-phenylpropyl)-3-oxoisoindoline-1-carboxamide;
N-benzyl-2-[(1-methyl-1H-pyrrol-2-yl)methyl]-3-oxoisoindoline-1-carboxamide;
N-benzyl-3-oxo-2-(1-phenyl-2-pyrrolidin-1-ylethyl)isoindoline-1-carboxamide;
N-benzyl-2-[2-(4-methoxyphenyl)-2-oxoethyl]-3-oxoisoindoline-1-carboxamide;
N-benzyl-2-[(1R)-1-(4-methoxyphenyl)ethyl]-3-oxoisoindoline-1-carboxamide;
N-benzyl-2-(3,4-difluorobenzyl)-3-oxoisoindoline-1-carboxamide;
N-benzyl-2-[(1R)-1-(3-methoxyphenyl)ethyl]-3-oxoisoindoline-1-carboxamide;
N-benzyl-2-(2,5-dimethylbenzyl)-3-oxoisoindoline-1-carboxamide;
N-benzyl-2-(1-methyl-3-phenylpropyl)-3-oxoisoindoline-1-carboxamide;
N-benzyl-3-oxo-2-{2-[3-(trifluoromethyl)phenyl]ethyl}isoindoline-1-carboxamide;
N-benzyl-2-[3,5-bis(trifluoromethyl)benzyl]-3-oxoisoindoline-1-carboxamide;
N-benzyl-2-[2-(6-chloro-1H-indol-3-yl)ethyl]-3-oxoisoindoline-1-carboxamide;
N,2-dibenzyl-3-oxoisoindoline-1-carboxamide;
N-benzyl-2-(cyclohexylmethyl)-3-oxoisoindoline-1-carboxamide;
N-benzyl-3-oxo-2-(2-thienylmethyl)isoindoline-1-carboxamide;
2-(1,3-benzodioxol-5-ylmethyl)-N-cyclohexyl-3-oxoisoindoline-1-carboxamide;
2-(2-methoxybenzyl)-N-(4-methylcyclohexyl)-3-oxoisoindoline-1-carboxamide;
tert-butyl N-{[3-oxo-2-(3-pyrrolidin-1-ylpropyl)-2,3-dihydro-1H-isoindol-1-yl]carbonyl}glycinate;
N-(tert-butyl)-2-(2-methoxybenzyl)-3-oxoisoindoline-1-carboxamide;
N-(1H-1,2,3-benzotriazol-1-ylmethyl)-2-(2-bromobenzyl)-3-oxoisoindoline-1-carboxamide;
2-[2-(4-chlorophenyl)ethyl]-N-cyclohexyl-3-oxoisoindoline-1-carboxamide;
N-(2,3-dimethylcyclohexyl)-3-oxo-2-(2-thienylmethyl)isoindoline-1-carboxamide;
N-(1H-1,2,3-benzotriazol-1-ylmethyl)-2-(biphenyl-2-ylmethyl)-3-oxoisoindoline-1-carboxamide;
2-(2-chlorobenzyl)-N-(4-methylcyclohexyl)-3-oxoisoindoline-1-carboxamide;
N-butyl-2-(2-cyclohex-1-en-1-ylethyl)-3-oxoisoindoline-1-carboxamide;
N-benzyl-2-[(2R)-2-hydroxy-1,2-diphenylethyl]-3-oxoisoindoline-1-carboxamide;
2-(biphenyl-2-ylmethyl)-N-butyl-3-oxoisoindoline-1-carboxamide;
2-(biphenyl-2-ylmethyl)-N-isopropyl-3-oxoisoindoline-1-carboxamide;
tert-butyl N-{[2-(2-bromobenzyl)-3-oxo-2,3-dihydro-1H-isoindol-1-yl]carbonyl}glycinate;
2-[2-(4-chlorophenyl)propyl]-N-isopropyl-3-oxoisoindoline-1-carboxamide;
methyl N-{[3-oxo-2-(2-phenylethyl)-2,3-dihydro-1H-isoindol-1-yl]carbonyl}glycinate;
N-(tert-butyl)-24 1-(2-naphthyl)ethyl]-3-oxoisoindoline-1-carboxamide;
N-(1H-1,2,3-benzotriazol-1-ylmethyl)-2-[1-(2-naphthyl)ethyl]-3-oxoisoindoline-1-carboxamide;
2-[2-(3,4-diethoxyphenyl)ethyl]-3-oxo-N-(2-phenylethyl)isoindoline-1-carboxamide;
2-benzyl-3-oxo-N-(2-phenylethyl)isoindoline-1-carboxamide;
N-(1H-1,2,3-benzotriazol-1-ylmethyl)-2-[4-(dimethylamino)benzyl]-3-oxoisoindoline-1-carboxamide;
N-benzyl-2-(3-methoxybenzyl)-3-oxoisoindoline-1-carboxamide;
2-(2-chloro-4-fluorobenzyl)-N-cyclopentyl-3-oxoisoindoline-1-carboxamide;
2-(2-chloro-4-fluorobenzyl)-3-oxo-N-(pyridin-3-ylmethyl)isoindoline-1-carboxamide;
2-(2,5-dimethoxybenzyl)-3-oxo-N-(2-phenylethyl)isoindoline-1-carboxamide;
N-(sec-butyl)-2-[2-(4-chlorophenyl)ethyl]-3-oxoisoindoline-1-carboxamide;
N-benzyl-2-(2,3-difluorobenzyl)-3-oxoisoindoline-1-carboxamide;
2-(2-chloro-4-fluorobenzyl)-3-oxo-N-(2-phenylethyl)isoindoline-1-carboxamide;
2-[2-(4-chlorophenyl)ethyl]-3-oxo-N-(2-phenylethyl)isoindoline-1-carboxamide;
N-(1H-1,2,3-benzotriazol-1-ylmethyl)-2-(2-methylbenzyl)-3-oxoisoindoline-1-carboxamide;
N-(tert-butyl)-2-(cyclohexylmethyl)-3-oxoisoindoline-1-carboxamide;
2-(1-methyl-3-phenylpropyl)-3-oxo-N-(2-phenylethyl)isoindoline-1-carboxamide;
N-benzyl-2-cyclohexyl-3-oxoisoindoline-1-carboxamide;
N-(tert-butyl)-2-(2-cyclohex-1-en-1-ylethyl)-3-oxoisoindoline-1-carboxamide;
N-(tert-butyl)-3-oxo-2-(1-phenylethyl)isoindoline-1-carboxamide;
tert-butyl N-{[2-(cyclohexylmethyl)-3-oxo-2,3-dihydro-1H-isoindol-1-yl]carbonyl}glycinate;
N-(1H-1,2,3-benzotriazol-1-ylmethyl)-2-(2-cyclohex-1-en-1-ylethyl)-3-oxoisoindoline-1-carboxamide;
tert-butyl N-{[2-(biphenyl-2-ylmethyl)-3-oxo-2,3-dihydro-1H-isoindol-1-yl]carbonyl}glycinate;
N-benzyl-2-(2,2-dimethylpropyl)-3-oxoisoindoline-1-carboxamide;
N-benzyl-2-(3-methylbutyl)-3-oxoisoindoline-1-carboxamide;
N-benzyl-3-oxo-2-[2-(2-thienyl)ethyl]isoindoline-1-carboxamide;
N-(1H-1,2,3-benzotriazol-1-ylmethyl)-3-oxo-2-(2-phenylethyl)isoindoline-1-carboxamide;
N-benzyl-2-[2-(4-chlorophenyl)propyl]-3-oxoisoindoline-1-carboxamide;
N-(tert-butyl)-2-[2-(4-chlorophenyl)propyl]-3-oxoisoindoline-1-carboxamide;
N-benzyl-2-(2-methylbenzyl)-3-oxoisoindoline-1-carboxamide;
N-benzyl-2-(2-methoxybenzyl)-3-oxoisoindoline-1-carboxamide;
N-benzyl-3-oxo-2-(2-phenylethyl)isoindoline-1-carboxamide;
N-benzyl-2-[1-(2-naphthyl)ethyl]-3-oxoisoindoline-1-carboxamide;
methyl N-({2-[2-(4-chlorophenyl)propyl]-3-oxo-2,3-dihydro-1H-isoindol-1-yl}carbonyl)glycinate;
tert-butyl N-({2-[1-(2-naphthyl)ethyl]-3-oxo-2,3-dihydro-1H-isoindol-1-yl}carbonyl)glycinate;
N-(1H-1,2,3-benzotriazol-1-ylmethyl)-2-[2-(4-chlorophenyl)propyl]-3-oxoisoindoline-1-carboxamide;
N-butyl-2-[1-(2-naphthyl)ethyl]-3-oxoisoindoline-1-carboxamide;
N-benzyl-2-(2-bromobenzyl)-3-oxoisoindoline-1-carboxamide;
N-benzyl-2-(2-cyclohex-1-en-1-ylethyl)-3-oxoisoindoline-1-carboxamide;
N-benzyl-2-(biphenyl-2-ylmethyl)-3-oxoisoindoline-1-carboxamide;
N-butyl-2-[2-(4-chlorophenyl)propyl]-3-oxoisoindoline-1-carboxamide;
N-butyl-2-[2-(4-chlorophenyl)-2-methyl-propyl]-3-oxo-1H-isoindole-1-carboxamide;
N-(tert-butyl)-2-[(4,4′-difluorobiphenyl-2-yl)methyl]-3-oxoisoindoline-1-carboxamide;
(1R or 1S)—N-(tert-butyl)-2-[(3′,4′-difluorobiphenyl-2-yl)methyl]-3-oxoisoindoline-1-carboxamide;
(1S or 1R)-N-(tert-butyl)-2-[(3′,4′-difluorobiphenyl-2-yl)methyl]-3-oxoisoindoline-1-carboxamide;
2-[2-(4-chlorophenyl)-2-methylpropyl]-N-[(1-isopropyl-5-oxopyrrolidin-3-yl)methyl]-3-oxoisoindoline-1-carboxamide;
2-[2-(4-chlorophenyl)propyl]-N-[(1-isopropyl-5-oxopyrrolidin-3-yl)methyl]-3-oxoisoindoline-1-carboxamide;
1H-isoindole-1-carboxamide, 2-[2-(4-chlorophenyl)propyl]-2,3-dihydro-N-[2-[(1-methylethyl)amino]-2-oxoethyl]-3-oxo-;
N-(tert-butyl)-2-[(4′,5-difluorobiphenyl-2-yl)methyl]-3-oxoisoindoline-1-carboxamide;
N-(tert-butyl)-2-[(5-fluorobiphenyl-2-yl)methyl]-3-oxoisoindoline-1-carboxamide;
N-(tert-butyl)-2-[(4-fluorobiphenyl-2-yl)methyl]-3-oxoisoindoline-1-carboxamide;
N-(tert-butyl)-2-[(4-fluorobiphenyl-2-yl)methyl]-3-oxoisoindoline-1-carboxamide;
(1R or 1S)—N-(tert-butyl)-2-[(3′,4′-difluorobiphenyl-2-yl)methyl]-3-oxoisoindoline-1-carboxamide;
(1S or 1R)—N-(tert-butyl)-2-[(3′,4′-difluorobiphenyl-2-yl)methyl]-3-oxoisoindoline-1-carboxamide;
2-[2-(4-chlorophenyl)propyl]-N-[(1-isopropyl-5-oxopyrrolidin-3-yl)methyl]-3-oxoisoindoline-1-carboxamide;
1H-isoindole-1-carboxamide, 2-[2-(4-chlorophenyl)propyl]-2,3-dihydro-N-[2-[(1-methylethyl)amino]-2-oxoethyl]-3-oxo-;
2-(2,2-dimethylpropyl)-6-fluoro-3-oxo-N-(1-phenylethyl)isoindoline-1-carboxamide;
2-(2,2-dimethylpropyl)-6-fluoro-N-(3-fluorobenzyl)-3-oxoisoindoline-1-carboxamide;
2-(2,2-dimethylpropyl)-6-fluoro-N-(2-fluorobenzyl)-3-oxoisoindoline-1-carboxamide;
2-(2,2-dimethylpropyl)-N-(3-fluorobenzyl)-3-oxoisoindoline-1-carboxamide;
2-(2,2-dimethylpropyl)-N-(2-fluorobenzyl)-3-oxoisoindoline-1-carboxamide;
2-(1-methyl-1-phenylethyl)-3-oxo-N-(1-phenylethyl)isoindoline-1-carboxamide;
6-fluoro-3-oxo-N,2-bis(1-phenylethyl)isoindoline-1-carboxamide;
N-(1-methyl-1-phenylethyl)-3-oxo-2-(1-phenylethyl)isoindoline-1-carboxamide;
3-oxo-N,2-bis(1-phenylethyl)isoindoline-1-carboxamide;
N-(3-fluorobenzyl)-3-oxo-2-(1-phenylethyl)isoindoline-1-carboxamide;
N-(2-fluorobenzyl)-3-oxo-2-(1-phenylethyl)isoindoline-1-carboxamide;
(1R or 1S)-2-[(2S or 2R)-2-(4-chlorophenyl)propyl]-3-oxo-N-propylisoindoline-1-carboxamide;
(1S or 1R)-2-[(2R or 2S)-2-(4-chlorophenyl)propyl]-3-oxo-N-propylisoindoline-1-carboxamide;
(1R or 1S)-2-[(2R or 2S)-2-(4-chlorophenyl)propyl]-3-oxo-N-propylisoindoline-1-carboxamide;
(R or S)2-(biphenyl-2-ylmethyl)-6-chloro-N-ethyl-3-oxoisoindoline-1-carboxamide;
(S or R)2-(biphenyl-2-ylmethyl)-6-chloro-N-ethyl-3-oxoisoindoline-1-carboxamide;
N-(4-fluorobenzyl)-2-[1-(4-fluorophenyl)ethyl]-3-oxoisoindoline-1-carboxamide;
2-[1-(4-chlorophenyl)ethyl]-N-(4-fluorobenzyl)-3-oxoisoindoline-1-carboxamide;
N-benzyl-2-[1-(4-fluorophenyl)ethyl]-3-oxoisoindoline-1-carboxamide;
2-[1-(4-chlorophenyl)-1-methylethyl]-N-(4-fluorobenzyl)-3-oxoisoindoline-1-carboxamide;
N-benzyl-6-fluoro-2-[1-(4-fluorophenyl)-1-methylethyl]-3-oxoisoindoline-1-carboxamide;
2-[1-(4-chlorophenyl)-1-methylethyl]-3-oxo-N-(4,4,4-trifluorobutyl)isoindoline-1-carboxamide;
N-(4-fluorobenzyl)-2-[1-(4-fluorophenyl)-1-methylethyl]-3-oxoisoindoline-1-carboxamide;
N-benzyl-6-fluoro-2-(1-methyl-1-phenylethyl)-3-oxoisoindoline-1-carboxamide;
2-[1-(4-fluorophenyl)-1-methylethyl]-3-oxo-N-(4,4,4-trifluorobutyl)isoindoline-1-carboxamide;
N-(4-fluorobenzyl)-2-(1-methyl-1-phenylethyl)-3-oxoisoindoline-1-carboxamide;
2-(1-methyl-1-phenylethyl)-3-oxo-N-(4,4,4-trifluorobutyl)isoindoline-1-carboxamide;
2-[2-(4-chlorophenyl)propyl]-N-(5-cyanopentyl)-6-fluoro-3-oxoisoindoline-1-carboxamide;
N-benzyl-6-bromo-3-oxo-2-(1-phenylpropyl)isoindoline-1-carboxamide;
N-benzyl-2-[2-(4-chlorophenyl)propyl]-4-fluoro-3-oxoisoindoline-1-carboxamide;
2-[2-(4-chlorophenyl)propyl]-N-(4,4-difluorobutyl)-4-fluoro-3-oxoisoindoline-1-carboxamide;
2-[2-(4-chlorophenyl)propyl]-4-fluoro-3-oxo-N-propylisoindoline-1-carboxamide;
2-[2-(4-chlorophenyl)propyl]-N-ethyl-4-fluoro-3-oxoisoindoline-1-carboxamide;
N-benzyl-2-(biphenyl-2-ylmethyl)-4-fluoro-3-oxoisoindoline-1-carboxamide;
2-(biphenyl-2-ylmethyl)-N-(4,4-difluorobutyl)-4-fluoro-3-oxoisoindoline-1-carboxamide;
2-(biphenyl-2-ylmethyl)-4-fluoro-3-oxo-N-propylisoindoline-1-carboxamide;
2-(biphenyl-2-ylmethyl)-N-ethyl-4-fluoro-3-oxoisoindoline-1-carboxamide; and
N-benzyl-4-fluoro-3-oxo-2-[(1R)-1-phenylethyl]isoindoline-1-carboxamide;
or a pharmaceutically acceptable salt thereof; or an enantiomer thereof.

60. A compound of Formula I 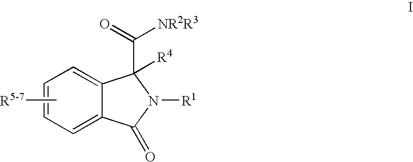
or a pharmaceutically acceptable salt thereof; wherein
R¹ represents C₁-C₁₂ alkyl (which alkyl group is optionally substituted by one or more groups selected from halogen, C₂-C₆ alkenyl, C₃-C₈ cycloalkyl, cyano, oxo, —OR⁸, —COR^{S}, —SR¹⁰, —COXR¹¹, —N(R^{12a})(R^{12b}), —N(R^{13a})C(O)OR^{13b}, —OC(O)N(R^{14a})(R^{14b}), —SO₂R¹⁵, aryl and Het¹); further R¹ represents aryl or Het²;
R⁸ to R¹¹, R^{13a}, R^{13b}, R¹⁵ independently represent, at each occurrence, hydrogen, C₁-C₆ alkyl, aryl or Het⁹ (which C₁-C₆ alkyl, aryl and Het⁹ groups are optionally substituted with one or more substituents selected from —OH, halogen, cyano, nitro, C₁-C₆ alkyl, aryl and Het¹⁰);
R^{12a} and R^{12b} independently represent, at each occurrence, hydrogen, C₁-C₆ alkyl, aryl or Het¹¹ (which C₁-C₆ alkyl, aryl and Het¹¹ groups are optionally substituted with one or more substituents selected from —OH, halogen, cyano, nitro, C₁-C₆ alkyl, aryl and Het¹²), or together represent C₃-C₆ alkylene, optionally interrupted by an O atom;
R^{14a} and R^{14b} independently represent, at each occurrence, hydrogen, C₁-C₆ alkyl, aryl or Het¹³ (which C₁-C₆ alkyl, aryl and Het¹³ groups are optionally substituted with one or more substituents selected from —OH, halogen, cyano, nitro, C₁-C₆ alkyl, aryl and Het¹⁴), or together represent C₃-C₆ alkylene, optionally interrupted by an O atom;
R² represents C₁-C₁₂ alkyl (which alkyl group is optionally substituted by one or more groups selected from halogen, —OR¹⁶, —COR¹⁷, C₂-C₆ alkenyl, C₃-C₈ cycloalkyl, cyano, trialkylsilyl, —COXR¹⁸, aryl and Het³); further R² represents —(CH₂)ₖN(R^{19a})(R^{19b}), —(CH₂)ₖNR^{20a}C(O)N(R^{20b})(R^{20c}), —(CH₂)ₙNR^{21a}SO₂R^{21b}, —(CH₂)ₙSO₂R²², —(CH₂)ₖN(R^{23a})C(O)OR^{23b}, —C(O)N(R^{24a})(R^{24b}), C³-C₈ cycloalkyl, aryl or Het⁴;
R¹⁶ to R¹⁸, R²¹, R^{22a}, R^{23a}, R^{23b}independently represent, at each occurrence, hydrogen, C₁-C₆ alkyl, aryl or Het¹⁵ (which C₁-C₆ alkyl, aryl and Het¹⁵ groups are optionally substituted with one or more substituents selected from —OH, halogen, cyano, nitro, C₁-C₆ alkyl, aryl and Het¹⁶);
R^{19a} and R^{19b} independently represent, at each occurrence, hydrogen, C₁-C₆ alkyl, aryl or Het¹⁹ (which C₁-C₆ alkyl, aryl and Het¹⁹ groups are optionally substituted with one or more substituents selected from —OH, halogen, cyano, nitro, C₁-C₆ alkyl, aryl and Het²⁰) or together represent C₃-C₆ alkylene, optionally interrupted by an O atom;
R^{20a}, R^{20b} and R^{20c} independently represent, at each occurrence, hydrogen, C₁-C₆ alkyl, aryl or Het²¹ (which C₁-C₆ alkyl, aryl and Het²¹ groups are optionally substituted with one or more substituents selected from —OH, halogen, cyano, nitro, C₁-C₆ alkyl, aryl and Het²²); or and R^{20c} may together represent C₃-C₆ alkylene, optionally interrupted by an O atom;
R³ represents hydrogen, or C₁-C₁₂ alkyl (which alkyl group is optionally substituted by one or more groups selected from halogen, —OR²⁵, —COR²⁶, C₂-C₆ alkenyl, C₃-C₈ cycloalkyl, trialkylsilyl, —COXR²⁷, aryl and Het⁵); further R³ represents —(CH₂)ₖN(R^{28a})(R^{28b}), —(CH₂)ₖN(R^{29a})C(O)N(R^{29b})(R^{29c}), —(CH₂)ₙNR^{30a}SO₂R^{30b}, —(CH₂)ₙSO₂R³¹, —(CH₂)ₖN(R^{32a})C(O)OR³², —OC(O)N(R^{33a})(R^{33b}), C₃-C₈ cycloalkyl, aryl or Het⁶;
R²⁵ to R²⁷, R³⁰, R³¹, R^{32a}, R^{32b}independently represent, at each occurrence, hydrogen, C₁-C₆ alkyl, aryl or Het²³ (which C₁-C₆ alkyl, aryl and Het²³ groups are optionally substituted with one or more substituents selected from —OH, halogen, cyano, nitro, C₁-C₆ alkyl, aryl and Het²⁴);
R^{28a} and R^{28b} independently represent, at each occurrence, hydrogen, C₁-C₆ alkyl, aryl or Het²⁵ (which C₁-C₆ alkyl, aryl and Het²⁵ groups are optionally substituted with one or more substituents selected from —OH, halogen, cyano, nitro, C₁-C₆ alkyl, aryl and Het²⁶), or together represent C₃-C₆ alkylene, optionally interrupted by an O atom;
R^{33a} and R^{33b} independently represent, at each occurrence, hydrogen, C₁-C₆ alkyl, aryl or Het²⁷ (which C₁-C₆ alkyl, aryl and Het²⁷ groups are optionally substituted with one or more substituents selected from —OH, halogen, cyano, nitro, C₁-C₆ alkyl, aryl and Het²⁸) or together represent C₃-C₆ alkylene, optionally interrupted by an O atom;
R^{29a}, R^{29b}, and R^{29c} independently represent, at each occurrence, hydrogen, C₁-C₆ alkyl, aryl or Het²⁹ (which C₁-C₆ alkyl, aryl and Het²⁹ groups are optionally substituted with one or more substituents selected from —OH, halogen, cyano, nitro, C₁-C₆ alkyl, aryl and Het³⁰); or R^{29b} and R^{29c} may together represent C₃-C₆ alkylene, optionally interrupted by an O atom;
R⁴ represents hydrogen, —OH, aryl, C₁-C₆ alkyl (which alkyl group is optionally substituted by one or more groups selected from halogen, hydroxy, C₂-C₄ alkenyl, and trialkylsilyl), —R³⁴, or —(CH₂)ₘR³⁵;
R³⁴ independently represent, at each occurrence, hydrogen, C₁-C₆ alkyl, aryl or Het³¹ (which C₁-C₆ alkyl, aryl and Het³¹ groups are optionally substituted with one or more substituents selected from —OH, halogen, cyano, nitro, C₁-C₆ alkyl, aryl and Het³²);
R³⁵ independently represent aryl or Het³³ (which aryl and Het³³ groups are optionally substituted with one or more substituents selected from —OH, halogen, cyano, nitro, C₁-C₆ alkyl, aryl and Het³⁴);
R⁵ to R⁷ independently represent, at each occurence, hydrogen, —OH, halogen, cyano, nitro, C₁₋₆ alkyl, —OR³⁶, —N(R^{37a})(R^{37b}), —C(O)R³⁸, —C(O)OR³⁹, —C(O)N(R^{40a})(R^{40b}), —NC(O)OR⁴¹, —OC(O)N(R^{42a})(R^{42b}), —N(R^{43a})C(O)R^{43b}, —N(R^{44a})S(O)₂R^{44b}, —S(O)₂R⁴⁵, —OS(O)₂R⁴⁶, —(CH₂)ₙN(R^{47a})(R^{47b}), —(CH₂)ₙNR^{48a}C(O)N(R^{48b})(R^{48c}), —(CH₂)ₙNR^{49a}SO₂R^{49b}, trialkylsilyl, aryl or Het⁷;
R³⁶, R³⁸, R³⁹, R⁴¹, R⁴³, R^{44a}, R^{44b}, R⁴⁵, R⁴⁶, R^{49a} and R^{49b} independently represent, at each occurrence, hydrogen, C₁-C₆ alkyl, aryl or Het³⁵ (which C₁-C₆ alkyl, aryl and Het³⁵ groups are optionally substituted with one or more substituents selected from —OH, halogen, cyano, nitro, C₁-C₆ alkyl, aryl and Het³⁶);
R^{37a} and R^{37b} independently represent, at each occurrence, hydrogen, C₁-C₆ alkyl, aryl or Het³⁷ (which C₁-C₆ alkyl, aryl and Het³⁷ groups are optionally substituted with one or more substituents selected from —OH, halogen, cyano, nitro, C₁-C₆ alkyl, aryl and Het³⁸), or together represent C₃-C₆ alkylene, optionally interrupted by an O atom;
R^{40a} and R^{40b} independently represent, at each occurrence, hydrogen, C₁-C₆ alkyl, aryl or Het³⁹ (which C₁-C₆ alkyl, aryl and Het³⁹ groups are optionally substituted with one or more substituents selected from —OH, halogen, cyano, nitro, C₁-C₆ alkyl, aryl and Het⁴⁰), or together represent C₃-C₆ alkylene, optionally interrupted by an O atom;
R^{42a} and R^{42b} independently represent, at each occurrence, hydrogen, C₁-C₆ alkyl, aryl or Het⁴¹ (which C₁-C₆ alkyl, aryl and Het⁴¹ groups are optionally substituted with one or more substituents selected from —OH, halogen, cyano, nitro, C₁-C₆ alkyl, aryl and Het⁴²), or together represent C₃-C₆ alkylene, optionally interrupted by an O atom;
R^{47a} and R^{47b} independently represent, at each occurrence, hydrogen, C₁-C₆ alkyl, aryl or Het⁴³ (which C₁-C₆ alkyl, aryl and Het⁴³ groups are optionally substituted with one or more substituents selected from —OH, halogen, cyano, nitro, C₁-C₆ alkyl, aryl and Het⁴⁴), or together represent C₃-C₆ alkylene, optionally interrupted by an O atom;
R^{48a}, R^{48b} and R^{48c} independently represent, at each occurrence, hydrogen, C₁-C₆ alkyl, aryl or Het⁴⁵ (which C₁-C₆ alkyl, aryl and Het⁴⁵ groups are optionally substituted with one or more substituents selected from —OH, halogen, cyano, nitro, C₁-C₆ alkyl, aryl and Het46); R^{48b} and R^{48c} may together represent C₃-C₆ alkylene, optionally interrupted by an O atom;
aryl is, at each occurrence, optionally substituted by —OH, halogen, cyano, nitro, C₁-C₆ alkyl, C₃-C₈ cycloalkyl, C₂-C₆ alkenyl, aryl, Het⁸, —R⁵⁰, —(CH₂)ₘR⁵¹, —SR⁵², —C(O)R⁵³, —COXR⁵⁴, —N(R^{55a})(R^{55b}), —SO₂R⁵⁶, —OS(O)₂R⁵⁷, —(CH₂)ₘN(R^{58a})(R^{58b}), —CH₂)ₘNR^{59a}C(O)N(R^{59b})(R^{59c}), —C(O)OR⁶⁰, —C(O)N(R^{61a})(R^{61b}), —N(R^{62a}C(O)R^{62b}, —N(R^{63a})C(O)OR^{63b}, —C(O)N(R^{64a})(R^{64b}), —N(R^{65a})S(O)₂R^{65b} or OC(O)R⁶⁶;
R⁵⁰ to R⁵⁴, R⁵⁶, R⁵⁷, R⁶⁰, R^{62a}, R^{62b}, R^{63a}, R^{63b}, R^{65a}, R^{65b} and R⁶⁶ independently represent, at each occurrence, hydrogen, C₁-C₆ alkyl, aryl or Het⁴⁷ (which C₁-C₆ alkyl, aryl and Het⁴⁷ groups are optionally substituted with one or more substituents selected from —OH, halogen, cyano, nitro, C₁-C₆ alkyl, aryl and Het⁴⁸);
R⁵¹ independently represent aryl or Het⁴⁹ (which aryl and Het⁴⁹ groups are optionally substituted with one or more substituents selected from —OH, halogen, cyano, nitro, C₁-C₆ alkyl, aryl and Het⁵⁰);
R^{55a} and R^{55b} independently represent, at each occurrence, hydrogen, C₁-C₆ alkyl, aryl or Het⁵¹ (which C₁-C₆ alkyl, aryl and Het⁵¹ groups are optionally substituted with one or more substituents selected from —OH, halogen, cyano, nitro, C₁-C₆ alkyl, aryl and Het⁵²), or together represent C₃-C₆ alkylene, optionally interrupted by an O atom;
R^{58a} and R^{58b} independently represent, at each occurrence, hydrogen, C₁-C₆ alkyl, aryl or Het⁵³ (which C₁-C₆ alkyl, aryl and Het⁵³ groups are optionally substituted with one or more substituents selected from —OH, halogen, cyano, nitro, C₁-C₆ alkyl, aryl and Het⁵⁴), or together represent C₃-C₆ alkylene, optionally interrupted by an O atom;
R^{59a}, R^{59b} and R^{59c} independently represent, at each occurrence, hydrogen, C₁-C₆ alkyl, aryl or Het⁵⁵ (which C₁-C₆ alkyl, aryl and Het⁵⁵ groups are optionally substituted with one or more substituents selected from —OH, halogen, cyano, nitro, C₁-C₆ alkyl, aryl and Het⁵⁶); or R^{59b} and R^{59c} may together represent C₃-C₆ alkylene, optionally interrupted by an O atom;
R^{61a} and R^{61b} independently represent, at each occurrence, hydrogen, C₁-C₆ alkyl, aryl or Het⁵⁷ (which C₁-C₆ alkyl, aryl and Het⁵⁷ groups are optionally substituted with one or more substituents selected from —OH, halogen, cyano, nitro, C₁-C₆ alkyl, aryl and Het⁵⁸); or together represent C₃-C₆ alkylene, optionally interrupted by an O atom;
R^{64a} and R^{64b} independently represent, at each occurrence, hydrogen, C₁-C₆ alkyl, aryl or Het⁵⁹ (which C₁-C₆ alkyl, aryl and Het⁵⁹ groups are optionally substituted with one or more substituents selected from —OH, halogen, cyano, nitro, C₁-C₆ alkyl, aryl and Het⁶⁰);
Het¹ to Het⁶⁰ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₂₋₆ alkenyl, aryl, a further Het, —R⁶⁷, —(CH₂)ₘR⁶⁸, —SR⁶⁹, —COXR⁷⁰, —N(R^{71a})(R^{71b}), —SO₂R⁷², —(CH₂)ₘN(R^{73a})(R^{73b}), —(CH₂)ₘNR^{74a}C(O)N(R^{74b})(R^{74c}), —C(O)R⁷⁵, —C(O)OR⁷⁶, —C(O)_{N}(R^{77a})(R⁷⁷), —N(R^{78a})C(O)R^{78b}), —N(R^{79a})S(O)₂R^{79b}, OC(O)_{R}⁸⁰, —NC(O)OR⁸¹, and —OC(O)N(R^{82a})(R^{82b});
R⁶⁷, R⁶⁹, R⁷⁰, R⁷², R⁷⁵, R⁷⁶, R^{78a}, R^{78b}, R^{79a}, R^{79b}, R⁸⁰ and R⁸¹ independently represent, at each occurrence, hydrogen, C₁-C₆ alkyl, aryl or Het⁶¹ (which C₁-C₆ alkyl, aryl and Het⁶¹ groups are optionally substituted with one or more substituents selected from —OH, halogen, cyano, nitro, C₁-C₆ alkyl, aryl and Het⁶²);
R⁶⁸ represents aryl or Het⁶³ (which aryl and Het⁶³ groups are optionally substituted with one or more substituents selected from —OH, halogen, cyano, nitro, C₁-C₆ alkyl, aryl and Het⁶⁴);
R^{71a} and R^{71b} independently represent, at each occurrence, hydrogen, C₁-C₆ alkyl, aryl or Het⁶⁵ (which C₁-C₆ alkyl, aryl and Het⁶⁵ groups are optionally substituted with one or more substituents selected from —OH, halogen, cyano, nitro, C₁-C₆ alkyl, aryl and Het⁶⁶), or together represent C₃-C₆ alkylene, optionally interrupted by an O atom;
R^{73a} and R^{73b} independently represent, at each occurrence, hydrogen, C₁-C₆ alkyl, aryl or Het⁶⁷ (which C₁-C₆ alkyl, aryl and Het⁶⁷ groups are optionally substituted with one or more substituents selected from —OH, halogen, cyano, nitro, C₁-C₆ alkyl, aryl and Het⁶⁸); or together represent C₃-C₆ alkylene, optionally interrupted by an O atom;
R^{74a}, and R^{74b} and R^{74c} independently represent, at each occurrence, hydrogen, C₁-C₆ alkyl, aryl or Het⁶⁹ (which C₁-C₆ alkyl, aryl and Het⁶⁹ groups are optionally substituted with one or more substituents selected from —OH, halogen, cyano, nitro, C₁-C₆ alkyl, aryl and Het⁷⁰); or R^{74b} and R^{74c} may together represent C₃-C₆ alkylene, optionally interrupted by an O atom;
R^{77a}, and R^{77b} independently represent, at each occurrence, hydrogen, C₁-C₆ alkyl, aryl or Het⁷¹ (which C₁-C₆ alkyl, aryl and Het⁷¹ groups are optionally substituted with one or more substituents selected from —OH, halogen, cyano, nitro, C₁-C₆ alkyl, aryl and Het⁷²); or together represent C₃-C₆ alkylene, optionally interrupted by an O atom;
R^{82a}, and R^{82b} independently represent, at each occurrence, hydrogen, C₁-C₆ alkyl, aryl or Het⁷³ (which C₁-C₆ alkyl, aryl and Het⁷³ groups are optionally substituted with one or more substituents selected from —OH, halogen, cyano, nitro, C₁-C₆ alkyl, aryl and Het⁷⁴) or together represent C₃-C₆ alkylene, optionally interrupted by an O atom;
Het⁶¹ to Het⁷⁴ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, and C₁₋₆ alkyl;
X represents a nitrogen or oxygen atom;
m is an integer of 0 to 10;
n is an integer of 0 to 4;
k is an integer of 1 to 5; provided that
a) R² or R³ do not represent a fragment of formula 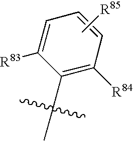
wherein
R⁸³ and R⁸⁴ represent independently, at each occurrence, halogen, C₁-C₁₂ alkyl, C₁-C₁₂ alkoxy, C₁-C₁₂ haloalkyl, C₁-C₁₂ haloalkoxy, cyano, —SR⁸⁶, —N(R^{87a})R^{87b}, C₂-C₆ alkynyl, aryl or Het⁷⁵;
R⁸⁵ represents hydrogen, C₁-C₁₂ alkyl group or C₁-C₁₂ alkoxy group (which C₁-C₁₂ alkyl and C₁-C₁₂ alkoxy groups are optionally substituted by one or more groups selected from halogen, C₂-C₆ alkenyl, C₂-C₆ alkynyl, cyano, oxo, aryl, Het⁷⁶, —OR⁸⁸, —SR⁸⁹, —COXR⁹⁰, —N(R^{91a})R^{91b}, and —SO₂R⁹²);
Het⁷⁵ to Het⁷⁶ independently represent, at each occurence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₁₋₆ alkoxy, aryl, aryloxy, —N(R^{93a})R^{93b}, —C(O)R^{93c}, —C(O)OR^{93d}, —C(O)N(R^{93e})R^{93f}, —N(R^{93g})C(O)R^{93h} and —N(R⁹³ⁱ)S(O)₂R^{93j}, OC(O)R^{93k} and a further Het;
R⁸⁶ to R⁹³ represent independently, at each occurrence, hydrogen or C₁₋₆ alkyl; and
b) the compound is not:
2-(2-ethoxyethyl)-N-isopropyl-3-oxoisoindoline-1-carboxamide;
N-(tert-butyl)-3-oxo-2-(3-pyrrolidin-1-ylpropyl)isoindoline-1-carboxamide;
N-(tert-butyl)-3-oxo-2-(tetrahydrofuran-2-ylmethyl)isoindoline-1-carboxamide;
2-[1-(hydroxymethyl)butyl]-N-isopropyl-3-oxoisoindoline-1-carboxamide;
N-isopropyl-2-(3-methylbutyl)-3-oxoisoindoline-1-carboxamide;
N-(tert-butyl)-2-cyclohexyl-3-oxoisoindoline-1-carboxamide;
N-(tert-butyl)-2-(3-methylbutyl)-3-oxoisoindoline-1-carboxamide;
methyl N-{[2-(3-methylbutyl)-3-oxo-2,3-dihydro-1H-isoindol-1-yl]carbonyl}glycinate;
tert-butyl N-({2-[1-(hydroxymethyl)butyl]-3-oxo-2,3-dihydro-1H-isoindol-1-yl}carbonyl)glycinate;
tert-butyl N-{[2-(3-methylbutyl)-3-oxo-2,3-dihydro-1H-isoindol-1-yl]carbonyl}glycinate;
N-(tert-butyl)-2-[1-(methoxymethyl)propyl]-3-oxoisoindoline-1-carboxamide;
N-(tert-butyl)-2-[2-(diethylamino)ethyl]-3-oxoisoindoline-1-carboxamide;
N-(tert-butyl)-2-[1-(hydroxymethyl)butyl]-3-oxoisoindoline-1-carboxamide;
tert-butyl N-{[3-oxo-2-(2-thienylmethyl)-2,3-dihydro-1H-isoindol-1-yl]carbonyl}glycinate;
tert-butyl N-({2-[2-(methylthio)ethyl]-3-oxo-2,3-dihydro-1H-isoindol-1-yl}carbonyl)glycinate;
methyl N-{[2-(cyclopropylmethyl)-3-oxo-2,3-dihydro-1H-isoindol-1-yl]carbonyl}glycinate; or
2-(2,2-dimethylpropyl)-3-oxo-N-(4,4,4-trifluorobutyl)isoindoline-1-carboxamide.
for use in therapy.

61-62. (canceled)

63. A pharmaceutical composition comprising as active ingredient a therapeutically effective amount of the compound according to claim 1 in association with one or more pharmaceutically acceptable diluents, excipients and/or inert carriers.

64. (canceled)

65. A method of treatment of an arrhythmia comprising administering to a mammal in need of such treatment, a therapeutically effective amount of a compound according to claim 1.

66. An agent for the treatment of an arrhythmia which comprises as active ingredient a compound of formula I, according to claim 1.

67. (canceled)

68. A process for preparing a compound of formula I as described herein.
